SEQUENCE LISTING (41 sequences)

SEQ ID NO: 1 - protein, 1304 aa, Synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 4 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 5 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 6 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 7 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 8 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 9 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 10 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 11 - protein, 10 aa, Synthetic construct
TKSLDKGYNK

SEQ ID NO: 12 - protein, 8 aa, Synthetic construct
SLDKGYNK

SEQ ID NO: 13 - protein, 437 aa, Synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 14 - protein, 439 aa, Synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 15 - protein, 856 aa, Synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 16 - protein, 7 aa, Synthetic construct
ENLYFQG

SEQ ID NO: 17 - protein, 6 aa, Synthetic construct
LVPRGS

SEQ ID NO: 18 - protein, 8 aa, Synthetic construct
LEVLFQGP

SEQ ID NO: 19 - skipped

SEQ ID NO: 20 - protein, 23 aa, Synthetic construct
GLFGAIAGFIENGWEGMIDGWYG

SEQ ID NO: 21-25 - skipped (5 entries)

SEQ ID NO: 26 - protein, 1314 aa, Synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 27 - protein, 1304 aa, Synthetic construct; identical to SEQ ID NO: 1

SEQ ID NO: 28 - protein, 1304 aa, Synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 29 - protein, 1286 aa, Synthetic construct
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 30 - DNA, 3888 bp, synthetic construct
  source 1..3888: mol_type = other DNA; note = Modified (Cationic) BoNT/A Cat-A
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 31 - protein, 1296 aa, synthetic construct
  source 1..1296: note = Modified (Cationic) BoNT/A Cat-A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 32 - DNA, 3888 bp, synthetic construct
  source 1..3888: mol_type = other DNA; note = Modified (Cationic) BoNT/A Cat-B
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaagaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 33 - protein, 1296 aa, synthetic construct
  source 1..1296: note = Modified (Cationic) BoNT/A Cat-B
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFKKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 34 - DNA, 3888 bp, synthetic construct
  source 1..3888: mol_type = other DNA; note = Modified (Cationic) BoNT/A Cat-C
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaagaagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 35 - protein, 1296 aa, synthetic construct
  source 1..1296: note = Modified (Cationic) BoNT/A Cat-C
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLKKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 36 - DNA, 3888 bp, synthetic construct
  source 1..3888: mol_type = other DNA; note = Modified (Cationic) BoNT/A Cat-D
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagcgtaaagagtaccgtctggcgaccaacgcttcgcaggcgggtgttgagaaaattctgagcgcgttggagatccctcgtgtccgtcgtctgagccaagtcgtggttatgaagagcaagaacgaccagggtatcactaacaagtgcaagatgaacctgcaagaccgtcgtggtaacgacatcggctttattggtttccaccagttcaacaatattgctaaactggtagcgagcaattggtacaatcgtcagattgagcgccgtagccgtcgtttgggctgtagctgggagtttatcccggtcgatgatggttggggcgaacgtccgctg

SEQ ID NO: 37 - protein, 1296 aa, synthetic construct
  source 1..1296: note = Modified (Cationic) BoNT/A Cat-D
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKRKEYRLATNASQAGVEKILSALEIPRVRRLSQVVVMKSKNDQGITNKCKMNLQDRRGNDIGFIGFHQFNNIAKLVASNWYNRQIERRSRRLGCSWEFIPVDDGWGERPL

SEQ ID NO: 38 - protein, 51 aa, synthetic construct
  source 1..51: note = Any one or more of the Gly residues at positions 1-20 and 32-51 may be deleted; note = Carrier component
GGGGGGGGGGGGGGGGGGGGRGRDDRRQRRRGGGGGGGGGGGGGGGGGGGG

SEQ ID NO: 39 - protein, 51 aa, synthetic construct; identical to SEQ ID NO: 38
  source 1..51: note = Any one or more of the Gly residues at positions 1-20 and 32-51 may be deleted; note = Carrier component

SEQ ID NO: 40 - protein, 49 aa, synthetic construct
  source 1..49: note = Any one or more of the Gly residues at positions 1-20 and 30-49 may be deleted
GGGGGGGGGGGGGGGGGGGGRKKRRQRRRGGGGGGGGGGGGGGGGGGGG

SEQ ID NO: 41 - protein, 35 aa, synthetic construct
  source 1..35: note = Carrier component
RKKRRQRRRGKKKKKKKKKKKKKKKGRKKRRQRRR

(19) United States
(12) Patent Application Publication
(10) Pub. No.: US 2026/0355295 A1
(43) Pub. Date: Nov. 12, 2026

(54) FORMULATION

(71) Applicant: IPSEN BIOPHARM LIMITED, Wrexham (GB)

(72) Inventors: Saif SHUBBER, Wrexham (GB); Robert KELLY, Wrexham (GB)

(21) Appl. No.: 19/705,351

(22) Filed: Jun. 11, 2026

Related U.S. Application Data
(63) Continuation of application No. PCT/GB2024/053076, filed on Dec. 11, 2024.

(30) Foreign Application Priority Data
Dec. 11, 2023 (GB) 2318884.0

(51) Int. Cl. A61K 38/48; A61K 9/16; A61K 9/19

(52) U.S. Cl. CPC A61K 38/4893; A61K 9/1611; A61K 9/1617; A61K 9/1623; A61K 9/1658; A61K 9/19

(57) ABSTRACT

The present invention relates to stabilised clostridial neurotoxin formulations, and methods for making and using the same.

### CROSS-REFERENCE TO RELATED APPLICATIONS

This application is a Continuation Application of International Application No. PCT/GB2024/053076, filed on Dec. 11, 2024, which claims priority to United Kingdom Application No. 2318884.0, filed on Dec. 11, 2023; the contents of each of which are hereby incorporated by reference in their entirety.

### INCORPORATION BY REFERENCE OF SEQUENCE LISTING

The instant application is being filed with an electronically filed Sequence Listing in XML format. The sequence listing file entitled pctgb2024053076-seql.xml, was created on Dec. 18, 2024, and is 75,937 bytes in size; the information in electronic format of the Sequence Listing is incorporated herein by reference in its entirety.

### FIELD OF THE INVENTION

The present invention relates to stabilised clostridial neurotoxin formulations, and methods for making and using the same.

### BACKGROUND OF THE INVENTION

Bacteria in the genus Clostridia produce highly potent and specific protein toxins, which can poison neurons and other cells to which they are delivered. Examples of such clostridial neurotoxins include the neurotoxins produced by C. tetani (TeNT) and by C. botulinum (BoNT) serotypes A-G, and X (see WO 2018/009903), as well as those produced by C. baratii and C. butyricum.

Among the clostridial neurotoxins are some of the most potent toxins known. By way of example, botulinum neurotoxins have median lethal dose (LD50) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies. By way of example, we refer to William J. Lipham, Cosmetic and Clinical Applications of Botulinum Toxin (Slack, Inc., 2004), which describes the use of clostridial neurotoxins, such as botulinum neurotoxins (BoNTs), BoNT/A, BONT/B, BONT/C1, BONT/D, BONT/E, BONT/F and BoNT/G, and tetanus neurotoxin (TeNT), to inhibit neuronal transmission in a number of therapeutic and cosmetic or aesthetic applications—for example, marketed botulinum toxin products are currently approved as therapeutics for indications including focal spasticity, upper limb spasticity, lower limb spasticity, cervical dystonia, blepharospasm, hemifacial spasm, hyperhidrosis of the axillae, chronic migraine, neurogenic detrusor overactivity, glabellar lines, and severe lateral canthal lines. In addition, clostridial neurotoxin therapies are described for treating neuromuscular disorders (see U.S. Pat. No. 6,872,397); for treating uterine disorders (see US 2004/0175399); for treating ulcers and gastroesophageal reflux disease (see US 2004/0086531); for treating dystonia (see U.S. Pat. No. 6,319,505); for treating eye disorders (see US 2004/0234532); for treating blepharospasm (see US 2004/0151740); for treating strabismus (see US 2004/0126396); for treating pain (see U.S. Pat. Nos. 6,869,610, 6,641,820, 6,464,986, and 6,113,915); for treating fibromyalgia (see U.S. Pat. No. 6,623,742, US 2004/0062776); for treating lower back pain (see US 2004/0037852); for treating muscle injuries (see U.S. Pat. No. 6,423,319); for treating sinus headache (see U.S. Pat. No. 6,838,434); for treating tension headache (see U.S. Pat. No. 6,776,992); for treating headache (see U.S. Pat. No. 6,458,365); for reduction of migraine headache pain (see U.S. Pat. No. 5,714,469); for treating cardiovascular diseases (see U.S. Pat. No. 6,767,544); for treating neurological disorders such as Parkinson's disease (see U.S. Pat. Nos. 6,620,415, 6,306,403); for treating neuropsychiatric disorders (see US 2004/0180061, US 2003/0211121); for treating endocrine disorders (see U.S. Pat. No. 6,827,931); for treating thyroid disorders (see U.S. Pat. No. 6,740,321); for treating cholinergic influenced sweat gland disorders (see U.S. Pat. No. 6,683,049); for treating diabetes (see U.S. Pat. Nos. 6,337,075, 6,416,765); for treating a pancreatic disorder (see U.S. Pat. Nos. 6,261,572, 6,143,306); for treating cancers such as bone tumours (see U.S. Pat. Nos. 6,565,870, 6,368,605, 6,139,845, US 2005/0031648); for treating otic disorders (see U.S. Pat. Nos. 6,358,926, 6,265,379); for treating autonomic disorders such as gastrointestinal muscle disorders and other smooth muscle dysfunction (see U.S. Pat. No. 5,437,291); for treatment of skin lesions associated with cutaneous cell-proliferative disorders (see U.S. Pat. No. 5,670,484); for management of neurogenic inflammatory disorders (see U.S. Pat. No. 6,063,768); for reducing hair loss and stimulating hair growth (see U.S. Pat. No. 6,299,893); for treating downturned mouth (see U.S. Pat. No. 6,358,917); for reducing appetite (see US 2004/40253274); for dental therapies and procedures (see US 2004/0115139); for treating neuromuscular disorders and conditions (see US 2002/0010138); for treating various disorders and conditions and associated pam (see US 2004/0013692); for treating conditions resulting from mucus hypersecretion such as asthma and COPD (see WO 00/10598); and for treating non-neuronal conditions such as inflammation, endocrine conditions, exocrine conditions, immunological conditions, cardiovascular conditions, bone conditions (see WO 01/21213). All of the above publications are hereby incorporated by reference in their entirety.

The use of non-cytotoxic proteases such as clostridial neurotoxins (e.g. BoNTs and TeNT) in therapeutic and cosmetic treatments of humans and other mammals is anticipated to expand to an ever-widening range of diseases and ailments that can benefit from the properties of these toxins.

Currently all approved drugs/cosmetic preparations comprising BoNTs contain naturally occurring neurotoxins purified from clostridial strains (BoNT/A in the case of DYSPORT® BOTOX® or XEOMIN®, and BoNT/B in the case of MYOBLOC®).

Recombinant technology offers the possibility of changing or optimising the properties of neurotoxins through the introduction of modifications to its sequence and/or structure. In particular, chimeric neurotoxins in which the H_{C} domain or the Hoc subdomain is replaced by a H_{C} domain or H_{CC} subdomain from a different neurotoxin have been produced. Examples of chimeric neurotoxins have been described in the art, including in WO 2017/191315, Rummel et al. 2011 (FEBS J. 278 (23): 4506-4515), Wang et al. 2008 (J. Biol. Chem. 283 (25): 16993-17002), Wang et al. 2012a (Biochem. J. 444 (1): 59-67) and Wang et al. 2012b (FASEB J. 26 (12): 5035-5048), all of which are incorporated by reference in their entirety.

Such recombinant chimeric toxins must also be processed to di-chain form, and so the activation process of natural clostridial neurotoxins must be reproduced during standard production of recombinant toxin production. Selection of a suitable exogenous protease may depend nature of the chimeric toxin, particularly on its cleavage site. Exogenous proteases such as trypsin or Lys-C are used for proteolytically activating single-chain clostridial neurotoxins in conventional production methods.

In vitro activation of clostridial neurotoxins may be associated with numerous disadvantages. There is a cost associated with the use of an exogenous protease (particularly GMP-grade protease), and its removal following activation of the clostridial neurotoxin. Dependence on a single or limited number of suppliers for GMP-grade protease can also create weakness in the supply/production chain. Purification of the activated clostridial neurotoxin from the activating exogenous protease can also affect production efficiency and yield. In addition, production of active di-chain clostridial neurotoxins according to conventional production methods necessitates strict safety and control procedures, also adding to production costs and time. Strict safety precautions are also required for practitioners working with active di-chain clostridial neurotoxins. More fundamentally, once activated, the clostridial neurotoxin must be stored before use, and conventional formulations can be associated with stability issues, where the active di-chain clostridial neurotoxin degrades over time. This is particularly an issue where compositions are stored at ambient temperatures. Reduced stability is disadvantageous, as it leads to clinical problems (difficulties in determining accurate doses for administration) and also economic issues, due to reduced shelf-life and increased costs, such as by requiring cold-storage or by increasing the need for stock replacement/rotation.

The present invention overcomes one or more of the above-mentioned problems. In particular, it is an object of the present invention to provide compositions of chimeric clostridial neurotoxins with improved stability.

### SUMMARY OF THE INVENTION

The present inventors have developed a new formulation for chimeric clostridial neurotoxins that provides improved stability compared with conventional formulations. In particular, the present inventors have surprisingly demonstrated that the use of a dual histidine buffer system comprising L-histidine and histidine monohydrochloride can be used to stabilise chimeric BoNT/AB in a solid (lyophilised) formulation that demonstrates improved stability compared with multiple solid and liquid formulations of recombinant BoNT/A and BoNT/A produced from C. botulinum. The use of a dual histidine buffer also allows the pH of the formulation to be precisely controlled, which, without being bound by theory, is believed to contribute to the stability of the formulations of the invention.

The present inventors obtained further improvements to stability using a non-reducing sugar, such as sucrose, as a stabilising agent in the chimeric clostridial neurotoxin formulations. Thus, the formulations of the invention can be stabilised without the use of lactose, which is present in some commercial botulinum neurotoxin preparations. Lactose can modify clostridial neurotoxins at key lysine residues, which can have a negative impact on clostridial toxin activity. In addition, formulations of the invention can be stabilised without the use of human albumin, which is an alternative common stabilising agent in botulinum neurotoxin preparations. As a human product, the use of albumin requires tight quality and safety controls to comply with GMP requirements.

As such, the present invention provides chimeric clostridial neurotoxin compositions which have a precisely controlled pH, and which contain a non-reducing sugar stabilising agent, such as sucrose. Such compositions typically do not comprise stabilising agents conventionally used in clostridial neurotoxin formulations, such as lactose and/or human albumin. The compositions of the invention typically achieve a precisely controlled pH using a dual histidine buffer.

Accordingly, the present invention provides a solid composition comprising or consisting of: (a) a chimeric clostridial neurotoxin; (b) L-histidine; and (c) histidine-HCl. Particularly the present invention provides a solid composition comprising or consisting of: (a) a chimeric botulinum neurotoxin (BoNT); (b) L-histidine; and (c) histidine-HCl.

Said composition may comprise: (a) a relative weight amount of L-histidine of between about 10% to about 20% (w/w); and/or (b) a relative weight amount of histidine-HCl of between about 5% to about 10% (w/w). Preferably said composition may comprise: (a) a relative weight amount of L-histidine of between about 10% to about 20% (w/w); and (b) a relative weight amount of histidine-HCl of between about 5% to about 10% (w/w).

Said solid composition may further comprise a stabiliser, which is preferably a disaccharide, more preferably sucrose. The disaccharide may be present at a relative weight amount of between about 70% to about 80% (w/w).

Alternatively or in addition, said solid composition may further comprise a surfactant, preferably a non-ionic surfactant. The non-ionic surfactant may be a polysorbate or a poloxamer, preferably polysorbate 80 or poloxamer-188. The surfactant may be present at a relative weight amount of between about 0.5% to about 1.0% (w/w).

Alternatively or in addition, said solid composition may further comprise a tonicity agent, preferably sodium chloride. The tonicity agent may be present at a relative weight amount of between about 2% to about 7% (w/w).

A solid composition of the invention may not comprise an additional excipient.

The chimeric BONT may be present in a solid composition of the invention at an absolute weight amount of between about 0.2 ng to about 500 ng, preferably between about 50 ng to about 300 ng, more preferably between about 50 ng to about 200 ng.

Preferably the chimeric clostridial neurotoxin or chimeric BONT present in a solid composition of the invention is a BoNT/AB chimera.

A solid composition of the invention may comprise or consist of: (a) a chimeric clostridial neurotoxin or chimeric BoNT, preferably a BoNT/AB chimera, optionally at an absolute weight amount of between about 0.2 ng to about 200 ng, preferably between about 5 ng to about 75 ng; (b) L-histidine, optionally at a relative weight a relative weight amount of between about 10% to about 20% (w/w), preferably between about 16% to about 17% (w/w); (c) histidine-HCl, optionally at a relative weight a relative weight amount of between about 5% to about 10% (w/w), preferably between about 7.5% to about 8% (w/w); (d) sucrose, optionally at a relative weight amount of between about 70% to about 80% (w/w), preferably between about 70% to about 72% (w/w); (e) polysorbate 80 or poloxamer-188, optionally at a relative weight amount of between about 0.5% to about 1.0% (w/w), preferably between about 0.70% to about 0.75% (w/w); and (f) sodium chloride, optionally at a relative weight amount of between about 2% to about 7% (w/w), preferably between about 4% to about 5%.

A solid composition of the invention may be a lyophilised or vacuum-dried powder.

A solid composition of the invention may have a pH between about 6.0 and about 7.0, preferably about 6.5, when reconstituted in saline and measured at 20-25 °C.

A solid composition of the invention, may be stable: (a) for at least 18 months at 25 °C; and/or (b) for at least 24 months at 5 °C.

The invention also provides a liquid composition comprising or consisting of: (a) a chimeric botulinum neurotoxin; (b) L-histidine; and (c) histidine-HCl. Particularly the present invention provides a liquid composition comprising or consisting of: (a) a chimeric botulinum neurotoxin (BoNT); (b) L-histidine; and (c) histidine-HCl.

Said liquid composition may comprise: (a) a chimeric BONT, preferably a BoNT/AB chimera, optionally at an amount of between about 50 ng to about 500 ng, preferably between about 50 ng to about 300 ng: (a) L-histidine, optionally at a concentration of between about 1.41 mg/mL to about 2.82 mg/mL preferably between about 2.0 mg/mL to about 2.5 mg/mL; (b) histidine-HCl, optionally at a concentration of between about 0.70 mg/mL to about 1.41 mg/mL, preferably between about 1.00 mg/mL to about 1.20 mg/mL; (c) sucrose, optionally at a concentration of between about 9.80 mg/mL to about 11.30 mg/mL, preferably between about 9.80 mg/mL to about 10.20 mg/mL; (d) polysorbate 80 or poloxamer-188, optionally at a concentration of between about 0.070 mg/mL to about 0.140 mg/mL, preferably between about 0.095 mg/mL to about 0.120 mg/mL; and (e) sodium chloride, optionally at a concentration of between about 0.28 mg/mL to about 11.7 mg/mL, optionally between about 5.8 mg/mL to about 11.7 mg/mL or between about 0.56 mg/mL to about 0.70 mg/mL; wherein the pH of the composition is between about 6.0 to about 7.0, preferably about 6.5.

A liquid composition of the invention may be stable for at least 6 months at 5 °C.

A liquid composition of the invention may be reconstituted from a solid composition of the invention using water or saline.

The invention also provides a solid composition or a liquid composition as described herein for use in therapy, wherein the solid composition is reconstituted in water or saline before use.

The invention also provides a method of treatment comprising administration of a solid composition or a liquid composition as described herein to a subject in need thereof, wherein the solid composition is reconstituted in water or saline before administration.

A solid composition or a liquid composition of the invention may be for use in treating a condition associated with unwanted neuronal activity, for example a condition selected from the group consisting of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhidrosis, excessive salivation, excessive gastrointestinal secretions, secretory disorders, pain from muscle spasms, headache pain, migraine and dermatological conditions.

The invention also provides a non-therapeutic use of a solid composition or a liquid composition as described herein, for treating an aesthetic or cosmetic condition, wherein the solid composition is reconstituted in water or saline before use.

The invention further provides a kit comprising a solid composition or a liquid composition as described herein and instructions for therapeutic or cosmetic administration of said composition to a subject in need thereof.

The invention also provides a method of producing a liquid composition as described herein, said method comprising or consisting of reconstitution of a solid composition as described herein in water or saline.

### BRIEF DESCRIPTION OF THE DRAWINGS

FIG. 1: Chimeric BoNT/AB relative recovery from short-term stability study (25 °C), as determined by chimeric BoNT/AB protein quantification ELISA.

FIG. 2: Chimeric BoNT/AB relative potency from short-term stability study, as determined by a cell-based assay (CBA).

FIG. 3: Quantification of chimeric BoNT/AB from short-term stability study, as determined by chimeric BoNT/AB protein quantification ELISA.

FIG. 4: Chimeric BoNT/AB relative potency from short-term stability study, as determined by a cell-based assay (CBA).

FIG. 5: Quantification of chimeric BoNT/AB from short-term stability study, as determined by chimeric BoNT/AB protein quantification ELISA.

FIG. 6: Chimeric BoNT/AB relative potency from short-term stability study, as determined by a cell-based assay (CBA).

FIG. 7: Chimeric BoNT/AB concentration from DP Lyophilised Long-Term Forced (5 °C) Stability Study, as Determined by CBA.

FIG. 8: % relative potency of chimeric BoNT/AB from DP Lyophilised Long-Term Forced (25 °C) Stability Study, as Determined by CBA.

FIG. 9: % relative potency of chimeric BoNT/AB from DP Lyophilised Long-Term Forced (40 °C) Stability Study, as Determined by CBA.

FIG. 10: Graph showing relative potency over time for dual L-Histidine chimeric BoNT/AB formulation compared with commercially available liquid BoNT/A1 (Alluzience), liquid long-acting BoNT/A (cationic BoNT/A), as well as lyophilised BoNT/A1 (Dysport).

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (+) 5%, preferably +4%, +3%, +2%, +1%, +0.5%, +0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “buffer”, “buffering agent” and “buffer system” refer interchangeably to one or more compound or agent which stabilise an active pharmaceutical ingredient (API) within a composition while maintaining its functional characteristics. Typically, a buffer ensures optimal biological activity and stability of a drug product at the targeted storage temperature over the duration required for clinical or commercial use.

As used herein, the terms “stabilising agent” and “stabiliser” refer interchangeably to a compound or agent which helps the API maintain its desirable properties within a composition until said composition is administered to an individual.

As used herein, the term “surfactant” and “surface active agent” are used interchangeably to refer to compounds or agents that tend to preferentially accumulate at the boundary (i.e., interface) between two phases. Surfactants tend to be characterised by having two distinct regions, one hydrophilic, one hydrophobic. Surfactants can facilitate emulsion formation/particle dispersal. Adsorption of surfactants on solid surfaces enables these surfaces to be more readily wetted, and the incorporation of insoluble compounds within micelles of the surfactant can lead to the production of clear solutions. Surfactants may be included in compositions to act as non-specific binding agents, as sacrificial excipients and/or as further stabilising agents.

As used herein, the term “tonicity agent” refers to a compound or agent that is added to injectable preparations to prevent osmotic shock at the site of injection upon administration, and thereby reduce local irritation.

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto. All references cited in this specification are herewith incorporated by reference with respect to their entire disclosure content and the disclosure content specifically mentioned in this specification.

### Chimeric Clostridial Neurotoxins

The invention relates to formulations of chimeric clostridial neurotoxins, referred to interchangeably as chimeric neurotoxins. A chimeric clostridial neurotoxin may comprise at least a portion of a light chain from one clostridial neurotoxin or subtype thereof, and at least a portion of a heavy chain from another clostridial neurotoxin or clostridial neurotoxin subtype. A chimeric clostridial neurotoxin may contain the entire light chain from one clostridial neurotoxin subtype and the heavy chain from another clostridial neurotoxin subtype. A chimeric clostridial neurotoxin may contain a portion (e.g. the binding domain) of the heavy chain of one clostridial neurotoxin subtype, with another portion of the heavy chain being from another clostridial neurotoxin subtype. A chimeric clostridial neurotoxin may contain the entire light chain from one clostridial neurotoxin subtype and a portion (e.g. the binding domain or translocation domain) of the heavy chain from another clostridial neurotoxin subtype. Preferably the invention relates to formulations of chimeric botulinum neurotoxins (BoNTs). The clostridial neurotoxins which provide the domains/portions thereof comprised in a chimeric clostridial neurotoxin may be referred to herein as the constituent clostridial neurotoxins.

In some preferred embodiments, the term “chimeric neurotoxin” as used herein means a neurotoxin comprising or consisting of an LHIN domain originating from a first neurotoxin and a H_{C} domain originating from a second neurotoxin. By way of non-limiting example, the term “chimeric BONT” as used herein means a BoNT comprising or consisting of an LH_{N} domain originating from a first BONT and a H_{C} domain originating from a second BONT.

A chimeric clostridial neurotoxin, particularly a chimeric BoNT, may be defined in terms of the serotype or sub-serotype of the four main domains of the neurotoxin: L-chain, H_{N}, H_{CN} and H_{CC} (as defined herein). For example, the LH_{N}/A1-H_{C}B1 chimera of SEQ ID NO: 1 may be described as an AABB chimera. Similarly or alternatively, the therapeutic element may comprise light chain portions from different clostridial neurotoxins. Such chimeric clostridial neurotoxins are useful, for example, as a means of delivering the therapeutic benefits of such clostridial neurotoxins to patients who are immunologically resistant to a given clostridial neurotoxin subtype, to patients who may have a lower than average concentration of receptors to a given clostridial neurotoxin heavy chain binding domain, or to patients who may have a protease-resistant variant of the membrane or vesicle toxin substrate (e.g., SNAP-25, VAMP and syntaxin). Chimeric clostridial neurotoxins are described, for example, in WO 2017/191315 and U.S. Pat. No. 8,071,110, which publications are hereby incorporated by reference in their entirety. Thus, a clostridial neurotoxin of the invention may be a chimeric clostridial neurotoxin.

In some preferred embodiments, a clostridial neurotoxin is BoNT/A comprising at least one domain from a non-BoNT/A clostridial neurotoxin (i.e. a BoNT/A chimera). For example, a chimeric clostridial neurotoxin comprised in a composition of the invention may comprise or consist of:
i. A BoNT/A L-chain and a non-BoNT/A H_{N} and H_{C} domain;
ii. A BONT/A H_{N} domain and a non-BoNT/A L-chain and H_{C} domain
iii. A BoNT/A H_{C} domain and a non-BoNT/A L-chain and H_{N} domain;
iv. A BoNT/A L-chain and H_{N} domain and a non-BoNT/A H_{C} domain
V. A BoNT/A L-chain and H_{C} domain and a non-BoNT/A H_{N} domain; or
vi. A BONT/A H_{N} domain and H_{C} domain and a non-BoNT/A L-chain.

By way of non-limiting example, a chimeric clostridial neurotoxin of the invention comprises a BoNT/A L-chain and H_{N} domain and a BoNT/B H_{C} domain (such as LH_{N}/A1-H_{C}/B1), such as defined herein. In other words, in preferred embodiments of the invention, the chimeric clostridial neurotoxin comprises a botulinum neurotoxin A (BoNT/A) light-chain and translocation domain (H_{N} domain), and a BoNT/B receptor binding domain (H_{C} domain). An exemplary LH_{N}/A1-H_{C}B1 chimera is given in SEQ ID NO: 1. Other non-limiting examples of LH_{N}/A1-H_{C}B1 chimera are given in SEQ ID NOs: 26 to 28. A non-limiting example of an LH_{N}/A1-H_{C}B1 chimera is given in SEQ ID NO: 29. A chimeric clostridial neurotoxin of the invention may comprise a BoNT/A L-chain and H_{N} domain and a BoNT/C1 H_{C} domain. A chimeric clostridial neurotoxin of the invention may comprise a BoNT/A L-chain and H_{N} domain and a BoNT/D H_{C} domain. A chimeric clostridial neurotoxin of the invention may comprise a BoNT/A L-chain and H_{N} domain and a BoNT/E H_{C} domain. A chimeric clostridial neurotoxin of the invention may comprise a BoNT/A L-chain and H_{N} domain and a BoNT/F H_{C} domain. A chimeric clostridial neurotoxin of the invention may comprise a BoNT/A L-chain and H_{N} domain and a BoNT/G H_{C} domain. A chimeric clostridial neurotoxin of the invention may comprise a BoNT/A L-chain and H_{N} domain and a BoNT/X H_{C} domain. A chimeric clostridial neurotoxin of the invention may comprise a BoNT/A L-chain and H_{N} domain and a TeNT H_{C} domain.

For example, a chimeric clostridial neurotoxin comprised in a composition of the invention may comprise or consist of:
i. A BoNT/B L-chain and a non-BoNT/B H_{N} and H_{C} domain;
ii. A BoNT/B H_{N} domain and a non-BoNT/B L-chain and H_{C} domain
iii. A BoNT/B H_{C} domain and a non-BoNT/B L-chain and H_{N} domain;
iv. A BoNT/B L-chain and H_{N} domain and a non-BoNT/B H_{C} domain
V. A BoNT/B L-chain and H_{C} domain and a non-BoNT/B H_{N} domain; or
vi. A BoNT/B H_{N} domain and H_{C} domain and a non-BoNT/B L-chain.

For example, a chimeric clostridial neurotoxin comprised in a composition of the invention may comprise or consist of:
i. A BoNT/C1 L-chain and a non-BoNT/C1 H_{N} and H_{C} domain;
ii. A BoNT/C1 H_{N} domain and a non-BoNT/C1 L-chain and H_{C} domain
iii. A BoNT/C1 H_{C} domain and a non-BoNT/C1 L-chain and H_{N} domain;
iv. A BoNT/C1 L-chain and Hy domain and a non-BoNT/C1 H_{C} domain
V. A BoNT/C1 L-chain and H_{C} domain and a non-BoNT/C1 H_{N} domain; or
vi. A BoNT/C1 H_{N} domain and H_{C} domain and a non-BoNT/C1 L-chain.

Non-limiting examples include BoNT/C1 chimeras where the non-BoNT/C1 element is from a BoNT/D (i.e. BoNT/CD chimeras).

For example, a chimeric clostridial neurotoxin comprised in a composition of the invention may comprise or consist of:
i. A BoNT/D L-chain and a non-BoNT/D H_{N} and H_{C} domain;
ii. A BoNT/D H_{N} domain and a non-BoNT/D L-chain and H_{C} domain
iii. A BoNT/D H_{C} domain and a non-BoNT/D L-chain and H_{N} domain;
iv. A BoNT/D L-chain and H_{N} domain and a non-BoNT/D H_{C} domain
V. A BoNT/D L-chain and H_{C} domain and a non-BoNT/D H_{N} domain; or
vi. A BoNT/D H_{N} domain and H_{C} domain and a non-BoNT/D L-chain.

Non-limiting examples include BoNT/D chimeras where the non-BoNT/D element is from BoNT/C1 (i.e. BoNT/DC1 chimeras).

For example, a chimeric clostridial neurotoxin comprised in a composition of the invention may comprise or consist of:
i. A BoNT/E L-chain and a non-BoNT/E H_{N} and H_{C} domain;
ii. A BONT/E H_{N} domain and a non-BoNT/E L-chain and H_{C} domain
iii. A BoNT/E H_{C} domain and a non-BoNT/E L-chain and H_{N} domain;
iv. A BoNT/E L-chain and Hy domain and a non-BoNT/E H_{C} domain
V. A BoNT/E L-chain and H_{C} domain and a non-BoNT/E H_{N} domain; or
vi. A BONT/E H_{N} domain and H_{C} domain and a non-BoNT/E L-chain.

For example, a chimeric clostridial neurotoxin comprised in a composition of the invention may comprise or consist of:
i. A BoNT/F L-chain and a non-BoNT/F H_{N} and H_{C} domain;
ii. A BONT/F H_{N} domain and a non-BoNT/F L-chain and H_{C} domain
iii. A BoNT/F H_{C} domain and a non-BoNT/F L-chain and H_{N} domain;
iv. A BoNT/F L-chain and H_{N} domain and a non-BoNT/F H_{C} domain
V. A BoNT/F L-chain and H_{C} domain and a non-BoNT/F H_{N} domain; or
vi. A BONT/F H_{N} domain and H_{C} domain and a non-BoNT/F L-chain.

For example, a chimeric clostridial neurotoxin comprised in a composition of the invention may comprise or consist of:
i. A BoNT/G L-chain and a non-BoNT/G H_{N} and H_{C} domain;
ii. A BoNT/G H_{N} domain and a non-BoNT/G L-chain and H_{C} domain
iii. A BoNT/G H_{C} domain and a non-BoNT/G L-chain and H_{N} domain;
iv. A BoNT/G L-chain and H_{N} domain and a non-BoNT/G H_{C} domain
V. A BoNT/G L-chain and H_{C} domain and a non-BoNT/G H_{N} domain; or
vi. A BoNT/G H_{N} domain and H_{C} domain and a non-BoNT/G L-chain.

For example, a chimeric clostridial neurotoxin comprised in a composition of the invention may comprise or consist of:
i. A BoNT/X L-chain and a non-BoNT/X H_{N} and H_{C} domain;
ii. A BoNT/X H_{N} domain and a non-BoNT/X L-chain and H_{C} domain
iii. A BoNT/X H_{C} domain and a non-BoNT/X L-chain and H_{N} domain;
iv. A BoNT/X L-chain and H_{N} domain and a non-BoNT/X H_{C} domain
V. A BoNT/X L-chain and H_{C} domain and a non-BoNT/X H_{N} domain; or
vi. A BoNT/X H_{N} domain and H_{C} domain and a non-BoNT/X L-chain.

For example, a chimeric clostridial neurotoxin comprised in a composition of the invention may comprise or consist of:
i. A TeNT L-chain and a non-TeNT H_{N} and H_{C} domain;
ii. A TeNT H_{N} domain and a non-TeNT L-chain and H_{C} domain
iii. A TeNT H_{C} domain and a non-TeNT L-chain and H_{N} domain;
iv. A TeNT L-chain and H_{N} domain and a non-TeNT H_{C} domain
V. A TeNT L-chain and H_{C} domain and a non-TeNT H_{N} domain; or
vi. A TeNT H_{N} domain and H_{C} domain and a non-TeNT L-chain.

Using an “XY” representation according to which X is the LH_{N} domain and Y is the H_{C} domain, the following chimeric neurotoxins are embodiments of the present invention:
AB, AC, AD, AE, AF, AG, ATX,
BA, BC, BD, BE, BF, BG, BTx,
CA, CB, CD, CE, CF, CG, CTx,
DA, DB, DC, DE, DF, DG, DTx,
EA, EB, EC, ED, EF, EG, ETx,
FA, FB, FC, FD, FE, FG, FTx,
GA, GB, GC, GD, GE, GF, FTx,
TxA,TxB,TxC,TxD,TxE,TxF,TxG,
   wherein A, B, C, D, E, F, G and Tx are respectively Botulinum Neurotoxin (BoNT) serotype A, serotype B, serotype C, serotype D, serotype E, serotype F, serotype G and TeNT. In some embodiments, using this same XY representation, the following chimeric neurotoxins are preferred: AB, AC, BA, BC, CA and CB.

The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO2244.1), which cleaves VAMP2 and SNAP25, the Chryseobacterium pipero encoded toxin (NCBI Ref. Seq: WP_034687872.1) and the mosquito BoNT-like protein PMP1 (NCBI Ref. Seq: QEZ70852.1).

The term “clostridial neurotoxin” is intended to embrace re-targeted chimeric clostridial neurotoxins. In a re-targeted chimeric clostridial neurotoxin, the chimeric clostridial neurotoxin is modified to include an exogenous ligand (i.e. not derived from a clostridial neurotoxin) known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety. Thus, a chimeric clostridial neurotoxin of the invention may be a re-targeted chimeric clostridial neurotoxin. The re-targeted chimeric clostridial neurotoxins of the invention may comprise TM that are presented at the N- or C-terminus of the single-chain neurotoxin, or the TM may be presented centrally within the single-chain neurotoxin. In some preferred embodiments, the re-targeted chimeric clostridial neurotoxins of the invention may comprise TM that are presented at the N- or C-terminus of the single-chain neurotoxin.

A chimeric clostridial neurotoxin may be engineered to comprise an exogenous cleavage site (i.e. an activation site that is not found in either the first or second clostridial neurotoxin) that can be used to activate the chimeric clostridial neurotoxin. Engineering chimeric clostridial neurotoxins in the way allows for chimeric clostridial neurotoxins to be re-targeted using TM that are susceptible to cleavage by proteases conventionally used to activate recombinantly produced re-targeted clostridial neurotoxins, such as trypsin, Lys-C and/or BONT hydrolase. Thus, engineering re-targeted chimeric clostridial neurotoxins to include an exogenous activation site may allow for further improvements in stability compared to a corresponding re-targeted chimeric clostridial neurotoxins which is activated by a conventional activating protease such as Lys-C, trypsin and/or BoNT hydrolase.

A chimeric clostridial neurotoxin of the present invention may lack a functional H_{C} domain of a clostridial neurotoxin and also lack any functionally equivalent TM. Accordingly, said polypeptides lack the natural binding function of a clostridial neurotoxin and are not able to bind rat synaptosomal membranes (via a clostridial H_{C} component, or via any functionally equivalent TM) in binding assays as described in Shone et al. (1985) Eur. J. Biochem. 151, 75-82. Preferably the TM is not a Wheat Germ Agglutinin (WGA) peptide. Thus, in some embodiments the chimeric clostridial neurotoxin is a re-targeted chimeric clostridial neurotoxin in which an endogenous clostridial neurotoxin H_{C} or H_{CC} is replaced by an exogenous TM.

A chimeric clostridial neurotoxin of the invention may comprise an LH_{N} polypeptide.

A chimeric clostridial neurotoxin may comprise an LH_{N} polypeptide and a targeting moiety (TM).

A di-chain chimeric clostridial neurotoxin of the invention may comprise an L-chain portion of a polypeptide sequence having at least 70%, 80%, 90%, 95%, 99.9%, or 100% sequence identity to any one of the sequences of a clostridial neurotoxin described herein (SEQ ID NOs: 1 to 10, particularly SEQ ID NO: 1) constituting a first chain of the di-chain chimeric clostridial neurotoxin, and may comprise the H_{N} and H_{C} domains of a polypeptide sequence having at least 70%, 80%, 90%, 95%, 99.9%, or 100% sequence identity to the sequences of a clostridial neurotoxin described herein (SEQ ID NOs: 1 to 10, particularly SEQ ID NO: 1), together constituting a second chain of the di-chain chimeric clostridial neurotoxin, wherein the first and second chains are joined together by a di-sulphide bond.

The present invention also embraces chimeric clostridial neurotoxins that have an additional non-native protease cleavage site. Such a site will require an exogenous protease for cleavage, which allows for improved control over the timing and location of cleavage events. Non-native protease cleavage sites that may be employed in clostridial neurotoxins include:

TEV(Tobacco Etch virus)
(SEQ ID NO: 16)
(ENLYFQ↓G)
Thrombin
(SEQ ID NO: 17)
(LVPR↓GS)
PreScission
(SEQ ID NO: 18)
(LEVLFQ↓GP).

Additional protease cleavage sites include recognition sequences that are cleaved by a non-cytotoxic protease, for example by the light chain of a clostridial neurotoxin. These include the SNARE (e.g. SNAP-25, syntaxin, VAMP) protein recognition sequences that are cleaved by non-cytotoxic proteases such as the light chain of a clostridial neurotoxin. Clostridial neurotoxins comprising non-native protease cleavage sites are described in U.S. Pat. No. 7,132,259, EP 1206554-B2 and US 2007/0166332, all of which are hereby incorporated by reference in their entirety. Also embraced by the term protease cleavage site is an intein, which is a self-cleaving sequence. The self-splicing reaction is controllable, for example by varying the concentration of reducing agent present.

The present invention also embraces chimeric clostridial neurotoxins comprising a “destructive cleavage site”. In said chimeric clostridial neurotoxins, a non-native protease cleavage site is incorporated into the clostridial neurotoxin, at a location chosen such that cleavage at said site will decrease the activity of, or inactivate, the chimeric clostridial neurotoxin. The destructive protease cleavage site can be susceptible to cleavage by a local protease, in the event that the clostridial neurotoxin, following administration, migrates to a non-target location. Suitable non-native protease cleavage sites include those described above. Chimeric clostridial neurotoxins comprising a destructive cleavage site are described in WO 2010/094905 and WO 2002/044199, both of which are hereby incorporated by reference in their entirety.

The chimeric clostridial neurotoxins of the present invention, especially the light chain component thereof, may be PEGylated—this may help to further increase stability, for example duration of action of the light chain component. PEGylation is particularly preferred when the light chain comprises a BoNT/A, B or C1 protease. PEGylation preferably includes the addition of PEG to the N-terminus of the light chain component. By way of example, the N-terminus of a light chain may be extended with one or more amino acid (e.g. cysteine) residues, which may be the same or different. One or more of said amino acid residues may have its own PEG molecule attached (e.g. covalently attached) thereto. An example of this technology is described in WO2007/104567, which is hereby incorporated by reference in its entirety.

A chimeric clostridial neurotoxin of the invention may not comprise a therapeutic or diagnostic agent (e.g. a nucleic acid, protein, peptide or small molecule therapeutic or diagnostic agent) additional to the light-chain and heavy-chain. For example, in one embodiment, the chimeric clostridial neurotoxin may not comprise a covalently or non-covalently associated therapeutic or diagnostic agent. Thus, a chimeric clostridial neurotoxin of the invention preferably does not function as a delivery vehicle for a further therapeutic or diagnostic agent.

In embodiments where a chimeric clostridial neurotoxin described herein has a tag for purification (e.g. a His-tag) and/or a linker, said tag and/or linker are optional.

The chimeric clostridial neurotoxins of the present invention may be free from the complexing proteins that are present in a naturally occurring clostridial neurotoxin complex.

The chimeric clostridial neurotoxins of the present invention are typically produced using recombinant nucleic acid technologies. Thus, a chimeric clostridial neurotoxin (as described above) may be a recombinant chimeric clostridial neurotoxin. A single-chain chimeric clostridial neurotoxin (as described herein) may be a recombinant single-chain chimeric neurotoxin.

A chimeric clostridial neurotoxin of the invention may have equivalent or increased potency compared with the potency of the corresponding constituent clostridial neurotoxins. The term “equivalent potency” as used herein means that a chimeric clostridial neurotoxin has a potency of at least about 80%, at least about 85%, at least about 90%, at least about 95%, at least about 99%, up to about 100% of the potency of a constituent clostridial neurotoxin. Preferably “equivalent potency” as used herein means that a chimeric clostridial neurotoxin has a potency of at least about 95%, at least about 99%, at least about 100%, at least about 101%, up to about 105% of the potency of the corresponding constituent clostridial neurotoxin. The term “increased potency” as used herein means that a chimeric clostridial neurotoxin has a potency of at least about 10%, at least about 15%, at least about 20%, at least about 25% greater potency compared with the potency of a corresponding constituent clostridial neurotoxin. Potency may be measured using any appropriate assay, conventional examples of which are described herein.

Also described are nucleic acids (for example, a DNA or RNA) comprising a nucleic acid sequence encoding a chimeric clostridial neurotoxin as described herein. The nucleic acid sequence may be prepared as part of an expression vector in which the nucleic acid is operably linked to a promoter. Preferably, the nucleic acid may be prepared as part of a DNA expression vector comprising a promoter and a terminator.

Preferably the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

Alternatively, a promoter may preferably be selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

Such nucleic acid molecules may be made using any suitable process known in the art. Thus, the nucleic acid molecules may be made using chemical synthesis techniques. Alternatively, the nucleic acid molecules may be made using molecular biology techniques.

The nucleic acid molecules and expression vectors may be preferably designed in silico, and then synthesised by conventional synthesis techniques, including conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” and “polynucleotide” are used synonymously herein. Preferably the nucleotide sequence is a DNA sequence.

The invention provides a method of producing compositions of the invention. Said methods may comprise the production of a single-chain chimeric clostridial neurotoxin protein, such as by expressing a polynucleotide or expression vector described herein in a suitable host cell, and recovering the expressed engineered clostridial neurotoxin. Recovering the expressed engineered clostridial neurotoxin may comprise lysing the host cell to provide a host cell homogenate containing the single-chain (engineered) clostridial neurotoxin protein, and/or isolating the single-chain (engineered) clostridial neurotoxin protein. Said method may further comprise a step of introducing the polynucleotide or expression vector described herein into the host cell. Suitable host cells include bacterial cell lines used for the recombinant production of clostridial neurotoxins, particularly Escherichia coli cells.

The methods of the invention may further comprise a step of proteolytically processing a chimeric clostridial neurotoxin of the present invention into a corresponding di-chain chimeric clostridial neurotoxin, such as by contacting the chimeric clostridial neurotoxin with an appropriate exogenous protease, thereby producing a di-chain chimeric clostridial neurotoxin (e.g., wherein the light chain and heavy chain are joined together by a disulphide bond).

The present invention therefore provides compositions comprising a di-chain chimeric clostridial neurotoxin obtainable by a method of the invention.

The term “obtainable” as used herein also encompasses the term “obtained”. Preferably the term “obtainable” means obtained.

### Clostridial Neurotoxins

The term “neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. It is this di-chain form that is the active form of the toxin. The two chains are termed the heavy chain (H-chain), which has a molecular mass of approximately 100 kDa, and the light chain (L-chain), which has a molecular mass of approximately 50 kDa.

A clostridial neurotoxin of the invention may be catalytically active also referred to as active) or catalytically inactive.

The terms “catalytically active” or “active” as used interchangeably herein refer to a clostridial neurotoxin L-chain (or a clostridial neurotoxin comprising such an L-chain) having non-cytotoxic protease activity. Specifically, active clostridial neurotoxin L-chain has endopeptidase activity and is capable of cleaving a protein of the exocytic fusion apparatus in a target cell. A protein of the exocytic fusion apparatus is preferably a SNARE protein, such as SNAP25, synaptobrevin/VAMP, or syntaxin.

The term “catalytically inactive” as used herein in respect of a clostridial neurotoxin L-chain means that said L-chain exhibits substantially no non-cytotoxic protease activity, preferably the term “catalytically inactive” as used herein in respect of a clostridial neurotoxin L-chain means that said L-chain exhibits no non-cytotoxic protease activity. In one embodiment, a catalytically inactive clostridial neurotoxin L-chain is one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain, for example less than 2%, 1% or preferably less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

As described herein, the present invention relates to chimeric clostridial neurotoxins, particularly stabilised compositions thereof. As described herein, said chimeric clostridial neurotoxins typically comprise or consist of domains or portions of domains from a two or more clostridial neurotoxins, e.g. a first clostridial neurotoxin and a second clostridial neurotoxin. The clostridial neurotoxins from which the domains or portions of domains comprised in the chimeric neurotoxin may be referred to as constituent clostridial neurotoxins. Preferably, the first and second constituent neurotoxins are clostridial neurotoxins, more preferably botulinum neurotoxins (BoNT). A first or second constituent neurotoxin may be BONT/A, BONT/B, BONT/C, BONT/D, BONT/E, BONT/F, BONT/G, BONT/X or TeNT. Typically the first and second constituent neurotoxins are different.

A clostridial neurotoxin (e.g. a constituent neurotoxin) may be BoNT/A. An exemplary reference BoNT/A sequence is shown as SEQ ID NO: 2.

A clostridial neurotoxin (e.g. a constituent neurotoxin) may be BoNT/B. An exemplary reference BoNT/B sequence is shown as SEQ ID NO: 3.

A clostridial neurotoxin (e.g. constituent neurotoxin) may be BoNT/C. An exemplary reference BoNT/C1 sequence is shown as SEQ ID NO: 4.

A clostridial neurotoxin (e.g. a constituent neurotoxin) may be BoNT/D. An exemplary reference BoNT/D sequence is shown as SEQ ID NO: 5.

A clostridial neurotoxin (e.g. a constituent neurotoxin) may be BoNT/E. An exemplary reference BoNT/E sequence is shown as SEQ ID NO: 6.

A clostridial neurotoxin (e.g. a constituent neurotoxin) may be BoNT/F. An exemplary reference BoNT/F sequence is shown as SEQ ID NO: 7.

A clostridial neurotoxin (e.g. a constituent neurotoxin) may be BoNT/G. An exemplary reference BoNT/G sequence is shown as SEQ ID NO: 8.

A clostridial neurotoxin (e.g. a constituent neurotoxin) may be BoNT/X. An exemplary reference BoNT/X sequence is shown as SEQ ID NO: 9.

A clostridial neurotoxin (e.g. a constituent neurotoxin) may be TeNT. An exemplary reference TeNT sequence is shown as SEQ ID NO: 10.

As discussed above, clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Examples of light chain reference sequences from constituent neurotoxins include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C₁ neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Alternatively, clostridial neurotoxin L-chains may be defined as the first amino acid (including or excluding an initial methionine residue) through to the first cysteine residue of the endogenous activation loop. In addition or alternatively, a clostridial neurotoxin L-chain may be defined as the amino acid sequence N-terminal to the cleavage site within the endogenous activation loop.

Clostridial neurotoxin L-chains may be defined as a clostridial neurotoxin domain which comprises the metal coordinating HExxH motif (SEQ ID NO: 19), which typically functions to cleave a SNARE protein substrate.

The term “light-chain” (or “L-chain”) encompasses variants and fragments thereof, provided said variants and fragments still demonstrate non-cytotoxic protease activity (which can be determined using standard assays known in the art, examples of which are described herein). By way of example, a variant may have at least 70%, preferably at least 80%, more preferably at least 90%, and most preferably at least 95% or at least 98% amino acid sequence homology with a reference L-chain. The term fragment, when used in relation to a L-chain, means a peptide having at least 200, preferably at least 250, more preferably at least 300, even more preferably at least 350, and most preferably at least 400 amino acid residues of the reference L-chain. In the case of a clostridial L-chain, the fragment preferably at least 300, more preferably at least 350, and most preferably at least 400 amino acid residues of the reference L-chain. L-chain ‘fragments’ of the present invention embrace fragments of variant L-chains based on the reference sequences.

A clostridial neurotoxin H-chains may be defined as the second cysteine of the endogenous activation loop through to the final amino acid. In addition or alternatively, a clostridial neurotoxin H-chain may be defined as the amino acid sequence C-terminal to the cleavage site within the endogenous activation loop.

A Translocation Domain is a molecule that enables translocation of a protease into a target cell such that a functional expression of protease activity occurs within the cytosol of the target cell. Whether any molecule (e.g. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored (see Shone C. (1987) Eur. J. Biochem; vol. 167 (1): pp. 175-180).

A further example is provided by Blaustein R. (1987), which describes a simple in vitro assay employing planar phospholipid bilayer membranes. The membranes are challenged with a test molecule and the requisite translocation function is confirmed by an increase in conductance across said membranes (see Blaustein (1987) FEBS Letts; vol. 226, no. 1: pp. 115-120).

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The present invention also embraces variants and/or fragments of translocation domains, so long as the variant domains still demonstrate the requisite translocation activity. By way of example, a variant may have at least 70%, preferably at least 80%, more preferably at least 90%, and most preferably at least 95% or at least 98% amino acid sequence homology with a reference translocation domain. The term fragment, when used in relation to a translocation domain, means a peptide having at least 20, preferably at least 40, more preferably at least 80, and most preferably at least 100 amino acid residues of the reference translocation domain. In the case of a clostridial translocation domain, the fragment preferably has at least 100, preferably at least 150, more preferably at least 200, and most preferably at least 250 amino acid residues of the reference translocation domain (e.g. H_{N} domain). Translocation ‘fragments’ of the present invention embrace fragments of variant translocation domains based on the reference sequences.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, the Translocation Domain may be a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, such as the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain. The H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, the H-chain may be incapable of binding to the Binding Site on a target cell to which native clostridial neurotoxin (i.e. holotoxin) binds.

Examples of suitable (reference) Translocation Domains from constituent neurotoxins include:
Botulinum type A neurotoxin-amino acid residues (449-871)
Botulinum type B neurotoxin-amino acid residues (441-858)
Botulinum type C neurotoxin-amino acid residues (442-866)
Botulinum type D neurotoxin-amino acid residues (446-862)
Botulinum type E neurotoxin-amino acid residues (423-845)
Botulinum type F neurotoxin-amino acid residues (440-864)
Botulinum type G neurotoxin-amino acid residues (442-863)
Botulinum type X neurotoxin-amino acid residues (461-890)
Tetanus neurotoxin-amino acid residues (458-879)

For recently identified BoNT/X, the translocation domain has been reported as corresponding to amino acids 460-890 thereof, with the L-chain and H_{C} boundaries potentially varying by approximately 10 amino acids (e.g. 461-889 or 454-891).

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin-amino acid residues (A449-K871)
Botulinum type B neurotoxin-amino acid residues (A442-S858)
Botulinum type C neurotoxin-amino acid residues (T450-N866)
Botulinum type D neurotoxin-amino acid residues (D446-N862)
Botulinum type E neurotoxin-amino acid residues (K423-K845)
Botulinum type F neurotoxin-amino acid residues (A440-K864)
Botulinum type G neurotoxin-amino acid residues (S447-S863)
Tetanus neurotoxin-amino acid residues (S458-V879)

In the context of the present invention, a variety of clostridial neurotoxin H_{N} regions comprising a translocation domain can be useful in aspects of the present invention with the proviso that these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, in the context of the present invention a translocation domain can include clostridial neurotoxin H_{N} regions comprising a translocation domain having a length of, for example, at least 350 amino acids, at least 375 amino acids, at least 400 amino acids and at least 425 amino acids. Also encompassed are clostridial neurotoxin H_{N} regions comprising translocation domain having a length of, for example, at most 350 amino acids, at most 375 amino acids, at most 400 amino acids and at most 425 amino acids.

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally occurring neurotoxin H_{N} portions, and modified H_{N} portions having amino acid sequences that do not occur in nature and/or synthetic amino acid residues, so long as the modified H_{N} portions still demonstrate the above-mentioned translocation function.

Alternatively, the Translocation Domain may be of a non-clostridial origin. Examples of non-clostridial (reference) Translocation Domain origins include, but not be restricted to, the translocation domain of diphtheria toxin (O'Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp. 25-51), the translocation domain of Pseudomonas exotoxin type A (Prior et al. Biochemistry (1992) 31, 3555-3559), the translocation domains of anthrax toxin (Blanke et al. Proc. Natl. Acad. Sci. USA (1996) 93, 8437-8442), a variety of fusogenic or hydrophobic peptides of translocating function (Plank et al. J. Biol. Chem. (1994) 269, 12918-12924; and Wagner et al (1992) PNAS, 89, pp. 7934-7938), and amphiphilic peptides (Murata et al (1992) Biochem., 31, pp. 1986-1992). The Translocation Domain may mirror the Translocation Domain present in a naturally occurring protein, or may include amino acid variations so long as the variations do not destroy the translocating ability of the Translocation Domain.

Particular examples of viral (reference) Translocation Domains suitable for use in the present invention include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded spike proteins have particular application in the context of the present invention, for example, the E1 protein of SFV and the G protein of the G protein of VSV.

Use of the (reference) Translocation Domains listed in Table 1 (below) includes use of sequence variants thereof. A variant may comprise one or more conservative nucleic acid substitutions and/or nucleic acid deletions, insertions or insertion-deletions (indels), with the proviso that the variant possesses the requisite translocating function. A variant may also comprise one or more amino acid substitutions and/or amino acid deletions or insertions, so long as the variant possesses the requisite translocating function.

TABLE 1
Non-clostridial translocation domains

| Translocation | | |
|---|---|---|
| Domain source | Amino acid residues | References |
| Diphtheria toxin | 194-380 | Silverman et al., 1994, J. Biol. Chem. |
| | | 269, 22524-22532 |
| | | London E., 1992, Biochem. Biophys. |
| | | Acta., 1113, 25-51 |
| Domain II of | 405-613 | Prior et al., 1992, Biochemistry 31, |
| pseudomonas | | 3555-3559 |
| exotoxin | | Kihara & Pastan, 1994, Bioconj Chem. 5, |
| | | 532-538 |
| Influenza virus | GLFGAIAGFIENGW | Plank et al., 1994, J. Biol. Chem. 269, |
| haemagglutinin | EGMIDGWYG (SEQ | 12918-12924 |
| | ID NO: 20), and | Wagner et al., 1992, PNAS, 89, |
| | Variants thereof | 7934-7938 |
| | | Murata et al., 1992, Biochemistry 31, |
| | | 1986-1992 |
| Semliki Forest | Translocation domain | Kielian et al., 1996, J Cell Biol. 134(4), |
| virus fusogenic | | 863-872 |
| protein | | |
| Vesicular | 118-139 | Yao et al., 2003, Virology 310(2), 319-332 |
| Stomatitis virus | | |
| glycoprotein G | | |
| SER virus F protein | Translocation domain | Seth et al., 2003, J Virol 77(11) |
| | | 6520-6527 |
| Foamy virus | Translocation domain | Picard-Maureau et al., 2003, J Virol. |
| envelope | | 77(8), 4722-4730 |
| glycoprotein | | |

Examples of clostridial neurotoxin H_{C} domain reference sequences from constituent neurotoxins include:
BONT/A-N872-L1296
BONT/B-E859-E1291
BONT/C1-N867-E1291
BoNT/D-S863-E1276
BONT/E-R846-K1252
BoNT/F-K865-E1274
BoNT/G-N864-E1297
TeNT-1880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

The clostridial neurotoxins described herein may further comprise a translocation facilitating domain. Said domain facilitates delivery of the non-cytotoxic protease into the cytosol of the target cell and are described, for example, in WO 08/008803 and WO 08/008805, each of which is herein incorporated by reference thereto.

By way of example, suitable translocation facilitating domains include an enveloped virus fusogenic peptide domain, for example, suitable fusogenic peptide domains include influenzavirus fusogenic peptide domain (e.g. influenza A virus fusogenic peptide domain of 23 amino acids), alphavirus fusogenic peptide domain (e.g. Semliki Forest virus fusogenic peptide domain of 26 amino acids), vesiculovirus fusogenic peptide domain (e.g. vesicular stomatitis virus fusogenic peptide domain of 21 amino acids), respirovirus fusogenic peptide domain (e.g. Sendai virus fusogenic peptide domain of 25 amino acids), morbiliivirus fusogenic peptide domain (e.g. Canine distemper virus fusogenic peptide domain of 25 amino acids), avulavirus fusogenic peptide domain (e.g. Newcastle disease virus fusogenic peptide domain of 25 amino acids), henipavirus fusogenic peptide domain (e.g. Hendra virus fusogenic peptide domain of 25 amino acids), metapneumovirus fusogenic peptide domain (e.g. Human metapneumovirus fusogenic peptide domain of 25 amino acids) or spumavirus fusogenic peptide domain such as simian foamy virus fusogenic peptide domain; or fragments or variants thereof.

By way of further example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples from constituent neurotoxins include:
Botulinum type A neurotoxin-amino acid residues (872-1110)
Botulinum type B neurotoxin-amino acid residues (859-1097)
Botulinum type C neurotoxin-amino acid residues (867-1111)
Botulinum type D neurotoxin-amino acid residues (863-1098)
Botulinum type E neurotoxin-amino acid residues (846-1085)
Botulinum type F neurotoxin-amino acid residues (865-1105)
Botulinum type G neurotoxin-amino acid residues (864-1105)
Botulinum type X neurotoxin-amino acid residues (890-1121)
Tetanus neurotoxin-amino acid residues (880-1127)

The above sequence positions may vary a little according to serotype/sub-type, and further examples of suitable (reference) clostridial neurotoxin H_{CN} domains include:
Botulinum type A neurotoxin-amino acid residues (874-1110)
Botulinum type B neurotoxin-amino acid residues (861-1097)
Botulinum type C neurotoxin-amino acid residues (869-1111)
Botulinum type D neurotoxin-amino acid residues (865-1098)
Botulinum type E neurotoxin-amino acid residues (848-1085)
Botulinum type F neurotoxin-amino acid residues (867-1105)
Botulinum type G neurotoxin-amino acid residues (866-1105)
Tetanus neurotoxin-amino acid residues (882-1127)

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin H_{CN} facilitating domain may be combined or with a clostridial translocation domain peptide, examples of which include:
Botulinum type A neurotoxin-amino acid residues (449-1110)
Botulinum type B neurotoxin-amino acid residues (442-1097)
Botulinum type C neurotoxin-amino acid residues (450-1111)
Botulinum type D neurotoxin-amino acid residues (446-1098)
Botulinum type E neurotoxin-amino acid residues (423-1085)
Botulinum type F neurotoxin-amino acid residues (440-1105)
Botulinum type G neurotoxin-amino acid residues (447-1105)
Tetanus neurotoxin-amino acid residues (458-1127)

In some embodiments the chimeric clostridial neurotoxins of the present invention may lack a functional H_{C} domain of a clostridial neurotoxin. Accordingly, said chimeric clostridial neurotoxins are not able to bind rat synaptosomal membranes (via a clostridial H_{C} component) in binding assays as described in Shone et al. (1985) Eur. J. Biochem. 151, 75-82. The clostridial neurotoxins may preferably lack the last 50 C-terminal amino acids of a clostridial neurotoxin holotoxin. The chimeric clostridial neurotoxins may preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). This fact is confirmed by the following publications, each of which is herein incorporated in its entirety by reference thereto: Umland TC (1997) Nat. Struct. Biol. 4:788-792; Herreros J (2000) Biochem. J. 347:199-204; Halpern J (1993) J. Biol. Chem. 268:15, pp. 11188-11192; Rummel A (2007) PNAS 104:359-364; Lacey DB (1998) Nat. Struct. Biol. 5:898-902; Knapp (1998) Am. Cryst. Assoc. Abstract Papers 25:90; Swaminathan and Eswaramoorthy (2000) Nat. Struct. Biol. 7:1751-1759; and Rummel A (2004) Mol. Microbiol. 51 (3), 631-643. Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region may be either partially or wholly deleted, or otherwise modified (e.g. through conventional chemical or proteolytic treatment) to inactivate its native binding ability for nerve terminals at the neuromuscular junction.

Thus, a clostridial neurotoxin H_{N} peptide of the present invention may be C-terminally extended, i.e. it may be associated with all or part of a clostridial neurotoxin H_{C} domain, e.g. the H_{CN}, H_{CC} or H_{C} domain. References herein to a clostridial neurotoxin H_{N} peptide of the present invention encompass such C-terminally extended H_{N} peptides, which comprise one or more amino acid residues from a clostridial neurotoxin H_{C} domain. Alternatively, a clostridial neurotoxin H_{N} peptide of the present invention may not be associated with (or lack) all or part of a clostridial neurotoxin H_{C} domain, e.g. the H_{CN}, H_{CC} or H_{C} domain.

Typically if a chimeric clostridial neurotoxin of the invention or a clostridial neurotoxin H_{N} peptide of the present invention lacks all or part of a C-terminal peptide portion (H_{CC}) of a clostridial neurotoxin it thus lacks the H_{C} binding function of native clostridial neurotoxin. By way of example, a C-terminally extended clostridial H_{N} peptide may lack the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. Alternatively, the clostridial H_{N} peptide of the present invention may lack the entire C-terminal peptide portion (H_{CC}) of a clostridial neurotoxin and thus lacks the H_{C} binding function of native clostridial neurotoxin. By way of example, the clostridial H_{N} peptide may lack the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention may lack a clostridial H_{CC} reference sequence selected from the group consisting of:
Botulinum type A neurotoxin-amino acid residues (Y1111-L1296)
Botulinum type B neurotoxin-amino acid residues (Y1098-E1291)
Botulinum type C neurotoxin-amino acid residues (Y1112-E1291)
Botulinum type D neurotoxin-amino acid residues (Y1099-E1276)
Botulinum type E neurotoxin-amino acid residues (Y1086-K1252)
Botulinum type F neurotoxin-amino acid residues (Y1106-E1274)
Botulinum type G neurotoxin-amino acid residues (Y1106-E1297)
Botulinum type X neurotoxin-amino acid residues (Y1122-D1306)
Tetanus neurotoxin-amino acid residues (Y1128-D1315).

The above-identified reference sequences should be considered a guide as slight variations may occur according to sub-serotypes.

The present invention is suitable for application to many different varieties of clostridial neurotoxin. Thus, in the context of the present invention, the term “clostridial neurotoxin” embraces toxins produced by C. botulinum (botulinum neurotoxin serotypes A, B, C1, D, E, F, G, H, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing. The term “clostridial neurotoxin” also embraces botulinum neurotoxin serotype H. Preferably the clostridial neurotoxin is BoNT/A, more preferably BoNT/A1.

Botulinum neurotoxin (BoNT) is produced by C. botulinum in the form of a large protein complex, consisting of BoNT itself complexed to a number of accessory proteins. There are at present nine different classes of botulinum neurotoxin, namely: botulinum neurotoxin serotypes A, B, C1, D, E, F, G, H, and X all of which share similar structures and modes of action. Different BONT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

The term “clostridial neurotoxin” is also intended to embrace modified clostridial neurotoxins and derivatives thereof, including but not limited to those described below, all of which may be a constituent neurotoxin to a chimeric clostridial neurotoxin of the invention. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, a chimeric clostridial neurotoxin of the invention may be a modified chimeric clostridial neurotoxin, or a modified chimeric clostridial neurotoxin derivative, or a chimeric clostridial neurotoxin derivative, or may comprise a domain or portion thereof of a constituent modified clostridial neurotoxin, a modified clostridial neurotoxin derivative or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B H_{CC} subdomain have been disclosed in WO 2013/180799 and in WO 2016/154534 (both herein incorporated by reference).

A suitable amino acid residue substitution, insertion, indel or deletion in the BoNT/B H_{CC} subdomain may include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B H_{CC} subdomain may further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191M and S1199Y. Such modifications are present in chimeric clostridial neurotoxin of SEQ ID NO: 1. E1191M may correspond to position 1204 of SEQ ID NO: 1 and S1199Y may correspond to position 1212. Thus, SEQ ID NO: 1 may comprise 1204M and 1212Y.

The modification may be a modification when compared to unmodified BoNT/B shown as SEQ ID NO: 3, wherein the amino acid residue numbering is determined by alignment with SEQ ID NO: 3. As the presence of a methionine residue at position 1 of SEQ ID NO: 3 (as well as the SEQ ID NOs corresponding to other clostridial neurotoxin polypeptides described herein, including chimeric clostridial neurotoxin polypeptides) is optional, the skilled person will take the presence/absence of the methionine residue into account when determining amino acid residue numbering. For example, where SEQ ID NO: 3 includes a methionine, the position numbering will be as defined above (e.g. E1191 will be E1191 of SEQ ID NO: 3). Alternatively, where the methionine is absent from SEQ ID NO: 3 the amino acid residue numbering should be modified by −1 (e.g. E1191 will be E1190 of SEQ ID NO: 3).

Accordingly, an initial methionine amino acid residue of a polypeptide sequence of the chimeric clostridial neurotoxin may be optional or absent. Similar considerations apply when the methionine at position 1 of the other polypeptide sequences described herein is present/absent, and the skilled person will readily determine the correct amino acid residue numbering using techniques routine in the art. Alignment may be carried out using any of the methods described herein for determining sequence homology and/or % sequence identity.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine-or tyrosine-based motif, wherein said motif increases or decreases the biological activity and/or the biological persistence of the modified clostridial neurotoxin. Suitable leucine-based motifs include xDxxxLL (SEQ ID NO: 21), xExxxLL (SEQ ID NO: 22), xExxxIL (SEQ ID NO: 23), and xExxxLM (SEQ ID NO: 24) (wherein x is any amino acid). Suitable tyrosine-based motifs include Y-x-x-Hy (SEQ ID NO: 25) (wherein Hy is a hydrophobic amino acid). Examples of modified clostridial neurotoxins comprising leucine- and tyrosine-based motifs are described in WO 2002/008268, which is hereby incorporated by reference in its entirety.

### Formulations

As described and exemplified herein, the present inventors have developed a new formulation for chimeric clostridial neurotoxins that provides improved stability compared with conventional formulations.

A composition of the invention may be a liquid composition (or formulation) or a solid composition (or formulation).

Compositions of the invention are typically produced in liquid form. In some preferred embodiments, this liquid form is a pre-lyophilisation solution. A pre-lyophilisation solution may be subjected to lyophilisation (freeze drying) to produce a solid composition (post-lyophilisation cake). Solid compositions of the invention, such as post-lyophilisation cakes, may be reconstituted to produce a post-reconstitution solution (liquid). Compositions of the invention are administered to individuals or otherwise used in liquid form. Thus, a liquid composition of the invention may be (i) a pre-lyophilisation solution, (ii) a post-reconstitution solution, or (iii) a solution which is not intended for lyophilisation and/or which has not undergone post-lyophilisation reconstitution. Liquid compositions of class (iii) may also be referred to as a “ready-to-use” compositions or “ready-to-use” solutions, as they are manufactured and formulated as a liquid and sold for use in liquid form. All disclosure herein in relation to liquid formulations applies to any liquid formulation, including pre-lyophilisation solutions, post-reconstitution solutions and ready-to-use compositions, unless expressly stated to the contrary.

At each stage in its preparation, a composition of the invention may have certain parameters which are fixed, and others which are variable. In other words, certain parameters may be set at a desired amount and the other parameters may be adjusted to facilitate achieving the desired amount of the fixed parameters. These are set out in Table 2 below.

TABLE 2
Fixed/Variable Parameters for different composition types

| | Pre-Lyophilisation | Post-Lyophilisation | Post-Reconstitution |
|---|---|---|---|
| | Solution | Cake | Solution |
| Fixed | Solution excipient | Cake excipient | None |
| Parameters | concentrations | proportions (% w/w) | |
| | (molarity, % w/w and | | |
| | mass/mL) | | |
| Variable | Excipient mass per | Excipient mass per | Excipient mass per |
| Parameters | vial | vial | vial |
| | Fill volume | Fill volume | Reconstitution |
| | Vial size | Vial size | volume |
| | API mass per vial | API mass per vial | Solution excipient |
| | | | concentrations |
| | | | Vial size |
| | | | API mass per vial |

By way of non-limiting example in the context of pre-lyophilisation solution, the mass of excipient may be varied depending on vial size so that the desired concentration of said excipient is achieved for the volume of the vial. By way of further non-limiting example, alternatively or in addition the fill volume of a vial may be varied so that the desired concentration of said excipient is achieved for the volume of the vial.

By way of non-limiting example, in the context of the post-lyophilisation cake, the mass of excipient and/or clostridial neurotoxin may be varied so that the desired proportion of excipient and/or clostridial neurotoxin is achieved.

By way of further non-limiting example, in the context of a post-reconstitution solution, the reconstitution volume may be varied.

Typically, formulations (solid or liquid) of the invention are stored in GMP compliant containers. In particular, formulations (solid or liquid) of the invention may be stored in injection vials, such as those complying with ISO 8362-4 (Injection containers and accessories—Part 4: Injection vials made of moulded glass, at the time of writing ISO 8362-4:2011 is the current version of this standard). A 2 mL vial complying with ISO 8362-4 is referred to in the art as a 2R vial. Similarly, a 6 mL vial complying with ISO 8362-4 is referred to in the art as a 6R vial. Thus, an XR vial indicates a vial complying with ISO 8362-4, where the X value indicates the volume of the vial.

In some embodiments, the invention provides formulations (solid or liquid) in a 2 mL, 3 mL, 4 mL, 5 mL, 6 mL, 7 mL, 8 mL, 9 mL or 10 mL container or vial (e.g. a 2R, 3R, 4R, 5R, 6R, 7R, 8R, 9R or 10R vial), particularly a 2 mL (e.g. a 2R) or 6 mL (e.g. a 6R) container or vial. As described herein, a liquid composition of the invention (e.g. a pre-lyophilisation solution) may be used to fill a container and vial and then lyophilised to produce a solid composition of the invention. In such embodiments, the absolute amount of any component of the liquid composition, or the solid composition following lyophilisation, will be determined by the concentration of the component in the pre-lyophilisation liquid, and the volume of the liquid composition used to fill the container.

Liquid compositions of the invention typically have a precisely controlled pH. Solid compositions of the invention will, on reconstitution, typically have a precisely controlled pH.

The formulations of the invention typically comprise a dual histidine buffer. Without being bound by theory, it is believed that the use of a dual histidine buffer may facilitate control of the formulation pH, whereas conventional formulations lacking a dual histidine buffer may require titration (e.g. with hydrochloric acid) to reach an appropriate pH range.

A composition of the invention typically comprises a chimeric clostridial neurotoxin (such as a chimeric BoNT) and a dual histidine buffer as described herein. A composition of the invention may further comprise additional components, as described herein.

A composition of the invention may additionally comprise one or more surfactants, one or more stabilising agent and/or one or more tonicity agent, or any combination thereof. As described herein, the inventors have surprisingly found that particular combinations of such agents provide improved stability to chimeric clostridial neurotoxin formulations compared with conventional formulations. Accordingly, the compositions of the invention may comprise one or more surfactant, one or more stabilising agent and/or one or more tonicity agent as described herein. The one or more surfactant, one or more stabilising agent and/or one or more tonicity agent and the respective concentrations of each may be independently selected from any of the one or more surfactants, one or more stabilising agent and/or one or more tonicity agent and concentrations described herein. Whilst some preferred formulations are individualised herein, the scope of the present invention is not limited to these specific combinations of surfactants, stabilising agents and/or tonicity agents, or to formulations with specific concentrations of these surfactants, stabilising agents and/or tonicity agents.

The invention provides a composition comprising a chimeric clostridial neurotoxin (such as a chimeric BoNT), a dual histidine buffer, a stabilising agent, a surfactant and/or a tonicity agent, each of which (and the concentration thereof) may be independently selected as described herein. By way of non-limiting example, the invention provides a composition comprising a chimeric clostridial neurotoxin (such as a chimeric BoNT), a dual histidine buffer, sucrose, PS80 or poloxamer-188 (preferably PS80), and/or NaCl, wherein the concentration of each may be independently selected as described herein.

Preferably, the invention provides a composition comprising or consisting of a chimeric clostridial neurotoxin (such as a chimeric BoNT), a dual histidine buffer, a stabilising agent, a surfactant and a tonicity agent, each of which (and the concentration thereof) may be independently selected as described herein. By way of non-limiting example, the invention provides a composition comprising or consisting of a chimeric clostridial neurotoxin (such as a chimeric BoNT), a dual histidine buffer, sucrose, PS80 or poloxamer-188 (preferably PS80) and, wherein the concentration of each may be independently selected as described herein.

A composition of the invention may not comprise an additional excipient. A composition of the invention does not comprise an antioxidant. Without being bound by theory, as described herein, the inventors have developed new formulations for chimeric clostridial neurotoxins that provide improved stability compared with conventional formulations, such that the presence of additional excipients and/or antioxidant agents is not required to stabilise chimeric clostridial neurotoxins. Thus, a composition of the invention may consist of a chimeric clostridial neurotoxin (such as a chimeric BoNT), a dual histidine buffer, one or more surfactant, one or more stabilising agent and/or one or more tonicity agent, as described and exemplified herein.

Non-limiting examples of stabilising agents, surfactants and tonicity agents are described below, together with non-limiting examples of suitable concentrations of dual histidine buffers, stabilising agents, surfactants and tonicity agents useful in compositions of the invention. Each of these components and the concentration thereof may be independently selected from the disclosure below.

### Dual Histidine Buffer

The dual histidine buffer used in compositions of the invention typically comprises or consists of L-histidine and histidine monohydrochloride. As such, compositions of the invention typically comprise a chimeric clostridial neurotoxin, particularly a chimeric BONT, as described herein and L-histidine and histidine monohydrochloride. In particular, the invention provides solid compositions which comprise a chimeric clostridial neurotoxin, particularly a chimeric BONT, as described herein and L-histidine and histidine monohydrochloride (histidine-HCl). Such solid compositions may be formed by lyophilisation of a liquid formulation. In other words, a solid composition of the invention may be a post-lyophilisation cake.

For the avoidance of doubt, the terms histidine-HCl, histidine monohydrochloride and histidine monohydrochloride monohydrate are used interchangeable unless stated otherwise.

A solid composition of the invention may comprise a relative weight amount of L-histidine of between about 5% to about 25% (w/w), such as between about 10% to about 20% (w/w), between about 14% to about 20% (w/w), between about 15% to about 18% (w/w), between about 15.5% to about 17.5% (w/w), between about 15.5% to about 16.5% (w/w), or between about 16.0% to about 16.5% (w/w). Preferably a solid composition of the invention may comprise a relative weight amount of L-histidine of between about 15.5% to about 17.5% (w/w), such as between about 15.5% to about 16.5% (w/w), or between about 16.0% to about 16.5% (w/w). In some preferred embodiments, the relative weight amount of L-histidine is 16.2% (w/w).

A solid composition of the invention may comprise a relative weight amount of histidine-HCl of between about 5% to about 15% (w/w), such as between about 5% to about 10% (w/w), between about 6% to about 9% (w/w), between about 6.5% to about 8.5% (w/w), between about 6.5% to about 8.0% (w/w), between about 7.0% to about 8.0% (w/w), or between about 7.5% to about 8.0% (w/w). Preferably a solid composition of the invention may comprise a relative weight amount of histidine-HCl of between about 6.5% to about 8.5% (w/w), such as between about 6.5% to about 8.0% (w/w), between about 7.0% to about 8.0% (w/w), or between about 7.5% to about 8.0% (w/w). In some preferred embodiments, the relative weight amount of histidine-HCl is 7.9% (w/w).

A solid composition of the invention may comprise: (i) a relative weight amount of L-histidine of between about 5% to about 25% (w/w), such as between about 10% to about 20% (w/w), between about 14% to about 20% (w/w), between about 15% to about 18% (w/w), between about 15.5% to about 17.5% (w/w), between about 15.5% to about 16.5% (w/w), or between about 16.0% to about 16.5% (w/w); and (ii) a relative weight amount of histidine-HCl of between about 5% to about 15% (w/w), such as between about 5% to about 10% (w/w), between about 6% to about 9% (w/w), between about 6.5% to about 8.5% (w/w), between about 6.5% to about 8.0% (w/w), between about 7.0% to about 8.0% (w/w), or between about 7.5% to about 8.0% (w/w). Preferably a solid composition of the invention may comprise: (i) a relative weight amount of L-histidine of between about 15.5% to about 17.5% (w/w), such as between about 15.5% to about 16.5% (w/w), or between about 16.0% to about 16.5% (w/w); and (ii) a relative weight amount of histidine-HCl of between about 6.5% to about 8.5% (w/w), such as between about 6.5% to about 8.0% (w/w), between about 7.0% to about 8.0% (w/w), or between about 7.5% to about 8.0% (w/w). In some preferred embodiments, the relative weight amount of L-histidine is 16.2% (w/w) and the relative weight amount of histidine-HCl is 7.9% (w/w). In some preferred embodiments, the relative weight amount of L-histidine is about 16.2% (w/w) and the relative weight amount of histidine-HCl is about 7.9% (w/w).

A solid formulation of the invention may comprise an absolute weight of L-histidine of between about 1.0 mg/container to about 6.0 mg/container, such as between about 1.0 mg/container to about 5.0 mg/container, between about 1.0 mg/container to about 4.0 mg/container, between about 1.0 mg/container to about 3.0 mg/container, between about 1.0 mg/container to about 3.5 mg/container, between about 1.0 mg/container to about 2.5 mg/container, between about 1.0 mg/container to about 2.0 mg/container, or between about 1.0 mg/container to about 1.5 mg/container. The absolute amount of L-histidine in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of L-histidine of between about 1.0 mg/container to about 2.0 mg/container, such as between about 1.0 mg/container to about 1.5 mg/container, between about 1.0 mg/container to about 1.2 mg/container or between about 1.00 mg/container to about 1.15 mg/container. In some preferred embodiments, the absolute weight amount of L-histidine is about 1.14 mg/container (2R).

When a fill volume of about 0.5 mL is used (e.g. to fill a 2 mL container (e.g. 2R vial), 3 mL container (e.g. 3R vial), 5 mL container (e.g. 5R vial) or 6 mL container (e.g. 6R vial), particularly a 2 mL container (e.g. 2R vial) or a 6 mL container (e.g. 6R vial)), a solid formulation of the invention may comprise an absolute weight of L-histidine of between about 1.0 mg/container to about 2.0 mg/container, such as between about 1.0 mg/container to about 1.5 mg/container, between about 1.0 mg/container to about 1.2 mg/container or between about 1.00 mg/container to about 1.15 mg/container. Preferably, the absolute weight amount of L-histidine is about 1.140 mg/container (2R).

When a fill volume of about 1.0 mL is used (e.g. to fill a 6 mL container (e.g. 6R vial), a solid formulation of the invention may comprise an absolute weight of L-histidine of between about 2.0 mg/container to about 4.0 mg/container, such as between about 2.0 mg/container to about 3.5 mg/container, or between about 2.5 mg/container to about 3.5 mg/container. In some preferred embodiments, when a fill volume of about 1.0 mL is used (e.g. to fill a 6 mL container (e.g. 6R vial), a solid formulation of the invention may comprise an absolute weight of L-histidine of between about 1.0 mg/container to about 3.5 mg/container, such as between about 1.5 mg/container to about 3.0 mg/container, or between about 2.0 mg/container to about 2.5 mg/container. In some particularly preferred embodiments, the absolute weight amount of L-histidine is about 2.28 mg/container (6R).

When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of L-histidine of between about 3.0 mg/container to about 5.0 mg/container, such as between about 3.0 mg/container to about 4.0 mg/container, between about 3.0 mg/container to about 3.5 mg/container or between about 3.25 mg/container to about 3.50 mg/container. In some preferred embodiments, the absolute weight amount of L-histidine is about 3.42 mg/container (6R). A solid formulation of the invention may comprise an absolute weight of histidine-HCl of between about 0.25 mg/container to about 3.00 mg/container, such as between about 0.25 mg/container to about 2.50 mg/container, between about 0.25 mg/container to about 2.00 mg/container, between about 0.25 mg/container to about 1.50 mg/container, between about 0.25 mg/container to about 1.00 mg/container, between about 0.25 mg/container to about 0.75 mg/container, between about 0.30 mg/container to about 0.70 mg/container, or between about 0.40 mg/container to about 0.60 mg/container. The absolute amount of histidine-HCl in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of histidine-HCl of between about 0.25 mg/container to about 1.00 mg/container, such as between about 0.25 mg/container to about 0.75 mg/container, between about 0.30 mg/container to about 0.65 mg/container or between about 0.40 mg/container to about 0.60 mg/container. In some preferred embodiments, the absolute weight amount of histidine-HCl is about 0.555 mg/container (2R).

When a fill volume of about 0.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of histidine-HCl of between about 0.25 mg/container to about 1.00 mg/container, such as between about 0.25 mg/container to about 0.75 mg/container, between about 0.30 mg/container to about 0.65 mg/container or between about 0.40 mg/container to about 0.60 mg/container. Preferably, the absolute weight amount of histidine-HCl is about 0.555 mg/container (2R).

When a fill volume of about 1.0 mL is used (e.g. to fill a 6 mL container (e.g. 6R vial)), a solid formulation of the invention may comprise an absolute weight of histidine-HCl of between about 0.50 mg/container to about 2.00 mg/container, such as between about 0.50 mg/container to about 1.75 mg/container, between about 0.75 mg/container to about 1.75 mg/container or between about 1.0 mg/container to about 1.65 mg/container. Preferably, the absolute weight amount of histidine-HCl is about 1.110 mg/container (6R).

When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of histidine-HCl of between about 1.00 mg/container to about 2.50 mg/container, such as between about 1.00 mg/container to about 2.00 mg/container, between about 1.25 mg/container to about 1.75 mg/container or between about 1.50 mg/container to about 1.75 mg/container. In some preferred embodiments, the absolute weight amount of histidine-HCl is about 1.665 mg/container (6R).

A solid composition of the invention may comprise: (i) an absolute weight of L-histidine of between about 1.0 mg/container to about 6.0 mg/container, such as between about 1.0 mg/container to about 5.0 mg/container, between about 1.0 mg/container to about 4.0 mg/container, between about 1.0 mg/container to about 3.0 mg/container, between about 1.0 mg/container to about 3.5 mg/container, between about 1.0 mg/container to about 2.5 mg/container, between about 1.0 mg/container to about 2.0 mg/container, or between about 1.0 mg/container to about 1.5 mg/container; and (ii) an absolute weight of histidine-HCl of between about 0.25 mg/container to about 3.00 mg/container, such as between about 0.25 mg/container to about 2.50 mg/container, between about 0.25 mg/container to about 2.00 mg/container, between about 0.25 mg/container to about 1.50 mg/container, between about 0.25 mg/container to about 1.00 mg/container, between about 0.25 mg/container to about 0.75 mg/container, between about 0.30 mg/container to about 0.70 mg/container, or between about 0.40 mg/container to about 0.60 mg/container.

Preferably, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise: (i) an absolute weight of L-histidine of between about 1.0 mg/container to about 2.0 mg/container, such as between about 1.0 mg/container to about 1.5 mg/container, between about 1.0 mg/container to about 1.2 mg/container or between about 1.00 mg/container to about 1.15 mg/container; and (ii) an absolute weight of histidine-HCl of between about 0.25 mg/container to about 1.00 mg/container, such as between about 0.25 mg/container to about 0.75 mg/container, between about 0.30 mg/container to about 0.65 mg/container or between about 0.40 mg/container to about 0.60 mg/container. For example, when a fill volume of about 0.5 mL is used (e.g. to fill a 2 mL container (e.g. 2R vial), a solid formulation of the invention may comprise: (i) an absolute weight of L-histidine of about 1.140 mg/container; and (ii) an absolute weight of histidine-HCl of about 0.555 mg/container.

Preferably, when a fill volume of about 1.0 mL is used (e.g. to fill a 6 mL container (e.g. 6R vial), a solid formulation of the invention may comprise an absolute weight of L-histidine of between about 1.0 mg/container to about 3.5 mg/container, such as between about 1.5 mg/container to about 3.0 mg/container, or between about 2.0 mg/container to about 2.5 mg/container; and (ii) histidine-HCl of between about 0.50 mg/container to about 2.00 mg/container, such as between about 0.50 mg/container to about 1.75 mg/container, between about 0.75 mg/container to about 1.75 mg/container or between about 1.0 mg/container to about 1.65 mg/container. For example, when a fill volume of about 1.0 mL is used (e.g. to fill a 6 mL container (e.g. 6R vial), a solid formulation of the invention may comprise: (i) an absolute weight of L-histidine of about 2.28 mg/container; and (ii) an absolute weight of histidine-HCl of about 1.110 mg/container.

Preferably, when a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise: (i) an absolute weight of L-histidine of between about 3.0 mg/container to about 5.0 mg/container, such as between about 3.0 mg/container to about 4.0 mg/container, between about 3.0 mg/container to about 3.5 mg/container or between about 3.25 mg/container to about 3.50 mg/container; and (ii) an absolute weight of histidine-HCl of between about 1.00 mg/container to about 2.50 mg/container, such as between about 1.00 mg/container to about 2.00 mg/container, between about 1.25 mg/container to about 1.75 mg/container or between about 1.50 mg/container to about 1.75 mg/container. For example, when a fill volume of about 1.5 mL is used (e.g. to fill a 6 mL container (e.g. 6R vial), a solid formulation of the invention may comprise: (i) an absolute weight of L-histidine of about 3.420 mg/container; and (ii) an absolute weight of histidine-HCl of about 1.665 mg/container.

In some preferred embodiments, the absolute weight amount of L-histidine is about 1.14 mg/container (2R) and the absolute weight amount of histidine-HCl is about 0.555 mg/container (2R) (see, for example, Table 13).

In some preferred embodiments, the absolute weight amount of L-histidine is about 2.28 mg/container (6R) and the absolute weight amount of histidine-HCl is about 1.110 mg/container (6R) (see, for example, Table 13).

In some preferred embodiments, the absolute weight amount of L-histidine is about 3.42 mg/container (6R) and the absolute weight amount of histidine-HCl is about 1.665 mg/container (6R) (see, for example, Table 13).

The amount of L-histidine and/or histidine-HCl in a solid formulation of the invention may be selected to give a desired concentration on reconstitution to a specified final volume. Thus, a solid formulation of the invention may comprise L-histidine sufficient to give rise to a concentration of between about 2 mM to about 30 mM, such as between about 2 mM to about 20 mM, between about 10 mM to about 20 mM, between about 10 mM to about 17 mM, between about 12 mM and about 17 mM, between about 13 mM and about 17 mM, between about 14 mM and about 17 mM, between about 14 mM and about 16 mM, between about 14 mM and about 15 mM, between about 14.5 mM and about 16 mM, or between about 14.5 mM and 15.5 mM following reconstitution, preferably between about 14 mM and about 17 mM, such as between about 14 mM and about 15 mM. Preferably a solid formulation of the invention may comprise L-histidine sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A solid formulation of the invention may comprise histidine-HCl sufficient to give rise to a concentration of between about 0.5 mM to about 15.0 mM, such as between about 0.5 mM to about 10.0 mM, between about 0.5 mM to about 7.0 mM, between about 1.0 mM and about 7.0 mM, between about 2.0 mM and about 7.0 mM, between about 2.5 mM and about 6.0 mM, between about 3.0 mM and about 6.0 mM, between about 4.0 mM and about 6.0 mM, between about 5.0 mM and about 6.0 mM, or between about 5.0 mM and 5.5 mM following reconstitution, preferably between about 4.0 mM and about 6.0 mM, such as between about 5.0 mM and about 6.0 mM. Preferably a solid formulation of the invention may comprise histidine-HCl sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.0 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A solid formulation of the invention may comprise: (i) L-histidine sufficient to give rise to a concentration of between about 2 mM to about 30 mM, such as between about 2 mM to about 20 mM, between about 10 mM to about 20 mM, between about 10 mM to about 17 mM, between about 12 mM and about 17 mM, between about 13 mM and about 17 mM, between about 14 mM and about 17 mM, between about 14 mM and about 16 mM, between about 14 mM and about 15 mM, between about 14.5 mM and about 16 mM, or between about 14.5 mM and 15.5 mM following reconstitution; and (ii) histidine-HCl sufficient to give rise to a concentration of between about 0.5 mM to about 15.0 mM, such as between about 0.5 mM to about 10.0, between about 0.5 mM to about 7.0 mM, between about 1.0 mM and about 7.0 mM, between about 2.0 mM and about 7.0 mM, between about 2.5 mM and about 6.0 mM, between about 3.0 mM and about 6.0 mM, between about 4.0 mM and about 6.0 mM, between about 5.0 mM and about 6.0 mM, or between about 5.0 mM and 5.5 mM following reconstitution. Preferably a solid formulation of the invention comprises (i) L-histidine sufficient to give rise to a concentration of between about 14 mM and about 17 mM, such as between about 14 mM and about 15 mM; and (ii) histidine-HCl sufficient to give rise to a concentration of between about 4.0 mM and about 6.0 mM, such as between about 5.0 mM and about 6.0 mM. In such preferred solid formulations, preferably the amount of L-histidine and histidine-HCl is sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention, such as a pre-lyophilisation solution, may comprise a relative weight amount of L-histidine of between about 0.05% to about 0.50% (w/w), such as between about 0.05% to about 0.40% (w/w), between about 0.10% to about 0.40% (w/w), between about 0.15% to about 0.40% (w/w), between about 0.15% to about 0.30% (w/w), between about 0.20% to about 0.30% (w/w), or between about 0.20% to about 0.25% (w/w). Preferably a liquid composition of the invention may comprise a relative weight amount of L-histidine of between about 0.20% to about 0.30% (w/w), such as between about 0.20% to about 0.25% (w/w), or between about 0.21% to about 0.23% (w/w).

A liquid composition of the invention, such as a pre-lyophilisation solution, may comprise a relative weight amount of histidine-HCl of between about 0.05% to about 0.50% (w/w), such as between about 0.05% to about 0.30% (w/w), between about 0.05% to about 0.25% (w/w), between about 0.05% to about 0.20% (w/w), between about 0.075% to about 0.20% (w/w), between about 0.10% to about 0.20% (w/w), or between about 0.10% to about 0.15% (w/w). Preferably a liquid composition of the invention may comprise a relative weight amount of histidine-HCl of between about 0.10% to about 0.20% (w/w), such as between about 0.10% to about 0.15% (w/w), between about 0.10% to about 0.13% (w/w), or between about 0.10% to about 0.12% (w/w).

A liquid composition of the invention, such as a pre-lyophilisation solution, may comprise: (i) a relative weight amount of L-histidine of between about 0.05% to about 0.50% (w/w), such as between about 0.05% to about 0.40% (w/w), between about 0.10% to about 0.40% (w/w), between about 0.15% to about 0.40% (w/w), between about 0.15% to about 0.30% (w/w), between about 0.20% to about 0.30% (w/w), or between about 0.20% to about 0.25% (w/w); and (ii) a relative weight amount of histidine-HCl of between about 0.05% to about 0.50% (w/w), such as between about 0.05% to about 0.30% (w/w), between about 0.05% to about 0.25% (w/w), between about 0.05% to about 0.20% (w/w), between about 0.075% to about 0.20% (w/w), between about 0.10% to about 0.20% (w/w), or between about 0.10% to about 0.15% (w/w). Preferably liquid composition of the invention, such as a pre-lyophilisation solution, may comprise: (i) a relative weight amount of L-histidine of between about 0.20% to about 0.30% (w/w), such as between about 0.20% to about 0.25% (w/w), or between about 0.21% to about 0.23% (w/w); and (ii) a relative weight amount of histidine-HCl of between about 0.10% to about 0.20% (w/w), such as between about 0.10% to about 0.15% (w/w), between about 0.10% to about 0.13% (w/w), or between about 0.10% to about 0.12% (w/w).

A liquid composition of the invention, such as a pre-lyophilisation solution, may comprise L-histidine at a concentration of between about 0.10% to about 1.0% (w/w), such as between about 0.10% to about 0.80% (w/w), between about 0.40% to about 0.60% (w/w), such as between about 0.40% to about 0.50% (w/w), or between about 0.42% to about 0.46% (w/w). A liquid composition of the invention, such as a pre-lyophilisation solution, may comprise L-histidine at a concentration of about 0.45% (w/w).

A liquid composition of the invention, such as a pre-lyophilisation solution, may comprise histidine-HCl at a concentration of between about 0.10% to about 1.0% (w/w), such as between about 0.10% to about 0.60% (w/w), between about 0.20% to about 0.40% (w/w), such as between about 0.20% to about 0.30% (w/w), between about 0.20% to about 0.26% (w/w), or between about 0.20% to about 0.24% (w/w). A liquid composition of the invention, such as a pre-lyophilisation solution, may comprise histidine-HCl at a concentration of 0.22% (w/w).

A liquid composition of the invention, such as a pre-lyophilisation solution, may comprise: (i) L-histidine at a concentration of between about 0.10% to about 1.0% (w/w), such as between about 0.10% to about 0.80% (w/w), between about 0.40% to about 0.60% (w/w), such as between about 0.40% to about 0.50% (w/w), or between about 0.42% to about 0.46% (w/w); and (ii) histidine-HCl at a concentration of between about 0.10% to about 1.0% (w/w), such as between about 0.10% to about 0.60% (w/w), between about 0.20% to about 0.40% (w/w), such as between about 0.20% to about 0.30% (w/w), between about 0.20% to about 0.26% (w/w), or between about 0.20% to about 0.24% (w/w). Preferably a liquid composition of the invention, such as a pre-lyophilisation solution, comprises: (i) L-histidine at a concentration of between about 0.20% to about 0.26% (w/w), such as between about 0.20% to about 0.24% (w/w); and (ii) histidine-HCl at a concentration of between about 0.20% to about 0.26% (w/w), such as between about 0.20% to about 0.24% (w/w). By way of example, a liquid composition of the invention, such as a pre-lyophilisation solution, may comprise: (i) L-histidine at a concentration of about 0.45% (w/w); and (ii) histidine-HCl at a concentration of about 0.22% (w/w).

A liquid composition of the invention, such as a pre-lyophilisation solution, may comprise L-histidine at a concentration of between about 10 mM to about 20 mM, such as between about 10 mM to about 17 mM, between about 12 mM and about 17 mM, between about 13 mM and about 17 mM, between about 14 mM and about 17 mM, between about 14 mM and about 16 mM, between about 14 mM and about 15 mM, between about 14.5 mM and about 16 mM, or between about 14.5 mM and 15.5 mM. Preferably a liquid composition of the invention, such as a pre-lyophilisation solution, comprises L-histidine at a concentration of between about 14 mM and about 17 mM, such as between about 14 mM and about 15 mM.

A liquid composition of the invention, such as a pre-lyophilisation solution, may comprise histidine-HCl at a concentration of between about 0.5 mM to about 10.0 mM, such as between about 0.5 mM to about 7.0 mM, between about 1.0 mM and about 7.0 mM, between about 2.0 mM and about 7.0 mM, between about 2.5 mM and about 6.0 mM, between about 3.0 mM and about 6.0 mM, between about 4.0 mM and about 6.0 mM, between about 5.0 mM and about 6.0 mM, or between about 5.0 mM and 5.5 mM. Preferably a liquid composition of the invention, such as a pre-lyophilisation solution, comprises histidine-HCl at a concentration of between about 4.0 mM and about 6.0 mM, such as between about 5.0 mM and about 6.0 mM.

A liquid composition of the invention, such as a pre-lyophilisation solution, may comprise: (i) L-histidine at a concentration of between about 10 mM to about 20 mM, such as between about 10 mM to about 17 mM, between about 12 mM and about 17 mM, between about 13 mM and about 17 mM, between about 14 mM and about 17 mM, between about 14 mM and about 16 mM, between about 14 mM and about 15 mM, between about 14.5 mM and about 16 mM, or between about 14.5 mM and 15.5 mM; and (ii) histidine-HCl at a concentration of between about 0.5 mM to about 10.0 mM, such as between about 0.5 mM to about 7.0 mM, between about 1.0 mM and about 7.0 mM, between about 2.0 mM and about 7.0 mM, between about 2.5 mM and about 6.0 mM, between about 3.0 mM and about 6.0 mM, between about 4.0 mM and about 6.0 mM, between about 5.0 mM and about 6.0 mM, or between about 5.0 mM and 5.5 mM. Preferably a liquid composition of the invention, such as a pre-lyophilisation solution, comprises: (i) L-histidine at a concentration of between about 14 mM and about 17 mM, such as between about 14 mM and about 15 mM; and (ii) histidine-HCl at a concentration of between about 4.0 mM and about 6.0 mM, such as between about 5.0 mM and about 6.0 mM.

A liquid composition of the invention, such as a pre-lyophilisation solution, may comprise L-histidine at a concentration of between about 20.0 mM to about 40.0 mM, such as between about 20.0 mM to about 30.0 mM, between about 25.0 mM to about 30.0 mM, between about 27.0 mM and about 29.5 mM, or between about 28.0 mM and about 29.5 mM. A liquid composition of the invention, such as a pre-lyophilisation solution, may comprise L-histidine at a concentration of about 29.4 mM.

A liquid composition of the invention, such as a pre-lyophilisation solution, may comprise histidine-HCl at a concentration of between about 1.0 mM to about 20.0 mM, such as between about 5.0 mM to about 15.0 mM, between about 7.0 mM to about 12.0 mM, between about 8.0 mM and about 11.0 mM, between about 9.0 mM and about 11.0 mM, or between about 10.0 mM and about 11.0 mM. A liquid composition of the invention, such as a pre-lyophilisation solution, may comprise histidine-HCl at a concentration of about 10.6 mM.

A liquid composition of the invention, such as a pre-lyophilisation solution, may comprise: (i) L-histidine at a concentration of between about 20.0 mM to about 40.0 mM, such as between about 20.0 mM to about 30.0 mM, between about 25.0 mM to about 30.0 mM, between about 27.0 mM and about 29.5 mM, or between about 28.0 mM and about 29.5 mM; and (ii) histidine-HCl at a concentration of between about 1.0 mM to about 20.0 mM, such as between about 5.0 mM to about 15.0 mM, between about 7.0 mM to about 12.0 mM, between about 8.0 mM and about 11.0 mM, between about 9.0 mM and about 11.0 mM, or between about 10.0 mM and about 11.0 mM. Preferably a liquid composition of the invention, such as a pre-lyophilisation solution, comprises: (i) L-histidine at a concentration of between about 27.0 mM and about 29.5 mM, such as between about 28.0 mM and about 29.5 mM; and (ii) histidine-HCl at a concentration of between about 9.0 mM and about 11.0 mM, such as between about 10.0 mM and about 11.0 mM. For example, a liquid composition of the invention, such as a pre-lyophilisation solution, comprises: (i) L-histidine at a concentration of about 29.4 mM; and (ii) histidine-HCl at a concentration of about 10.6 mM.

A liquid composition of the invention, such as a pre-lyophilisation solution, may comprise L-histidine at a concentration of between about 1.0 mg/mL to about 5.0 mg/mL, such as between about 1.0 mg/mL to about 4.0 mg/mL, between about 1.0 mg/mL to about 3.0 mg/mL, between about 1.5 mg/mL to about 3.0 mg/mL, between about 2.0 mg/mL to about 3.0 mg/mL, between about 1.5 mg/mL to about 2.5 mg/mL, between about 2.0 mg/mL to about 2.5 mg/mL, between about 2.0 mg/mL to about 2.3 mg/mL, or between about 2.1 mg/mL to about 2.3 mg/mL. Preferably a liquid composition of the invention, such as a pre-lyophilisation solution, comprises L-histidine at a concentration of between about 1.5 mg/mL to about 3.0 mg/mL, such as between about 2.0 mg/mL to about 2.5 mg/mL, or between about 2.0 mg/mL to about 2.3 mg/mL.

A liquid composition of the invention, such as a pre-lyophilisation solution, may comprise histidine-HCl at a concentration of between about 0.01 mg/mL to about 5.00 mg/mL, such as between about 0.01 mg/mL to about 2.00 mg/mL, between about 0.50 mg/mL to about 2.00 mg/mL, between about 0.50 mg/mL to about 1.75 mg/mL, between about 0.75 mg/mL to about 1.75 mg/mL, between about 0.75 mg/mL to about 1.50 mg/mL, between about 0.75 mg/mL to about 1.25 mg/mL, or between about 1.00 mg/mL to about 1.25 mg/mL. Preferably a liquid composition of the invention, such as a pre-lyophilisation solution, comprises histidine-HCl at a concentration of between about 0.75 mg/mL to about 1.75 mg/mL, such as between about 0.75 mg/mL to about 1.25 mg/mL, or between about 1.00 mg/mL to about 1.25 mg/mL.

A liquid composition of the invention, such as a pre-lyophilisation solution, may comprise: (i) L-histidine at a concentration of between about 1.0 mg/mL to about 5.0 mg/mL, such as between about 1.0 mg/mL to about 4.0 mg/mL, between about 1.0 mg/mL to about 3.0 mg/mL, between about 1.5 mg/mL to about 3.0 mg/mL, between about 2.0 mg/mL to about 3.0 mg/mL, between about 1.5 mg/mL to about 2.5 mg/mL, between about 2.0 mg/mL to about 2.5 mg/mL, between about 2.0 mg/mL to about 2.3 mg/mL, or between about 2.1 mg/mL to about 2.3 mg/mL; and (ii) histidine-HCl at a concentration of between about 0.01 mg/mL to about 5.00 mg/mL, such as between about 0.01 mg/mL to about 2.00 mg/mL, between about 0.50 mg/mL to about 2.00 mg/mL, between about 0.50 mg/mL to about 1.75 mg/mL, between about 0.75 mg/mL to about 1.75 mg/mL, between about 0.75 mg/mL to about 1.50 mg/mL, between about 0.75 mg/mL to about 1.25 mg/mL, or between about 1.00 mg/mL to about 1.25 mg/mL. Preferably a liquid composition of the invention, such as a pre-lyophilisation solution, comprises: (i) L-histidine at a concentration of between about 1.5 mg/mL to about 3.0 mg/mL, such as between about 2.0 mg/mL to about 2.5 mg/mL, or between about 2.0 mg/mL to about 2.3 mg/mL; and (ii) histidine-HCl at a concentration of between about 0.75 mg/mL to about 1.75 mg/mL, such as between about 0.75 mg/mL to about 1.25 mg/mL, or between about 1.00 mg/mL to about 1.25 mg/mL.

A liquid composition of the invention, such as a pre-lyophilisation solution, may comprise L-histidine at a concentration of between about 0.02 mg/mL to about 10.0 mg/mL, such as between about 0.02 mg/mL to about 4.00 mg/mL, between about 4.0 mg/mL to about 5.0 mg/mL, between about 4.0 mg/mL to about 4.6 mg/mL, or between about 4.2 mg/mL to about 4.6 mg/mL. A liquid composition of the invention, such as a pre-lyophilisation solution, may comprise L-histidine at a concentration of about 4.56 mg/mL.

A liquid composition of the invention, such as a pre-lyophilisation solution, may comprise histidine-HCl at a concentration of between about 0.02 mg/mL to about 10.0 mg/mL, such as between about 0.02 mg/mL to about 4.00 mg/mL, between about 1.50 mg/mL to about 3.50 mg/mL, such as between about 1.50 mg/mL to about 2.50 mg/mL, or between about 2.00 mg/mL to about 2.50 mg/mL. A liquid composition of the invention, such as a pre-lyophilisation solution, may comprise histidine-HCl at a concentration of about 2.22 mg/mL.

A liquid composition of the invention, such as a pre-lyophilisation solution, may comprise: (i) L-histidine at a concentration of between about 0.02 mg/mL to about 10.0 mg/mL, such as between about 0.02 mg/mL to about 4.00 mg/mL, between about 4.0 mg/mL to about 5.0 mg/mL, between about 4.0 mg/mL to about 4.6 mg/mL, or between about 4.2 mg/mL to about 4.6 mg/mL; and (ii) histidine-HCl at a concentration of between about 0.10% to about 1.0% (w/w), such as between about 0.10% to about 0.60% (w/w), between about 0.20% to about 0.40% (w/w), such as between about 0.20% to about 0.30% (w/w), between about 0.20% to about 0.26% (w/w), or between about 0.20% to about 0.24% (w/w). Preferably a liquid composition of the invention, such as a pre-lyophilisation solution, comprises: (i) L-histidine at a concentration of between about 4.0 mg/mL to about 4.6 mg/mL, such as between about 4.2 mg/mL to about 4.6 mg/mL; and (ii) histidine-HCl at a concentration of between about 0.20% to about 0.26% (w/w), such as between about 0.20% to about 0.24% (w/w). For example, a liquid composition of the invention, such as a pre-lyophilisation solution, may comprise: (i) L-histidine at a concentration of about 4.56 mg/mL; and (ii) histidine-HCl at a concentration of about 2.22 mg/mL.

### Stabilising Agents

As described herein, the present inventors obtained further improvements to stability using a non-reducing sugar, such as sucrose, as a stabilising agent in the chimeric clostridial neurotoxin formulations. Accordingly, compositions of the invention typically comprise a non-reducing sugar. Non-limiting examples of non-reducing sugars include sucrose, trehalose, raffinose, stachyose and verbascose. Preferably compositions of the invention which comprise a non-reducing sugar comprise a disaccharide, particularly sucrose. All references herein to non-reducing sugars apply equally and without reservation to disaccharides (e.g. sucrose) and all references herein to disaccharides and/or sucrose apply equally and without reservation to non-reducing sugars, unless explicitly stated to the contrary.

Compositions of the invention may not comprise one or more stabilising agent that is conventionally used for stabilising clostridial neurotoxin (e.g. BoNT) compositions. In particular, compositions of the invention may not comprise a reducing sugar (e.g. lactose). Alternatively or in addition, a composition of the invention may not comprise a protein stabilising agent, such as albumin (e.g. human albumin). Typically such conventional stabilising agents can be omitted from compositions of the invention when a non-reducing sugar is used as a stabilising agent.

As such, the present invention provides chimeric clostridial neurotoxin compositions which have a precisely controlled pH, and which contain a non-reducing sugar stabilising agent, particularly a disaccharide such as sucrose. Preferably, such compositions do not comprise trehalose (e.g., trehalose dihydrate). Such compositions typically do not comprise stabilising agents conventionally used in clostridial neurotoxin formulations, such as lactose and/or human albumin.

A solid composition of the invention may comprise a relative weight amount of a disaccharide (e.g. sucrose) of between about 50% to about 90% (w/w), such as between about 50% to about 80% (w/w), between about 60% to about 80% (w/w), between about 60% to about 80% (w/w), between about 65% to about 75% (w/w), or between about 70% to about 75% (w/w). Preferably a solid composition of the invention may comprise a relative weight amount of a disaccharide (e.g. sucrose) of between about 65% to about 75% (w/w), such as between about 70% to about 75% (w/w), or between about 70% to about 72% (w/w).

A solid formulation of the invention may comprise an absolute weight of a disaccharide (e.g. sucrose) of between about 1.0 mg/container to about 25.0 mg/container, such as between about 1.0 mg/container to about 20.0 mg/container, between about 1.0 mg/container to about 17.5 mg/container, between about 1.0 mg/container to about 15.0 mg/container, between about 1.0 mg/container to about 10.0 mg/container, between about 1.0 mg/container to about 7.5 mg/container, between about 2.0 mg/container to about 6.0 mg/container, or between about 2.5 mg/container to about 5.5 mg/container. The absolute amount of a disaccharide (e.g. sucrose) in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a disaccharide (e.g. sucrose) of between about 1.0 mg/container to about 10.0 mg/container, such as between about 1.0 mg/container to about 7.5 mg/container, between about 2.0 mg/container to about 6.0 mg/container, between about 2.5 mg/container to about 5.5 mg/container, or between about 4.5 mg/container to about 5.5 mg/container.

When a fill volume of about 0.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a disaccharide (e.g. sucrose) of between about 1.0 mg/container to about 10.0 mg/container, such as between about 1.0 mg/container to about 7.5 mg/container, between about 2.0 mg/container to about 6.0 mg/container, between about 2.5 mg/container to about 5.5 mg/container, or between about 4.5 mg/container to about 5.5 mg/container. When a fill volume of about 1.0 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a disaccharide (e.g. sucrose) of between about 5.0 mg/container to about 15.0 mg/container, such as between about 7.5 mg/container to about 12.5 mg/container, between about 8.5 mg/container to about 11.0 mg/container or between about 9.0 mg/container to about 11.0 mg/container. When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a disaccharide (e.g. sucrose) of between about 10.0 mg/container to about 20.0 mg/container, such as between about 10.0 mg/container to about 17.5 mg/container, between about 12.5 mg/container to about 17.5 mg/container or between about 14.0 mg/container to about 16.0 mg/container.

The amount of disaccharide (e.g. sucrose) in a solid formulation of the invention may be selected to give a desired concentration on reconstitution to a specified final volume. Thus, a solid formulation of the invention may comprise a disaccharide (e.g. sucrose) sufficient to give rise to a concentration of between about 5 mM to about 60 mM, such as between about 5 mM to about 50 mM, between about 10 mM to about 50 mM, between about 10 mM to about 40 mM, between about 15 mM and about 40 mM, between about 15 mM and about 35 mM, between about 20 mM and about 35 mM, between about 25 mM and about 35 mM, between about 27.5 mM and about 32.5 mM, or between about 28.5 mM and about 31.5 mM following reconstitution, preferably between about 25 mM and about 35 mM, such as between about 27.5 mM and about 32.5 mM, or between about 28.5 mM and about 31.5 mM. Preferably a solid formulation of the invention may comprise a disaccharide (e.g. sucrose) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention, such as a pre-lyophilisation solution, may comprise a relative weight amount of a disaccharide (e.g. sucrose) of between about 0.10% to about 2.50% (w/w), such as between about 0.10% to about 2.00% (w/w), between about 0.10% to about 1.50% (w/w), between about 0.25% to about 1.50% (w/w), between about 0.50% to about 1.50% (w/w), between about 0.75% to about 1.25% (w/w), between about 0.90% to about 1.10% (w/w). Preferably a liquid composition of the invention may comprise a relative weight amount of a disaccharide (e.g. sucrose) of between about 0.75% to about 1.25% (w/w), such as between about 0.90% to about 1.10% (w/w), or between about 0.95% to about 1.05% (w/w).

A liquid composition of the invention, such as a pre-lyophilisation solution, may comprise a relative weight amount of a disaccharide (e.g. sucrose) of between about 0.20% to about 5.00% (w/w), such as between about 0.20% to about 4.00% (w/w), between about 0.20% to about 3.00% (w/w), between about 0.50% to about 3.00% (w/w), between about 1.00% to about 3.00% (w/w), between about 1.50% to about 2.50% (w/w), between about 1.80% to about 2.20% (w/w). Preferably a liquid composition of the invention may comprise a relative weight amount of a disaccharide (e.g. sucrose) of between about 1.50% to about 2.50% (w/w), such as between about 1.80% to about 2.20% (w/w), or between about 1.90% to about 2.10% (w/w). A liquid composition of the invention, such as a pre-lyophilisation solution, may comprise a relative weight amount of a disaccharide (e.g. sucrose) of about 1.98% (w/w).

A liquid composition of the invention, such as a pre-lyophilisation solution, may comprise a disaccharide (e.g. sucrose) at a concentration of between about 10 mM to about 50 mM, such as between about 10 mM to about 40 mM, between about 15 mM and about 40 mM, between about 15 mM and about 35 mM, between about 20 mM and about 35 mM, between about 25 mM and about 35 mM, between about 27.5 mM and about 32.5 mM, or between about 28.5 mM and about 31.5 mM. Preferably a liquid composition of the invention, such as a pre-lyophilisation solution, comprises a disaccharide (e.g. sucrose) at a concentration of between about 25 mM and about 35 mM, such as between about 27.5 mM and about 32.5 mM, or between about 28.5 mM and about 31.5 mM.

A liquid composition of the invention, such as a pre-lyophilisation solution, may comprise a disaccharide (e.g. sucrose) at a concentration of between about 20 mM to about 100 mM, such as between about 20 mM to about 80 mM, between about 30 mM and about 80 mM, between about 40 mM and about 70 mM, between about 50 mM and about 70 mM, between about 55 mM and about 65 mM, between about 57 mM and about 63 mM, or between about 58 mM and about 60 mM. Preferably a liquid composition of the invention, such as a pre-lyophilisation solution, comprises a disaccharide (e.g. sucrose) at a concentration of between about 57 mM and about 63 mM, such as between about 58 mM and about 60 mM or between about 58 mM and about 59 mM. A liquid composition of the invention, such as a pre-lyophilisation solution, may comprise a disaccharide (e.g. sucrose) at a concentration of about 58.4 mM.

A liquid composition of the invention, such as a pre-lyophilisation solution, may comprise a disaccharide (e.g. sucrose) at a concentration of between about 1.0 mg/mL to about 20.0 mg/mL, such as between about 1.0 mg/mL to about 15.0 mg/mL, between about 2.5 mg/mL to about 15.0 mg/mL, between about 5.0 mg/mL to about 15.0 mg/mL, between about 5.0 mg/mL to about 12.5 mg/mL, between about 7.5 mg/mL to about 12.5 mg/mL, between about 8.0 mg/mL to about 12.0 mg/mL, between about 9.0 mg/mL to about 11.0 mg/mL, or between about 9.50 mg/mL to about 10.5 mg/mL. Preferably a liquid composition of the invention, such as a pre-lyophilisation solution, comprises a disaccharide (e.g. sucrose) at a concentration of between about 7.5 mg/mL to about 12.5 mg/mL, such as between about 8.0 mg/mL to about 12.0 mg/mL, or between about 9.0 mg/mL to about 11.0 mg/mL.

A liquid composition of the invention, such as a pre-lyophilisation solution, may comprise a disaccharide (e.g. sucrose) at a concentration of between about 2.0 mg/mL to about 40.0 mg/mL, such as between about 2.0 mg/mL to about 30.0 mg/mL, between about 5.0 mg/mL to about 30.0 mg/mL, between about 10.0 mg/mL to about 30.0 mg/mL, between about 10.0 mg/mL to about 25.0 mg/mL, between about 15.0 mg/mL to about 25.0 mg/mL, between about 16.0 mg/mL to about 24.0 mg/mL, between about 18.0 mg/mL to about 22.0 mg/mL, or between about 19.0 mg/mL to about 21.0 mg/mL. Preferably a liquid composition of the invention, such as a pre-lyophilisation solution, comprises a disaccharide (e.g. sucrose) at a concentration of between about 15.0 mg/mL to about 25.0 mg/mL, such as between about 16.0 mg/mL to about 24.0 mg/mL, or between about 18.0 mg/mL to about 22.0 mg/mL. A liquid composition of the invention, such as a pre-lyophilisation solution, may comprise a disaccharide (e.g. sucrose) at a concentration of about 20 mg/mL.

Preferably, the composition does not further comprise additional stabilising agents, such as a charged carrier component. For example, preferably, the clostridial neurotoxin comprised in the composition is not non-covalently associated with a charged carrier molecule (e.g., a positively charged carrier molecule such as a positively charged carrier polypeptide). For example, the composition preferably does not comprise a carrier component comprising a positively charged polylysine backbone. Preferably, the clostridial neurotoxin comprised in the composition is not non-covalently associated with a positively charged polylysine backbone having covalently attached thereto one or more positively charged efficiency groups having an amino acid sequence of G_{X}-RGRDDRRQRRR-G_{y} (SEQ ID NO: 38), G_{X}-YGRKKRRQRRR-G_{y} (SEQ ID NO: 39) or G_{X}-RKKRRQRRR-G_{y} (SEQ ID NO: 40), wherein the subscripts x and y are each independently an integer of from 0 to 20. Preferably, the clostridial neurotoxin comprised in the composition is not non-covalently associated with a carrier component having the amino acid sequence of: RKKRRQRRRG-(K)ₙ-GRKKRRQRRR, wherein n is 15 (SEQ ID NO: 41).

### Surfactants

As described herein, the present inventors obtained further improvements to stability using a specific surfactant in the chimeric clostridial neurotoxin formulations. As described herein, compositions of the invention may comprise a surfactant, particularly a non-ionic surfactant. Non-limiting examples of surfactants include polysorbates such as polysorbate 20 or polysorbate 80, block copolymers such as poloxamer (i.e. copolymers of polyethylene and propylene glycol) and sorbitan fatty acid esters.

A composition of the invention may comprise a non-ionic surfactant, particularly a polysorbate, such as polysorbate having a mean polymerisation degree ranging from 20 to 100 monomer units, and may for example be polysorbate 80. In a preferred embodiment, the polysorbate is vegetable-derived. A composition of the invention may comprise a non-ionic surfactant, particularly a polysorbate, such as polysorbate having a mean polymerisation degree ranging from 20 to 100 monomer units, and may for example be polysorbate 80 (PS80). In a preferred embodiment, the polysorbate is vegetable-derived. A composition of the invention may comprise a poloxamer, such as poloxamer 188. In some preferred embodiments, the surfactant is not Tertiary-Dodecyl Mercaptan (TDM) and/or n-dodecyl-β-D-maltoside (DDM).

A solid composition of the invention may comprise a relative weight amount of a surfactant (e.g. PS80) of between about 0.1% to about 2.0%, such as between about 0.1% to about 1.5% (w/w), between about 0.1% to about 1.0% (w/w), between about 0.10% to about 0.90% (w/w), between about 0.20% to about 0.80% (w/w), between about 0.5% to about 1.0% (w/w), or between about 0.50% to about 0.80% (w/w). Preferably a solid composition of the invention may comprise a relative weight amount of a surfactant (e.g. PS80) of between about 0.1% to about 1.0% (w/w), such as between about 0.20% to about 0.90% (w/w), between about 0.5% to about 1.0% (w/w), or between about 0.50% to about 0.80% (w/w).

A solid formulation of the invention may comprise an absolute weight of a surfactant (e.g. PS80) of between about 0.010 mg/container to about 1.00 mg/container, such as between about 0.010 mg/container to about 0.500 mg/container, between about 0.010 mg/container to about 0.250 mg/container, between about 0.010 mg/container to about 0.100 mg/container, between about 0.010 mg/container to about 0.075 mg/container, between about 0.025 mg/container to about 0.075 mg/container, or between about 0.030 mg/container to about 0.060 mg/container. The absolute amount of a surfactant (e.g. PS80) in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a surfactant (e.g. PS80) of between about 0.010 mg/container to about 0.100 mg/container, such as between about 0.010 mg/container to about 0.075 mg/container, between about 0.025 mg/container to about 0.075 mg/container, or between about 0.030 mg/container to about 0.060 mg/container.

When a fill volume of about 0.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a surfactant (e.g. PS80) of between about 0.010 mg/container to about 0.100 mg/container, such as between about 0.010 mg/container to about 0.075 mg/container, between about 0.025 mg/container to about 0.075 mg/container, or between about 0.030 mg/container to about 0.060 mg/container. When a fill volume of about 1.0 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a surfactant (e.g. PS80) of between about 0.010 mg/container to about 0.500 mg/container, such as between about 0.010 mg/container to about 0.250 mg/container, between about 0.025 mg/container to about 0.250 mg/container, between about 0.050 mg/container to about 0.200 mg/container, or between about 0.075 mg/container to about 0.175 mg/container. When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a surfactant (e.g. PS80) of between about 0.010 mg/container to about 1.000 mg/container, such as between about 0.010 mg/container to about 0.500 mg/container, between about 0.025 mg/container to about 0.250 mg/container, between about 0.050 mg/container to about 0.200 mg/container, or between about 0.075 mg/container to about 0.175 mg/container.

The amount of surfactant (e.g. PS80) in a solid formulation of the invention may be selected to give a desired concentration on reconstitution to a specified final volume. Thus, a solid formulation of the invention may comprise a surfactant (e.g. PS80) sufficient to give rise to a concentration of between about 0.001 mM to about 1.000 mM, such as between about 0.010 mM to about 1.000 mM, between about 0.010 mM to about 0.50 mM, between about 0.010 mM and about 0.100 mM, between about 0.025 mM and about 0.100 mM, between about 0.050 mM and about 0.100 mM, or between about 0.060 mM and about 0.090 mM following reconstitution, preferably between about 0.050 mM and about 0.100 mM, such as between about 0.050 mM and about 0.090 mM or between about 0.060 mM and between about 0.090 mM. Preferably a solid formulation of the invention may comprise a surfactant (e.g. PS80) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention, such as a pre-lyophilisation solution, may comprise a relative weight amount of a surfactant (e.g. PS80) of between about 0.001% to about 1.000% (w/w), such as between about 0.001% to about 0.100% (w/w), between about 0.001% to about 0.750% (w/w), between about 0.001% to about 0.500% (w/w), between about 0.001% to about 0.100% (w/w), between about 0.0025% to about 0.075% (w/w), between about 0.005% to about 0.050% (w/w), between about 0.005% to about 0.025% (w/w) or between about 0.0075% to about 0.025% (w/w). Preferably a liquid composition of the invention may comprise a relative weight amount of a surfactant (e.g. PS80) of between about 0.005% to about 0.050% (w/w), such as between about 0.005% to about 0.025% (w/w), or between about 0.008% to about 0.020% (w/w).

A liquid composition of the invention, such as a pre-lyophilisation solution, may comprise a relative weight amount of a surfactant (e.g. PS80) of between about 0.002% to about 2.000% (w/w), such as between about 0.002% to about 0.200% (w/w), between about 0.002% to about 1.500% (w/w), between about 0.002% to about 1.000% (w/w), between about 0.002% to about 0.200% (w/w), between about 0.0050% to about 0.150% (w/w), between about 0.010% to about 0.1000% (w/w), between about 0.010% to about 0.500% (w/w) or between about 0.0150% to about 0.5000% (w/w). Preferably a liquid composition of the invention may comprise a relative weight amount of a surfactant (e.g. PS80) of between about 0.010% to about 0.100% (w/w), such as between about 0.010% to about 0.500% (w/w), or between about 0.0160% to about 0.400% (w/w). A liquid composition of the invention, such as a pre-lyophilisation solution, may comprise a relative weight amount of a surfactant (e.g. PS80) of about 0.02% (w/w).

A liquid composition of the invention, such as a pre-lyophilisation solution, may comprise a surfactant (e.g. PS80) at a concentration of between about 0.010 mM to about 1.000 mM, such as between about 0.010 mM to about 0.50 mM, between about 0.010 mM and about 0.100 mM, between about 0.025 mM and about 0.100 mM, between about 0.050 mM and about 0.100 mM, or between about 0.060 mM and about 0.090 mM. Preferably a liquid composition of the invention, such as a pre-lyophilisation solution, comprises a surfactant (e.g. PS80) at a concentration of between about 0.050 mM and about 0.100 mM, such as between about 0.050 mM and about 0.090 mM or between about 0.060 mM and about 0.090 mM.

A liquid composition of the invention, such as a pre-lyophilisation solution, may comprise a surfactant (e.g. PS80) at a concentration of between about 0.020 mM to about 2.000 mM, such as between about 0.020 mM to about 1.00 mM, between about 0.020 mM to about 0.200 mM, between about 0.050 mM to about 0.200 mM, between about 0.100 mM to about 0.200 mM, or between about 0.120 mM to about 0.180 mM. Preferably a liquid composition of the invention, such as a pre-lyophilisation solution, comprises a surfactant (e.g. PS80) at a concentration of between about 0.100 mM to about 0.200 mM, such as between about 0.100 mM to about 0180 mM or between about 0.120 mM to about 0.180 mM. A liquid composition of the invention, such as a pre-lyophilisation solution, may comprise a surfactant (e.g. PS80) at a concentration of about 0.160 mM.

A liquid composition of the invention, such as a pre-lyophilisation solution, may comprise a surfactant (e.g. PS80) at a concentration of between about 0.01 mg/mL to about 2.00 mg/mL, such as between about 0.01 mg/mL to about 1.00 mg/mL, between about 0.01 mg/mL to about 0.50 mg/mL, between about 0.05 mg/mL to about 0.50 mg/mL, between about 0.075 mg/mL to about 0.250 mg/mL, between about 0.08 mg/mL to about 0.20 mg/mL, or between about 0.09 mg/mL to about 0.15 mg/mL. Preferably a liquid composition of the invention, such as a pre-lyophilisation solution, comprises a surfactant (e.g. PS80) at a concentration of between about 0.05 mg/mL to about 0.50 mg/mL, such as between about 0.075 mg/mL to about 0.250 mg/mL, or between about 0.08 mg/mL to about 0.20 mg/mL.

A liquid composition of the invention, such as a pre-lyophilisation solution, may comprise a surfactant (e.g. PS80) at a concentration of between about 0.02 mg/mL to about 4.00 mg/mL, such as between about 0.02 mg/mL to about 2.00 mg/mL, between about 0.02 mg/mL to about 1.00 mg/mL, between about 0.10 mg/mL to about 1.00 mg/mL, between about 0.15 mg/mL to about 0.50 mg/mL, between about 0.16 mg/mL to about 0.40 mg/mL, or between about 0.18 mg/mL to about 0.30 mg/mL. Preferably a liquid composition of the invention, such as a pre-lyophilisation solution, comprises a surfactant (e.g. PS80) at a concentration of between about 0.10 mg/mL to about 1.00 mg/mL, such as between about 0.15 mg/mL to about 0.500 mg/mL, or between about 0.16 mg/mL to about 0.40 mg/mL. A liquid composition of the invention, such as a pre-lyophilisation solution, may comprise a surfactant (e.g. PS80) at a concentration of about 0.20 mg/mL.

### Tonicity Agents

As described herein, compositions of the invention may comprise a tonicity agent. Non-limiting examples of tonicity agents include sodium chloride (NaCl, e.g. NaCl_{aq}, also referred to as saline), glycerin, mannitol, dextrose and trehalose.

A solid composition of the invention may comprise a relative weight amount of a tonicity agent (e.g. NaCl) of between about 0.5% to about 20.0%, such as between about 0.5% to about 10.0% (w/w), between about 0.5% to about 7.5% (w/w), between about 1.0% to about 7.5% (w/w), between about 2.5% to about 7.5% (w/w), between about 2% to about 7% (w/w), between about 2.5% to about 6.0% (w/w), or between about 3.0% to about 5.0% (w/w). Preferably a solid composition of the invention may comprise a relative weight amount of a tonicity agent (e.g. NaCl) of between about 2.5% to about 7.5% (w/w), such as between about 2.5% to about 6.0% (w/w), or between about 3.0% to about 5.0% (w/w).

A solid formulation of the invention may comprise an absolute weight of a tonicity agent (e.g. NaCl) of between about 0.010 mg/container to about 1.50 mg/container, between about 0.010 mg/container to about 1.00 mg/container, such as between about 0.010 mg/container to about 0.500 mg/container, between about 0.100 mg/container to about 0.500 mg/container, between about 0.150 mg/container to about 0.500 mg/container, between about 0.200 mg/container to about 0.500 mg/container, between about 0.200 mg/container to about 0.400 mg/container, or between about 0.250 mg/container to about 0.350 mg/container. The absolute amount of a tonicity agent (e.g. NaCl) in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a tonicity agent (e.g. NaCl) of between about 0.010 mg/container to about 0.500 mg/container, such as about between about 0.20 mg/container to about 0.50 mg/container, or between about 0.20 mg/container to about 0.40 mg/container.

When a fill volume of about 0.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a tonicity agent (e.g. NaCl) of between about 0.010 mg/container to about 0.500 mg/container, such as about between about 0.20 mg/container to about 0.50 mg/container, or between about 0.20 mg/container to about 0.40 mg/container. When a fill volume of about 1.0 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a tonicity agent (e.g. NaCl) of between about 0.020 mg/container to about 1.000 mg/container, such as about between about 0.40 mg/container to about 1.00 mg/container, about between about 0.50 mg/container to about 1.00 mg/container or between about 0.75 mg/container to about 1.00 mg/container. When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a tonicity agent (e.g. NaCl) of between about 0.50 mg/container to about 1.50 mg/container, such as between about 0.75 mg/container to about 1.50 mg/container, between about 0.75 mg/container to about 1.25 mg/container, or between about 0.75 mg/container to about 1.00 mg/container.

The amount of tonicity agent (e.g. NaCl) in a solid formulation of the invention may be selected to give a desired concentration on reconstitution to a specified final volume. Thus, a solid formulation of the invention may comprise a tonicity agent (e.g. NaCl) sufficient to give rise to a concentration of between about 1.0 mM to about 170.0 mM, such as between about 1.0 mM to about 20.0 mM, between about 1.0 mM to about 15.0 mM, between about 2.5 mM and about 12.5 mM, between about 5.0 mM and about 12.5 mM, between about 7.5 mM and about 12.5 mM, or between about 9.0 mM and about 11.0 mM following reconstitution, preferably between about 7.5 mM and about 12.5 mM, such as between about 9.0 mM and about 11.0 mM. Preferably a solid formulation of the invention may comprise a tonicity agent (e.g. NaCl) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention, such as a pre-lyophilisation solution, may comprise a relative weight amount of a tonicity agent (e.g. NaCl) of between about 0.01% to about 0.50% (w/w), such as between about 0.01% to about 0.10% (w/w), between about 0.01% to about 0.075% (w/w), between about 0.020% to about 0.075% (w/w), or between about 0.04% to about 0.070% (w/w). Preferably a liquid composition of the invention may comprise a relative weight amount of a tonicity agent (e.g. NaCl) of between about 0.02% to about 0.075% (w/w), such as between about 0.04% to about 0.070% (w/w).

A liquid composition of the invention, such as a pre-lyophilisation solution, may comprise a relative weight amount of a tonicity agent (e.g. NaCl) of between about 0.02% to about 1.00% (w/w), such as between about 0.02% to about 0.20% (w/w), between about 0.02% to about 0.150% (w/w), between about 0.04% to about 0.150% (w/w), or between about 0.08% to about 0.140% (w/w). Preferably a liquid composition of the invention may comprise a relative weight amount of a tonicity agent (e.g. NaCl) of between about 0.04% to about 0.150% (w/w), such as between about 0.08% to about 0.140% (w/w). A liquid composition of the invention, such as a pre-lyophilisation solution, may comprise a relative weight amount of a tonicity agent (e.g. NaCl) of about 0.116% (w/w).

A liquid composition of the invention, such as a pre-lyophilisation solution, may comprise a tonicity agent (e.g. NaCl) at a concentration of between about 1.0 mM to about 20.0 mM, such as between about 1.0 mM to about 15.0 mM, between about 2.5 mM and about 12.5 mM, between about 5.0 mM and about 12.5 mM, between about 7.5 mM and about 12.5 mM, or between about 9.0 mM and about 11.0 mM. Preferably a liquid composition of the invention, such as a pre-lyophilisation solution, comprises a tonicity agent (e.g. NaCl) at a concentration of between about 7.5 mM and about 12.5 mM, such as between about 9.0 mM and about 11.0 mM.

A liquid composition of the invention, such as a pre-lyophilisation solution, may comprise a tonicity agent (e.g. NaCl) at a concentration of between about 2.0 mM to about 40.0 mM, such as between about 2.0 mM to about 30.0 mM, between about 5.0 mM and about 25.0 mM, between about 10.0 mM and about 25.0 mM, between about 15.0 mM and about 25.0 mM, or between about 18.0 mM and about 22.0 mM. Preferably a liquid composition of the invention, such as a pre-lyophilisation solution, comprises a tonicity agent (e.g. NaCl) at a concentration of between about 15.0 mM and about 25.0 mM, such as between about 18.0 mM and about 22.0 mM. A liquid composition of the invention, such as a pre-lyophilisation solution, may comprise a tonicity agent (e.g. NaCl) at a concentration of about 20.0 mM.

A liquid composition of the invention, such as a pre-lyophilisation solution, may comprise a tonicity agent (e.g. NaCl) at a concentration of between about 0.1 mg/mL to about 5.0 mg/mL, such as between about 0.1 mg/mL to about 1.0 mg/mL, between about 0.1 mg/mL to about 0.75 mg/mL, between about 0.20 mg/mL to about 0.75 mg/mL, or between about 0.40 mg/mL to about 0.60 mg/mL. Preferably a liquid composition of the invention, such as a pre-lyophilisation solution, comprises a tonicity agent (e.g. NaCl) at a concentration of between about 0.2 mg/mL to about 0.75 mg/mL, such as between about 0.40 mg/mL to about 0.60 mg/mL.

A liquid composition of the invention, such as a pre-lyophilisation solution, may comprise a tonicity agent (e.g. NaCl) at a concentration of between about 0.20 mg/mL to about 10.0 mg/mL, such as between about 0.20 mg/mL to about 2.0 mg/mL, between about 0.20 mg/mL to about 1.50 mg/mL, between about 0.40 mg/mL to about 1.50 mg/mL, or between about 0.80 mg/mL to about 1.20 mg/mL. Preferably a liquid composition of the invention, such as a pre-lyophilisation solution, comprises a tonicity agent (e.g. NaCl) at a concentration of between about 0.40 mg/mL to about 1.50 mg/mL, such as between about 0.80 mg/mL to about 1.20 mg/mL. A liquid composition of the invention, such as a pre-lyophilisation solution, may comprise a tonicity agent (e.g. NaCl) at a concentration of about 1.16 mg/mL.

A liquid composition of the invention, such as a post-reconstitution solution, may comprise a tonicity agent (e.g. NaCl) at a concentration of between about 5.8 mg/mL to about 11.7 mg/mL, such as between about 7.3 mg/mL to about 11.7 mg/mL, between about 7.3 mg/mL to about 10.2 mg/mL, between about 8.8 mg/mL to about 10.2 mg/mL, between about 9.1 mg/mL to about 10.2 mg/mL, between about 9.4 mg/mL to about 10.2 mg/mL, between about 9.4 mg/mL to about 9.9 mg/mL, or between about 9.4 mg/mL to about 9.6 mg/mL. Preferably a liquid composition of the invention, such as a post-reconstitution solution, comprises a tonicity agent (e.g. NaCl) at a concentration of between about 9.4 mg/mL to about 9.9 mg/mL (e.g. about 9.4 mg/mL, about 9.5 mg/mL, about 9.6 mg/mL, about 9.7 mg/mL, about 9.8 mg/mL or about 9.9 mg/mL), such as between about 9.4 mg/mL to about 9.6 mg/mL (e.g. about 9.4 mg/mL, about 9.5 mg/mL or about 9.6 mg/mL), particularly about 9.58 mg/mL.

A liquid composition of the invention, such as a post-reconstitution solution, may comprise a tonicity agent (e.g. NaCl) at a concentration of between about 100 mM to about 200 mM, such as between about 125 mM to about 200 mM, between about 125 mM and about 175 mM, between about 150 mM and about 175 mM, between about 155 mM and about 175 mM, between about 160 mM and about 175 mM, between about 160 mM and about 170 mM, or between about 160 mM and about 165 mM. Preferably a liquid composition of the invention, such as a post-reconstitution solution, comprises a tonicity agent (e.g. NaCl) at a concentration of between about 160 mM and about 170 mM (e.g. about 160 mM, about 161 mM, about 162 mM, about 163 mM, about 164 mM, about 165 mM, about 166 mM, about 167 mM, about 168 mM, about 169 mM or about 170 mM), such as between about 160 mM and about 165 mM mM (e.g. about 160 mM, about 161 mM, about 162 mM, about 163 mM, about 164 mM or about 165 mM). Preferably a liquid composition of the invention, such as a post-reconstitution solution, comprises a tonicity agent (e.g. NaCl) at a concentration of between about 150 mM and about 165 mM (e.g. about 150 mM, about 151 mM, about 152 mM, about 153 mM, about 154 mM, about 155 mM, about 156 mM, about 157 mM, about 158 mM, about 159 mM, about 160 mM about 161 mM, about 162 mM, about 163 mM, about 164 mM, or about 165 mM), such as between about 160 mM and about 165 mM mM (e.g. about 160 mM, about 161 mM, about 162 mM, about 163 mM, about 164 mM or about 165 mM).

### Chimeric Clostridial Neurotoxins

A composition of the invention may comprise any chimeric clostridial neurotoxin, particularly a chimeric BONT, as described herein.

In some preferred embodiments, a composition of the invention comprises a chimeric clostridial neurotoxin which comprises at least one domain from a BoNT/A and at least one domain from a non-BoNT/A clostridial neurotoxin (i.e. a BoNT/A chimera). Examples of BoNT/A chimeras are described herein. In particular, a composition of the invention may comprise a BoNT/AB chimera, such as a chimeric BONT which comprises a BoNT/A L-chain and H_{N} domain and a BoNT/B H_{C} domain (such as LH_{N}/A1-H_{C}/B1). An exemplary LH_{N}/A1-H_{C}B1 chimera is given in SEQ ID NO: 1.

A di-chain chimeric clostridial neurotoxin of the invention may comprise an L-chain portion of a polypeptide sequence having at least 70%, 80%, 90%, 95%, 99.9%, or 100% sequence identity to SEQ ID NO: 1 constituting a first chain of the di-chain chimeric clostridial neurotoxin, and may comprise the H_{N} and H_{C} domains of a polypeptide sequence having at least 70%, 80%, 90%, 95%, 99.9%, or 100% sequence identity to SEQ ID NO: 1 together constituting a second chain of the di-chain chimeric clostridial neurotoxin, wherein the first and second chains are joined together by a di-sulphide bond.

Where cleavage occurs at more than one position (preferably at two positions) within the activation loop of a chimeric clostridial neurotoxin comprising a polypeptide sequence having at least 70%, 80%, 90%, 95%, 99.9%, or 100% sequence identity to SEQ ID NO: 1, a small fragment of the C-terminal L-chain portion of the sequence having at least 70%, 80%, 90%, 95%, 99.9%, or 100% sequence identity to SEQ ID NO: 1 may be absent from the di-chain chimeric clostridial neurotoxin. In view of this, the sequence of the di-chain chimeric clostridial neurotoxin (e.g. comprising a polypeptide sequence having at least 70%, 80%, 90%, 95%, 99.9%, or 100% sequence identity to SEQ ID NO: 1) may be slightly different to that of the corresponding single-chain chimeric clostridial neurotoxin comprising a polypeptide sequence having at least 70%, 80%, 90%, 95%, 99.9%, or 100% sequence identity to SEQ ID NO: 1. The small fragment may be 1-15 amino acids. In particular, in one embodiment, when Lys-C is used to covert a single-chain chimeric clostridial neurotoxin into a di-chain clostridial neurotoxin, the small fragment of the C-terminal L-chain portion of the sequence that is absent may be SEQ ID NO: 11 or 12.

In a particularly preferred embodiment, a di-chain chimeric clostridial neurotoxin comprises (or consists of) a light-chain comprising a polypeptide sequence having at least 70%, 80%, 90%, 95%, or 99.9% sequence identity to SEQ ID NO: 13 or 14 (preferably SEQ ID NO: 13) and a heavy-chain comprising a polypeptide sequence having at least 70%, 80%, 90%, 95%, or 99.9% sequence identity to SEQ ID NO: 15, wherein the light-chain and heavy-chain are joined together by a di-sulphide bond. More preferably, a di-chain chimeric clostridial neurotoxin comprises (or consists of) a light-chain comprising SEQ ID NO: 13 or 14 (preferably SEQ ID NO: 13) and a heavy-chain comprising SEQ ID NO: 15, wherein the light-chain and heavy-chain are joined together by a di-sulphide bond. Even more preferably, a di-chain chimeric clostridial neurotoxin comprises (or consists of) a light-chain having SEQ ID NO: 13 and a heavy-chain having SEQ ID NO: 15, wherein the light-chain and heavy-chain are joined together by a di-sulphide bond. The di-sulphide bond is preferably formed by and/or is between cysteine residue 429 of SEQ ID NO: 13 or 14 and cysteine residue 6 of SEQ ID NO: 15.

Any appropriate amount of chimeric clostridial neurotoxin (e.g. BoNT/AB chimera) may be comprised in a composition of the invention. Typically, a composition of the invention comprises an amount of chimeric clostridial neurotoxin (e.g. BoNT/AB chimera) sufficient to provide a therapeutically or cosmetically effective dose of the chimeric clostridial neurotoxin (e.g. BoNT/AB chimera) when administered to an individual. Thus, the amount of chimeric clostridial neurotoxin (e.g. BoNT/AB chimera) within a composition of the invention may depend on the intended therapeutic or cosmetic use of the composition.

A solid composition of the invention may comprise an absolute weight of a chimeric clostridial neurotoxin (e.g. BoNT/AB chimera) of between about 0.05 ng/container and about 500 ng/container, such as between about 0.05 ng/container and about 300 ng/container, between about 0.05 ng/container and about 250 ng/container, between about 0.10 ng/container and about 250 ng/container, between about 0.05 ng/container and about 500 ng/container, between about 0.10 ng/container and about 100 ng/container, between about 0.10 ng/container and about 20 ng/container, between about 1.0 ng/container and about 100 ng/container, between about 5 ng/container and about 75 ng/container, between about 5 ng/container and about 50 ng/container, between about 5 ng/container and about 40 ng/container, between about 50 ng/container and about 300 ng/container, between about 50 ng/container and about 250 ng/container, between about 50 ng/container and about 200 ng/container, between about 50 ng/container and about 150 ng/container, between about 75 ng/container and about 300 ng/container, between about 75 ng/container and about 250 ng/container, between about 75 ng/container and about 200 ng/container, between about 75 ng/container and about 200 ng/container, between about 90 ng/container and about 300 ng/container, between about 90 ng/container and about 250 ng/container, between about 90 ng/container and about 200 ng/container, or between about 90 ng/container and about 150 ng/container. In some preferred embodiments, a solid composition of the invention may comprise between about 90 ng/container and about 200 ng/container of a chimeric clostridial neurotoxin (e.g. BoNT/AB chimera). The absolute amount of a chimeric clostridial neurotoxin (e.g. BoNT/AB chimera) in a solid composition will typically depend on the volume of the composition used to fill a given container, the intended reconstitution volume and/or on the intended field of use (e.g. therapeutic or cosmetic/aesthetic).

A solid composition of the invention may comprise an absolute weight of a chimeric clostridial neurotoxin (e.g. BoNT/AB chimera) at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container.

In some embodiments, a solid composition of the invention may comprise an absolute weight of a chimeric clostridial neurotoxin (e.g. BoNT/AB chimera) of between about 50 ng/container to about 500 ng/container.

In some preferred embodiments, a solid composition of the invention may comprise an absolute weight of a chimeric clostridial neurotoxin (e.g. BoNT/AB chimera) of between about 50 ng/container to about 300 ng/container.

In some embodiments, a solid composition of the invention may comprise an absolute weight of a chimeric clostridial neurotoxin (e.g. BoNT/AB chimera) of between about 50 ng/container to about 200 ng/container.

In some preferred embodiments, a solid composition of the invention may comprise an absolute weight of a chimeric clostridial neurotoxin (e.g. BoNT/AB chimera) of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container.

In some preferred embodiments, a solid composition of the invention may comprise an absolute weight of a chimeric clostridial neurotoxin (e.g. BoNT/AB chimera) of between about 50 ng/container to about 450 ng/container, between about 100 ng/container to about 400 ng/container, between about 150 ng/container to about 350 ng/container, particularly between about 200 ng/container to about 300 ng/container.

In some particularly preferred embodiments, a solid composition of the invention may comprise an absolute weight of a chimeric clostridial neurotoxin (e.g. BoNT/AB chimera) of between about 90 ng/container to about 200 ng/container.

In some particularly preferred embodiments, a solid composition of the invention may comprise an absolute weight of a chimeric clostridial neurotoxin (e.g. BoNT/AB chimera) of between about 200 ng/container to about 300 ng/container.

The absolute amount of a chimeric clostridial neurotoxin (e.g. BoNT/AB chimera) in a solid composition will typically depend on the volume of the composition used to fill a given container, the intended reconstitution volume and/or on the intended field of use (e.g. therapeutic or cosmetic/aesthetic).

By way of non-limiting example, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a chimeric clostridial neurotoxin (e.g. BoNT/AB chimera) between about 0.05 ng/container and about 300 ng/container, such as between about 0.05 ng/container and about 250 ng/container, between about 0.05 ng/container and about 200 ng/container, between about 0.05 ng/container and about 150 ng/container. between about 0.05 ng/container and about 100 ng/container, between about 50 ng/container and about 300 ng/container, between about 50 ng/container and about 250 ng/container, between about 50 ng/container and about 200 ng/container, between about 50 ng/container and about 150 ng/container, between about 50 ng/container and about 100 ng/container, between about 0.10 ng/container and about 50 ng/container, between about 1.0 ng/container and about 50 ng/container, between about 2.5 ng/container and about 50 ng/container, between about 5 ng/container and about 40 ng/container. In particular, when the solid composition of the invention is intended for reconstitution in a volume of about 1 mL, the absolute amount of a chimeric clostridial neurotoxin (e.g. BoNT/AB chimera) in a solid composition may be between about 0.05 ng/container and about 300 ng/container, such as between about 0.05 ng/container and about 250 ng/container, between about 0.05 ng/container and about 200 ng/container, between about 0.05 ng/container and about 150 ng/container, between about 0.05 ng/container and about 100 ng/container, between about 50 ng/container and about 300 ng/container, between about 50 ng/container and about 250 ng/container, between about 50 ng/container and about 200 ng/container, between about 50 ng/container and about 150 ng/container, between about 50 ng/container and about 100 ng/container, between about 0.10 ng/container and about 50 ng/container, between about 1 ng/container and about 50 ng/container, preferably between about 2 ng/container and about 25 ng/container, or between about 2.5 ng/container and about 20 ng/container. When the solid composition of the invention is intended for reconstitution in a volume of about 2 mL, the absolute amount of a chimeric clostridial neurotoxin (e.g. BoNT/AB chimera) in a solid composition may be between about 0.05 ng/container and about 300 ng/container, such as between about 0.05 ng/container and about 250 ng/container, between about 0.05 ng/container and about 200 ng/container, between about 0.05 ng/container and about 150 ng/container, between about 0.05 ng/container and about 100 ng/container. between about 50 ng/container and about 300 ng/container, between about 50 ng/container and about 250 ng/container, between about 50 ng/container and about 200 ng/container, between about 50 ng/container and about 150 ng/container, between about 50 ng/container and about 100 ng/container, between about 5 ng/container and about 100 ng/container, preferably between about 5 ng/container and about 75 ng/container, such as between about 5 ng/container and about 65 ng/container, or between about 7.5 ng/container and about 65 ng/container. In some preferred embodiments, a solid composition of the invention may comprise between about 90 ng/container and about 200 ng/container a chimeric clostridial neurotoxin (e.g. BoNT/AB chimera). Such solid compositions may be particularly intended for cosmetic use.

By way of further non-limiting example, when a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a chimeric clostridial neurotoxin (e.g. BoNT/AB chimera) between about 0.05 ng/container and about 500 ng/container, such as between about 0.05 ng/container and about 250 ng/container, between about 0.05 ng/container and about 200 ng/container, between about 0.05 ng/container and about 150 ng/container. between about 0.05 ng/container and about 100 ng/container, between about 50 ng/container and about 300 ng/container, between about 50 ng/container and about 250 ng/container, between about 50 ng/container and about 200 ng/container, between about 50 ng/container and about 150 ng/container, between about 50 ng/container and about 100 ng/container, between about 1 ng/container and about 300 ng/container, between about 1.0 ng/container and about 200 ng/container, between about 2.5 ng/container and about 100 ng/container, between about 5 ng/container and about 75 ng/container. In particular, when the solid composition of the invention is intended for reconstitution in a volume of about 1.5 mL, the absolute amount of a chimeric clostridial neurotoxin (e.g. BoNT/AB chimera) in a solid composition may be between about 0.05 ng/container and about 500 ng/container, such as between about 0.05 ng/container and about 250 ng/container, between about 0.05 ng/container and about 200 ng/container, between about 0.05 ng/container and about 150 ng/container. between about 0.05 ng/container and about 100 ng/container, between about 50 ng/container and about 300 ng/container, between about 50 ng/container and about 250 ng/container, between about 50 ng/container and about 200 ng/container, between about 50 ng/container and about 150 ng/container, between about 50 ng/container and about 100 ng/container, between about 1 ng/container and about 300 ng/container, between about 1.0 ng/container and about 200 ng/container, preferably between about 2.5 ng/container and about 100 ng/container, or between about 5 ng/container and about 75 ng/container. In some preferred embodiments, a solid composition of the invention may comprise between about 90 ng/container and about 200 ng/container of a chimeric clostridial neurotoxin (e.g. BoNT/AB chimera). Such solid compositions may be particularly intended for therapeutic use.

By way of non-limiting example, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a chimeric clostridial neurotoxin (e.g. BoNT/AB chimera) between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container.

By way of further non-limiting example, when a fill volume of about 1 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a chimeric clostridial neurotoxin (e.g. BoNT/AB chimera) between about 50 ng/container to about 450 ng/container, between about 100 ng/container to about 400 ng/container, between about 150 ng/container to about 350 ng/container, particularly between about 200 ng/container to about 300 ng/container.

By way of further non-limiting example, when a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of a chimeric clostridial neurotoxin (e.g. BoNT/AB chimera) between about 50 ng/container to about 450 ng/container, between about 100 ng/container to about 400 ng/container, between about 150 ng/container to about 350 ng/container, particularly between about 200 ng/container to about 300 ng/container.

The amount of chimeric clostridial neurotoxin (e.g. BoNT/AB chimera) in a solid formulation of the invention may be selected to give a desired concentration on reconstitution to a specified final volume. Thus, a solid formulation of the invention may comprise a chimeric clostridial neurotoxin (e.g. BoNT/AB chimera) sufficient to give rise to a concentration of between about 0.05 ng/ml and about 500 ng/mL, such as between about 0.05 ng/ml and about 250 ng/mL, between about 0.05 ng/mL and about 200 ng/mL, between about 0.05 ng/mL and about 150 ng/mL, between about 0.05 ng/ml and about 100 ng/ml, between about 50 ng/ml and about 300 ng/mL, between about 50 ng/mL and about 250 ng/mL, between about 50 ng/mL and about 200 ng/mL, between about 50 ng/mL and about 150 ng/container, between about 50 ng/ml and about 100 ng/ml, between about 0.05 ng/ml and about 75 ng/ml, between about 0.05 ng/ml and about 50 ng/mL, between about 0.10 ng/ml and about 50 ng/ml, between about 1 ng/mL and about 50 ng/mL, or between about 2 ng/ml and about 50 ng/mL, preferably between about 2.5 ng/ml and about 30 ng/ml, such as between about 2.5 ng/ml and about 20 ng/mL, or between about 5 ng/mL and about 15 ng/ml. Preferably a solid formulation of the invention may comprise a chimeric clostridial neurotoxin (e.g. BoNT/AB chimera) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. In some preferred embodiments, a solid composition of the invention may give a concentration of a chimeric clostridial neurotoxin (e.g. BoNT/AB chimera) between about 90 ng/ml and about 200 ng/mL on reconstitution.

A liquid composition of the invention, such as a pre-lyophilisation solution, may comprise a chimeric clostridial neurotoxin (e.g. BoNT/AB chimera) at a concentration of between about 0.1 ng/mL and about 500 ng/mL, such as between about 0.05 ng/mL and about 250 ng/ml, between about 0.05 ng/mL and about 200 ng/mL, between about 0.05 ng/mL and about 150 ng/ml, between about 0.05 ng/ml and about 100 ng/ml, between about 50 ng/ml and about 300 ng/mL, between about 50 ng/mL and about 250 ng/mL, between about 50 ng/mL and about 200 ng/mL, between about 50 ng/ml and about 150 ng/container, between about 50 ng/mL and about 100 ng/ml, between about 0.1 ng/ml and about 150 ng/ml, between about 1 ng/ml and about 100 ng/mL, between about 5 ng/ml and about 100 ng/ml, between about 5 ng/mL and about 75 ng/mL, or between about 5 ng/ml and about 65 ng/mL. Preferably a liquid composition of the invention, such as a pre-lyophilisation solution, comprises a chimeric clostridial neurotoxin (e.g. BoNT/AB chimera) at a concentration of between about 5 ng/ml and about 75 ng/mL, such as between about 5 ng/ml and about 65 ng/ml, or between about 10 ng/mL and about 60 ng/mL. In some preferred embodiments, a liquid composition of the invention has a concentration of a chimeric clostridial neurotoxin (e.g. BoNT/AB chimera) of between about 90 ng/ml and about 200 ng/mL.

### Solid Compositions

In some preferred embodiments, the compositions of the invention are solid compositions. Particularly, the invention provides lyophilised compositions of chimeric clostridial neurotoxins.

The solid compositions of the invention may therefore be in the form of lyophilised powder. Alternatively, the solid compositions of the invention may be in the form of vacuum-dried powder. Methods of lyophilisation and vacuum drying are well-known in the field of pharmaceutical formulation and within the routine skill of one of ordinary skill in the art.

The solid composition may be packaged as a unit dose. The term “unit dose” as used herein refers to an amount of a composition that provide exactly one dose. For the chimeric clostridial neurotoxins of the invention, said one dose may be administered to an individual by a single injection, or by multiple injections (e.g. between about 2 to 10 injections), in a single administration, optionally to the same target site or location. Preferably, however, the solid composition is packaged based on the amount (particularly the absolute weight) of the chimeric clostridial neurotoxin of the invention, as described herein.

Non-limiting examples of stabilising agents, surfactants and tonicity agents are described above, together with non-limiting examples of suitable concentrations of dual histidine buffers, stabilising agents, surfactants and tonicity agents useful in solid compositions of the invention. Each of these components and the concentration thereof for a solid composition may be independently selected from the disclosure above.

The invention provides a solid composition which comprises or consists of a chimeric clostridial neurotoxin (e.g. a chimeric BoNT), a dual histidine buffer, a stabilising agent, a surfactant and a tonicity agent. The dual histidine buffer typically comprises or consists of L-histidine and histidine-HCl, as described herein. The stabilising agent may be sucrose. The surfactant is typically a non-ionic surfactant, such as PS80 or poloxamer-188. The tonicity agent may be NaCl. Thus, the invention provides a solid composition which comprises or consists of a chimeric clostridial neurotoxin (e.g. a chimeric BoNT), L-histidine and histidine-HCl, sucrose, PS80 or poloxamer-188 and NaCl. In some preferred embodiments, the chimeric BONT is a BoNT/AB chimera as described herein.

Preferred examples of solid compositions of the invention are described below.

A solid composition of the invention may comprise or consist of: (i) a chimeric clostridial neurotoxin, particularly a chimeric BoNT, preferably a BoNT/AB chimera, at an absolute weight of between about 0.05 ng/container and about 500 ng/container (e.g. between about 50 ng/container and about 250 ng/container); (ii) L-histidine at a relative weight a relative weight amount of between about 10% to about 20% (w/w); (iii) histidine-HCl at a relative weight a relative weight amount of between about 5% to about 10% (w/w); (iv) sucrose at a relative weight amount of between about 70% to about 80% (w/w); (v) PS80 or poloxamer-188 (preferably PS80) at a relative weight amount of between about 0.5% to about 1.0% (w/w); and/or (vi) NaCl at a relative weight amount of between about 2% to about 7% (w/w).

In some preferred embodiments, a solid composition of the invention may comprise or consist of: (i) a chimeric clostridial neurotoxin, particularly a chimeric BoNT, preferably a BoNT/AB chimera, at an absolute weight of between about 5 ng/container and about 250 ng/container (e.g. between about 50 ng/container and about 250 ng/container, particularly between about 90 ng/container and about 200 ng/container); (ii) L-histidine at a relative weight amount of between about 16% to about 17% (w/w); (iii) histidine-HCl at a relative weight amount of between about 7.5% to about 8.0% (w/w); (iv) sucrose at a relative weight amount of between about 70% to about 72% (w/w); (v) PS80 or poloxamer-188 (preferably PS80) at a relative weight amount of between about 0.70% to about 0.75% (w/w); and/or (vi) NaCl at a relative weight amount of between about 4.0% to about 5.0% (w/w).

A solid composition of the invention may comprise or consist of: (i) a chimeric clostridial neurotoxin, particularly a chimeric BoNT, preferably a BoNT/AB chimera, at an absolute weight of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a relative weight amount of between about 16% to about 17% (w/w); (iii) histidine-HCl at a relative weight amount of between about 7.5% to about 8.0% (w/w); (iv) sucrose at a relative weight amount of between about 70% to about 72% (w/w); (v) PS80 or poloxamer-188 (preferably PS80) at a relative weight amount of between about 0.70% to about 0.75% (w/w); and/or (vi) NaCl at a relative weight amount of between about 4.0% to about 5.0% (w/w).

A solid composition of the invention may comprise or consist of: (i) a chimeric clostridial neurotoxin, particularly a chimeric BoNT, preferably a BoNT/AB chimera, at an absolute weight of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a relative weight amount of about 16.2% (w/w); (iii) histidine-HCl at a relative weight a relative weight amount of about 7.9% (w/w); (iv) sucrose at a relative weight amount of about 71.1% (w/w); (v) PS80 or poloxamer-188 (preferably PS80) at a relative weight amount of about 0.7% (w/w); and/or (vi) NaCl at a relative weight amount of about 4.1% (w/w).

A solid composition of the invention may comprise or consist of: (i) a chimeric clostridial neurotoxin, particularly a chimeric BONT, preferably a BoNT/AB chimera, at an absolute weight of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a relative weight amount of between about 16% to about 17% (w/w); (iii) histidine-HCl at a relative weight amount of between about 7.5% to about 8.0% (w/w); (iv) sucrose at a relative weight amount of between about 70% to about 72% (w/w); (v) PS80 or poloxamer-188 (preferably PS80) at a relative weight amount of between about 0.70% to about 0.75% (w/w); and/or (vi) NaCl at a relative weight amount of between about 4.0% to about 5.0% (w/w).

A solid composition of the invention may comprise or consist of: (i) a chimeric clostridial neurotoxin, particularly a chimeric BoNT, preferably a BoNT/AB chimera, at an absolute weight of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a relative weight amount of about 16.2% (w/w); (iii) histidine-HCl at a relative weight a relative weight amount of about 7.9% (w/w); (iv) sucrose at a relative weight amount of about 71.1% (w/w); (v) PS80 or poloxamer-188 (preferably PS80) at a relative weight amount of about 0.7% (w/w); and/or (vi) NaCl at a relative weight amount of about 4.1% (w/w).

A solid composition of the invention may comprise or consist of: (i) a chimeric clostridial neurotoxin, particularly a chimeric BONT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a relative weight amount of between about 16% to about 17% (w/w); (iii) histidine-HCl at a relative weight amount of between about 7.5% to about 8.0% (w/w); (iv) sucrose at a relative weight amount of between about 70% to about 72% (w/w); (v) PS80 or poloxamer-188 (preferably PS80) at a relative weight amount of between about 0.70% to about 0.75% (w/w); and/or (vi) NaCl at a relative weight amount of between about 4.0% to about 5.0% (w/w).

A solid composition of the invention may comprise or consist of: (i) a chimeric clostridial neurotoxin, particularly a chimeric BoNT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a relative weight amount of about 16.2% (w/w); (iii) histidine-HCl at a relative weight a relative weight amount of about 7.9% (w/w); (iv) sucrose at a relative weight amount of about 71.1% (w/w); (v) PS80 or poloxamer-188 (preferably PS80) at a relative weight amount of about 0.7% (w/w); and/or (vi) NaCl at a relative weight amount of about 4.1% (w/w).

A solid composition of the invention may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at an absolute weight of between about 1.0 mg/container to about 3.5 mg/container; (iii) histidine-HCl at an absolute weight amount of between about 0.25 mg/container to about 2.00 mg/container; (iv) sucrose at an absolute weight amount of between about 1.0 mg/container to about 15.0 mg/container; (v) PS80 or poloxamer-188 (preferably PS80) at an absolute weight amount of between about 0.025 mg/container to about 0.200 mg/container; and/or (vi) NaCl at an absolute weight amount of between about 0.20 mg/container to about 1.00 mg/container.

A solid composition of the invention may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container (ii) L-histidine at an absolute weight of about 1.14 mg/container; (iii) histidine-HCl at an absolute weight amount of about 0.555 mg/container; (iv) sucrose at an absolute weight amount of about 5 mg/container; (v) PS80 or poloxamer-188 (preferably PS80) at an absolute weight amount of about 0.05 mg/container; and/or (vi) NaCl at an absolute weight amount of about 0.29 mg/container.

A solid composition of the invention may comprise or consist of: (i) a chimeric clostridial neurotoxin, particularly a chimeric BoNT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at an absolute weight of between about 1.0 mg/container to about 3.5 mg/container; (iii) histidine-HCl at an absolute weight amount of between about 0.25 mg/container to about 2.00 mg/container; (iv) sucrose at an absolute weight amount of between about 1.0 mg/container to about 15.0 mg/container; (v) PS80 or poloxamer-188 (preferably PS80) at an absolute weight amount of between about 0.025 mg/container to about 0.200 mg/container; and/or (vi) NaCl at an absolute weight amount of between about 0.20 mg/container to about 1.00 mg/container.

A solid composition of the invention may comprise or consist of: (i) a chimeric clostridial neurotoxin, particularly a chimeric BONT, preferably a BoNT/AB chimera, at a concentration of between about between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at an absolute weight of about 1.14 mg/container; (iii) histidine-HCl at an absolute weight amount of about 0.555 mg/container; (iv) sucrose at an absolute weight amount of about 5 mg/container; (v) PS80 or poloxamer-188 (preferably PS80) at an absolute weight amount of about 0.05 mg/container; and/or (vi) NaCl at an absolute weight amount of about 0.29 mg/container.

A solid composition of the invention may comprise or consist of: (i) a chimeric clostridial neurotoxin, particularly a chimeric BoNT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of between about 1.0 mg/container to about 3.5 mg/container; (iii) histidine-HCl at an absolute weight amount of between about 0.25 mg/container to about 2.00 mg/container; (iv) sucrose at an absolute weight amount of between about 1.0 mg/container to about 15.0 mg/container; (v) PS80 or poloxamer-188 (preferably PS80) at an absolute weight amount of between about 0.025 mg/container to about 0.200 mg/container; and/or (vi) NaCl at an absolute weight amount of between about 0.20 mg/container to about 1.00 mg/container.

A solid composition of the invention may comprise or consist of: (i) a chimeric clostridial neurotoxin, particularly a chimeric BoNT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of about 1.14 mg/container; (iii) histidine-HCl at an absolute weight amount of about 0.555 mg/container; (iv) sucrose at an absolute weight amount of about 5 mg/container; (v) PS80 or poloxamer-188 (preferably PS80) at an absolute weight amount of about 0.05 mg/container; and/or (vi) NaCl at an absolute weight amount of about 0.29 mg/container.

A solid composition of the invention may comprise or consist of: (i) a chimeric clostridial neurotoxin, particularly a chimeric BoNT, preferably a BoNT/AB chimera, at an absolute weight of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at an absolute weight of between about 0.8 mg/container to about 1.5 mg/container; (iii) histidine-HCl at an absolute weight amount of between about 0.25 mg/container to about 0.75 mg/container; (iv) sucrose at an absolute weight amount of between about 2.0 mg/container to about 8.0 mg/container; (v) PS80 or poloxamer-188 (preferably PS80) at an absolute weight amount of between about 0.025 mg/container to about 0.1 mg/container; and/or (vi) NaCl at an absolute weight amount of between about 0.20 mg/container to about 0.5 mg/container.

A solid composition of the invention may comprise or consist of: (i) a chimeric clostridial neurotoxin, particularly a chimeric BoNT, preferably a BoNT/AB chimera, at an absolute weight of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at an absolute weight of about 1.14 mg/container; (iii) histidine-HCl at an absolute weight amount of about 0.555 mg/container; (iv) sucrose at an absolute weight amount of about 5 mg/container; (v) PS80 or poloxamer-188 (preferably PS80) at an absolute weight amount of about 0.05 mg/container; and/or (vi) NaCl at an absolute weight amount of about 0.29 mg/container. In some embodiments, a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)).

A solid composition of the invention may comprise or consist of: (i) a chimeric clostridial neurotoxin, particularly a chimeric BoNT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of between about 1.0 mg/container to about 3.5 mg/container; (iii) histidine-HCl at an absolute weight amount of between about 0.25 mg/container to about 2.00 mg/container; (iv) sucrose at an absolute weight amount of between about 1.0 mg/container to about 15.0 mg/container; (v) PS80 or poloxamer-188 (preferably PS80) at an absolute weight amount of between about 0.025 mg/container to about 0.200 mg/container; and/or (vi) NaCl at an absolute weight amount of between about 0.20 mg/container to about 1.00 mg/container.

A solid composition of the invention may comprise or consist of: (i) a chimeric clostridial neurotoxin, particularly a chimeric BoNT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of about 2.28 mg/container; (iii) histidine-HCl at an absolute weight amount of about 1.11 mg/container; (iv) sucrose at an absolute weight amount of about 10 mg/container; (v) PS80 or poloxamer-188 (preferably PS80) at an absolute weight amount of about 0.1 mg/container; and/or (vi) NaCl at an absolute weight amount of about 0.58 mg/container. In some embodiments, a fill volume of about 1 mL is used (e.g. to fill a 3 mL container (e.g. 3R vial), 5 mL container (e.g. 5R vial) or 6 mL container (e.g. 6R vial), particularly a 6 mL container (e.g. 6R vial)).

A solid composition of the invention may comprise or consist of: (i) a chimeric clostridial neurotoxin, particularly a chimeric BoNT, preferably a BoNT/AB chimera, at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of about 3.42 mg/container; (iii) histidine-HCl at an absolute weight amount of about 1.665 mg/container; (iv) sucrose at an absolute weight amount of about 15 mg/container; (v) PS80 or poloxamer-188 (preferably PS80) at an absolute weight amount of about 0.15 mg/container; and/or (vi) NaCl at an absolute weight amount of about 0.87 mg/container. In some embodiments, a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)).

In some particularly preferred embodiments, a solid composition of the invention may comprise or consist of: (i) a chimeric clostridial neurotoxin, particularly a chimeric BONT, preferably a BoNT/AB chimera, at an absolute weight of between about 5 ng/container and about 250 ng/container (e.g. between about 50 ng/container and about 250 ng/container, particularly between about 90 ng/container and about 200 ng/container); (ii) L-histidine at a relative weight amount of about 16.2% (w/w); (iii) histidine-HCl at a relative weight a relative weight amount of about 7.9% (w/w); (iv) sucrose at a relative weight amount of about 71.1% (w/w); (v) PS80 or poloxamer-188 (preferably PS80) at a relative weight amount of about 0.7% (w/w); and/or (vi) NaCl at a relative weight amount of about 4.1% (w/w).

A solid composition of the invention may comprise or consist of: (i) a chimeric clostridial neurotoxin, particularly a chimeric BoNT, preferably a BoNT/AB chimera, at an absolute weight of between about 0.05 ng/container and about 500 ng/container (e.g. between about 50 ng/container and about 250 ng/container); (ii) L-histidine at an absolute weight of between about 1.0 mg/container to about 6.0 mg/container; (iii) histidine-HCl at an absolute weight amount of between about 0.25 mg/container to about 3.00 mg/container; (iv) sucrose at an absolute weight amount of between about 1.0 mg/container to about 25.0 mg/container; (v) PS80 or poloxamer-188 (preferably PS80) at an absolute weight amount of between about 0.010 mg/container to about 1.00 mg/container; and/or (vi) NaCl at an absolute weight amount of between about 0.010 mg/container to about 1.00 mg/container.

In some preferred embodiments, a solid composition of the invention may comprise or consist of: (i) a chimeric clostridial neurotoxin, particularly a chimeric BONT, preferably a BoNT/AB chimera, at an absolute weight of between about 5 ng/container and about 250 ng/container (e.g. between about 50 ng/container and about 250 ng/container, particularly between about 90 ng/container and about 200 ng/container); (ii) L-histidine at an absolute weight of between about 1.0 mg/container to about 3.5 mg/container; (iii) histidine-HCl at an absolute weight amount of between about 0.25 mg/container to about 2.00 mg/container; (iv) sucrose at an absolute weight amount of between about 1.0 mg/container to about 15.0 mg/container; (v) PS80 or poloxamer-188 (preferably PS80) at an absolute weight amount of between about 0.025 mg/container to about 0.200 mg/container; and/or (vi) NaCl at an absolute weight amount of between about 0.20 mg/container to about 1.00 mg/container.

In some particularly preferred embodiments, a solid composition of the invention may comprise or consist of: (i) a chimeric clostridial neurotoxin, particularly a chimeric BONT, preferably a BoNT/AB chimera, at an absolute weight of between about 5 ng/container and about 250 ng/container (e.g. between about 50 ng/container and about 250 ng/container, particularly between about 90 ng/container and about 200 ng/container); (ii) L-histidine at an absolute weight of about 1.14 mg/container; (iii) histidine-HCl at an absolute weight amount of about 0.555 mg/container; (iv) sucrose at an absolute weight amount of about 5 mg/container; (v) PS80 or poloxamer-188 (preferably PS80) at an absolute weight amount of about 0.05 mg/container; and/or (vi) NaCl at an absolute weight amount of about 0.29 mg/container.

When a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise or consist of: (i) a chimeric clostridial neurotoxin, particularly a chimeric BoNT, preferably a BoNT/AB chimera, at an absolute weight of between about 0.05 ng/container and about 250 ng/container (e.g. between about 50 ng/container and about 250 ng/container, particularly between about 90 ng/container and about 200 ng/container); (ii) L-histidine at an absolute weight of between about 1.0 mg/container to about 2.0 mg/container; (iii) histidine-HCl at an absolute weight amount of between about 0.25 mg/container to about 1.00 mg/container; (iv) sucrose at an absolute weight amount of between about 1.0 mg/container to about 7.5 mg/container; (v) PS80 or poloxamer-188 (preferably PS80) at an absolute weight amount of between about 0.010 mg/container to about 0.100 mg/container; and/or (vi) NaCl at an absolute weight amount of between about 0.010 mg/container to about 0.500 mg/container. An exemplary combination of components is shown in Table 13.

In some preferred embodiments, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise or consist of: (i) a chimeric clostridial neurotoxin, particularly a chimeric BONT, preferably a BoNT/AB chimera at an absolute weight of between about 5 ng/container and about 250 ng/container (e.g. between about 50 ng/container and about 250 ng/container, particularly between about 90 ng/container and about 200 ng/container); (ii) L-histidine at an absolute weight of between about 1.0 mg/container to about 1.5 mg/container; (iii) histidine-HCl at an absolute weight amount of between about 0.30 mg/container to about 0.65 mg/container; (iv) sucrose at an absolute weight amount of between about 2.0 mg/container to about 6.0 mg/container; (v) PS80 or poloxamer-188 (preferably PS80) at an absolute weight amount of between about 0.025 mg/container to about 0.075 mg/container; and/or (vi) NaCl at an absolute weight amount of between about 0.20 mg/container to about 0.50 mg/container.

In some preferred embodiments, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise or consist of: (i) a chimeric clostridial neurotoxin, particularly a chimeric BONT, preferably a BoNT/AB chimera at an absolute weight of between about 5 ng/container and about 250 ng/container (e.g. between about 50 ng/container and about 250 ng/container, particularly between about 90 ng/container and about 200 ng/container); (ii) L-histidine at an absolute weight of about 1.14 mg/container; (iii) histidine-HCl at an absolute weight amount of about 0.555 mg/container; (iv) sucrose at an absolute weight amount of about 5.0 mg/container; (v) PS80 or poloxamer-188 (preferably PS80) at an absolute weight amount of about 0.050 mg/container; and/or (vi) NaCl at an absolute weight amount of about 0.29 mg/container.

When a fill volume of about 1 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of between about 1.0 mg/container to about 3.5 mg/container; (iii) histidine-HCl at an absolute weight amount of between about 0.25 mg/container to about 2.00 mg/container; (iv) sucrose at an absolute weight amount of between about 1.0 mg/container to about 15.0 mg/container; (v) PS80 or poloxamer-188 (preferably PS80) at an absolute weight amount of between about 0.025 mg/container to about 0.200 mg/container; and/or (vi) NaCl at an absolute weight amount of between about 0.20 mg/container to about 1.00 mg/container. An exemplary combination of components is shown in Table 13.

In some preferred embodiments, when a fill volume of about 1 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BoNT, preferably a BoNT/AB chimera, at a concentration of between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of between about 2.0 mg/container to about 2.5 mg/container; (iii) histidine-HCl at an absolute weight amount of between about 1.00 mg/container to about 1.50 mg/container; (iv) sucrose at an absolute weight amount of between about 8.00 mg/container to about 12.50 mg/container; (v) PS80 or poloxamer-188 (preferably PS80) at an absolute weight amount of between about 0.05 mg/container to about 0.15 mg/container; and/or (vi) NaCl at an absolute weight amount of between about 0.25 mg/container to about 0.75 mg/container.

In some preferred embodiments, when a fill volume of about 1 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BONT, preferably a BoNT/AB chimera, at a concentration of between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of about 2.28 mg/container; (iii) histidine-HCl at an absolute weight amount of about 1.11 mg/container; (iv) sucrose at an absolute weight amount of about 10.00 mg/container; (v) PS80 or poloxamer-188 (preferably PS80) at an absolute weight amount of about 0.10 mg/container; and/or (vi) NaCl at an absolute weight amount of about 0.58 mg/container.

When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BONT, preferably a BoNT/AB chimera at an absolute weight of between about 5 ng/container and about 500 ng/container (e.g. between about 50 ng/container and about 250 ng/container); (ii) L-histidine at an absolute weight of between about 3.0 mg/container to about 5.0 mg/container; (iii) histidine-HCl at an absolute weight amount of between about 1.00 mg/container to about 2.00 mg/container; (iv) sucrose at an absolute weight amount of between about 10.0 mg/container to about 20.0 mg/container; (v) PS80 or poloxamer-188 (preferably PS80) at an absolute weight amount of between about 0.010 mg/container to about 0.500 mg/container; and/or (vi) NaCl at an absolute weight amount of between about 0.50 mg/container to about 1.50 mg/container. An exemplary combination of components is shown in Table 13. When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BONT, preferably a BoNT/AB chimera at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of between about 3.0 mg/container to about 5.0 mg/container; (iii) histidine-HCl at an absolute weight amount of between about 1.00 mg/container to about 2.00 mg/container; (iv) sucrose at an absolute weight amount of between about 10.0 mg/container to about 20.0 mg/container; (v) PS80 or poloxamer-188 (preferably PS80) at an absolute weight amount of between about 0.010 mg/container to about 0.500 mg/container; and/or (vi) NaCl at an absolute weight amount of between about 0.50 mg/container to about 1.50 mg/container. An exemplary combination of components is shown in Table 13.

In some preferred embodiments, when a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BONT, preferably a BoNT/AB chimera at an absolute weight of between about 5 ng/container and about 250 ng/container (e.g. between about 50 ng/container and about 250 ng/container, particularly between about 90 ng/container and about 200 ng/container); (ii) L-histidine at an absolute weight of between about 3.0 mg/container to about 3.5 mg/container; (iii) histidine-HCl at an absolute weight amount of between about 1.25 mg/container to about 1.75 mg/container; (iv) sucrose at an absolute weight amount of between about 12.5 mg/container to about 17.5 mg/container; (v) PS80 or poloxamer-188 (preferably PS80) at an absolute weight amount of between about 0.075 mg/container to about 0.175 mg/container; and/or (vi) NaCl at an absolute weight amount of between about 0.75 mg/container to about 1.25 mg/container.

In some preferred embodiments, when a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise or consist of: (i) a chimeric clostridial neurotoxin, particularly a chimeric BONT, preferably a BoNT/AB chimera at an absolute weight of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at an absolute weight of about 3.42 mg/container; (iii) histidine-HCl at an absolute weight amount of about 1.665 mg/container; (iv) sucrose at an absolute weight amount of about 15.0 mg/container; (v) PS80 or poloxamer-188 (preferably PS80) at an absolute weight amount of about 0.150 mg/container; and/or (vi) NaCl at an absolute weight amount of about 0.87 mg/container.

In some particularly preferred embodiments, a solid composition of the invention may comprise or consist of: (i) a chimeric clostridial neurotoxin, particularly a chimeric BONT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at an absolute weight of about 3.42 mg/container; (iii) histidine-HCl at an absolute weight amount of about 1.665 mg/container; (iv) sucrose at an absolute weight amount of about 15 mg/container; (v) PS80 or poloxamer-188 (preferably PS80) at an absolute weight amount of about 0.15 mg/container; and/or (vi) NaCl at an absolute weight amount of about 0.87 mg/container.

The amount of the various components in a solid formulation of the invention may be selected to give a desired concentration on reconstitution to a specified final volume. Thus, a solid composition of the invention may comprise or consist of: (i) a chimeric clostridial neurotoxin, particularly a chimeric BoNT, preferably a BoNT/AB chimera sufficient to give rise to a concentration of between about 0.10 ng/ml and about 250 ng/mL (e.g. between about 50 ng/ml and about 250 ng/ml, particularly between about 90 ng/ml and about 200 ng/ml); (ii) L-histidine sufficient to give rise to a concentration of between about 2 mM to about 20 mM; (iii) histidine-HCl sufficient to give rise to a concentration of between about 0.5 mM to about 10.0 mM; (iv) sucrose sufficient to give rise to a concentration of between about 10 mM to about 50 mM; (v) PS80 or poloxamer-188 (preferably PS80) sufficient to give rise to a concentration of between about 0.010 mM to about 1.000 mM; and/or (vi) NaCl sufficient to give rise to a concentration of between about 1.0 mM to about 20.0 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL.

In some preferred embodiments, a solid composition of the invention may comprise or consist of: (i) a chimeric clostridial neurotoxin, particularly a chimeric BoNT, preferably a BoNT/AB chimera sufficient to give rise to a concentration of between about 5.0 ng/mL and about 250 ng/ml (e.g. between about 50 ng/ml and about 250 ng/mL, particularly between about 90 ng/ml and about 200 ng/mL); (ii) L-histidine sufficient to give rise to a concentration of between about 14 mM to about 17 mM; (iii) histidine-HCl sufficient to give rise to a concentration of between about 4.0 mM to about 6.0 mM; (iv) sucrose sufficient to give rise to a concentration of between about 25 mM to about 35 mM; (v) PS80 or poloxamer-188 (preferably PS80) sufficient to give rise to a concentration of between about 0.05 mM to about 0.10 mM; and/or (vi) NaCl sufficient to give rise to a concentration of between about 7.5 mM to about 12.5 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL.

In some particularly preferred embodiments, a solid composition of the invention may comprise or consist of: (i) a chimeric clostridial neurotoxin, particularly a chimeric BONT, preferably a BoNT/AB chimera sufficient to give rise to a concentration of between about 5.0 ng/ml and about 250 ng/mL (e.g. between about 50 ng/mL and about 250 ng/mL, particularly between about 90 ng/mL and about 200 ng/mL); (ii) L-histidine sufficient to give rise to a concentration of about 14.7 mM; (iii) histidine-HCl sufficient to give rise to a concentration of about 5.3 mM; (iv) sucrose sufficient to give rise to a concentration of about 29.2 mM; (v) PS80 or poloxamer-188 (preferably PS80) sufficient to give rise to a concentration of about 0.08 mM; and/or (vi) NaCl sufficient to give rise to a concentration of about 10 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL.

In some particularly preferred embodiments, a solid composition of the invention may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine sufficient to give rise to a concentration of about 14.7 mM; (iii) histidine-HCl sufficient to give rise to a concentration of about 5.3 mM; (iv) sucrose sufficient to give rise to a concentration of about 29.2 mM; (v) PS80 or poloxamer-188 (preferably PS80) sufficient to give rise to a concentration of about 0.08 mM; and/or (vi) NaCl sufficient to give rise to a concentration of about 10 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.25 mL to about 2 mL, preferably between about 0.25 mL to about 2 mL.

As described herein, one advantage provided by the present invention is that the pH of chimeric clostridial neurotoxin compositions can be precisely controlled, Accordingly, a solid composition of the invention when reconstituted may have a pH of between about 6.0 to about 7.0, preferably about 6.5. The precisely controlled pH may be obtained when a solid composition is reconstituted and/or pH measured at room temperature, particularly at 20-25 °C. Typically, a solid composition of the invention is reconstituted in water or saline, particularly 0.5%-1.0% (e.g. 0.9% (w/v)) saline. Thus, in some preferred embodiments, a solid composition of the invention when reconstituted may have a pH of between about 6.0 to about 7.0, preferably about 6.5, when the solid composition is reconstituted in water or saline, particularly 0.5%-1.0% (e.g. 0.9% (w/v)) saline and pH is measured at 20-25 °C.

A solid composition of the invention is typically more stable than (solid or liquid, particularly solid) compositions of chimeric clostridial neurotoxins known in the art. In particular, solid compositions of the invention are typically more stable than chimeric clostridial neurotoxins (solid or liquid, particularly solid) compositions which do not comprise a dual histidine buffer, i.e. which do not comprise histidine-HCl and/or L-histidine.

A solid composition of the invention may have improved ambient stability compared with chimeric clostridial neurotoxins (solid or liquid, particularly solid) compositions which do not comprise a dual histidine buffer. Thus, a solid composition of the invention may be stable for at least 12 months, at least 15 months, at least 18 months, at least 20 months, at least 24 months or more at 25 °C. In some preferred embodiments, a solid composition of the invention is stable for at least 18 months at 25 °C.

Alternatively or in addition, a solid composition of the invention may have improved cold-storage stability compared with chimeric clostridial neurotoxins (solid or liquid, particularly solid) compositions which do not comprise a dual histidine buffer. Thus, a solid composition of the invention may be stable for at least 18 months, at least 20 months, at least 24 months, at least 30 months, at least 36 months or more at 5 °C. In some preferred embodiments, a solid composition of the invention is stable for at least 24 months at 5 °C.

### Pre-Lyophilisation Liquid Formulations

In some preferred embodiments, the compositions of the invention are liquid compositions. Particularly, the invention provides liquid compositions of chimeric clostridial neurotoxins. These liquid compositions may be pre-lyophilised solutions. In other words, these compositions may be formulated such that they are capable of undergoing lyophilisation to produce a solid composition, particularly a solid composition of the invention.

The liquid composition may be packaged as a unit dose. The term “unit dose” as used herein refers to an amount of a composition that provide exactly one dose. For the chimeric clostridial neurotoxins of the invention, said one dose may be administered to an individual by a single injection, or by multiple injections (e.g. between about 2 to 10 injections), in a single administration, optionally to the same target site or location. Preferably, however, the liquid composition is packaged based on the amount (particularly the absolute weight) of the chimeric clostridial neurotoxin of the invention, as described herein. The liquid composition may be packaged to allow for up to 15 injections to be administered from a single container.

Non-limiting examples of stabilising agents, surfactants and tonicity agents are described above, together with non-limiting examples of suitable concentrations of dual histidine buffers, stabilising agents, surfactants and tonicity agents useful in liquid compositions of the invention. Each of these components and the concentration thereof for a liquid composition may be independently selected from the disclosure above.

The invention provides a liquid composition which comprises or consists of a chimeric clostridial neurotoxin (e.g. a chimeric BoNT), a dual histidine buffer, a stabilising agent, a surfactant and a tonicity agent. The dual histidine buffer typically comprises or consists of L-histidine and histidine-HCl, as described herein. The stabilising agent may be sucrose. The surfactant is typically a non-ionic surfactant, such as PS80 or poloxamer-188. The tonicity agent may be NaCl. Thus, the invention provides a liquid composition which comprises or consists of a chimeric clostridial neurotoxin (e.g. a chimeric BoNT), L-histidine and histidine-HCl, sucrose, PS80 or poloxamer-188 and NaCl. In some preferred embodiments, the chimeric BONT is a BoNT/AB chimera as described herein.

Preferred examples of liquid compositions of the invention, particularly pre-lyophilisation solutions, are described below.

A liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.1 ng/ml and about 200 ng/ml; (ii) L-histidine at a relative weight amount of between about 0.05% to about 0.50% (w/w); (iii) histidine-HCl at a relative weight amount of between about 0.05% to about 0.50% (w/w); (iv) sucrose at a relative weight amount of between about 0.10% to about 2.50% (w/w); (v) PS80 or poloxamer-188 (preferably PS80) at a relative weight amount of between about 0.001% to about 0.100% (w/w); and/or (vi) NaCl at a relative weight amount of between about 0.01% to about 0.50% (w/w).

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 1 ng/ml and about 100 ng/ml; (ii) L-histidine at a relative weight amount of between about 0.20% to about 0.30% (w/w); (iii) histidine-HCl at a relative weight amount of between about 0.10% to about 0.20% (w/w); (iv) sucrose at a relative weight amount of between about 0.75% to about 1.25% (w/w); (v) PS80 or poloxamer-188 (preferably PS80) at a relative weight amount of between about 0.005% to about 0.050% (w/w); and/or (vi) NaCl at a relative weight amount of between about 0.02% to about 0.075% (w/w).

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/ml and about 75 ng/ml; (ii) L-histidine at a relative weight amount of about 0.227% (w/w); (iii) histidine-HCl at a relative weight a relative weight amount of about 0.110% (w/w); (iv) sucrose at a relative weight amount of about 0.99% (w/w); (v) PS80 or poloxamer-188 (preferably PS80) at a relative weight amount of about 0.01% (w/w); and/or (vi) NaCl at a relative weight amount of about 0.058% (w/w).

A liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a relative weight amount of between about 0.05% to about 0.50% (w/w); (iii) histidine-HCl at a relative weight amount of between about 0.05% to about 0.50% (w/w); (iv) sucrose at a relative weight amount of between about 0.10% to about 2.50% (w/w); (v) PS80 or poloxamer-188 (preferably PS80) at a relative weight amount of between about 0.001% to about 0.100% (w/w); and/or (vi) NaCl at a relative weight amount of between about 0.01% to about 0.50% (w/w).

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a relative weight amount of between about 0.20% to about 0.30% (w/w); (iii) histidine-HCl at a relative weight amount of between about 0.10% to about 0.20% (w/w); (iv) sucrose at a relative weight amount of between about 0.75% to about 1.25% (w/w); (v) PS80 or poloxamer-188 (preferably PS80) at a relative weight amount of between about 0.005% to about 0.050% (w/w); and/or (vi) NaCl at a relative weight amount of between about 0.02% to about 0.075% (w/w).

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a relative weight amount of about 0.227% (w/w); (iii) histidine-HCl at a relative weight a relative weight amount of about 0.110% (w/w); (iv) sucrose at a relative weight amount of about 0.99% (w/w); (v) PS80 or poloxamer-188 (preferably PS80) at a relative weight amount of about 0.01% (w/w); and/or (vi) NaCl at a relative weight amount of about 0.058% (w/w).

A liquid composition of the invention, such as a pre-lyophilisation solution, may comprise or consist of: (i) a chimeric clostridial neurotoxin, particularly a chimeric BONT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a relative weight amount of between about 0.05% to about 0.50% (w/w); (iii) histidine-HCl at a relative weight amount of between about 0.05% to about 0.50% (w/w); (iv) sucrose at a relative weight amount of between about 0.10% to about 2.50% (w/w); (v) PS80 or poloxamer-188 (preferably PS80) at a relative weight amount of between about 0.001% to about 0.100% (w/w); and/or (vi) NaCl at a relative weight amount of between about 0.01% to about 0.50% (w/w).

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a relative weight amount of between about 0.20% to about 0.30% (w/w); (iii) histidine-HCl at a relative weight amount of between about 0.10% to about 0.20% (w/w); (iv) sucrose at a relative weight amount of between about 0.75% to about 1.25% (w/w); (v) PS80 or poloxamer-188 (preferably PS80) at a relative weight amount of between about 0.005% to about 0.050% (w/w); and/or (vi) NaCl at a relative weight amount of between about 0.02% to about 0.075% (w/w).

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a relative weight amount of about 0.227% (w/w); (iii) histidine-HCl at a relative weight a relative weight amount of about 0.110% (w/w); (iv) sucrose at a relative weight amount of about 0.99% (w/w); (v) PS80 or poloxamer-188 (preferably PS80) at a relative weight amount of about 0.01% (w/w); and/or (vi) NaCl at a relative weight amount of about 0.058% (w/w).

A liquid composition of the invention, such as a pre-lyophilisation solution, may comprise or consist of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a relative weight amount of between about 0.05% to about 0.50% (w/w); (iii) histidine-HCl at a relative weight amount of between about 0.05% to about 0.50% (w/w); (iv) sucrose at a relative weight amount of between about 0.10% to about 2.50% (w/w); (v) PS80 or poloxamer-188 (preferably PS80) at a relative weight amount of between about 0.001% to about 0.100% (w/w); and/or (vi) NaCl at a relative weight amount of between about 0.01% to about 0.50% (w/w).

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a relative weight amount of between about 0.20% to about 0.30% (w/w); (iii) histidine-HCl at a relative weight amount of between about 0.10% to about 0.20% (w/w); (iv) sucrose at a relative weight amount of between about 0.75% to about 1.25% (w/w); (v) PS80 or poloxamer-188 (preferably PS80) at a relative weight amount of between about 0.005% to about 0.050% (w/w); and/or (vi) NaCl at a relative weight amount of between about 0.02% to about 0.075% (w/w).

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a relative weight amount of about 0.227% (w/w); (iii) histidine-HCl at a relative weight a relative weight amount of about 0.110% (w/w); (iv) sucrose at a relative weight amount of about 0.99% (w/w); (v) PS80 or poloxamer-188 (preferably PS80) at a relative weight amount of about 0.01% (w/w); and/or (vi) NaCl at a relative weight amount of about 0.058% (w/w).

A liquid composition of the invention such as a pre-lyophilisation solution, may comprise or consist of: (i) a chimeric clostridial neurotoxin, particularly a chimeric BONT, preferably a BoNT/AB chimera, at a concentration of between about 0.1 ng/ml and about 200 ng/mL; (ii) L-histidine at a concentration of between about 10 mM to about 20 mM; (iii) histidine-HCl at a concentration of between about 0.5 mM to about 10.0 mM; (iv) sucrose at concentration of between about 10 mM to about 50 mM; (v) PS80 or poloxamer-188 (preferably PS80) at a concentration of between about 0.010 mM to about 1.000 mM; and/or (vi) NaCl at a concentration of between about 1.0 mM to about 20.0 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 1 ng/ml and about 100 ng/ml; (ii) L-histidine at a concentration of between about 14 mM to about 17 mM; (iii) histidine-HCl at a concentration of between about 4.0 mM to about 6.0 mM; (iv) sucrose at a concentration of between about 25 mM to about 35 mM; (v) PS80 or poloxamer-188 (preferably PS80) at a concentration of between about 0.050 mM to about 0.100 mM; and/or (vi) NaCl at a concentration of between about 7.5 mM to about 12.5 mM.

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/ml and about 75 ng/ml; (ii) L-histidine at a concentration of about 14.7 mM; (iii) histidine-HCl at a concentration of about 5.3 mM; (iv) sucrose at concentration of about 29.2 mM; (v) PS80 or poloxamer-188 (preferably PS80) at a concentration of about 0.08 mM; and/or (vi) NaCl at a concentration of about 10 mM.

A liquid composition of the invention such as a pre-lyophilisation solution, may comprise or consist of: (i) a chimeric clostridial neurotoxin, particularly a chimeric BONT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of between about 10 mM to about 20 mM; (iii) histidine-HCl at a concentration of between about 0.5 mM to about 10.0 mM; (iv) sucrose at a concentration of between about 10 mM to about 50 mM; (v) PS80 or poloxamer-188 (preferably PS80) at a concentration of between about 0.010 mM to about 1.000 mM; and/or (vi) NaCl at a concentration of between about 1.0 mM to about 20.0 mM.

In some preferred embodiments, a liquid composition of the invention, such as a pre-lyophilisation solution, may comprise or consist of: (i) a chimeric clostridial neurotoxin, particularly a chimeric BONT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of between about 14 mM to about 17 mM; (iii) histidine-HCl at a concentration of between about 4.0 mM to about 6.0 mM; (iv) sucrose at a concentration of between about 25 mM to about 35 mM; (v) PS80 or poloxamer-188 (preferably PS80) at a concentration of between about 0.050 mM to about 0.100 mM; and/or (vi) NaCl at a concentration of between about 7.5 mM to about 12.5 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of between about 1 mM to about 30 mM; (iii) histidine-HCl at a concentration of between about 4.0 mM to about 15.0 mM; (iv) sucrose at a concentration of between about 25 mM to about 60 mM; (v) PS80 or poloxamer-188 (preferably PS80) at a concentration of between about 0.050 mM to about 0.200 mM; and/or (vi) NaCl at a concentration of between about 7.5 mM to about 30 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of about 14.7 mM; (iii) histidine-HCl at a concentration of about 5.3 mM; (iv) sucrose at concentration of about 29.2 mM; (v) PS80 or poloxamer-188 (preferably PS80) at a concentration of about 0.08 mM; and/or (vi) NaCl at a concentration of about 10 mM.

A liquid composition of the invention such as a pre-lyophilisation solution, may comprise or consist of: (i) a chimeric clostridial neurotoxin, particularly a chimeric BONT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of about 29.40 mM; (iii) histidine-HCl at a concentration of about 10.6 mM; (iv) sucrose at a concentration of about 58.40 mM; (v) PS80 or poloxamer-188 (preferably PS80) at a concentration of about 0.1600 mM; and/or (vi) NaCl at a concentration of about 20 mM.

A liquid composition of the invention such as a pre-lyophilisation solution, may comprise or consist of: (i) a chimeric clostridial neurotoxin, particularly a chimeric BONT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 10 mM to about 20 mM; (iii) histidine-HCl at a concentration of between about 0.5 mM to about 10.0 mM; (iv) sucrose at a concentration of between about 10 mM to about 50 mM; (v) PS80 or poloxamer-188 (preferably PS80) at a concentration of between about 0.010 mM to about 1.000 mM; and/or (vi) NaCl at a concentration of between about 1.0 mM to about 20.0 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 14 mM to about 17 mM; (iii) histidine-HCl at a concentration of between about 4.0 mM to about 6.0 mM; (iv) sucrose at a concentration of between about 25 mM to about 35 mM; (v) PS80 or poloxamer-188 (preferably PS80) at a concentration of between about 0.050 mM to about 0.100 mM; and/or (vi) NaCl at a concentration of between about 7.5 mM to about 12.5 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of about 14.7 mM; (iii) histidine-HCl at a concentration of about 5.3 mM; (iv) sucrose at concentration of about 29.2 mM; (v) PS80 or poloxamer-188 (preferably PS80) at a concentration of about 0.08 mM; and/or (vi) NaCl at a concentration of about 10 mM.

A liquid composition of the invention such as a pre-lyophilisation solution, may comprise or consist of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 10 mM to about 20 mM; (iii) histidine-HCl at a concentration of between about 0.5 mM to about 10.0 mM; (iv) sucrose at a concentration of between about 10 mM to about 50 mM; (v) PS80 or poloxamer-188 (preferably PS80) at a concentration of between about 0.010 mM to about 1.000 mM; and/or (vi) NaCl at a concentration of between about 1.0 mM to about 20.0 mM.

In some preferred embodiments, a liquid composition of the invention, such as a pre-lyophilisation solution, may comprise or consist of: (i) a chimeric clostridial neurotoxin, particularly a chimeric BONT, preferably a BoNT/AB chimera, at a concentration between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 14 mM to about 17 mM; (iii) histidine-HCl at a concentration of between about 4.0 mM to about 6.0 mM; (iv) sucrose at a concentration of between about 25 mM to about 35 mM; (v) PS80 or poloxamer-188 (preferably PS80) at a concentration of between about 0.050 mM to about 0.100 mM; and/or (vi) NaCl at a concentration of between about 7.5 mM to about 12.5 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 14 mM to about 17 mM; (ii) L-histidine at a concentration of between about 1 mM to about 30 mM; (iii) histidine-HCl at a concentration of between about 4.0 mM to about 15.0 mM; (iv) sucrose at a concentration of between about 25 mM to about 60 mM; (v) PS80 or poloxamer-188 (preferably PS80) at a concentration of between about 0.050 mM to about 0.200 mM; and/or (vi) NaCl at a concentration of between about 7.5 mM to about 30 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of about 14.7 mM; (iii) histidine-HCl at a concentration of about 5.3 mM; (iv) sucrose at concentration of about 29.2 mM; (v) PS80 or poloxamer-188 (preferably PS80) at a concentration of about 0.08 mM; and/or (vi) NaCl at a concentration of about 10 mM.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of about 29.40 mM; (iii) histidine-HCl at a concentration of about 10.6 mM; (iv) sucrose at a concentration of about 58.40 mM; (v) PS80 or poloxamer-188 (preferably PS80) at a concentration of about 0.1600 mM; and/or (vi) NaCl at a concentration of about 20 mM.

A liquid composition of the invention such as a pre-lyophilisation solution, may comprise or consist of: (i) a chimeric clostridial neurotoxin, particularly a chimeric BONT, preferably a BoNT/AB chimera, at a concentration of between about 0.1 ng/ml and about 200 ng/ml; (ii) L-histidine at a concentration of between about 1.0 mg/mL to about 5.0 mg/mL; (iii) histidine-HCl at a concentration of between about 0.01 mg/mL to about 5.00 mg/mL; (iv) sucrose at a concentration of between about 1.0 mg/mL to about 20.0 mg/mL; (v) PS80 or poloxamer-188 (preferably PS80) at a concentration of between about 0.01 mg/mL to about 2.00 mg/mL; and/or (vi) NaCl at a concentration of between about 0.1 mg/mL to about 5.0 mg/mL.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of between about 1.5 mg/mL to about 3.0 mg/mL; (iii) histidine-HCl at a concentration of between about 0.75 mg/mL to about 1.75 mg/mL; (iv) sucrose at a concentration of between about 7.5 mg/mL to about 12.5 mg/mL; (v) PS80 or poloxamer-188 (preferably PS80) at a concentration of between about 0.05 mg/mL to about 0.50 mg/mL; and/or (vi) NaCl at a concentration of between about 0.2 mg/mL to about 0.75 mg/mL.

A liquid composition of the invention, such as a pre-lyophilisation solution, may comprise or consist of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 1.0 mg/mL to about 5.0 mg/mL; (iii) histidine-HCl at a concentration of between about 0.01 mg/mL to about 5.00 mg/mL; (iv) sucrose at a concentration of between about 1.0 mg/mL to about 20.0 mg/mL; (v) PS80 or poloxamer-188 (preferably PS80) at a concentration of between about 0.01 mg/mL to about 2.00 mg/mL; and/or (vi) NaCl at a concentration of between about 0.1 mg/mL to about 5.0 mg/mL.

A liquid composition of the invention, such as a pre-lyophilisation solution, may comprise or consist of: (i) a chimeric clostridial neurotoxin, particularly a chimeric BONT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container: (ii) L-histidine at a concentration of between about 1.0 mg/mL to about 5.0 mg/mL; (iii) histidine-HCl at a concentration of between about 0.01 mg/mL to about 5.00 mg/mL; (iv) sucrose at a concentration of between about 1.0 mg/mL to about 20.0 mg/mL; (v) PS80 or poloxamer-188 (preferably PS80) at a concentration of between about 0.01 mg/mL to about 2.00 mg/mL; and/or (vi) NaCl at a concentration of between about 0.1 mg/mL to about 5.0 mg/mL.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 1 ng/mL and about 100 ng/mL; (ii) L-histidine at a concentration of between about 1.5 mg/mL to about 3.0 mg/mL; (iii) histidine-HCl at a concentration of between about 0.75 mg/mL to about 1.75 mg/mL; (iv) sucrose at a concentration of between about 7.5 mg/mL to about 12.5 mg/mL; (v) PS80 or poloxamer-188 (preferably PS80) at a concentration of between about 0.05 mg/mL to about 0.50 mg/mL; and/or (vi) NaCl at a concentration of between about 0.2 mg/mL to about 0.75 mg/mL.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of between about 1.5 mg/mL to about 3.0 mg/mL; (iii) histidine-HCl at a concentration of between about 0.75 mg/mL to about 1.75 mg/mL; (iv) sucrose at a concentration of between about 7.5 mg/mL to about 12.5 mg/mL; (v) PS80 or poloxamer-188 (preferably PS80) at a concentration of between about 0.05 mg/mL to about 0.50 mg/mL; and/or (vi) NaCl at a concentration of between about 0.2 mg/mL to about 0.75 mg/mL.

A liquid composition of the invention, such as a pre-lyophilisation solution, may comprise or consist of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 1.5 mg/mL to about 3.0 mg/mL; (iii) histidine-HCl at a concentration of between about 0.75 mg/mL to about 1.75 mg/mL; (iv) sucrose at a concentration of between about 7.5 mg/mL to about 12.5 mg/mL; (v) PS80 or poloxamer-188 (preferably PS80) at a concentration of between about 0.05 mg/mL to about 0.50 mg/mL; and/or (vi) NaCl at a concentration of between about 0.2 mg/mL to about 0.75 mg/mL.

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 1.5 mg/mL to about 3.0 mg/mL; (iii) histidine-HCl at a concentration of between about 0.75 mg/mL to about 1.75 mg/mL; (iv) sucrose at a concentration of between about 7.5 mg/mL to about 12.5 mg/mL; (v) PS80 or poloxamer-188 (preferably PS80) at a concentration of between about 0.05 mg/mL to about 0.50 mg/mL; and/or (vi) NaCl at a concentration of between about 0.2 mg/mL to about 0.75 mg/mL.

In some particularly preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/mL and about 75 ng/ml; (ii) L-histidine at a concentration of about 2.28 mg/mL; (iii) histidine-HCl at a concentration of about 1.11 mg/mL; (iv) sucrose at a concentration of about 10 mg/mL; (v) PS80 or poloxamer-188 (preferably PS80) at a concentration of about 0.1 mg/mL; and/or (vi) NaCl at a concentration of about 0.58 mg/mL.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of about 2.28 mg/mL; (iii) histidine-HCl at a concentration of about 1.11 mg/mL; (iv) sucrose at a concentration of about 10 mg/mL; (v) PS80 or poloxamer-188 (preferably PS80) at a concentration of about 0.1 mg/mL; and/or (vi) NaCl at a concentration of about 0.58 mg/mL.

In some preferred embodiments, a liquid composition of the invention, such as a pre-lyophilisation solution, may comprise or consist of: (i) a chimeric clostridial neurotoxin, particularly a chimeric BoNT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of about 3.42 mg/mL; (iii) histidine-HCl at a concentration of about 1.665 mg/mL; (iv) sucrose at a concentration of about 15 mg/ml; (v) PS80 or poloxamer-188 (preferably PS80) at a concentration of about 0.15 mg/mL; and/or (vi) NaCl at a concentration of about 0.87 mg/mL.

As described herein, one advantage provided by the present invention is that the pH of chimeric clostridial neurotoxin compositions can be precisely controlled, Accordingly, a liquid composition (e.g. a pre-lyophilisation solution) may have a pH of between about 6.0 to about 7.0, preferably about 6.5. The precisely controlled pH may be measured at room temperature, particularly at 20-25 °C.

A liquid composition (e.g. pre-lyophilisation solution) of the invention may be more stable than liquid compositions (e.g. pre-lyophilisation solutions) of chimeric clostridial neurotoxins known in the art. In particular, liquid compositions (e.g. pre-lyophilisation solutions) of the invention are typically more stable than chimeric clostridial neurotoxins liquid compositions (e.g. pre-lyophilisation solutions) which do not comprise a dual histidine buffer, i.e. which do not comprise histidine-HCl and/or L-histidine.

A liquid composition (e.g. a pre-lyophilisation solution) of the invention may have improved ambient stability compared with chimeric clostridial neurotoxins liquid compositions (e.g. pre-lyophilisation solutions) which do not comprise a dual histidine buffer. Thus, a liquid composition (e.g. a pre-lyophilisation solution) of the invention may be stable for at least 1 month, at least 2 months, at least 3 months, at least 4 months, at least 6 months,, or more at 25 °C. In some preferred embodiments, a liquid composition (e.g. a pre-lyophilisation solution) of the invention is stable for at least 2 months at 25 °C.

Alternatively or in addition, a liquid composition (e.g. pre-lyophilisation solution) of the invention may have improved cold-storage stability compared with chimeric clostridial neurotoxins compositions (e.g. pre-lyophilisation solutions) which do not comprise a dual histidine buffer. Thus, a liquid composition (e.g. a pre-lyophilisation solution) of the invention may be stable for at least 2 months, at least 3 months, at least 4 months, at least 6 months, at least 9 months, at least 12 months, or more at 5 °C. In some preferred embodiments, a liquid composition (e.g. a pre-lyophilisation solution) of the invention is stable for at least 6 months at 5 °C.

### Post-Reconstitution Liquid Formulations

The invention provides a liquid composition (e.g. a post-reconstitution solution) comprising (a) a chimeric clostridial neurotoxin (e.g. a chimeric BoNT); (b) L-histidine; and (c) histidine-HCl. In particular, the invention provides a liquid composition (e.g. a post-reconstitution solution) which comprises or consists of a chimeric clostridial neurotoxin (e.g. a chimeric BoNT), a dual histidine buffer, a stabilising agent, a surfactant and a tonicity agent. Said liquid composition may be reconstituted from a solid composition of the invention using a suitable solvent (herein a reconstitution solvent).

Thus, the invention further provides a method of producing a liquid composition (e.g. a post-reconstitution solution), comprising reconstitution of a solid composition of the invention. Said method may comprise reconstitution in any suitable solvent, as described herein, such as saline or water.

Non-limiting examples of stabilising agents, surfactants and tonicity agents are described above, together with non-limiting examples of suitable concentrations of dual histidine buffers, stabilising agents, surfactants and tonicity agents useful in liquid compositions of the invention. Each of these components and the concentration thereof for a liquid composition may be independently selected from the disclosure above. The dual histidine buffer typically comprises or consists of L-histidine and histidine-HCl, as described herein. The stabilising agent may be sucrose. The surfactant is typically a non-ionic surfactant, such as PS80 or poloxamer-188. The tonicity agent may be NaCl. Thus, the invention provides a liquid composition (e.g. a post-reconstitution solution) which comprises or consists of a chimeric clostridial neurotoxin (e.g. a chimeric BoNT), L-histidine and histidine-HCl, sucrose, PS80 or poloxamer-188 and NaCl. In some preferred embodiments, the chimeric BONT is a BoNT/AB chimera as described herein.

As described herein, a solid composition of the invention may be reconstituted in any appropriate solvent and to any desired volume. Suitable solvents and reconstitution volumes may be readily determined by one of ordinary skill in the art. As such, a post-reconstitution liquid composition of the invention may be produced with a desired concentration of any of the components, including L-histidine and/or histidine-HCl by using an appropriate solvent volume for reconstitution. For example, reconstitution in the same volume of solvent and the fill volume of the pre-lyophilisation solution will produce a post-reconstitution liquid formulation having the same concentrations of components, including L-histidine and/or histidine-HCl as described herein. The concentration of components post-reconstitution will be inversely proportional to the volume used for reconstitution. By way of non-limiting example, if a solid composition of the invention is reconstituted in a volume double that of the pre-lyophilisation solution (e.g. 1.0 mL compared with 0.5 mL), then the concentration of each component in the post-reconstitution liquid composition will be half that of the concentration of the corresponding component in the pre-lyophilisation liquid composition. By way of further non-limiting example, if a solid composition of the invention is reconstituted in a volume four times that of the pre-lyophilisation solution (e.g. 2.0 mL compared with 0.5 mL), then the concentration of each component in the post-reconstitution liquid composition will be a quarter of the concentration of the corresponding component in the pre-lyophilisation liquid composition. By way of further non-limiting example, if a solid composition of the invention is reconstituted in a volume the same as that of the pre-lyophilisation solution (e.g. 0.5 mL compared with 0.5 mL), then the concentration of each component in the post-reconstitution liquid composition will be the same as the concentration of the corresponding component in the pre-lyophilisation liquid composition. The physician or practitioner will understand that the concentrations of the components within a composition of the invention on reconstitution will depend on the volume used for reconstitution, and will be able to determine appropriate reconstitution volumes to achieve the desired concentrations. Thus, it is within the routine skill of a physician or other practitioner administering a composition of the invention to reconstitute a solid composition as described herein to achieve a concentration of the components of the composition, particularly the chimeric clostridial neurotoxin, that is appropriate for the intended use.

Any appropriate solvent may be used for reconstituting a solid composition of the invention. Suitable examples of reconstituting solvents are known in the art and may be readily selected by one of ordinary skill without undue burden. The concentration of the reconstituting solvent may also be readily selected by one of ordinary skill without undue burden, and will typically depend on the particular reconstituting solvent selected.

Non-limiting examples of solvents which may be used for reconstituting a solid composition of the invention include saline and/or water. In some preferred embodiments, saline is used for reconstitution. Saline at a concentration of between about 0.1% to about 2% (w/v), such as between about 0.5% to about 1.5% (w/v), between about 0.5% to about 1.0% (w/v), or between about 0.75% to about 1.0% (w/v) may be used. Preferably, saline at a concentration of 0.75% to about 1.0% (w/v) may be used, particularly saline at a concentration of about 0.9% (w/v).

A solid composition of the invention may be reconstituted in a volume of reconstitution solvent of between about 0.1 mL to about 10 mL, such as between about 0.1 mL to about 7.5 mL, between about 0.25 mL to about 7.5 mL, between about 0.5 mL to about 7.5 mL, between about 0.5 mL to about 5 mL, between about 0.5 mL to about 2.5 mL, between about 0.5 mL to about 2.0 mL, between about 0.5 mL to about 1.5 mL or between about 0.5 mL to about 1.0 mL. Preferably, a solid composition of the invention may be reconstituted in a volume of reconstitution solvent of between about 0.5 mL to about 7.5 mL, such as between about 0.5 mL to about 2.0 mL, between about 0.5 mL to about 1.5 mL or between about 0.5 mL to about 1.0 mL. In other words, a solid composition of the invention may be reconstituted to a volume of between about 0.1 mL to about 10 mL, such as between about 0.1 mL to about 7.5 mL, between about 0.25 mL to about 7.5 mL, between about 0.5 mL to about 7.5 mL, between about 0.5 mL to about 5 mL, between about 0.5 mL to about 2.5 mL, between about 0.5 mL to about 2.0 mL, between about 0.5 mL to about 1.5 mL or between about 0.5 mL to about 1.0 mL. Preferably, a solid composition of the invention may be reconstituted to a volume of between about 0.5 mL to about 7.5 mL, such as between about 0.5 mL to about 2.0 mL, between about 0.5 mL to about 1.5 mL or between about 0.5 mL to about 1.0 mL. In some preferred embodiments, a solid composition of the invention may be reconstituted in a volume of reconstitution solvent (i.e. to a volume) of about 0.5 mL, about 1 mL, about 1.5 mL, about 2 mL or about 6 mL.

A solid composition of the invention may be reconstituted to a volume of between about 0.25 mL to about 10 mL, such as between about 0.5 mL to about 8.5 mL, between about 0.5 mL to about 6 mL, between about 1 mL to about 6 mL, or between about 1 mL to about 4 mL.

As described herein, the intended reconstitution volume may determine the amount of one or more of the components comprised in a solid composition of the invention, or in a liquid (particularly a pre-lyophilisation solution) composition of the invention, as described herein. The intended reconstitution volume may determine the container used to aliquot the solid composition of the invention, or in a liquid (particularly a pre-lyophilisation solution) composition of the invention.

As described herein, the intended use of the composition (e.g. therapeutic or cosmetic) may determine the intended reconstitution volume. By way of non-limiting example, a fill volume of about 0.5 mL of a liquid (e.g. pre-lyophilisation solution) may be used to fill a 1 mL container (e.g. 1R vial), 2 mL container (e.g. 2R vial) or 3 mL container (e.g. 3R vial), particularly a 2 mL container (e.g. 2R vial). Following lyophilisation, this composition may be reconstituted in a volume of between about 0.25 mL to about 2.5 mL, particularly a volume of 0.5 mL, 1 mL or 2 mL. Such fill volumes, containers and/or reconstitution volumes are typically preferred for compositions of the invention intended for cosmetic use. By way of a further non-limiting example, a fill volume of about 1.5 mL of a liquid (e.g. pre-lyophilisation solution) may be used to fill a 3 mL container (e.g. 3R vial), 5 mL container (e.g. 5R vial) or 6 mL container (e.g. 6R vial), particularly a 6 mL container (e.g. 6R vial). Following lyophilisation, this composition may be reconstituted in a volume of between about 2.5 mL to about 7.5 mL, particularly a volume of 5 mL, 6 mL or 7 mL. Such fill volumes, containers and/or reconstitution volumes are typically preferred for compositions of the invention intended for therapeutic use.

Preferred examples of liquid compositions of the invention, particularly post-reconstitution solutions, are described below.

A liquid composition of the invention, such as a post-reconstitution solution, which may be produced by reconstitution of a solid composition of the invention, may comprise or consist of: (i) a chimeric clostridial neurotoxin, particularly a chimeric BoNT, preferably a BoNT/AB chimera, at a concentration of between about 0.1 ng/ml and about 50 ng/ml; (ii) L-histidine at a relative weight amount of between about 0.01% to about 0.50% (w/v); (iii) histidine-HCl at a relative weight amount of between about 0.01% to about 0.50% (w/v); (iv) sucrose at a relative weight amount of between about 0.10% to about 2.50% (w/v); (v) PS80 or poloxamer-188 (preferably PS80) at a relative weight amount of between about 0.001% to about 0.100% (w/v); and/or (vi) NaCl at a relative weight amount of between about 0.1% to about 1.5% (w/v).

In some embodiments, a liquid composition of the invention, such as a post-reconstitution solution, which may be produced by reconstitution of a solid composition of the invention in a fill volume of about 0.5 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a relative weight amount of between about 0.2% to about 0.5% (w/v); (iii) histidine-HCl at a relative weight amount of between about 0.10% to about 0.25% (w/v); (iv) sucrose at a relative weight amount of between about 0.75% to about 1.25% (w/v); (v) PS80 or poloxamer-188 (preferably PS80) at a relative weight amount of between about 0.005% to about 0.050% (w/v); and/or (vi) NaCl at a relative weight amount of between about 0.75% to about 1.0% (w/v).

In some preferred embodiments, a liquid composition of the invention, such as a post-reconstitution solution, which may be produced by reconstitution of a solid composition of the invention in a fill volume of about 0.5 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a relative weight amount of about 0.228% (w/v); (iii) histidine-HCl at a relative weight amount of about 0.111% (w/v); (iv) sucrose at a relative weight amount of about 1.0% (w/v); (v) PS80 or poloxamer-188 (preferably PS80) at a relative weight amount of about 0.01% (w/v); and/or (vi) NaCl at a relative weight amount of about 0.958% (w/v).

In some embodiments, a liquid composition of the invention, such as a post-reconstitution solution, which may be produced by reconstitution of a solid composition of the invention in a fill volume of about 1.0 mL of 0.9% (w/v) saline, may comprise or consist of: (i) a chimeric clostridial neurotoxin, particularly a chimeric BoNT, preferably a BoNT/AB chimera, at a concentration of between about 1 ng/mL and about 50 ng/ml; (ii) L-histidine at a relative weight amount of between about 0.075% to about 0.20% (w/v); (iii) histidine-HCl at a relative weight amount of between about 0.025% to about 0.075% (w/v); (iv) sucrose at a relative weight amount of between about 0.25% to about 0.75% (w/v); (v) PS80 or poloxamer-188 (preferably PS80) at a relative weight amount of between about 0.0025% to about 0.0075% (w/v); and/or (vi) NaCl at a relative weight amount of between about 0.25% to about 0.5% (w/v).

In some preferred embodiments, a liquid composition of the invention, such as a post-reconstitution solution, which may be produced by reconstitution of a solid composition of the invention in a fill volume of about 1.0 mL of 0.9% (w/v) saline, may comprise or consist of: (i) a chimeric clostridial neurotoxin, particularly a chimeric BoNT, preferably a BoNT/AB chimera, at a concentration of between about 1 ng/mL and about 50 ng/ml; (ii) L-histidine at a relative weight amount of about 0.114% (w/v); (iii) histidine-HCl at a relative weight amount of about 0.0555% (w/v); (iv) sucrose at a relative weight amount of about 0.5% (w/v); (v) PS80 or poloxamer-188 (preferably PS80) at a relative weight amount of about 0.005% (w/v); and/or (vi) NaCl at a relative weight amount of about 0.479% (w/v).

In some embodiments, a liquid composition of the invention, such as a post-reconstitution solution, which may be produced by reconstitution of a solid composition of the invention in a fill volume of about 2.0 mL of 0.9% (w/v) saline, may comprise or consist of: (i) a chimeric clostridial neurotoxin, particularly a chimeric BONT, preferably a BoNT/AB chimera, at a concentration of between about 1 ng/ml and about 50 ng/ml; (ii) L-histidine at a relative weight amount of between about 0.01% to about 0.075% (w/v); (iii) histidine-HCl at a relative weight amount of between about 0.01% to about 0.05% (w/v); (iv) sucrose at a relative weight amount of between about 0.1% to about 0.5% (w/v); (v) PS80 or poloxamer-188 (preferably PS80) at a relative weight amount of between about 0.001% to about 0.005% (w/v); and/or (vi) NaCl at a relative weight amount of between about 0.1% to about 0.3% (w/v).

In some preferred embodiments, a liquid composition of the invention, such as a post-reconstitution solution, which may be produced by reconstitution of a solid composition of the invention in a fill volume of about 2.0 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a relative weight amount of about 0.057% (w/v); (iii) histidine-HCl at a relative weight amount of about 0.02775% (w/v); (iv) sucrose at a relative weight amount of about 0.25% (w/v); (v) PS80 or poloxamer-188 (preferably PS80) at a relative weight amount of about 0.0025% (w/v); and/or (vi) NaCl at a relative weight amount of about 0.2395% (w/v).

A liquid composition of the invention, such as a post-reconstitution solution, which may be produced by reconstitution of a solid composition of the invention, may comprise or consist of: (i) a chimeric clostridial neurotoxin, particularly a chimeric BoNT, preferably a BoNT/AB chimera, at a concentration of between about 0.1 ng/ml and about 50 ng/ml; (ii) L-histidine at a concentration of between about 0.1 mg/mL to about 3 mg/mL; (iii) histidine-HCl at a concentration of between about 0.1 mg/mL to about 1.5 mg/mL; (iv) sucrose at a concentration of between about 1 mg/mL to about 15 mg/mL; (v) PS80 or poloxamer-188 (preferably PS80) at a concentration of between about 0.01 mg/mL to about 0.5 mg/mL; and/or (vi) NaCl at a concentration of between about 7.5 mg/mL to about 10 mg/mL.

In some embodiments, a liquid composition of the invention, such as a post-reconstitution solution, which may be produced by reconstitution of a solid composition of the invention in a fill volume of about 0.5 mL of 0.9% (w/v) saline, may comprise or consist of: (i) a chimeric clostridial neurotoxin, particularly a chimeric BONT, preferably a BoNT/AB chimera, at a concentration of between about 1 ng/ml and about 50 ng/ml; (ii) L-histidine at a concentration of between about 1.4 mg/mL to about 2.8 mg/mL; (iii) histidine-HCl at a concentration of between about 0.75 mg/mL to about 1.25 mg/mL; (iv) sucrose at a concentration of between about 7.5 mg/mL to about 12.5 mg/mL; (v) PS80 or poloxamer-188 (preferably PS80) at a concentration of between about 0.05 mg/mL to about 0.25 mg/ml; and/or (vi) NaCl at a concentration of between about 9.5 mg/mL to about 10.0 mg/mL.

In some preferred embodiments, a liquid composition of the invention, such as a post-reconstitution solution, which may be produced by reconstitution of a solid composition of the invention in a fill volume of about 0.5 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/mL; (ii) L-histidine at a concentration of about 2.28 mg/mL; (iii) histidine-HCl at a concentration of about 1.11 mg/mL; (iv) sucrose at a concentration of about 10 mg/mL; (v) PS80 or poloxamer-188 (preferably PS80) at a concentration of about 0.1 mg/mL; and/or (vi) NaCl at a concentration of about 9.58 mg/mL.

In some embodiments, a liquid composition of the invention, such as a post-reconstitution solution, which may be produced by reconstitution of a solid composition of the invention in a fill volume of about 1.0 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a concentration of between about 0.75 mg/mL to about 1.5 mg/mL; (iii) histidine-HCl at a concentration of between about 0.25 mg/mL to about 0.75 mg/mL; (iv) sucrose at a concentration of between about 2.5 mg/mL to about 7.5 mg/mL; (v) PS80 or poloxamer-188 (preferably PS80) at a concentration of between about 0.01 mg/mL and about 0.075 mg/ml; and/or (vi) NaCl at a concentration of between about 9.25 mg/mL to about 9.5 mg/mL.

In some preferred embodiments, a liquid composition of the invention, such as a post-reconstitution solution, which may be produced by reconstitution of a solid composition of the invention in a fill volume of about 1.0 mL of 0.9% (w/v) saline, may comprise or consist of: (i) a chimeric clostridial neurotoxin, particularly a chimeric BONT, preferably a BoNT/AB chimera, at a concentration of between about 1 ng/ml and about 50 ng/mL; (ii) L-histidine at a concentration of about 1.14 mg/mL; (iii) histidine-HCl at a concentration of about 0.555 mg/mL; (iv) sucrose at a concentration of about 5 mg/mL; (v) PS80 or poloxamer-188 (preferably PS80) at a concentration of about 0.05 mg/mL; and/or (vi) NaCl at a concentration of about 9.29 mg/mL.

In some embodiments, a liquid composition of the invention, such as a post-reconstitution solution, which may be produced by reconstitution of a solid composition of the invention in a fill volume of about 2.0 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a concentration of between about 0.25 mg/mL to about 0.75 mg/mL; (iii) histidine-HCl at a concentration of between about 0.1 mg/mL to about 0.3 mg/mL; (iv) sucrose at a concentration of between about 1 mg/mL to about 5 mg/mL; (v) PS80 or poloxamer-188 (preferably PS80) at a concentration of between about 0.01 mg/mL and about 0.05 mg/mL; and/or (vi) NaCl at a concentration of between about 9.0 mg/mL to about 9.25 mg/mL.

In some preferred embodiments, a liquid composition of the invention, such as a post-reconstitution solution, which may be produced by reconstitution of a solid composition of the invention in a fill volume of about 2.0 mL of 0.9% (w/v) saline, may comprise or consist of: (i) a chimeric clostridial neurotoxin, particularly a chimeric BONT, preferably a BoNT/AB chimera, at a concentration of between about 1 ng/ml and about 50 ng/mL; (ii) L-histidine at a concentration of about 0.57 mg/mL; (iii) histidine-HCl at a concentration of about 0.2775 mg/mL; (iv) sucrose at a concentration of about 2.5 mg/mL; (v) PS80 or poloxamer-188 (preferably PS80) at a concentration of about 0.025 mg/mL; and/or (vi) NaCl at a concentration of about 9.145 mg/mL.

A liquid composition of the invention, such as a post-reconstitution solution, which may be produced by reconstitution of a solid composition of the invention, may comprise or consist of: (i) a chimeric clostridial neurotoxin, particularly a chimeric BoNT, preferably a BoNT/AB chimera, at a concentration of between about 0.1 ng/ml and about 50 ng/mL; (ii) L-histidine, optionally at a concentration of between about 1.41 mg/mL to about 2.82 mg/mL preferably between about 2.0 mg/mL to about 2.5 mg/mL; (iii) histidine-HCl, optionally at a concentration of between about 0.70 mg/mL to about 1.41 mg/mL, preferably between about 1.00 mg/mL to about 1.20 mg/mL; (iv) sucrose, optionally at a concentration of between about 9.80 mg/mL to about 11.30 mg/mL, preferably between about 9.80 mg/mL to about 10.20 mg/mL; (v) polysorbate 80 or poloxamer-188, optionally at a concentration of between about 0.070 mg/mL to about 0.140 mg/mL, preferably between about 0.095 mg/mL to about 0.120 mg/mL; and (vi) sodium chloride, optionally at a concentration of between about 0.28 mg/ml to about 1.00 mg/mL, preferably between about 0.56 mg/mL to about 0.70 mg/mL.

A liquid composition of the invention, such as a post-reconstitution solution, which may be produced by reconstitution of a solid composition of the invention, may comprise or consist of: (i) a chimeric clostridial neurotoxin, particularly a chimeric BoNT, preferably a BoNT/AB chimera, at a concentration of between about 0.1 ng/ml and about 50 ng/ml; (ii) L-histidine at a concentration of between about 2 mM to about 17 mM; (iii) histidine-HCl at a concentration of between about 1 mM to about 10 mM; (iv) sucrose at a concentration of between about 1 mM to about 50 mM; (v) PS80 or poloxamer-188 (preferably PS80) at a concentration of between about 0.01 mM to about 0.1 mM; and/or (vi) NaCl at a concentration of between about 125 mM to about 175 mM.

In some embodiments, a liquid composition of the invention, such as a post-reconstitution solution, which may be produced by reconstitution of a solid composition of the invention in a fill volume of about 0.5 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a concentration of between about 14 mM to about 17 mM; (iii) histidine-HCl at a concentration of between about 4 mM to about 7 mM; (iv) sucrose at a concentration of between about 25 mM to about 35 mM; (v) PS80 or poloxamer-188 (preferably PS80) at a concentration of between about 0.05 mM to about 0.1 mM; and/or (vi) NaCl at a concentration of between about 160 mM to about 170 mM.

In some preferred embodiments, a liquid composition of the invention, such as a post-reconstitution solution, which may be produced by reconstitution of a solid composition of the invention in a fill volume of about 0.5 mL of 0.9% (w/v) saline, may comprise or consist of: (i) a chimeric clostridial neurotoxin, particularly a chimeric BONT, preferably a BoNT/AB chimera, at a concentration of between about 1 ng/mL and about 50 ng/ml; (ii) L-histidine at a concentration of about 14.7 mM; (iii) histidine-HCl at a concentration of about 5.3 mM; (iv) sucrose at a concentration of about 29.2 mM; (v) PS80 or poloxamer-188 (preferably PS80) at a concentration of about 0.08 mM; and/or (vi) NaCl at a concentration of about 164 mM.

In some embodiments, a liquid composition of the invention, such as a post-reconstitution solution, which may be produced by reconstitution of a solid composition of the invention in a fill volume of about 1.0 mL of 0.9% (w/v) saline, may comprise or consist of: (i) a chimeric clostridial neurotoxin, particularly a chimeric BONT, preferably a BoNT/AB chimera, at a concentration of between about 1 ng/ml and about 50 ng/ml; (ii) L-histidine at a concentration of between about 5 mM to about 10 mM; (iii) histidine-HCl at a concentration of between about 2 mM to about 4 mM; (iv) sucrose at a concentration of between about 10 mM to about 20 mM; (v) PS80 or poloxamer-188 (preferably PS80) at a concentration of between about 0.03 mM to about 0.06 mM; and/or (vi) NaCl at a concentration of between about 155 mM to about 165 mM.

In some preferred embodiments, a liquid composition of the invention, such as a post-reconstitution solution, which may be produced by reconstitution of a solid composition of the invention in a fill volume of about 1.0 mL of 0.9% (w/v) saline, may comprise or consist of: (i) a chimeric clostridial neurotoxin, particularly a chimeric BONT, preferably a BoNT/AB chimera, at a concentration of between about 1 ng/mL and about 50 ng/ml; (ii) L-histidine at a concentration of about 7.35 mM; (iii) histidine-HCl at a concentration of about 2.65 mM; (iv) sucrose at a concentration of about 14.6 mM; (v) PS80 or poloxamer-188 (preferably PS80) at a concentration of about 0.04 mM; and/or (vi) NaCl at a concentration of about 159 mM.

In some embodiments, a liquid composition of the invention, such as a post-reconstitution solution, which may be produced by reconstitution of a solid composition of the invention in a fill volume of about 2.0 mL of 0.9% (w/v) saline, may comprise or consist of: (i) a chimeric clostridial neurotoxin, particularly a chimeric BONT, preferably a BoNT/AB chimera, at a concentration of between about 1 ng/ml and about 50 ng/ml; (ii) L-histidine at a concentration of between about 2 mM to about 5 mM; (iii) histidine-HCl at a concentration of between about 1 mM to about 2 mM; (iv) sucrose at a concentration of between about 5 mM to about 10 mM; (v) PS80 or poloxamer-188 (preferably PS80) at a concentration of between about 0.01 mM to about 0.03 mM; and/or (vi) NaCl at a concentration of between about 150 mM to about 160 mM.

In some preferred embodiments, a liquid composition of the invention, such as a post-reconstitution solution, which may be produced by reconstitution of a solid composition of the invention in a fill volume of about 2.0 mL of 0.9% (w/v) saline, may comprise or consist of: (i) a chimeric clostridial neurotoxin, particularly a chimeric BoNT, preferably a BoNT/AB chimera, at a concentration of between about 1 ng/ml and about 50 ng/ml; (ii) L-histidine at a concentration of about 3.675 mM; (iii) histidine-HCl at a concentration of about 1.325 mM; (iv) sucrose at a concentration of about 7.3 mM; (v) PS80 or poloxamer-188 (preferably PS80) at a concentration of about 0.02 mM; and/or (vi) NaCl at a concentration of about 156 mM.

As described herein, one advantage provided by the present invention is that the pH of chimeric clostridial neurotoxin compositions can be precisely controlled, Accordingly, a liquid composition (e.g. a post-reconstitution solution) may have a pH of between about 6.0 to about 7.0, preferably about 6.5. The precisely controlled pH may be measured at room temperature, particularly at 20-25 °C.

A liquid composition (e.g. a post-reconstitution solution) of the invention may be more stable than liquid compositions (e.g. post-reconstitution solutions) of chimeric clostridial neurotoxins known in the art. In particular, liquid compositions (e.g. post-reconstitution solutions) of the invention are typically more stable than chimeric clostridial neurotoxins liquid compositions (e.g. post-reconstitution solutions) which do not comprise a dual histidine buffer, i.e. which do not comprise histidine-HCl and/or L-histidine.

A liquid composition (e.g. a post-reconstitution solution) of the invention may have improved ambient stability compared with chimeric clostridial neurotoxins liquid compositions (e.g. post-reconstitution solutions) which do not comprise a dual histidine buffer. Thus, a liquid composition (e.g. a post-reconstitution solution) of the invention may be stable for at least 1 month, at least 2 months, at least 3 months, at least 4 months, at least 6 months, at least 9 months, or more at 25 °C. In some preferred embodiments, a liquid composition of the invention is stable for at least 2 months at 25 °C.

Alternatively or in addition, a liquid composition (e.g. a post-reconstitution solution) of the invention may have improved cold-storage stability compared with chimeric clostridial neurotoxins compositions (e.g. post-reconstitution solutions) which do not comprise a dual histidine buffer. Thus, a liquid composition (e.g. a post-reconstitution solution) of the invention may be stable for at least 2 months, at least 3 months, at least 4 months, at least 6 months, at least 9 months, at least 12 months, or more at 5 °C. In some preferred embodiments, a liquid composition (e.g. a post-reconstitution solution) of the invention is stable for at least 6 months at 5 °C.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 0.05 ng/container to about 500 ng/container, such as between about 0.10 ng/container to about 500 ng/container, between about 1 ng/container to about 500 ng/container, between about 5 ng/container to about 500 ng/container, between about 10 ng/container to about 500 ng/container, between about 15 ng/container to about 500 ng/container, between about 25 ng/container to about 500 ng/container, between about 50 ng/container to about 500 ng/container, between about 0.05 ng/container to about 300 ng/container, between about 0.05 ng/container to about 250 ng/container, between about 0.10 ng/container to about 250 ng/container, between about 1 ng/container to about 250 ng/container, between about 5 ng/container to about 250 ng/container, between about 10 ng/container to about 250 ng/container, between about 25 ng/container to about 250 ng/container, between about 50 ng/container to about 450 ng/container, between about 50 ng/container to about 300 ng/container, between about 50 ng/container to about 250 ng/container, between about 75 ng/container to about 250 ng/container, between about 100 ng/container to about 300 ng/container between about 100 ng/container to about 250 ng/container, between about 90 ng/container to about 200 ng/container, between about 100 ng/container to about 200 ng/container, between about 140 ng/container to about 200 ng/container, between about 75 ng/container to about 500 ng/container, between about 100 ng/container to about 450 ng/container, between about 100 ng/container to about 400 ng/container, between about 150 ng/container to about 400 ng/container, between about 150 ng/container to about 350 ng/container, between about 150 ng/container to about 300 ng/container or between about 200 ng/container to about 300 ng/container; (ii) L-histidine at a concentration of between about 0.5 mM to about 40 mM; (iii) histidine-HCl at a concentration of between about 0.2 mM to about 15 mM; (iv) sucrose at a concentration of between about 1 mM to about 75 mM; (v) PS80 or poloxamer-188 (preferably PS80) at a concentration of between about 0.001 mM to about 0.20 mM; and/or (vi) NaCl at a concentration of between about 150 mM to about 175 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 0.5 mM to about 40 mM; (iii) histidine-HCl at a concentration of between about 0.2 mM to about 15 mM; (iv) sucrose at a concentration of between about 1 mM to about 75 mM; (v) PS80 or poloxamer-188 (preferably PS80) at a concentration of between about 0.001 mM to about 0.20 mM; and/or (vi) NaCl at a concentration of between about 150 mM to about 175 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 0.7 mM to about 30 mM; (iii) histidine-HCl at a concentration of between about 0.25 mM to about 11 mM; (iv) sucrose at a concentration of between about 1.2 mM to about 60 mM; (v) PS80 or poloxamer-188 (preferably PS80) at a concentration of between about 0.003 mM to about 0.175 mM; and/or (vi) NaCl at a concentration of between about 150 mM to about 165 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 0.5 mM to about 1.0 mM; (iii) histidine-HCl at a concentration of between about 0.1 mM to about 0.5 mM; (iv) sucrose at a concentration of between about 1.0 mM to about 2.0 mM; (v) PS80 or poloxamer-188 (preferably PS80) at a concentration of between about 0.002 mM to about 0.005 mM; and/or (vi) NaCl at a concentration of between about 140 mM to about 160 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of about 0.735 mM; (iii) histidine-HCl at a concentration of about 0.265 mM; (iv) sucrose at a concentration of about 1.46 mM; (v) PS80 or poloxamer-188 (preferably PS80) at a concentration of about 0.004 mM; and/or (vi) NaCl at a concentration of about 154.5 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of between about 25 mM to about 30 mM; (iii) histidine-HCl at a concentration of between about 8 mM to about 11 mM; (iv) sucrose at a concentration of between about 55 mM to about 60 mM; (v) PS80 or poloxamer-188 (preferably PS80) at a concentration of between about 0.05 mM to about 0.175 mM; and/or (vi) NaCl at a concentration of between about 160 mM to about 165 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration between about 50 ng/container to about 300 ng/container, between about 50 ng/container to about 250 ng/container, between about 75 ng/container to about 250 ng/container, between about 75 ng/container to about 200 ng/container, particularly between about 90 ng/container to about 200 ng/container; (ii) L-histidine at a concentration of about 29.4 mM; (iii) histidine-HCl at a concentration of about 10.6 mM; (iv) sucrose at a concentration of about 58.4 mM; (v) PS80 or poloxamer-188 (preferably PS80) at a concentration of about 0.160 mM; and/or (vi) NaCl at a concentration of about 163.92 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container (ii) L-histidine at a concentration of between about 0.5 mM to about 40 mM; (iii) histidine-HCl at a concentration of between about 0.2 mM to about 15 mM; (iv) sucrose at a concentration of between about 1 mM to about 75 mM; (v) PS80 or poloxamer-188 (preferably PS80) at a concentration of between about 0.001 mM to about 0.20 mM; and/or (vi) NaCl at a concentration of between about 150 mM to about 175 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 0.7 mM to about 30 mM; (iii) histidine-HCl at a concentration of between about 0.25 mM to about 11 mM; (iv) sucrose at a concentration of between about 1.2 mM to about 60 mM; (v) PS80 or poloxamer-188 (preferably PS80) at a concentration of between about 0.003 mM to about 0.175 mM; and/or (vi) NaCl at a concentration of between about 150 mM to about 165 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 0.5 mM to about 1.0 mM; (iii) histidine-HCl at a concentration of between about 0.1 mM to about 0.5 mM; (iv) sucrose at a concentration of between about 1.0 mM to about 2.0 mM; (v) PS80 or poloxamer-188 (preferably PS80) at a concentration of between about 0.002 mM to about 0.005 mM; and/or (vi) NaCl at a concentration of between about 140 mM to about 160 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 0.7 mM to about 1.5 mM; (iii) histidine-HCl at a concentration of between about 0.25 mM to about 0.75 mM; (iv) sucrose at a concentration of between about 1.2 mM to about 2 mM; (v) PS80 or poloxamer-188 (preferably PS80) at a concentration of between about 0.003 mM to about 0.01 mM; and/or (vi) NaCl at a concentration of between about 150 mM to about 158 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of about 0.735 mM; (iii) histidine-HCl at a concentration of about 0.265 mM; (iv) sucrose at a concentration of about 1.46 mM; (v) PS80 or poloxamer-188 (preferably PS80) at a concentration of about 0.004 mM; and/or (vi) NaCl at a concentration of about 154.5 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of between about 25 mM to about 30 mM; (iii) histidine-HCl at a concentration of between about 8 mM to about 11 mM; (iv) sucrose at a concentration of between about 55 mM to about 60 mM; (v) PS80 or poloxamer-188 (preferably PS80) at a concentration of between about 0.05 mM to about 0.175 mM; and/or (vi) NaCl at a concentration of between about 160 mM to about 165 mM.

In some embodiments, a liquid composition of the invention, such as a post-reconstitution solution, which may be produced by reconstitution of a solid composition of the invention in a volume of 0.9% (w/v) saline sufficient to result in the liquid composition comprising or consisting of: (i) a chimeric clostridial neurotoxin, particularly a chimeric BONT, preferably a BoNT/AB chimera, at a concentration of between about 5 ng/container to about 500 ng/container, between about 50 ng/container to about 450 ng/container, between about 100 ng/container to about 400 ng/container, between about 150 ng/container to about 350 ng/container, particularly between about 200 ng/container to about 300 ng/container; (ii) L-histidine at a concentration of about 29.4 mM; (iii) histidine-HCl at a concentration of about 10.6 mM; (iv) sucrose at a concentration of about 58.4 mM; (v) PS80 or poloxamer-188 (preferably PS80) at a concentration of about 0.160 mM; and/or (vi) NaCl at a concentration of about 163.92 mM.

In some preferred embodiments, the fill volume is in the range of about 0.5 mL to about 1.5 mL. In particularly preferred embodiments, the fill volume is selected from about 0.5 mL, about 1 mL and about 1.5 mL. For example, the fill volume may be about 0.5 mL. For example, the fill volume may be about 1 mL. For example, the fill volume may be about 1.5 mL.

### Formulations of Alternative Clostridial Neurotoxins

Any and all disclosure above relating to chimeric clostridial neurotoxins (e.g., in relation to formulations, stabilising agents, solid compositions, post-reconstitution liquid formulations, stability, therapeutic and cosmetic indications) applies equally and without reservation to the formulations for other modified clostridial neurotoxins (e.g., modified BoNT/A as defined herein).

Suitable modified BoNT/A polypeptides (and nucleotide sequences encoding the same, where present) are described in WO 2015/004461 A1 and WO 2017/191315, both of which are incorporated herein by reference in their entirety.

A preferred modified BoNT/A is one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such modified BoNT/A demonstrates a reduction in, or absence of, side effects compared to the use of known BoNT/A. The increased tissue retention properties of the modified BoNT/A of the invention also provides increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 2, wherein the amino acid residue numbering is determined by alignment with SEQ ID NO: 2. As the presence of a methionine residue at position 1 of SEQ ID NO: 2 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 2 includes a methionine, the position numbering will be as defined above (e.g. ASN 886 will be ASN 886 of SEQ ID NO: 2). Alternatively, where the methionine is absent from SEQ ID NO: 2 the amino acid residue numbering should be modified by −1 (e.g. ASN 886 will be ASN 885 of SEQ ID NO: 2). Similar considerations apply when the methionine at position 1 of the other polypeptide sequences described herein is present/absent, and the skilled person will readily determine the correct amino acid residue numbering using techniques routine in the art.

The amino acid residue(s) indicated for modification are surface exposed amino acid residue(s).

A modified BoNT/A may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277. The modified BoNT/A may be encoded by a nucleic acid sequence having at least 70% sequence identity to a nucleic acid sequence selected from SEQ ID NOs: 30, 32, 34 and 36. For example, a nucleic acid sequence having at least 80%, 90%, 95% or 99.9% sequence identity to a nucleic acid sequence selected from SEQ ID NOs: 30, 32, 34 and 36. Preferably, a modified BoNT/A for use in the invention may be encoded by a nucleic acid comprising (or consisting of) SEQ ID NOs: 30, 32, 34 and 36. The modified BoNT/A may comprise a polypeptide sequence having at least 70% sequence identity to a polypeptide sequence selected from SEQ ID NOs: 31, 33, 35 and 37. For example, a polypeptide sequence having at least 80%, 90%, 95% or 99.9% sequence identity to a polypeptide sequence selected from SEQ ID NOs: 31, 33, 35 and 37. Preferably, a modified BoNT/A for use in the invention may comprise (more preferably consist of) a polypeptide sequence selected from SEQ ID NOs: 31, 33, 35 and 37.

The term “one or more amino acid residue(s)” when used in the context of modified BoNT/A preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A may comprise 1-30, 3-20, or 5-10 amino acid modifications. More preferably, the term “one or more amino acid residue(s)” when used in the context of modified BoNT/A means all of the indicated amino acid residue(s).

Preferably, beyond the one or more amino acid modification(s) at the indicated amino acid residue(s), the modified BoNT/A does not contain any further amino acid modifications when compared to SEQ ID NO: 2.

Most preferably, a modified BoNT/A comprises (more preferably consists of) a modification at one or more amino acid residue(s) selected from: ASN 886, ASN 930, SER 955, GLN 991, ASN 1026, ASN 1052, and GLN 1229. The modified BoNT/A may be encoded by a nucleic acid sequence having at least 70% sequence identity to SEQ ID NO: 30. For example, a nucleic acid sequence having at least 80%, 90%, 95% or 99.9% sequence identity to SEQ ID NO: 30. Preferably, a modified BoNT/A for use in the invention may be encoded by a nucleic acid comprising (or consisting of) SEQ ID NO: 30. The modified BoNT/A may comprise a polypeptide sequence having at least 70% sequence identity to SEQ ID NO: 31. For example, a polypeptide sequence having at least 80%, 90%, 95% or 99.9% sequence identity to SEQ ID NO: 31. Preferably, a modified BoNT/A for use in the invention may comprise (more preferably consist of) SEQ ID NO: 31.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
V. deletion of an acidic surface exposed amino acid residue.

A modification as indicated above results in a modified BoNT/A that has an increased positive surface charge and increased isoelectric point when compared to the corresponding unmodified BoNT/A.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated.

The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified BoNT/A of the invention may have a pI value that is at least 0.2, 0.4, 0.5 or 1 pI units higher than that of an unmodified BoNT/A (e.g. SEQ ID NO: 2). Preferably, a modified BoNT/A may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A. In an amino acid substitution, an amino acid residue that forms part of the BoNT/A polypeptide sequence is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Following modification in accordance with the invention, the modified BoNT/A is capable of binding to the target cell receptors that unmodified BoNT/A (e.g. SEQ ID NO: 2) binds.

A composition of the invention may be a liquid composition or formulation (e.g., a pre-lyophilisation formulation or a post-reconstitution formulation) or a solid composition. Accordingly, the present invention provides a solid or liquid composition comprising or consisting of: (a) a modified BoNT/A as defined above; and (b) L-histidine and/or histidine-HCl. Particularly the present invention provides a solid or liquid composition comprising or consisting of: (a) a modified BoNT/A; and (b) L-histidine.

The invention provides a composition comprising a modified BoNT/A as described above, a histidine buffer (e.g. L-histidine), a stabilising agent, a surfactant and/or a tonicity agent, each of which (and the concentration thereof) may be independently selected as described herein. By way of non-limiting example, the invention provides a composition comprising a modified BoNT/A, a histidine buffer (e.g. L-histidine), sucrose, PS80 or poloxamer-188 (preferably PS80), and/or NaCl, wherein the concentration of each may be independently selected as described herein.

Preferably, the invention provides a composition comprising a modified BoNT/A as described above, L-histidine, a stabilising agent, a surfactant and/or a tonicity agent, each of which (and the concentration thereof) may be independently selected as described herein. By way of non-limiting example, the invention provides a composition comprising a modified BoNT/A, L-histidine, sucrose, PS80 or poloxamer-188 (preferably PS80), and/or NaCl, wherein the concentration of each may be independently selected as described herein.

A solid composition of the invention (e.g. comprising a modified BoNT/A) may comprise a relative weight amount of L-histidine of between about 5% to about 25% (w/w), such as between about 15% to about 25% (w/w), between about 17.5% to about 25% (w/w), between about 20% to about 25% (w/w), between about 21% to about 24% (w/w), between about 22% to about 24% (w/w), or between about 22% to about 23% (w/w). Preferably a solid composition of the invention may comprise a relative weight amount of L-histidine of between about 20% to about 25% (w/w), such as between about 21% to about 24% (w/w), or between about 22% to about 23% (w/w). In some preferred embodiments, the relative weight amount of L-histidine is about 22.5% (w/w).

A solid formulation of the invention (e.g. comprising a modified BoNT/A) may comprise an absolute weight of L-histidine of between about 1.0 mg/container to about 6.0 mg/container, such as between about 1.0 mg/container to about 5.0 mg/container, between about 1.0 mg/container to about 4.0 mg/container, between about 1.0 mg/container to about 3.5 mg/container, between about 1.0 mg/container to about 3.0 mg/container, between about 1.0 mg/container to about 2.5 mg/container, between about 1.0 mg/container to about 2.0 mg/container, or between about 1.2 mg/container to about 1.8 mg/container. The absolute amount of L-histidine in a solid composition will typically depend on the volume of the composition used to fill a given container.

Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of L-histidine of between about 1.0 mg/container to about 6.0 mg/container, such as between about 1.0 mg/container to about 5.0 mg/container, between about 1.0 mg/container to about 4.0 mg/container, between about 1.0 mg/container to about 3.5 mg/container, between about 1.0 mg/container to about 3.0 mg/container, between about 1.0 mg/container to about 2.5 mg/container, between about 1.0 mg/container to about 2.0 mg/container, or between about 1.2 mg/container to about 1.8 mg/container. In some preferred embodiments, the absolute weight amount of L-histidine is about 1.55 mg/container (2R).

When a fill volume of about 1.0 mL is used (e.g. to fill a 6 mL container (e.g. 6R vial), a solid formulation of the invention may comprise an absolute weight of L-histidine of between about 2.0 mg/container to about 12.0 mg/container, such as between about 2.0 mg/container to about 10.0 mg/container, between about 2.0 mg/container to about 8.0 mg/container, between about 2.0 mg/container to about 7 mg/container, between about 2.0 mg/container to about 6.0 mg/container, between about 2.0 mg/container to about 5 mg/container, between about 2.0 mg/container to about 4.0 mg/container, or between about 2.4 mg/container to about 3.6 mg/container. In some preferred embodiments, the absolute weight amount of L-histidine is about 3.10 mg/container (6R).

When a fill volume of about 1.5 mL is used (e.g. to fill a 3 mL container (e.g. 3R vial), 5 mL container (e.g. 5R vial) or 6 mL container (e.g. 6R vial), particularly a 6 mL container (e.g. 6R vial)), a solid formulation of the invention may comprise an absolute weight of L-histidine of between about 3.0 mg/container to about 18.0 mg/container, such as between about 3.0 mg/container to about 15.0 mg/container, between about 3.0 mg/container to about 12.0 mg/container, between about 3.0 mg/container to about 10.5 mg/container, between about 3.0 mg/container to about 9.0 mg/container, between about 3.0 mg/container to about 7.5 mg/container, between about 3.0 mg/container to about 6.0 mg/container, or between about 3.6 mg/container to about 5.4 mg/container. In some preferred embodiments, the absolute weight amount of L-histidine is about 4.65 mg/container (6R).

In some preferred embodiments, the absolute weight amount of L-histidine is about 1.55 mg/container.

In some preferred embodiments, the absolute weight amount of L-histidine is about 3.10 mg/container.

In some preferred embodiments, the absolute weight amount of L-histidine is about 4.65 mg/container.

The amount of L-histidine (and/or histidine-HCl) in a solid formulation of the invention (e.g. comprising a modified BoNT/A) may be selected to give a desired concentration on reconstitution to a specified final volume.

Thus, a solid formulation of the invention may comprise L-histidine sufficient to give rise to a concentration of between about 2 mM to about 30 mM, such as between about 2 mM to about 25 mM, between about 10 mM to about 25 mM, between about 15 mM to about 25 mM, between about 15 mM and about 24 mM, between about 15 mM and about 23 mM, between about 15 mM and about 22 mM, between about 15 mM and about 21 mM, between about 17 mM and about 21 mM, between about 19 mM and about 21 mM, or between about 19.5 mM and 20.5 mM following reconstitution, preferably between about 15 mM and about 21 mM, such as between about 19 mM and about 21 mM. Preferably a solid formulation of the invention may comprise L-histidine sufficient to give rise a concentration of about 20 mM. Preferably a solid formulation of the invention may comprise L-histidine sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention (e.g. comprising a modified BoNT/A), such as a pre-lyophilisation solution, may comprise L-histidine at a concentration of between about 2 mM to about 30 mM, such as between about 2 mM to about 25 mM, between about 10 mM to about 25 mM, between about 15 mM to about 25 mM, between about 15 mM and about 24 mM, between about 15 mM and about 23 mM, between about 15 mM and about 22 mM, between about 15 mM and about 21 mM, between about 17 mM and about 21 mM, between about 19 mM and about 21 mM, or between about 19.5 mM and 20.5 mM following reconstitution, preferably between about 15 mM and about 21 mM, such as between about 19 mM and about 21 mM. Preferably a liquid composition of the invention, such as a pre-lyophilisation solution, comprises L-histidine at a concentration of between about 15 mM and about 21 mM, such as between about 19 mM and about 21 mM, more preferably about 20 mM.

A liquid composition of the invention (e.g. comprising a modified BoNT/A), such as a pre-lyophilisation solution, may comprise L-histidine at a concentration of between about 2.0 mg/mL to about 12.0 mg/mL, such as between about 2.0 mg/mL to about 10.0 mg/mL, between about 2.0 mg/mL to about 8.0 mg/mL, between about 2.0 mg/mL to about 7 mg/mL, between about 2.0 mg/mL to about 6.0 mg/mL, between about 2.0 mg/mL to about 5 mg/mL, between about 2.0 mg/mL to about 4.0 mg/mL, or between about 2.4 mg/container to about 3.6 mg/mL. In some preferred embodiments, the concentration of L-histidine is about 3.10 mg/mL.

A solid composition of the invention (e.g. comprising a modified BoNT/A) may comprise a relative weight amount of a disaccharide (e.g. sucrose) of between about 50% to about 90% (w/w), such as between about 50% to about 80% (w/w), between about 60% to about 80% (w/w), between about 60% to about 80% (w/w), between about 65% to about 75% (w/w), or between about 70% to about 75% (w/w). Preferably a solid composition of the invention (e.g. comprising a modified BoNT/A) may comprise a relative weight amount of a disaccharide (e.g. sucrose) of between about 65% to about 75% (w/w), such as between about 70% to about 75% (w/w), or between about 70% to about 73% (w/w). A solid composition of the invention (e.g. comprising a modified BoNT/A) may comprise a relative weight amount of a disaccharide (e.g. sucrose) of about 72.6% (w/w).

A solid formulation of the invention (e.g. comprising a modified BoNT/A) may comprise an absolute weight of a disaccharide (e.g. sucrose) of between about 1.0 mg/container to about 25.0 mg/container, such as between about 1.0 mg/container to about 20.0 mg/container, between about 1.0 mg/container to about 17.5 mg/container, between about 1.0 mg/container to about 15.0 mg/container, between about 1.0 mg/container to about 10.0 mg/container, between about 1.0 mg/container to about 7.5 mg/container, between about 2.0 mg/container to about 6.0 mg/container, or between about 2.5 mg/container to about 5.5 mg/container. The absolute amount of a disaccharide (e.g. sucrose) in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a disaccharide (e.g. sucrose) of between about 1.0 mg/container to about 10.0 mg/container, such as between about 1.0 mg/container to about 7.5 mg/container, between about 2.0 mg/container to about 6.0 mg/container, between about 2.5 mg/container to about 5.5 mg/container, or between about 4.5 mg/container to about 5.5 mg/container. A solid formulation of the invention (e.g. comprising a modified BoNT/A) may comprise an absolute weight of a disaccharide (e.g. sucrose) of about 5.0 mg/container.

The amount of disaccharide (e.g. sucrose) in a solid formulation of the invention (e.g. comprising a modified BoNT/A) may be selected to give a desired concentration on reconstitution to a specified final volume. Thus, a solid formulation of the invention may comprise a disaccharide (e.g. sucrose) sufficient to give rise to a concentration of between about 5 mM to about 60 mM, such as between about 5 mM to about 50 mM, between about 10 mM to about 50 mM, between about 10 mM to about 40 mM, between about 15 mM and about 40 mM, between about 15 mM and about 35 mM, between about 20 mM and about 35 mM, between about 25 mM and about 35 mM, between about 27.5 mM and about 32.5 mM, or between about 28.5 mM and about 31.5 mM following reconstitution, preferably between about 25 mM and about 35 mM, such as between about 27.5 mM and about 32.5 mM, or between about 28.5 mM and about 31.5 mM. A solid formulation of the invention may comprise a disaccharide (e.g. sucrose) sufficient to give rise to a concentration of 29.2 mM. Preferably a solid formulation of the invention may comprise a disaccharide (e.g. sucrose) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention (e.g. comprising a modified BoNT/A), such as a pre-lyophilisation or post-reconstitution solution, may comprise a disaccharide (e.g. sucrose) at a concentration of between about 10 mM to about 50 mM, such as between about 10 mM to about 40 mM, between about 15 mM and about 40 mM, between about 15 mM and about 35 mM, between about 20 mM and about 35 mM, between about 25 mM and about 35 mM, between about 27.5 mM and about 32.5 mM, or between about 28.5 mM and about 31.5 mM. Preferably a liquid composition of the invention, such as a pre-lyophilisation solution, comprises a disaccharide (e.g. sucrose) at a concentration of between about 25 mM and about 35 mM, such as between about 27.5 mM and about 32.5 mM, or between about 28.5 mM and about 31.5 mM. A liquid composition of the invention (e.g. comprising a modified BoNT/A), such as a pre-lyophilisation solution, may comprise a disaccharide (e.g. sucrose) at a concentration of about 29.2 mM

A liquid composition of the invention (e.g. comprising a modified BoNT/A), such as a pre-lyophilisation or post-reconstitution solution, may comprise a disaccharide (e.g. sucrose) at a concentration of between about 1.0 mg/mL to about 20.0 mg/mL, such as between about 1.0 mg/mL to about 15.0 mg/mL, between about 2.5 mg/mL to about 15.0 mg/mL, between about 5.0 mg/mL to about 15.0 mg/mL, between about 5.0 mg/mL to about 12.5 mg/mL, between about 7.5 mg/mL to about 12.5 mg/mL, between about 8.0 mg/mL to about 12.0 mg/mL, between about 9.0 mg/mL to about 11.0 mg/mL, or between about 9.50 mg/mL to about 10.5 mg/mL. Preferably a liquid composition of the invention, such as a pre-lyophilisation solution, comprises a disaccharide (e.g. sucrose) at a concentration of between about 7.5 mg/mL to about 12.5 mg/mL, such as between about 8.0 mg/mL to about 12.0 mg/mL, or between about 9.0 mg/mL to about 11.0 mg/mL. A liquid composition of the invention (e.g. comprising a modified BoNT/A), such as a pre-lyophilisation solution, may comprise a disaccharide (e.g. sucrose) at a concentration of about 10 mg/mL

Preferably, the composition does not further comprise additional stabilising agents, such as a charged carrier component, as described elsewhere herein.

A solid composition of the invention (e.g. comprising a modified BoNT/A), may comprise a relative weight amount of a surfactant (e.g. PS80) of between about 0.1% to about 2.0%, such as between about 0.1% to about 1.5% (w/w), between about 0.1% to about 1.0% (w/w), between about 0.10% to about 0.90% (w/w), between about 0.20% to about 0.80% (w/w), between about 0.5% to about 1.0% (w/w), or between about 0.50% to about 0.80% (w/w). Preferably a solid composition of the invention may comprise a relative weight amount of a surfactant (e.g. PS80) of between about 0.1% to about 1.0% (w/w), such as between about 0.20% to about 0.90% (w/w), between about 0.5% to about 1.0% (w/w), or between about 0.50% to about 0.80% (w/w). A solid composition of the invention (e.g. comprising a modified BoNT/A) may comprise a relative weight amount of a surfactant (e.g. PS80) of about 0.7% (w/w).

A solid formulation of the invention (e.g. comprising a modified BoNT/A), may comprise an absolute weight of a surfactant (e.g. PS80) of between about 0.010 mg/container to about 1.00 mg/container, such as between about 0.010 mg/container to about 0.500 mg/container, between about 0.010 mg/container to about 0.250 mg/container, between about 0.010 mg/container to about 0.100 mg/container, between about 0.010 mg/container to about 0.075 mg/container, between about 0.025 mg/container to about 0.075 mg/container, or between about 0.030 mg/container to about 0.060 mg/container. The absolute amount of a surfactant (e.g. PS80) in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention may comprise an absolute weight of a surfactant (e.g. PS80) of between about 0.010 mg/container to about 0.100 mg/container, such as between about 0.010 mg/container to about 0.075 mg/container, between about 0.025 mg/container to about 0.075 mg/container, or between about 0.030 mg/container to about 0.060 mg/container. A solid composition of the invention (e.g. comprising a modified BoNT/A) may comprise an absolute weight of a surfactant (e.g. PS80) of about 0.050 mg/container.

The amount of surfactant (e.g. PS80) in a solid formulation of the invention (e.g. comprising a modified BoNT/A) may be selected to give a desired concentration on reconstitution to a specified final volume. Thus, a solid formulation of the invention may comprise a surfactant (e.g. PS80) sufficient to give rise to a concentration of between about 0.001 mM to about 1.000 mM, such as between about 0.010 mM to about 1.000 mM, between about 0.010 mM to about 0.50 mM, between about 0.010 mM and about 0.100 mM, between about 0.025 mM and about 0.100 mM, between about 0.050 mM and about 0.100 mM, or between about 0.060 mM and about 0.090 mM following reconstitution, preferably between about 0.050 mM and about 0.100 mM, such as between about 0.050 mM and about 0.090 mM or between about 0.060 mM and between about 0.090 mM. A solid formulation of the invention may comprise a surfactant (e.g. PS80) sufficient to give rise a concentration of about 0.08 mM when reconstituted. Preferably a solid formulation of the invention may comprise a surfactant (e.g. PS80) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention (e.g. comprising a modified BoNT/A), such as a pre-lyophilisation or post-reconstitution solution, may comprise a surfactant (e.g. PS80) at a concentration of between about 0.010 mM to about 1.000 mM, such as between about 0.010 mM to about 0.50 mM, between about 0.010 mM and about 0.100 mM, between about 0.025 mM and about 0.100 mM, between about 0.050 mM and about 0.100 mM, or between about 0.060 mM and about 0.090 mM. Preferably a liquid composition of the invention e.g. comprising a modified BoNT/A), such as a pre-lyophilisation solution, comprises a surfactant (e.g. PS80) at a concentration of between about 0.050 mM and about 0.100 mM, such as between about 0.050 mM and about 0.090 mM or between about 0.060 mM and about 0.090 mM. A liquid composition of the invention, such as a pre-lyophilisation or post-reconstitution solution, may comprise a surfactant (e.g. PS80) at a concentration of about 0.08 mM.

A liquid composition of the invention (e.g. comprising a modified BoNT/A), such as a pre-lyophilisation solution, may comprise a surfactant (e.g. PS80) at a concentration of between about 0.01 mg/mL to about 2.00 mg/mL, such as between about 0.01 mg/mL to about 1.00 mg/mL, between about 0.01 mg/mL to about 0.50 mg/mL, between about 0.05 mg/mL to about 0.50 mg/mL, between about 0.075 mg/mL to about 0.250 mg/mL, between about 0.08 mg/mL to about 0.20 mg/mL, or between about 0.09 mg/mL to about 0.15 mg/mL. Preferably a liquid composition of the invention, such as a pre-lyophilisation solution, comprises a surfactant (e.g. PS80) at a concentration of between about 0.05 mg/mL to about 0.50 mg/mL, such as between about 0.075 mg/mL to about 0.250 mg/mL, or between about 0.08 mg/mL to about 0.20 mg/mL. A liquid composition of the invention, such as a pre-lyophilisation or post-reconstitution solution, may comprise a surfactant (e.g. PS80) at a concentration of about 0.10 mg/mL.

A solid composition of the invention (e.g. comprising a modified BoNT/A) may comprise a relative weight amount of a tonicity agent (e.g. NaCl) of between about 0.5% to about 20.0%, such as between about 0.5% to about 10.0% (w/w), between about 0.5% to about 7.5% (w/w), between about 1.0% to about 7.5% (w/w), between about 2.5% to about 7.5% (w/w), between about 2% to about 7% (w/w), between about 2.5% to about 6.0% (w/w), or between about 3.0% to about 5.0% (w/w). Preferably a solid composition of the invention (e.g. comprising a modified BoNT/A) may comprise a relative weight amount of a tonicity agent (e.g. NaCl) of between about 2.5% to about 7.5% (w/w), such as between about 2.5% to about 6.0% (w/w), or between about 3.0% to about 5.0% (w/w). A solid composition of the invention (e.g. comprising a modified BoNT/A) may comprise a relative weight amount of a tonicity agent (e.g. NaCl) of about 4.2% (w/w).

A solid formulation of the invention (e.g. comprising a modified BoNT/A) may comprise an absolute weight of a tonicity agent (e.g. NaCl) of between about 0.010 mg/container to about 1.50 mg/container, between about 0.010 mg/container to about 1.00 mg/container, such as between about 0.010 mg/container to about 0.500 mg/container, between about 0.100 mg/container to about 0.500 mg/container, between about 0.150 mg/container to about 0.500 mg/container, between about 0.200 mg/container to about 0.500 mg/container, between about 0.200 mg/container to about 0.400 mg/container, or between about 0.250 mg/container to about 0.350 mg/container. The absolute amount of a tonicity agent (e.g. NaCl) in a solid composition will typically depend on the volume of the composition used to fill a given container. Thus, when a fill volume of about 0.5 mL is used (e.g. to fill a 1 mL container (e.g. 1R vial), 2 mL container (e.g. 2R vial) or 3 mL container (e.g. 3R vial), particularly a 2 mL container (e.g. 2R vial)), a solid formulation of the invention (e.g. comprising a modified BoNT/A) may comprise an absolute weight of a tonicity agent (e.g. NaCl) of between about 0.010 mg/container to about 0.500 mg/container, such as about between about 0.20 mg/container to about 0.50 mg/container, or between about 0.20 mg/container to about 0.40 mg/container. The absolute amount of a tonicity agent (e.g. NaCl) in a solid composition will typically depend on the volume of the composition used to fill a given container. A solid composition of the invention (e.g. comprising a modified BoNT/A) may comprise an absolute weight amount of a tonicity agent (e.g. NaCl) of about 0.29 mg/container.

The amount of tonicity agent (e.g. NaCl) in a solid formulation of the invention (e.g. comprising a modified BoNT/A) may be selected to give a desired concentration on reconstitution to a specified final volume. Thus, a solid formulation of the invention may comprise a tonicity agent (e.g. NaCl) sufficient to give rise to a concentration of between about 1.0 mM to about 170.0 mM, such as between about 1.0 mM to about 20.0 mM, between about 1.0 mM to about 15.0 mM, between about 2.5 mM and about 12.5 mM, between about 5.0 mM and about 12.5 mM, between about 7.5 mM and about 12.5 mM, or between about 9.0 mM and about 11.0 mM following reconstitution, preferably between about 7.5 mM and about 12.5 mM, such as between about 9.0 mM and about 11.0 mM. A solid formulation of the invention may comprise a tonicity agent (e.g. NaCl) sufficient to give rise to a concentration of 10 mM. Preferably a solid formulation of the invention may comprise a tonicity agent (e.g. NaCl) sufficient to give rise to the desired concentration when reconstituted to a volume of between about 0.25 mL to about 5 mL, such as between about 0.25 mL to about 3 mL, between about 0.50 mL to about 2.00 mL, preferably between about 0.50 mL to about 2.00 mL, such as about 0.5 mL to about 1.00 mL. The reconstitution volume typically depends on the volume of container and the fill volume used. By way of non-limiting example, for a 2 mL container (e.g. 2R vial), a reconstitution volume of between about 0.4 mL to about 0.6 mL may be used.

A liquid composition of the invention (e.g. comprising a modified BoNT/A), such as a pre-lyophilisation solution, may comprise a tonicity agent (e.g. NaCl) at a concentration of between about 1.0 mM to about 20.0 mM, such as between about 1.0 mM to about 15.0 mM, between about 2.5 mM and about 12.5 mM, between about 5.0 mM and about 12.5 mM, between about 7.5 mM and about 12.5 mM, or between about 9.0 mM and about 11.0 mM. Preferably a liquid composition of the invention, such as a pre-lyophilisation solution, comprises a tonicity agent (e.g. NaCl) at a concentration of between about 7.5 mM and about 12.5 mM, such as between about 9.0 mM and about 11.0 mM. A liquid composition of the invention, such as a pre-lyophilisation solution, comprises a tonicity agent (e.g. NaCl) at a concentration of about 10 mM.

A liquid composition of the invention (e.g. comprising a modified BoNT/A), such as a pre-lyophilisation solution, may comprise a tonicity agent (e.g. NaCl) at a concentration of between about 0.1 mg/mL to about 5.0 mg/mL, such as between about 0.1 mg/mL to about 1.0 mg/mL, between about 0.1 mg/mL to about 0.75 mg/mL, between about 0.20 mg/mL to about 0.75 mg/mL, or between about 0.40 mg/mL to about 0.60 mg/mL. Preferably a liquid composition of the invention, such as a pre-lyophilisation solution, comprises a tonicity agent (e.g. NaCl) at a concentration of between about 0.2 mg/mL to about 0.75 mg/mL, such as between about 0.40 mg/mL to about 0.60 mg/mL. A liquid composition of the invention, such as a pre-lyophilisation solution, comprises a tonicity agent (e.g. NaCl) at a concentration of about 0.58 mg/mL.

Any appropriate amount of modified BoNT/A may be comprised in a composition of the invention. Typically, a composition of the invention comprises an amount of modified BoNT/A sufficient to provide a therapeutically or cosmetically effective dose of the modified BoNT/A when administered to an individual. Thus, the amount of modified BoNT/A within a composition of the invention may depend on the intended therapeutic or cosmetic use of the composition.

A solid composition of the invention may comprise an absolute weight of a modified BoNT/A of between about 0.01 ng/container to about 500 ng/container, such as between about 0.01 ng/container to about 450 ng/container, between about 0.01 ng/container to about 400 ng/container, between about 0.01 ng/container to about 350 ng/container, between about 0.01 ng/container to about 300 ng/container, between about 0.01 ng/container to about 250 ng/container, between about 0.01 ng/container to about 200 ng/container, between about 0.01 ng/container to about 150 ng/container, between about 0.01 ng/container to about 100 ng/container, between about 0.01 ng/container to about 50 ng/container, between about 0.01 ng/container to about 25 ng/container, between about 0.01 ng/container to about 15 ng/container, between about 0.01 ng/container to about 10 ng/container, between about 0.01 ng/container to about 5 ng/container, between about 0.01 ng/container to about 2.5 ng/container, or between about 0.01 ng/container to about 2 ng/container.

A solid composition of the invention may comprise an absolute weight of a modified BoNT/A of between about 0.05 ng/container to about 500 ng/container, such as between about 0.05 ng/container to about 450 ng/container, between about 0.05 ng/container to about 400 ng/container, between about 0.05 ng/container to about 350 ng/container, between about 0.05 ng/container to about 300 ng/container, between about 0.05 ng/container to about 250 ng/container, between about 0.05 ng/container to about 200 ng/container, between about 0.05 ng/container to about 150 ng/container, between about 0.05 ng/container to about 100 ng/container, between about 0.05 ng/container to about 50 ng/container, between about 0.05 ng/container to about 25 ng/container, between about 0.05 ng/container to about 15 ng/container, between about 0.05 ng/container to about 10 ng/container, between about 0.05 ng/container to about 5 ng/container, between about 0.05 ng/container to about 2.5 ng/container, or between about 0.05 ng/container to about 2 ng/container.

A solid composition of the invention may comprise an absolute weight of a modified BoNT/A of between about 0.1 ng/container to about 500 ng/container, such as between about 0.1 ng/container to about 450 ng/container, between about 0.1 ng/container to about 400 ng/container, between about 0.1 ng/container to about 350 ng/container, between about 0.1 ng/container to about 300 ng/container, between about 0.1 ng/container to about 250 ng/container, between about 0.1 ng/container to about 200 ng/container, between about 0.1 ng/container to about 150 ng/container, between about 0.1 ng/container to about 100 ng/container, between about 0.1 ng/container to about 50 ng/container, between about 0.1 ng/container to about 25 ng/container, between about 0.1 ng/container to about 15 ng/container, between about 0.1 ng/container to about 10 ng/container, between about 0.1 ng/container to about 5 ng/container, between about 0.1 ng/container to about 2.5 ng/container, or between about 0.1 ng/container to about 2 ng/container.

A solid composition of the invention may comprise an absolute weight of a modified BoNT/A of between about 0.5 ng/container to about 500 ng/container, such as between about 0.5 ng/container to about 450 ng/container, between about 0.5 ng/container to about 400 ng/container, between about 0.5 ng/container to about 350 ng/container, between about 0.5 ng/container to about 300 ng/container, between about 0.5 ng/container to about 250 ng/container, between about 0.5 ng/container to about 200 ng/container, between about 0.5 ng/container to about 150 ng/container, between about 0.5 ng/container to about 100 ng/container, between about 0.5 ng/container to about 50 ng/container, between about 0.5 ng/container to about 25 ng/container, between about 0.5 ng/container to about 15 ng/container, between about 0.5 ng/container to about 10 ng/container, between about 0.5 ng/container to about 5 ng/container, between about 0.5 ng/container to about 2.5 ng/container, or between about 0.5 ng/container to about 2 ng/container.

A solid composition of the invention may comprise an absolute weight of a modified BoNT/A of between about 1 ng/container to about 500 ng/container, such as between about 1 ng/container to about 450 ng/container, between about 1 ng/container to about 400 ng/container, between about 1 ng/container to about 350 ng/container, between about 1 ng/container to about 300 ng/container, between about 1 ng/container to about 250 ng/container, between about 1 ng/container to about 200 ng/container, between about 1 ng/container to about 150 ng/container, between about 1 ng/container to about 100 ng/container, between about 1 ng/container to about 50 ng/container, between about 1 ng/container to about 25 ng/container, between about 1 ng/container to about 15 ng/container, between about 1 ng/container to about 10 ng/container, between about 1 ng/container to about 5 ng/container, between about 1 ng/container to about 2.5 ng/container, or between about 1 ng/container to about 2 ng/container.

A solid composition of the invention may comprise an absolute weight of a modified BoNT/A of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container.

In preferred embodiments, a solid composition of the invention may comprise an absolute weight of a modified BoNT/A of about 1 ng/container, about 1.4 ng/container or about 1.8 ng/container.

The absolute amount of a modified BoNT/A in a solid composition will typically depend on the volume of the composition used to fill a given container, the intended reconstitution volume and/or on the intended field of use (e.g. therapeutic or cosmetic/aesthetic).

A liquid formulation (e.g., a pre-lyophilisation formulation) of the invention may comprise an absolute weight of a modified BoNT/A of between about 0.01 ng/container to about 500 ng/container, such as between about 0.01 ng/container to about 450 ng/container, between about 0.01 ng/container to about 400 ng/container, between about 0.01 ng/container to about 350 ng/container, between about 0.01 ng/container to about 300 ng/container, between about 0.01 ng/container to about 250 ng/container, between about 0.01 ng/container to about 200 ng/container, between about 0.01 ng/container to about 150 ng/container, between about 0.01 ng/container to about 100 ng/container, between about 0.01 ng/container to about 50 ng/container, between about 0.01 ng/container to about 25 ng/container, between about 0.01 ng/container to about 15 ng/container, between about 0.01 ng/container to about 10 ng/container, between about 0.01 ng/container to about 5 ng/container, between about 0.01 ng/container to about 2.5 ng/container, or between about 0.01 ng/container to about 2 ng/container.

A liquid formulation (e.g., a pre-lyophilisation formulation) of the invention may comprise an absolute weight of a modified BoNT/A of between about 0.05 ng/container to about 500 ng/container, such as between about 0.05 ng/container to about 450 ng/container, between about 0.05 ng/container to about 400 ng/container, between about 0.05 ng/container to about 350 ng/container, between about 0.05 ng/container to about 300 ng/container, between about 0.05 ng/container to about 250 ng/container, between about 0.05 ng/container to about 200 ng/container, between about 0.05 ng/container to about 150 ng/container, between about 0.05 ng/container to about 100 ng/container, between about 0.05 ng/container to about 50 ng/container, between about 0.05 ng/container to about 25 ng/container, between about 0.05 ng/container to about 15 ng/container, between about 0.05 ng/container to about 10 ng/container, between about 0.05 ng/container to about 5 ng/container, between about 0.05 ng/container to about 2.5 ng/container, or between about 0.05 ng/container to about 2 ng/container.

A liquid formulation (e.g., a pre-lyophilisation formulation) of the invention may comprise an absolute weight of a modified BoNT/A of between about 0.1 ng/container to about 500 ng/container, such as between about 0.1 ng/container to about 450 ng/container, between about 0.1 ng/container to about 400 ng/container, between about 0.1 ng/container to about 350 ng/container, between about 0.1 ng/container to about 300 ng/container, between about 0.1 ng/container to about 250 ng/container, between about 0.1 ng/container to about 200 ng/container, between about 0.1 ng/container to about 150 ng/container, between about 0.1 ng/container to about 100 ng/container, between about 0.1 ng/container to about 50 ng/container, between about 0.1 ng/container to about 25 ng/container, between about 0.1 ng/container to about 15 ng/container, between about 0.1 ng/container to about 10 ng/container, between about 0.1 ng/container to about 5 ng/container, between about 0.1 ng/container to about 2.5 ng/container, or between about 0.1 ng/container to about 2 ng/container.

A liquid formulation (e.g., a pre-lyophilisation formulation) of the invention may comprise an absolute weight of a modified BoNT/A of between about 0.5 ng/container to about 500 ng/container, such as between about 0.5 ng/container to about 450 ng/container, between about 0.5 ng/container to about 400 ng/container, between about 0.5 ng/container to about 350 ng/container, between about 0.5 ng/container to about 300 ng/container, between about 0.5 ng/container to about 250 ng/container, between about 0.5 ng/container to about 200 ng/container, between about 0.5 ng/container to about 150 ng/container, between about 0.5 ng/container to about 100 ng/container, between about 0.5 ng/container to about 50 ng/container, between about 0.5 ng/container to about 25 ng/container, between about 0.5 ng/container to about 15 ng/container, between about 0.5 ng/container to about 10 ng/container, between about 0.5 ng/container to about 5 ng/container, between about 0.5 ng/container to about 2.5 ng/container, or between about 0.5 ng/container to about 2 ng/container.

A liquid formulation (e.g., a pre-lyophilisation formulation) of the invention may comprise an absolute weight of a modified BoNT/A of between about 1 ng/container to about 500 ng/container, such as between about 1 ng/container to about 450 ng/container, between about 1 ng/container to about 400 ng/container, between about 1 ng/container to about 350 ng/container, between about 1 ng/container to about 300 ng/container, between about 1 ng/container to about 250 ng/container, between about 1 ng/container to about 200 ng/container, between about 1 ng/container to about 150 ng/container, between about 1 ng/container to about 100 ng/container, between about 1 ng/container to about 50 ng/container, between about 1 ng/container to about 25 ng/container, between about 1 ng/container to about 15 ng/container, between about 1 ng/container to about 10 ng/container, between about 1 ng/container to about 5 ng/container, between about 1 ng/container to about 2.5 ng/container, or between about 1 ng/container to about 2 ng/container.

A liquid formulation (e.g., a pre-lyophilisation formulation) of the invention may comprise an absolute weight of a modified BoNT/A of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container.

In preferred embodiments, a liquid formulation (e.g., a pre-lyophilisation formulation) of the invention may comprise an absolute weight of a modified BoNT/A of about 1 ng/container, about 1.4 ng/container or about 1.8 ng/container.

A liquid formulation (e.g., a post-reconstitution formulation) of the invention may comprise an absolute weight of a modified BoNT/A of between about 0.01 ng/container to about 500 ng/container, such as between about 0.01 ng/container to about 450 ng/container, between about 0.01 ng/container to about 400 ng/container, between about 0.01 ng/container to about 350 ng/container, between about 0.01 ng/container to about 300 ng/container, between about 0.01 ng/container to about 250 ng/container, between about 0.01 ng/container to about 200 ng/container, between about 0.01 ng/container to about 150 ng/container, between about 0.01 ng/container to about 100 ng/container, between about 0.01 ng/container to about 50 ng/container, between about 0.01 ng/container to about 25 ng/container, between about 0.01 ng/container to about 15 ng/container, between about 0.01 ng/container to about 10 ng/container, between about 0.01 ng/container to about 5 ng/container, between about 0.01 ng/container to about 2.5 ng/container, or between about 0.01 ng/container to about 2 ng/container.

A liquid formulation (e.g., a post-reconstitution formulation) of the invention may comprise an absolute weight of a modified BoNT/A of between about 0.05 ng/container to about 500 ng/container, such as between about 0.05 ng/container to about 450 ng/container, between about 0.05 ng/container to about 400 ng/container, between about 0.05 ng/container to about 350 ng/container, between about 0.05 ng/container to about 300 ng/container, between about 0.05 ng/container to about 250 ng/container, between about 0.05 ng/container to about 200 ng/container, between about 0.05 ng/container to about 150 ng/container, between about 0.05 ng/container to about 100 ng/container, between about 0.05 ng/container to about 50 ng/container, between about 0.05 ng/container to about 25 ng/container, between about 0.05 ng/container to about 15 ng/container, between about 0.05 ng/container to about 10 ng/container, between about 0.05 ng/container to about 5 ng/container, between about 0.05 ng/container to about 2.5 ng/container, or between about 0.05 ng/container to about 2 ng/container.

A liquid formulation (e.g., a post-reconstitution formulation) of the invention may comprise an absolute weight of a modified BoNT/A of between about 0.1 ng/container to about 500 ng/container, such as between about 0.1 ng/container to about 450 ng/container, between about 0.1 ng/container to about 400 ng/container, between about 0.1 ng/container to about 350 ng/container, between about 0.1 ng/container to about 300 ng/container, between about 0.1 ng/container to about 250 ng/container, between about 0.1 ng/container to about 200 ng/container, between about 0.1 ng/container to about 150 ng/container, between about 0.1 ng/container to about 100 ng/container, between about 0.1 ng/container to about 50 ng/container, between about 0.1 ng/container to about 25 ng/container, between about 0.1 ng/container to about 15 ng/container, between about 0.1 ng/container to about 10 ng/container, between about 0.1 ng/container to about 5 ng/container, between about 0.1 ng/container to about 2.5 ng/container, or between about 0.1 ng/container to about 2 ng/container.

A liquid formulation (e.g., a post-reconstitution formulation) of the invention may comprise an absolute weight of a modified BoNT/A of between about 0.5 ng/container to about 500 ng/container, such as between about 0.5 ng/container to about 450 ng/container, between about 0.5 ng/container to about 400 ng/container, between about 0.5 ng/container to about 350 ng/container, between about 0.5 ng/container to about 300 ng/container, between about 0.5 ng/container to about 250 ng/container, between about 0.5 ng/container to about 200 ng/container, between about 0.5 ng/container to about 150 ng/container, between about 0.5 ng/container to about 100 ng/container, between about 0.5 ng/container to about 50 ng/container, between about 0.5 ng/container to about 25 ng/container, between about 0.5 ng/container to about 15 ng/container, between about 0.5 ng/container to about 10 ng/container, between about 0.5 ng/container to about 5 ng/container, between about 0.5 ng/container to about 2.5 ng/container, or between about 0.5 ng/container to about 2 ng/container.

A liquid formulation (e.g., a post-reconstitution formulation) of the invention may comprise an absolute weight of a modified BoNT/A of between about 1 ng/container to about 500 ng/container, such as between about 1 ng/container to about 450 ng/container, between about 1 ng/container to about 400 ng/container, between about 1 ng/container to about 350 ng/container, between about 1 ng/container to about 300 ng/container, between about 1 ng/container to about 250 ng/container, between about 1 ng/container to about 200 ng/container, between about 1 ng/container to about 150 ng/container, between about 1 ng/container to about 100 ng/container, between about 1 ng/container to about 50 ng/container, between about 1 ng/container to about 25 ng/container, between about 1 ng/container to about 15 ng/container, between about 1 ng/container to about 10 ng/container, between about 1 ng/container to about 5 ng/container, between about 1 ng/container to about 2.5 ng/container, or between about 1 ng/container to about 2 ng/container.

A liquid formulation (e.g., a post-reconstitution formulation) of the invention may comprise an absolute weight of a modified BoNT/A of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container.

In preferred embodiments, a liquid formulation (e.g., a post-reconstitution formulation) of the invention may comprise an absolute weight of a modified BoNT/A of about 1 ng/container, about 1.4 ng/container or about 1.8 ng/container.

Preferred examples of solid compositions of the invention are described below.

A solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at between about 20% to about 25% (w/w); (iii) sucrose at a relative weight amount of between about 65% to about 75% (w/w); (iv) PS80 or poloxamer-188 (preferably PS80) at a relative weight amount of between about 0.1% to about 1.0% (w/w); and/or (v) NaCl at a relative weight amount of between about 2.5% to about 7.5% (w/w).

In some particularly preferred embodiments, a solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at a relative weight amount of about 22.5% (w/w); (iii) sucrose at a relative weight amount of about 72.6% (w/w); (iv) PS80 or poloxamer-188 (preferably PS80) at a relative weight amount of about 0.7% (w/w); and/or (vi) NaCl at a relative weight amount of about 4.2% (w/w).

A solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at an absolute weight of between about 1.0 mg/container to about 6.0 mg/container; (iii) sucrose at an absolute weight amount of between about 1.0 mg/container to about 25.0 mg/container; (iv) PS80 or poloxamer-188 (preferably PS80) at an absolute weight amount of between about 0.010 mg/container to about 1.00 mg/container; and/or (v) NaCl at an absolute weight amount of between about 0.010 mg/container to about 1.00 mg/container.

In some particularly preferred embodiments, a solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at an absolute weight of about 1.55 mg/container; (iii) sucrose at an absolute weight amount of about 5 mg/container; (iv) PS80 or poloxamer-188 (preferably PS80) at an absolute weight amount of about 0.05 mg/container; and/or (v) NaCl at an absolute weight amount of about 0.29 mg/container.

In some particularly preferred embodiments, a solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at an absolute weight of about 3.10 mg/container; (iii) sucrose at an absolute weight amount of about 10 mg/container; (iv) PS80 or poloxamer-188 (preferably PS80) at an absolute weight amount of about 0.1 mg/container; and/or (v) NaCl at an absolute weight amount of about 0.58 mg/container.

The amount of the various components in a solid formulation of the invention may be selected to give a desired concentration on reconstitution to a specified final volume. Thus, a solid composition of the invention may comprise or consist of: (i) a modified BoNT/A sufficient to give rise to a concentration of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine sufficient to give rise to a concentration of between about 2 mM to about 30 mM; (iii) sucrose sufficient to give rise to a concentration of between about 5 mM to about 50 mM; (iv) PS80 or poloxamer-188 (preferably PS80) sufficient to give rise to a concentration of between about 0.010 mM to about 1.000 mM; and/or (vi) NaCl sufficient to give rise to a concentration of between about 1 mM to about 170 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.5 mL to about 2 mL, preferably between about 0.5 mL to about 2 mL, such as about 0.5 mL to about 1 mL.

The amount of the various components in a solid formulation of the invention may be selected to give a desired concentration on reconstitution to a specified final volume. Thus, a solid composition of the invention may comprise or consist of: (i) a modified BoNT/A sufficient to give rise to a concentration of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine sufficient to give rise to a concentration of between about 2 mM to about 30 mM; (iii) sucrose sufficient to give rise to a concentration of between about 5 mM to about 50 mM; (iv) PS80 or poloxamer-188 (preferably PS80) sufficient to give rise to a concentration of between about 0.010 mM to about 1.000 mM; and/or (vi) NaCl sufficient to give rise to a concentration of between about 1 mM to about 20 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.5 mL to about 2 mL, preferably between about 0.5 mL to about 2 mL, such as about 0.5 mL to about 1 mL.

In some particularly preferred embodiments, a solid composition of the invention may comprise or consist of: (i) a modified BoNT/A sufficient to give rise to a concentration of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine sufficient to give rise to a concentration of about 20 mM; (iii) sucrose sufficient to give rise to a concentration of about 29.2 mM; (v) PS80 or poloxamer-188 (preferably PS80) sufficient to give rise to a concentration of about 0.08 mM; and/or (vi) NaCl sufficient to give rise to a concentration of about 10 mM. Preferably a solid formulation of the invention may comprise amounts of the various components sufficient to give rise to the desired concentration when reconstituted to a volume of between about 0.25 mL to about 5 mL, such as between about 0.25 mL to about 3 mL, between about 0.5 mL to about 2 mL, preferably between about 0.5 mL to about 2 mL, such as about 0.5 mL to about 1 mL.

In some particularly preferred embodiments, a liquid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at a concentration of between about 2.0 mg/mL to about 10.0 mg/mL; (iii) sucrose at a concentration of between about 1.0 mg/mL to about 15.0 mg/mL; (iv) PS80 or poloxamer-188 (preferably PS80) at a concentration of between about 0.01 mg/mL to about 1.00 mg/mL; and/or (v) NaCl at a concentration of between about 0.1 mg/mL to about 1.0 mg/mL.

In some particularly preferred embodiments, a solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at a concentration of about 3.10 mg/mL; (iii) sucrose at a concentration of about 1.0 mg/mL; (iv) PS80 or poloxamer-188 (preferably PS80) at a concentration of about 0.1 mg/ml; and/or (v) NaCl at a concentration of about 0.58 mg/mL.

In some particularly preferred embodiments, a liquid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at a concentration of between about 2 mM to about 25 mM; (iii) sucrose at a concentration of between about 10 mM to about 40 mM; (iv) PS80 or poloxamer-188 (preferably PS80) at a concentration of between about 0.010 mM to about 0.50 mM; and/or (v) NaCl at a concentration of between about 1.0 mM to about 15.0 mM.

In some particularly preferred embodiments, a solid composition of the invention may comprise or consist of: (i) a modified BoNT/A, at an absolute weight of between about 0.01 ng/container to about 500 ng/container, between about 0.1 ng/container to about 500 ng/container, between about 0.1 ng/container to about 250 ng/container, between about 0.1 ng/container to about 100 ng/container, between about 0.5 ng/container to about 100 ng/container, between about 0.5 ng/container to about 50 ng/container, between about 0.5 ng/container to about 25 ng/container, between about 0.5 ng/container to about 10 ng/container, between about 0.5 ng/container to about 5 ng/container, between about 0.5 ng/container to about 2.5 ng/container or between about 1 ng/container to about 2.5 ng/container; (ii) L-histidine at a concentration of about 20 mM; (iii) sucrose at a concentration of about 29.2 mM; (iv) PS80 or poloxamer-188 (preferably PS80) at a concentration of about 0.08 mM; and/or (v) NaCl at a concentration of about 10 mM.

In some preferred embodiments, the compositions of the invention are liquid compositions. Particularly, the invention provides liquid compositions of modified BoNT/A as described above. These liquid compositions may be pre-lyophilised solutions. In other words, these compositions may be formulated such that they are capable of undergoing lyophilisation to produce a solid composition, particularly a solid composition of the invention.

As described herein, one advantage provided by the present invention is that the pH of chimeric clostridial neurotoxin compositions can be precisely controlled, Accordingly, a solid composition of the invention comprising modified BoNT/A when reconstituted may have a pH of between about 6.0 to about 8.0, preferably between about 7.0 to about 8.0, more preferably between about 7.5 to about 8.0, even more preferably about 7.6. The precisely controlled pH may be obtained when a solid composition is reconstituted and/or pH measured at room temperature, particularly at 20-25 °C. Typically, a solid composition of the invention is reconstituted in water or saline, particularly 0.5%-1.0% (e.g. 0.9% (w/v)) saline. Thus, in some preferred embodiments, a solid composition of the invention when reconstituted may have a pH of between about 6.0 to about 8.0, preferably between about 7.0 to about 8.0, more preferably between about 7.5 to about 8.0, even more preferably about 7.6, when the solid composition is reconstituted in water or saline, particularly 0.5%-1.0% (e.g. 0.9% (w/v)) saline and pH is measured at 20-25 °C.

### Stability

As described herein, a composition of the invention is typically more stable than corresponding compositions of chimeric clostridial neurotoxins known in the art. In particular, compositions of the invention are typically more stable than corresponding chimeric clostridial neurotoxins compositions which do not comprise a dual histidine buffer, i.e. which do not comprise histidine-HCl and/or L-histidine.

A composition of the invention may have improved ambient stability compared with corresponding chimeric clostridial neurotoxins compositions which do not comprise a dual histidine buffer, as described herein.

Alternatively or in addition, a composition of the invention may have improved cold-storage stability compared with corresponding chimeric clostridial neurotoxins compositions which do not comprise a dual histidine buffer.

Stability of a composition of the invention may be quantified in terms of any appropriate parameter. For example, stability may be quantified in terms of the potency of the chimeric clostridial neurotoxin and/or in terms of the % recovery of the chimeric clostridial neurotoxin over time. By way of non-limiting example, a composition of the invention may be deemed stable if it exhibits at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more of its potency when compared to a suitable reference or control (e.g. its initial or baseline potency, or the potency of a reference standard for that chimeric clostridial neurotoxin).

### Therapeutic and Cosmetic Indications

The compositions of the invention suitably find utility in medicine and/or in cosmetics. Typically, solid compositions are reconstituted (e.g. in water or saline) before use, as described herein.

The compositions of the invention may be used to prevent or treat certain medical diseases and conditions, and/or certain cosmetic conditions. Thus, the present invention provides a composition as described above, for use in medicine or for cosmetic use. Liquid compositions of the invention may be directly administered to a subject. Typically, solid compositions are reconstituted (e.g. in water or saline) before administration to a subject

The present invention also provides compositions as described above, for use in medicine.

Accordingly, the present invention provides a composition as described above, for use in the prevention or treatment of a disease or condition associated with unwanted neuronal activity. A composition of the invention may be used in the prevention or treatment of a disease or condition selected from: a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder. In some instances, the condition may be selected from phantom pain (e.g. phantom limb pain) and bladder pain syndrome. Preferably, a composition of the invention may be used in the prevention or treatment of a disease or condition selected from: limb spasticity (upper or lower); cervical dystonia; headache disorders (preferably migraine); blepharospasm; hemifacial spasm; and lower urinary tract disorders (e.g. bladder pain syndrome (preferably interstitial cystitis); overactive bladder; and detrusor overactivity (e.g. neurogenic detrusor overactivity.

The invention also provides the use of a composition as described above, in the manufacture of a medicament for use in a method for preventing or treating a disease or disorder as described herein.

The invention also provides a method of treating a disease or disorder as described herein, said method comprising administering a therapeutically effective amount of a composition as described above to a subject in need thereof.

The invention also provides the use of a composition as described herein for preventing or alleviating a disease or condition for which the application of a botulinum neurotoxin is indicated.

The invention also provides the non-therapeutic use of a composition as described herein for treating an aesthetic or cosmetic condition. For cosmetic or aesthetic use, the individual to be treated is preferably not suffering from a disease or disorder, such as those associated with unwanted neuronal activity and described above. More preferably, said individual is a healthy individual, i.e. an individual which is not suffering from any disease. Preferably, a composition of the invention may be used in the prevention or treatment of upper facial lines-glabellar lines, lateral canthal lines and/or intrathecal lines.

The compositions of the invention may be pharmaceutical or cosmetic. In addition to the components described herein, a pharmaceutical composition of the invention may further comprise a pharmaceutically acceptable carrier, excipient, adjuvant, propellant and/or salt. A cosmetic or aesthetic composition of the invention may further comprise a cosmetically or aesthetically acceptable carrier, excipient, adjuvant, propellant and/or salt.

The invention also provides the use of a cosmetic composition as described herein for preventing or alleviating a cosmetic indication for which the application of a botulinum neurotoxin is indicated.

The compositions of the invention may be used for oral, parenteral, continuous infusion, inhalation or topical application. Compositions suitable for injection may be in the form of solutions, suspensions or emulsions, or dry powders which are dissolved or suspended in a suitable vehicle prior to use. Typically the compositions of the invention are suitable for injection. Solid compositions of the invention may be reconstituted as described herein to render them suitable for injection.

In the case of a composition that is to be delivered locally, the composition may be for sub-dermal injection, or further formulated as a cream (e.g. for topical application).

Local delivery means may include an aerosol, or other spray (e.g. a nebuliser). In this regard, an aerosolization of a composition of the invention enables delivery to the lungs and/or other nasal and/or bronchial or airway passages.

Compositions of the invention may be administered to a patient by intrathecal or epidural injection in the spinal column at the level of the spinal segment involved in the innervation of an affected organ.

A preferred route of administration is via laproscopic and/or localised, particularly intramuscular, injection.

The dosage ranges for administration of the compositions of the present invention are those to produce the desired therapeutic effect. A therapeutically effective dose refers to an amount of the chimeric neurotoxin, to be used in a composition of the present invention which prevents, ameliorates or treats the symptoms accompanying a disease or condition referred herein. Therapeutic efficacy and toxicity of the compound are typically determined in the art by standard pharmaceutical procedures in cell cultures or experimental animals, e.g., ED₅₀ (the dose therapeutically effective in 50% of the population) and LD50 (the dose lethal to 50% of the population). The dose ratio between therapeutic and toxic effects is the therapeutic index, and it can be expressed as the ratio, LD50/ED₅₀. In general terms, it will be appreciated that the dosage range required for a given pharmaceutical depends on the precise nature of the composition, the route of administration, the nature of the formulation, the age of the patient, the nature, extent or severity of the patient's condition, contraindications, if any, and the judgement of the attending physician.

In relation to the chimeric neurotoxin compositions of the invention, suitable single unit doses (also referred to as unit doses), i.e. the dose administered per injection site are described in the art, such as in WO2021/186160, WO2021/186167, WO2023/047127, WO2023/089343 and WO2023/041934, each of which is herein incorporated by reference in its entirety. By way of non-limiting example, a single unit dose is 15,000 μg of chimeric neurotoxin, 25,000 pg of chimeric neurotoxin or 36,000 μg of chimeric neurotoxin.

A treatment may comprise injections at multiple injection sites (typically no more than 20, preferably no more than 15 injection sites), with a single unit dose injected at each injection site.

Liquid compositions may be sterilised by filtration through a sterile filter using aseptic techniques before filling into suitable sterile containers (e.g. vials or ampoules) and sealing. Alternatively, if solution stability is adequate, the solution in its sealed containers may be sterilised by autoclaving. Additives such as preservative or bactericidal, suspending or emulsifying agents and or local anaesthetic agents may be dissolved in the vehicle.

Solid formulations (e.g. dry powders), which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then lyophilised, and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, viral delivery systems or high-pressure aerosol impingement.

The invention also provides a kit comprising a (solid or liquid) composition as described herein and instructions for therapeutic or cosmetic administration of said composition to a subject in need thereof. More precisely, the invention relates to a kit comprising a composition of the invention and instructions for therapeutic or cosmetic administration of said composition to an individual in need thereof As used herein, the term “instructions” refers to a publication, a recording, a diagram, or any other medium of expression which can be used to communicate how to perform a method or use of the invention, such as therapeutic or cosmetic administration of said composition to an individual in need thereof Said instructions can, for example, be affixed to a container which comprises said composition or said kit.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264 (4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8 (5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20 (9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 1 | 1 | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}\begin{matrix}length of the longer sequence plus the \\ number of gaps introduced into the longer\end{matrix} \\ sequence in order to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### SEQUENCE INFORMATION

Where an initial Met amino acid residue or a corresponding initial codon is indicated in any of the following SEQ ID NOs, said residue/codon is optional.

| | |
|---|---|
| SEQ ID NO: 1 | BoNT/AB chimera amino acid sequence (mrBoNT/AB) |
| SEQ ID NO: 2 | Reference BoNT/A amino acid sequence |
| SEQ ID NO: 3 | Reference BoNT/B amino acid sequence |
| SEQ ID NO: 4 | Reference BoNT/C1 amino acid sequence |
| SEQ ID NO: 5 | Reference BoNT/D amino acid sequence |
| SEQ ID NO: 6 | Reference BoNT/E amino acid sequence |
| SEQ ID NO: 7 | Reference BoNT/F amino acid sequence |
| SEQ ID NO: 8 | Reference BoNT/G amino acid sequence |
| SEQ ID NO: 9 | Reference BoNT/X amino acid sequence |
| SEQ ID NO: 10 | Reference TeNT amino acid sequence |
| SEQ ID NO: 11 | C-terminal L-chain fragment |
| SEQ ID NO: 12 | C-terminal L-chain fragment 2 |
| SEQ ID NO: 13 | Di-Chain L-Chain 1 |
| SEQ ID NO: 14 | Di-Chain L-Chain 2 |
| SEQ ID NO: 15 | Di-Chain H-Chain |
| SEQ ID NO: 16 | TEV cleavage site |
| SEQ ID NO: 17 | Thrombin cleavage site |
| SEQ ID NO: 18 | PreScission cleavage site |
| SEQ ID NO: 19 | HExxH motif |
| SEQ ID NO: 20 | Influenza virus haemagglutinin translocation domain |
| SEQ ID NO: 21 | xDxxxLL motif |
| SEQ ID NO: 22 | xExxxLL motif |
| SEQ ID NO: 23 | xExxxIL motif |
| SEQ ID NO: 24 | xExxxLM motif |
| SEQ ID NO: 25 | Y-x-x-Hy motif |
| SEQ ID NO: 26 | exemplary BoNT/AB chimera amino acid sequence (mrBoNT/AB) |
| SEQ ID NO: 27 | exemplary BoNT/AB chimera amino acid sequence (mrBoNT/AB) |
| SEQ ID NO: 28 | exemplary BoNT/AB chimera amino acid sequence (mrBoNT/AB) |
| SEQ ID NO: 29 | exemplary BoNT/BC chimera amino acid sequence (mrBoNT/BC) |
| SEQ ID NO: 30 | nucleotide sequence of modified (cationic) BoNT/A “Cat-A” |
| SEQ ID NO: 31 | polypeptide sequence of modified (cationic) BoNT/A “Cat-A” |
| SEQ ID NO: 32 | nucleotide sequence of modified (cationic) BoNT/A “Cat-B” |
| SEQ ID NO: 33 | polypeptide sequence of modified (cationic) BoNT/A “Cat-B” |
| SEQ ID NO: 34 | nucleotide sequence of modified (cationic) BoNT/A “Cat-C” |
| SEQ ID NO: 35 | polypeptide sequence of modified (cationic) BoNT/A “Cat-C” |
| SEQ ID NO: 36 | nucleotide sequence of modified (cationic) BoNT/A “Cat-D” |
| SEQ ID NO: 37 | polypeptide sequence of modified (cationic) BoNT/A “Cat-D” |
| SEQ ID NO: 38 | Carrier component |
| SEQ ID NO: 39 | Carrier component |
| SEQ ID NO: 40 | Carrier component |
| SEQ ID NO: 41 | Carrier component |

SEQ ID NO: 1 - Polypeptide Sequence of Chimeric Clostridial Neurotoxin 1
(mrBoNT/AB)
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NILNNIILNLRYKDNNLIDLSGYGAKVEVYDGVELNDKNQFKLTSSANSKIRVTQNQ
NIIFNSVFLDFSVSFWIRIPKYKNDGIQNYIHNEYTIINCMKNNSGWKISIRGNRIIWTLI
DINGKTKSVFFEYNIREDISEYINRWFFVTITNNLNNAKIYINGKLESNTDIKDIREVIA
NGEIIFKLDGDIDRTQFIWMKYFSIFNTELSQSNIEERYKIQSYSEYLKDFWGNPLMYN
KEYYMFNAGNKNSYIKLKKDSPVGEILTRSKYNQNSKYINYRDLYIGEKFIIRRKSNS
QSINDDIVRKEDYIYLDFFNLNQEWRVYTYKYFKKEEMKLFLAPIYDSDEFYNTIQIK
EYDEQPTYSCQLLFKKDEESTDEIGLIGIHRFYESGIVFEEYKDYFCISKWYLKEVKRK
PYNLKLGCNWQFIPKDEGWTE
SEQ ID NO: 2 - Polypeptide Sequence of Native BoNT/A (rBoNT/A)
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NIINTSILNLRYESNHLIDLSRYASKINIGSKVNFDPIDKNQIQLFNLESSKIEVILKNAIV
YNSMYENFSTSFWIRIPKYFNSISLNNEYTIINCMENNSGWKVSLNYGEIIWTLQDTQ
EIKQRVVFKYSQMINISDYINRWIFVTITNNRLNNSKIYINGRLIDQKPISNLGNIHASN
NIMFKLDGCRDTHRYIWIKYFNLFDKELNEKEIKDLYDNQSNSGILKDFWGDYLQYD
KPYYMLNLYDPNKYVDVNNVGIRGYMYLKGPRGSVMTTNIYLNSSLYRGTKFIIKK
YASGNKDNIVRNNDRVYINVVVKNKEYRLATNASQAGVEKILSALEIPDVGNLSQV
VVMKSKNDQGITNKCKMNLQDNNGNDIGFIGFHQFNNIAKLVASNWYNRQIERSSR
TLGCSWEFIPVDDGWGERPL
SEQ ID NO: 3 - Polypeptide Sequence of BoNT/B
MPVTINNFNYNDPIDNNNIIMMEPPFARGTGRYYKAFKITDRIWIIPERYTFGYKPEDF
NKSSGIFNRDVCEYYDPDYLNTNDKKNIFLQTMIKLFNRIKSKPLGEKLLEMIINGIPY
LGDRRVPLEEFNTNIASVTVNKLISNPGEVERKKGIFANLIIFGPGPVLNENETIDIGIQ
NHFASREGFGGIMQMKFCPEYVSVENNVQENKGASIFNRRGYFSDPALILMHELIHV
LHGLYGIKVDDLPIVPNEKKFFMQSTDAIQAEELYTFGGQDPSIITPSTDKSIYDKVLQ
NFRGIV
DRLNKVLVCISDPNININIYKNKFKDKYKFVEDSEGKYSIDVESFDKLYKSLMFGFTE
TNIAENYKIKTRASYFSDSLPPVKIKNLLDNEIYTIEEGFNISDKDMEKEYRGQNKAIN
KQAYEEISKEHLAVYKIQMCKSVKAPGICIDVDNEDLFFIADKNSFSDDLSKNERIEY
NTQSNYIENDFPINELILDTDLISKIELPSENTESLTDFNVDVPVYEKQPAIKKIFTDENT
IFQYLYSQTFPLDIRDISLTSSFDDALLFSNKVYSFFSMDYIKTANKVVEAGLFAGWV
KQIVND
FVIEANKSNTMDKIADISLIVPYIGLALNVGNETAKGNFENAFEIAGASILLEFIPELLIP
VVGAFLLESYIDNKNKIIKTIDNALTKRNEKWSDMYGLIVAQWLSTVNTQFYTIKEG
MYKALNYQAQALEEIIKYRYNIYSEKEKSNINIDENDINSKLNEGINQAIDNINNFING
CSVSYLMKKMIPLAVEKLLDFDNTLKKNLLNYIDENKLYLIGSAEYEKSKVNKYLKT
IMPFDLSIYTNDTILIEMENKYNSEILNNIILNLRYKDNNLIDLSGYGAKVEVYDGVEL
NDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSYS
EYLKDFWGNPLMYNKEYYMFNAGNKNSYIKLKKDSPVGEILTRSKYNQNSKYINYR
DLYIGEKFIIRRKSNSQSINDDIVRKEDYIYLDFFNLNQEWRVYTYKYFKKEEEKLFL
APISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 4 - Polypeptide Sequence of BoNT/C₁
MPITINNFNYSDPVDNKNILYLDTHLNTLANEPEKAFRITGNIWVIPDRFSRNSNPNLN
KPPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTFKLTNNT
FAAQEGFGALSIISISPRFMLTYSNATNDVGEGRFSKSEFCMDPILILMHELNHAMHN
LYGIAIPNDQTISSVTSNIFYSQYNVKLEYAEIYAFGGPTIDLIPKSARKYFEEKALDYY
RSI
AKRLNSITTANPSSFNKYIGEYKQKLIRKYRFVVESSGEVTVNRNKFVELYNELTQIF
TEFNYAKIYNVQNRKIYLSNVYTPVTANILDDNVYDIQNGFNIPKSNLNVLFMGQNL
SRNPALRKVNPENMLYLFTKFCHKAIDGRSLYNKTLDCRELLVKNTDLPFIGDISDV
KTDIFLRKDINEETEVIYYPDNVSVDQVILSKNTSEHGQLDLLYPSIDSESEILPGENQV
FYDNRTQNVDYLNSYYYLESQKLSDNVEDFTFTRSIEEALDNSAKVYTYFPTLANKV
NAGVQGGLFLM
WANDVVEDFTTNILRKDTLDKISDVSAIIPYIGPALNISNSVRRGNFTEAFAVTGVTIL
LEAFPEFTIPALGAFVIYSKVQERNEIIKTIDNCLEQRIKRWKDSYEWMMGTWLSRIIT
QFNNISYQMYDSLNYQAGAIKAKIDLEYKKYSGSDKENIKSQVENLKNSLDVKISEA
MNNINKFIRECSVTYLFKNMLPKVIDELNEFDRNTKAKLINLIDSHNIILVGEVDKLK
AKVNNSFQNTIPFNIFSYTNNSLLKDIINEYFNNINDSKILSLQNRKNTLVDTSGYNAE
VSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMM
GNMKIYINGKLIDTIKVKELTGINFSKTITFEINKIPDTGLITSDSDNINMWIRDFYIFAK
ELDGKDINILFNSLQYTNVVKDYWGNDLRYNKEYYMVNIDYLNRYMYANSRQIVF
NTRRNNNDFNEGYKIIIKRIRGNTNDTRVRGGDILYFDMTINNKAYNLFMKNETMYA
DNHSTEDIYA
IGLREQTKDINDNIIFQIQPMNNTYYYASQIFKSNFNGENISGICSIGTYRFRLGGDWY
RHNYLVPTVKQGNYASLLESTSTHWGFVPVSE
SEQ ID NO: 5 - Polypeptide Sequence of BoNT/D
MTWPVKDFNYSDPVNDNDILYLRIPQNKLITTPVKAFMITQNIWVIPERFSSDTNPSLS
KPPRPTSKYQSYYDPSYLSTDEQKDTFLKGIIKLFKRINERDIGKKLINYLVVGSPFMG
DSSTPEDTFDFTRHTTNIAVEKFENGSWKVTNIITPSVLIFGPLPNILDYTASLTLQGQQ
SNPSFEGFGTLSILKVAPEFLLTFSDVTSNQSSAVLGKSIFCMDPVIALMHELTHSLHQ
LYGINIPSDKRIRPQVSEGFFSQDGPNVQFEELYTFGGLDVEIIPQIERSQLREKALGHY
KDI
AKRLNNINKTIPSSWISNIDKYKKIFSEKYNFDKDNTGNFVVNIDKFNSLYSDLTNVM
SEVVYSSQYNVKNRTHYFSRHYLPVFANILDDNIYTIRDGFNLTNKGFNIENSGQNIE
RNPALQKLSSESVVDLFTKVCLRLTKNSRDDSTCIKVKNNRLPYVADKDSISQEIFEN
KIITDETNVQNYSDKFSLDESILDGQVPINPEIVDPLLPNVNMEPLNLPGEEIVFYDDIT
KYVDYLNSYYYLESQKLSNNVENITLTTSVEEALGYSNKIYTFLPSLAEKVNKGVQA
GLFLNWANE
VVEDFTTNIMKKDTLDKISDVSVIIPYIGPALNIGNSALRGNFNQAFATAGVAFLLEGF
PEFTIPALGVFTFYSSIQEREKIIKTIENCLEQRVKRWKDSYQWMVSNWLSRITTQFNH
INYQMYDSLSYQADAIKAKIDLEYKKYSGSDKENIKSQVENLKNSLDVKISEAMNNI
NKFIRECSVTYLFKNMLPKVIDELNKFDLRTKTELINLIDSHNIILVGEVDRLKAKVNE
SFENTMPFNIFSYTNNSLLKDIINEYFNSINDSKILSLQNKKNALVDTSGYNAEVRVGD
NVQLNTI
YTNDFKLSSSGDKIIVNLNNNILYSAIYENSSVSFWIKISKDLTNSHNEYTIINSIEQNSG
WKLCIRNGNIEWILQDVNRKYKSLIFDYSESLSHTGYTNKWFFVTITNNIMGYMKLYI
NGELKQSQKIEDLDEVKLDKTIVFGIDENIDENQMLWIRDFNIFSKELSNEDINIVYEG
QILRNVIKDYWGNPLKFDTEYYIINDNYIDRYIAPESNVLVLVQYPDRSKLYTGNPITI
KSVSDKNPYSRILNGDNIILHMLYNSRKYMIIRDTDTIYATQGGECSQNCVYALKLQS
NLGNY
GIGIFSIKNIVSKNKYCSQIFSSFRENTMLLADIYKPWRFSFKNAYTPVAVTNYETKLL
STSSFWKFISRDPGWVE
SEQ ID NO: 6 - Polypeptide Sequence of BoNT/E
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDN
TPDNQFHIGDASAVEIKFSNGSQDILLPNVIIMGAEPDLFETNSSNISLRNNYMPSNHR
FGSIAIVTFSPEYSFRFNDNCMNEFIQDPALTLMHELIHSLHGLYGAKGITTKYTITQK
QNPLITNIRGTNIEEFLTFGGTDLNIITSAQSNDIYTNLLADYKKIASKLSKVQVSNPLL
NPYK
DVFEAKYGLDKDASGIYSVNINKENDIFKKLYSFTEFDLRTKFQVKCRQTYIGQYKY
FKLSNLLNDSIYNISEGYNINNLKVNFRGQNANLNPRIITPITGRGLVKKIIRFCKNIVS
VKGIRKSICIEINNGELFFVASENSYNDDNINTPKEIDDTVTSNNNYENDLDQVILNEN
SESAPGLSDEKLNLTIQNDAYIPKYDSNGTSDIEQHDVNELNVFFYLDAQKVPEGEN
NVNLTSSIDTALLEQPKIYTFFSSEFINNVNKPVQAALFVSWIQQVLVDFTTEANQKST
VDKIADIS
IVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIK
TIIESKYNSYTLEEKNELTNKYDIKQIENELNQKVSIAMNNIDRFLTESSISYLMKIINE
VKINKLREYDENVKTYLLNYIIQHGSILGESQQELNSMVTDTLNNSIPFKLSSYTDDKI
LISYFNKFFKRIKSSSVLNMRYKNDKYVDTSGYDSNININGDVYKYPTNKNQFGIYN
DKLSEVNI
SQNDYIIYDNKYKNFSISFWVRIPNYDNKIVNVNNEYTIINCMRDNNSGWKVSLNHN
EIIWTFEDNRGINQKLAFNYGNANGISDYINKWIFVTITNDRLGDSKLYINGNLIDQKS
ILNLGNIHVSDNILFKIVNCSYTRYIGIRYFNIFDKELDETEIQTLYSNEPNTNILKDFW
GNYLLYDKEYYLLNVLKPNNFIDRRKDSTLSINNIRSTILLANRLYSGIKVKIQRVNNS
STNDNLVRKNDQVYINFVASKTHLFPLYADTATTNKEKTIKISSSGNRFNQVVVMNS
VGNCTMNF
KNNNGNNIGLLGFKADTVVASTWYYTHMRDHTNSNGCFWNFISEEHGWQEK
SEQ ID NO: 7 - Polypeptide Sequence of BoNT/F
MPVVINSFNYNDPVNDDTILYMQIPYEEKSKKYYKAFEIMRNVWIIPERNTIGTDPSD
FDPPASLENGSSAYYDPNYLTTDAEKDRYLKTTIKLFKRINSNPAGEVLLQEISYAKP
YLGNEHTPINEFHPVTRTTSVNIKSSTNVKSSIILNLLVLGAGPDIFENSSYPVRKLMDS
GGVYDPSNDGFGSINIVTFSPEYEYTFNDISGGYNSSTESFIADPAISLAHELIHALHGL
YGARGVTYKETIKVKQAPLMIAEKPIRLEEFLTFGGQDLNIITSAMKEKIYNNLLANY
EKIATR
LSRVNSAPPEYDINEYKDYFQWKYGLDKNADGSYTVNENKFNEIYKKLYSFTEIDLA
NKFKVKCRNTYFIKYGFLKVPNLLDDDIYTVSEGFNIGNLAVNNRGQNIKLNPKIIDSI
PDKGLVEKIVKFCKSVIPRKGTKAPPRLCIRVNNRELFFVASESSYNENDINTPKEIDD
TTNLNNNYRNNLDEVILDYNSETIPQISNQTLNTLVQDDSYVPRYDSNGTSEIEEHNV
VDLNVFFYLHAQKVPEGETNISLTSSIDTALSEESQVYTFFSSEFINTINKPVHAALFIS
WINQVIR
DFTTEATQKSTFDKIADISLVVPYVGLALNIGNEVQKENFKEAFELLGAGILLEFVPEL
LIPTILVFTIKSFIGSSENKNKIIKAINNSLMERETKWKEIYSWIVSNWLTRINTQFNKR
KEQMYQALQNQVDAIKTVIEYKYNNYTSDERNRLESEYNINNIREELNKKVSLAME
NIERFITESSIFYLMKLINEAKVSKLREYDEGVKEYLLDYISEHRSILGNSVQELNDLV
TSTLNNSIPFELSSYTNDKILILYFNKLYKKIKDNSILDMRYENNKFIDISGYGSNISING
DVYIY
STNRNQFGIYSSKPSEVNIAQNNDIIYNGRYQNFSISFWVRIPKYFNKVNLNNEYTIID
CIRNNNSGWKISLNYNKIIWTLQDTAGNNQKLVFNYTQMISISDYINKWIFVTITNNR
LGNSRIYINGNLIDEKSISNLGDIHVSDNILFKIVGCNDTRYVGIRYFKVFDTELGKTEI
ETLYSDEPDPSILKDFWGNYLLYNKRYYLLNLLRTDKSITQNSNFLNINQQRGVYQK
PNIFSNTRLYTGVEVIIRKNGSTDISNTDNFVRKNDLAYINVVDRDVEYRLYADISIAK
PEKIIKL
IRTSNSNNSLGQIIVMDSIGNNCTMNFQNNNGGNIGLLGFHSNNLVASSWYYNNIRK
NTSSNGCFWSFISKEHGWQEN
SEQ ID NO: 8 - Polypeptide Sequence of BoNT/G
MPVNIKNFNYNDPINNDDIIMMEPFNDPGPGTYYKAFRIIDRIWIVPERFTYGFQPDQF
NASTGVFSKDVYEYYDPTYLKTDAEKDKFLKTMIKLFNRINSKPSGQRLLDMIVDAI
PYLGNASTPPDKFAANVANVSINKKIIQPGAEDQIKGLMTNLIIFGPGPVLSDNFTDSM
IMNGHSPISEGFGARMMIRFCPSCLNVFNNVQENKDTSIFSRRAYFADPALTLMHELI
HVLHGLYGIKISNLPITPNTKEFFMQHSDPVQAEELYTFGGHDPSVISPSTDMNIYNK
ALQNFQDIANRLNIVSSAQGSGIDISLYKQIYKNKYDFVEDPNGKYSVDKDKFDKLY
KALMFGFTETNLAGEYGIKTRYSYFSEYLPPIKTEKLLDNTIYTQNEGENIASKNLKTE
FNGQNKAVNKEAYEEISLEHLVIYRIAMCKPVMYKNTGKSEQCIIVNNEDLFFIANK
DSFSKDLAKAETIAYNTQNNTIENNFSIDQLILDNDLSSGIDLPNENTEPFTNFDDIDIP
VYIKQSALKKIFVDGDSLFEYLHAQTFPSNIENLQLTNSLNDALRNNNKVYTFFSTNL
VEKANTVVGASLFVNWVKGVIDDFTSESTQKSTIDKVSDVSIIIPYIGPALNVGNETA
KENFKNAFEIGGAAILMEFIPELIVPIVGFFTLESYVGNKGHIIMTISNALKKRDQKWT
DMYGLIVSQWLSTVNTQFYTIKERMYNALNNQSQAIEKIIEDQYNRYSEEDKMNINI
DENDIDFKLNQSINLAINNIDDFINQCSISYLMNRMIPLAVKKLKDFDDNLKRDLLEYI
DTNELYLLDEVNILKSKVNRHLKDSIPFDLSLYTKDTILIQVENNYISNISSNAILSLSY
RGGRLIDSSGYGATMNVGSDVIFNDIGNGQFKLNNSENSNITAHQSKFVVYDSMFDN
FSINFWVRTPKYNNNDIQTYLQNEYTIISCIKNDSGWKVSIKGNRIIWTLIDVNAKSKS
IFFEYSIKDNISDYINKWFSITITNDRLGNANIYINGSLKKSEKILNLDRINSSNDIDFKLI
NCTDTTKFVWIKDFNIFGRELNATEVSSLYWIQSSTNTLKDFWGNPLRYDTQYYLFN
QGMQNIYIKYFSKASMGETAPRTNFNNAAINYQNLYLGLRFIIKKASNSRNINNDNIV
REGDYIYLNIDNISDESYRVYVLVNSKEIQTQLFLAPINDDPTFYDVLQIKKYYEKTTY
NCQILCEKDTKTFGLFGIGKFVKDYGYVWDTYDNYFCISQWYLRRISENINKLRLGC
NWQFIPVDEGWTE
SEQ ID NO: 9 - Polypeptide Sequence of BoNT/X
MKLEINKFNYNDPIDGINVITMRPPRHSDKINKGKGPFKAFQVIKNIWIVPERYNFTN
NTNDLNIPSEPIMEADAIYNPNYLNTPSEKDEFLQGVIKVLERIKSKPEGEKLLELISSSI
PLPLVSNGALTLSDNETIAYQENNNIVSNLQANLVIYGPGPDIANNATYGLYSTPISNG
EGTLSEVSFSPFYLKPFDESYGNYRSLVNIVNKFVKREFAPDPASTLMHELVHVTHNL
YGISNRNFYYNFDTGKIETSRQQNSLIFEELLTFGGIDSKAISSLIIKKIIETAKNNYTTLI
SE
RLNTVTVENDLLKYIKNKIPVQGRLGNFKLDTAEFEKKLNTILFVLNESNLAQRFSIL
VRKHYLKERPIDPIYVNILDDNSYSTLEGFNISSQGSNDFQGQLLESSYFEKIESNALR
AFIKICPRNGLLYNAIYRNSKNYLNNIDLEDKKTTSKTNVSYPCSLLNGCIEVENKDL
FLISNKDSLNDINLSEEKIKPETTVFFKDKLPPQDITLSNYDFTEANSIPSISQQNILERN
EELYEPIRNSLFEIKTIYVDKLTTFHFLEAQNIDESIDSSKIRVELTDSVDEALSNPNKV
YSPF
KNMSNTINSIETGITSTYIFYQWLRSIVKDFSDETGKIDVIDKSSDTLAIVPYIGPLLNIG
NDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNI
DDKAKIKNAISETEILLNKSVEQAMKNTEKFMIKLSNSYLTKEMIPKVQDNLKNFDL
ETKKTLDKFIKEKEDILGTNLSSSLRRKVSIRLNKNIAFDINDIPFSEFDDLINQYKNEIE
DYEVLNL
GAEDGKIKDLSGTTSDINIGSDIELADGRENKAIKIKGSENSTIKIAMNKYLRFSATDN
FSISFWIKHPKPTNLLNNGIEYTLVENFNQRGWKISIQDSKLIWYLRDHNNSIKIVTPD
YIAFNGWNLITITNNRSKGSIVYVNGSKIEEKDISSIWNTEVDDPIIFRLKNNRDTQAFT
LLDQFSIYRKELNQNEVVKLYNYYFNSNYIRDIWGNPLQYNKKYYLQTQDKPGKGL
IREYWSSFGYDYVILSDSKTITFPNNIRYGALYNGSKVLIKNSKKLDGLVRNKDFIQLE
IDGYNMG
ISADRFNEDTNYIGTTYGTTHDLTTDFEIIQRQEKYRNYCQLKTPYNIFHKSGLMSTET
SKPTEHDYRDWVYSSAWYFQNYENLNLRKHTKTNWYFIPKDEGWDED
SEQ ID NO: 10 - Polypeptide Sequence of TeNT
MPITINNFRYSDPVNNDTIIMMEPPYCKGLDIYYKAFKITDRIWIVPERYEFGTKPEDF
NPPSSLIEGASEYYDPNYLRTDSDKDRFLQTMVKLFNRIKNNVAGEALLDKIINAIPY
LGNSYSLLDKFDTNSNSVSFNLLEQDPSGATTKSAMLTNLIIFGPGPVLNKNEVRGIV
LRVDNKNYFPCRDGFGSIMQMAFCPEYVPTFDNVIENITSLTIGKSKYFQDPALLLMH
ELIHVLHGLYGMQVSSHEIIPSKQEIYMQHTYPISAEELFTFGGQDANLISIDIKNDLYE
KTLNDYK
AIANKLSQVTSCNDPNIDIDSYKQIYQQKYQFDKDSNGQYIVNEDKFQILYNSIMYGF
TEIELGKKFNIKTRLSYFSMNHDPVKIPNLLDDTIYNDTEGFNIESKDLKSEYKGQNM
RVNTNAFRNVDGSGLVSKLIGLCKKIIPPTNIRENLYNRTASLTDLGGELCIKIKNEDL
TFIAEKNSFSEEPFQDEIVSYNTKNKPLNFNYSLDKIIVDYNLQSKITLPNDRTTPVTK
GIPYAPEYKSNAASTIEIHNIDDNTIYQYLYAQKSPTTLQRITMTNSVDDALINSTKIYS
YFPSVI
SKVNQGAQGILFLQWVRDIIDDFTNESSQKTTIDKISDVSTIVPYIGPALNIVKQGYEG
NFIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINN
LKNKLEEKANKAMININIFMRESSRSFLVNQMINEAKKQLLEFDTQSKNILMQYIKA
NSKFIGITELKKLESKINKVFSTPIPFSYSKNLDCWVDNEEDIDVILKKSTILNLDINNDI
ISDIS
GFNSSVITYPDAQLVPGINGKAIHLVNNESSEVIVHKAMDIEYNDMFNNFTVSFWLR
VPKVSASHLEQYGTNEYSIISSMKKHSLSIGSGWSVSLKGNNLIWTLKDSAGEVRQIT
FRDLPDKFNAYLANKWVFITITNDRLSSANLYINGVLMGSAEITGLGAIREDNNITLK
LDRCNNNNQYVSIDKFRIFCKALNPKEIEKLYTSYLSITFLRDFWGNPLRYDTEYYLIP
VASSSKDVQLKNITDYMYLTNAPSYTNGKLNIYYRRLYNGLKFIIKRYTPNNEIDSFV
KSGDFIKLYV
SYNNNEHIVGYPKDGNAFNNLDRILRVGYNAPGIPLYKKMEAVKLRDLKTYSVQLK
LYDDKNASLGLVGTHNGQIGNDPNRDILIASNWYFNHLKDKILGCDWYFVPTDEGW
TND
SEQ ID NO: 11 - C-terminal L-chain Fragment
TKSLDKGYNK
SEQ ID NO: 12 - C-terminal L-chain Fragment 2
SLDKGYNK
SEQ ID NO: 13 - Di-Chain L-Chain 1
PFVNKQFNYKDPVNGVDIAYIKIPNAGQMQPVKAFKIHNKIWVIPERDTFTNPEEGDL
NPPPEAKQVPVSYYDSTYLSTDNEKDNYLKGVTKLFERIYSTDLGRMLLTSIVRGIPF
WGGSTIDTELKVIDTNCINVIQPDGSYRSEELNLVIIGPSADIIQFECKSFGHEVLNLTR
NGYGSTQYIRFSPDFTFGFEESLEVDTNPLLGAGKFATDPAVTLAHELIHAGHRLYGI
AINPNRVFKVNTNAYYEMSGLEVSFEELRTFGGHDAKFIDSLQENEFRLYYYNKFKD
IASTLNKAKSIVGTTASLQYMKNVFKEKYLLSEDTSGKFSVDKLKFDKLYKMLTEIY
TEDNFVKFFKVLNRKTYLNFDKAVFKINIVPKVNYTIYDGFNLRNTNLAANFNGQNT
EINNMNFTKLKNFTGLFEFYKLLCVRGIITSK
SEQ ID NO: 14 - Di-Chain L-Chain 2
PFVNKQFNYKDPVNGVDIAYIKIPNAGQMQPVKAFKIHNKIWVIPERDTFTNPEEGDL
NPPPEAKQVPVSYYDSTYLSTDNEKDNYLKGVTKLFERIYSTDLGRMLLTSIVRGIPF
WGGSTIDTELKVIDTNCINVIQPDGSYRSEELNLVIIGPSADIIQFECKSFGHEVLNLTR
NGYGSTQYIRFSPDFTFGFEESLEVDTNPLLGAGKFATDPAVTLAHELIHAGHRLYGI
AINPNRVFKVNTNAYYEMSGLEVSFEELRTFGGHDAKFIDSLQENEFRLYYYNKFKD
IASTLNKAKSIVGTTASLQYMKNVFKEKYLLSEDTSGKFSVDKLKFDKLYKMLTEIY
TEDNFVKFFKVLNRKTYLNFDKAVFKINIVPKVNYTIYDGFNLRNTNLAANFNGQNT
EINNMNFTKLKNFTGLFEFYKLLCVRGIITSKTK
SEQ ID NO: 15 - Di-Chain H-Chain
ALNDLCIKVNNWDLFFSPSEDNFTNDLNKGEEITSDTNIEAAEENISLDLIQQYYLTFN
FDNEPENISIENLSSDIIGQLELMPNIERFPNGKKYELDKYTMFHYLRAQEFEHGKSRI
ALTNSVNEALLNPSRVYTFFSSDYVKKVNKATEAAMFLGWVEQLVYDFTDETSEVS
TTDKIADITIIIPYIGPALNIGNMLYKDDFVGALIFSGAVILLEFIPEIAIPVLGTFALVSYI
ANKVLTVQTIDNALSKRNEKWDEVYKYIVTNWLAKVNTQIDLIRKKMKEALENQA
EATKAIINYQYNQYTEEEKNNINFNIDDLSSKLNESINKAMININKFLNQCSVSYLMN
SMIPYGVKRLEDFDASLKDALLKYIYDNRGTLIGQVDRLKDKVNNTLSTDIPFQLSK
YVDNQRLLSTFTEYIKNILNNIILNLRYKDNNLIDLSGYGAKVEVYDGVELNDKNQF
KLTSSANSKIRVTQNQNIIFNSVFLDFSVSFWIRIPKYKNDGIQNYIHNEYTIINCMKNN
SGWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSYS
EYLKDFWGNPLMYNKEYYMFNAGNKNSYIKLKKDSPVGEILTRSKYNQNSKYINYR
DLYIGEKFIIRRKSNSQSINDDIVRKEDYIYLDFFNLNQEWRVYTYKYFKKEEMKLFL
APIYDSDEFYNTIQIKEYDEQPTYSCQLLFKKDEESTDEIGLIGIHRFYESGIVFEEYKD
YFCISKWYLKEVKRKPYNLKLGCNWQFIPKDEGWTE
SEQ ID NO: 16 - TEV cleavage site
ENLYFQG
SEQ ID NO: 17 - Thrombin cleavage site
LVPRGS
SEQ ID NO: 18 - PreScission cleavage site
LEVLFQGP
SEQ ID NO: 19 - HExxH motif
HEXXH
where each X is independently selected from any amino acid
SEQ ID NO: 20 - Influenza virus haemagglutinin translocation domain
GLFGAIAGFIENGWEGMIDGWYG
SEQ ID NO: 21 - xDxxxLL motif
XDXXXLL
where each X is independently selected from any amino acid
SEQ ID NO: 22 - xExxxLL motif
XEXXXLL
where each X is independently selected from any amino acid
SEQ ID NO: 23 - xExxxIL motif
XEXXXIL
where each X is independently selected from any amino acid
SEQ ID NO: 24 - xExxxLM motif
XEXXXLM
where each X is independently selected from any amino acid
SEQ ID NO: 25 -Y-x-x-Hy motif
Y-X-X-Hy
where each X is independently selected from any amino acid, and Hy is any
hydrophobic amino acid
SEQ ID NO: 26 - exemplary BoNT/AB chimera amino acid sequence (mrBoNT/AB)
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NILNNIILNLRYKDNNLIDLSGYGAKVEVYDGVELNDKNQFKLTSSANSKIRVTQNQ
NIIFNSVFLDFSVSFWIRIPKYKNDGIQNYIHNEYTIINCMKNNSGWKISIRGNRIIWTLI
DINGKTKSVFFEYNIREDISEYINRWFFVTITNNLNNAKIYINGKLESNTDIKDIREVIA
NGEIIFKLDGDIDRTQFIWMKYFSIFNTELSQSNIEERYKIQSYSEYLKDFWGNPLMYN
KEYYMFNAGNKNSYIKLKKDSPVGEILTRSKYNQNSKYINYRDLYIGEKFIIRRKSNS
QSINDDIVRKEDYIYLDFFNLNQEWRVYTYKYFKKEEMKLFLAPIYDSDEFYNTIQIK
EYDEQPTYSCQLLFKKDEESTDEIGLIGIHRFYESGIVFEEYKDYFCISKWYLKEVKRK
PYNLKLGCNWQFIPKDEGWTEHHHHHHHHHH
SEQ ID NO: 27 - exemplary BoNT/AB chimera amino acid sequence (mrBoNT/AB)
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKA
KSIVGTTASLQYMKNVFKEKYLLSEDTSGKFSVDKLKFDKLYKMLTEIYTEDNFVKF
FKVLNRKTYLNFDKAVFKINIVPKVNYTIYDGFNLRNTNLAANFNGQNTEINNMNFT
KLKNFTGLFEFYKLLCVRGIITSKTKSLDKGYNKALNDLCIKVNNWDLFFSPSEDNFT
NDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDY
VKKVNKATEA
AMFLGWVEQLVYDFTDETSEVSTTDKIADITIIIPYIGPALNIGNMLYKDDFVGALIFS
GAVILLEFIPEIAIPVLGTFALVSYIANKVLTVQTIDNALSKRNEKWDEVYKYIVTNWL
AKVNTQIDLIRKKMKEALENQAEATKAIINYQYNQYTEEEKNNINFNIDDLSSKLNES
INKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKNILNNIILNLRYKDNNLIDLS
GYGAKVEV
YDGVELNDKNQFKLTSSANSKIRVTQNQNIIFNSVFLDFSVSFWIRIPKYKNDGIQNYI
HNEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTR
SKYNQNSKYINYRDLYIGEKFIIRRKSNSQSINDDIVRKEDYIYLDFFNLNQEWRVYT
YKYFK
KEEMKLFLAPIYDSDEFYNTIQIKEYDEQPTYSCQLLFKKDEESTDEIGLIGIHRFYESG
IVFEEYKDYFCISKWYLKEVKRKPYNLKLGCNWQFIPKDEGWTE
SEQ ID NO: 28 - exemplary BoNT/AB chimera amino acid sequence (mrBoNT/AB)
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKA
KSIVGTTASLQYMKNVFKEKYLLSEDTSGKFSVDKLKFDKLYKMLTEIYTEDNFVKF
FKVLNRKTYLNFDKAVFKINIVPKVNYTIYDGFNLRNTNLAANFNGQNTEINNMNFT
KLKNFTGLFEFYKLLCVRGIITSKTKSLDKGYNKALNDLCIKVNNWDLFFSPSEDNFT
NDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDY
VKKVNKATEA
AMFLGWVEQLVYDFTDETSEVSTTDKIADITIIIPYIGPALNIGNMLYKDDFVGALIFS
GAVILLEFIPEIAIPVLGTFALVSYIANKVLTVQTIDNALSKRNEKWDEVYKYIVTNWL
AKVNTQIDLIRKKMKEALENQAEATKAIINYQYNQYTEEEKNNINFNIDDLSSKLNES
INKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKNILNNIILNLRYKDNNLIDLS
GYGAKVEV
YDGVELNDKNQFKLTSSANSKIRVTQNQNIIFNSVFLDFSVSFWIRIPKYKNDGIQNYI
HNEYTIINCMKNNSGWKISIRGNRIIWTLIDINGKTKSVFFEYNIREDISEYINRWFFVT
ITNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTR
SKYNQNSKYINYRDLYIGEKFIIRRKSNSQSINDDIVRKEDYIYLDFFNLNQEWRVYT
YKYFK
KEEEKLFLAPISDSDEFYNTIQIKEYDEQPTYSCQLLFKKDEESTDEIGLIGIHRFYESGI
VFEEYKDYFCISKWYLKEVKRKPYNLKLGCNWQFIPKDEGWTE
SEQ ID NO: 29 - exemplary BoNT/BC chimera amino acid sequence (mrBoNT/BC)
MPVTINNFNYNDPIDNNNIIMMEPPFARGTGRYYKAFKITDRIWIIPERYTFGYKPEDF
NKSSGIFNRDVCEYYDPDYLNTNDKKNIFLQTMIKLFNRIKSKPLGEKLLEMIINGIPY
LGDRRVPLEEFNTNIASVTVNKLISNPGEVERKKGIFANLIIFGPGPVLNENETIDIGIQ
NHFASREGFGGIMQMKFCPEYVSVENNVQENKGASIFNRRGYFSDPALILMHELIHV
LHGLYGIKVDDLPIVPNEKKFFMQSTDAIQAEELYTFGGQDPSIITPSTDKSIYDKVLQ
NFRGIV
DRLNKVLVCISDPNININIYKNKFKDKYKFVEDSEGKYSIDVESFDKLYKSLMFGFTE
TNIAENYKIKTRASYFSDSLPPVKIKNLLDNEIYTIEEGFNISDKDMEKEYRGQNKAIN
KQAYEEISKEHLAVYKIQMCKSVKAPGICIDVDNEDLFFIADKNSFSDDLSKNERIEY
NTQSNYIENDFPINELILDTDLISKIELPSENTESLTDFNVDVPVYEKQPAIKKIFTDENT
IFQYLYSQTFPLDIRDISLTSSFDDALLFSNKVYSFFSMDYIKTANKVVEAGLFAGWV
KQIVND
FVIEANKSNTMDKIADISLIVPYIGLALNVGNETAKGNFENAFEIAGASILLEFIPELLIP
VVGAFLLESYIDNKNKIIKTIDNALTKRNEKWSDMYGLIVAQWLSTVNTQFYTIKEG
MYKALNYQAQALEEIIKYRYNIYSEKEKSNINIDENDINSKLNEGINQAIDNINNFING
CSVSYLMKKMIPLAVEKLLDFDNTLKKNLLNYIDENKLYLIGSAEYEKSKVNKYLKT
IMPFDLSIYTNDTILIEMENKYNSEILNINDSKILSLQNRKNTLVDTSGYNAEVSEEGD
VQLNPIF
PFDFKLGSSGEDRGKVIVTQNENIVYNSMYESFSISFWIRINKWVSNLPGYTIIDSVKN
NSGWSIGIISNFLVFTLKQNEDSEQSINFSYDISNNAPGYNKWFFVTVTNNMMGNMKI
YINGKLIDTIKVKELTGINFSKTITFEINKIPDTGLITSDSDNINMWIRDFYIFAKELDGK
DINILFNSLQYTNVVKDYWGNDLRYNKEYYMVNIDYLNRYMYANSRQIVENTRRN
NNDFNEGYKIIIKRIRGNTNDTRVRGGDILYFDMTINNKAYNLFMKNETMYADNHST
EDIYAIGLRE
QTKDINDNIIFQIQPMNNTYYYASQIFKSNFNGENISGICSIGTYRFRLGGDWYRHNYL
VPTVKQGNYASLLESTSTHWGFVPVSE
SEQ ID NO: 30 - Nucleotide Sequence of Modified (Cationic) BoNT/A “Cat-A”
ATGCCATTCGTCAACAAGCAATTCAACTACAAAGACCCAGTCAACGGCGTCGAC
ATCGCATACATCAAGATTCCGAACGCCGGTCAAATGCAGCCGGTTAAGGCTTTTA
AGATCCACAACAAGATTTGGGTTATCCCGGAGCGTGACACCTTCACGAACCCGG
AAGAAGGCGATCTGAACCCGCCACCGGAAGCGAAGCAAGTCCCTGTCAGCTACT
ACGATTCGACGTACCTGAGCACGGATAACGAAAAAGATAACTACCTGAAAGGTG
TGACCAAGCTGTTCGAACGTATCTACAGCACGGATCTGGGTCGCATGCTGCTGAC
TAGCATTGTTCGCGGTATCCCGTTCTGGGGGGTAGCACGATTGACACCGAACTG
AAGGTTATCGACACTAACTGCATTAACGTTATTCAACCGGATGGTAGCTATCGTA
GCGAAGAGCTGAATCTGGTCATCATTGGCCCGAGCGCAGACATTATCCAATTCG
AGTGCAAGAGCTTTGGTCACGAGGTTCTGAATCTGACCCGCAATGGCTATGGTAG
CACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTG
TCACGCTGGCCCATGAACTGATCCACGCAGGCCACCGCCTGTACGGCATTGCCAT
CAACCCAAACCGTGTGTTCAAGGTTAATACGAATGCATACTACGAGATGAGCGG
CCTGGAAGTCAGCTTCGAAGAACTGCGCACCTTCGGTGGCCATGACGCTAAATTC
ATTGACAGCTTGCAAGAGAATGAGTTCCGTCTGTACTACTATAACAAATTCAAAG
ACATTGCAAGCACGTTGAACAAGGCCAAAAGCATCGTTGGTACTACCGCGTCGT
TGCAGTATATGAAGAATGTGTTTAAAGAGAAGTACCTGCTGTCCGAGGATACCTC
CGGCAAGTTTAGCGTTGATAAGCTGAAGTTTGACAAACTGTACAAGATGCTGAC
CGAGATTTACACCGAGGACAACTTTGTGAAATTCTTCAAAGTGTTGAATCGTAAA
ACCTATCTGAATTTTGACAAAGCGGTTTTCAAGATTAACATCGTGCCGAAGGTGA
ACTACACCATCTATGACGGTTTTAACCTGCGTAACACCAACCTGGCGGCGAACTT
TAACGGTCAGAATACGGAAATCAACAACATGAATTTCACGAAGTTGAAGAACTT
CACGGGTCTGTTCGAGTTCTATAAGCTGCTGTGCGTGCGCGGTATCATCACCAGC
AAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACG
ACCTGAACAAGGGTGAAGAAATCACCAGCGATACGAATATTGAAGCAGCGGAA
GAGAATATCAGCCTGGATCTGATCCAGCAGTACTATCTGACCTTTAACTTCGACA
ATGAACCGGAGAACATTAGCATTGAGAATCTGAGCAGCGACATTATCGGTCAGC
TGGAACTGATGCCGAATATCGAACGTTTCCCGAACGGCAAAAAGTACGAGCTGG
ACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTG
TCTATACCTTCTTCAGCAGCGACTATGTTAAGAAAGTGAACAAAGCCACTGAGGC
CGCGATGTTCCTGGGCTGGGTGGAACAGCTGGTATATGACTTCACGGACGAGAC
GAGCGAAGTGAGCACTACCGACAAAATTGCTGATATTACCATCATTATCCCGTAT
ATTGGTCCGGCACTGAACATTGGCAACATGCTGTACAAAGACGATTTTGTGGGTG
CCCTGATCTTCTCCGGTGCCGTGATTCTGCTGGAGTTCATTCCGGAGATTGCGATC
CCGGTGTTGGGTACCTTCGCGCTGGTGTCCTACATCGCGAATAAGGTTCTGACGG
TTCAGACCATCGATAACGCGCTGTCGAAACGTAATGAAAAATGGGACGAGGTTT
ACAAATACATTGTTACGAATTGGCTGGCGAAAGTCAATACCCAGATCGACCTGA
TCCGTAAGAAAATGAAAGAGGCGCTGGAGAATCAGGCGGAGGCCACCAAAGCA
ATTATCAACTACCAATACAACCAGTACACGGAAGAAGAGAAGAATAACATTAAC
TTCAATATCGATGATTTGAGCAGCAAGCTGAATGAATCTATCAACAAAGCGATG
ATCAATATCAACAAGTTTTTGAATCAGTGTAGCGTTTCGTACCTGATGAATAGCA
TGATTCCGTATGGCGTCAAACGTCTGGAGGACTTCGACGCCAGCCTGAAAGATG
CGTTGCTGAAATACATTTACGACAATCGTGGTACGCTGATTGGCCAAGTTGACCG
CTTGAAAGACAAAGTTAACAATACCCTGAGCACCGACATCCCATTTCAACTGAG
CAAGTATGTTGATAATCAACGTCTGTTGAGCACTTTCACCGAGTATATCAAAAAC
ATCATCAATACTAGCATTCTGAACCTGCGTTACGAGAGCAAGCATCTGATTGATC
TGAGCCGTTATGCTAGCAAGATCAACATCGGTAGCAAGGTCAATTTTGACCCGAT
CGATAAGAACCAGATCCAGCTGTTTAATCTGGAATCGAGCAAAATTGAGGTTAT
CCTGAAAAAGGCCATTGTCTACAACTCCATGTACGAGAATTTCTCCACCAGCTTC
TGGATTCGCATCCCGAAATACTTCAACAAGATTAGCCTGAACAACGAGTATACTA
TCATCAACTGTATGGAGAACAACAGCGGTTGGAAGGTGTCTCTGAACTATGGTG
AGATCATTTGGACCTTGCAGGACACCAAAGAGATCAAGCAGCGCGTCGTGTTCA
AGTACTCTCAAATGATCAACATTTCCGATTACATTAATCGTTGGATCTTCGTGAC
CATTACGAATAACCGTCTGAATAAGAGCAAGATTTACATCAATGGTCGCTTGATC
GATCAGAAACCGATTAGCAACCTGGGTAATATCCACGCAAGCAACAAGATTATG
TTCAAATTGGACGGTTGCCGCGATACCCATCGTTATATCTGGATCAAGTATTTCA
ACCTGTTTGATAAAGAACTGAATGAGAAGGAGATCAAAGATTTGTATGACAACC
AATCTAACAGCGGCATTTTGAAGGACTTCTGGGGCGATTATCTGCAATACGATAA
GCCGTACTATATGCTGAACCTGTATGATCCGAACAAATATGTGGATGTCAATAAT
GTGGGTATTCGTGGTTACATGTATTTGAAGGGTCCGCGTGGCAGCGTTATGACGA
CCAACATTTACCTGAACTCTAGCCTGTACCGTGGTACGAAATTCATCATTAAGAA
ATATGCCAGCGGCAACAAAGATAACATTGTGCGTAATAACGATCGTGTCTACAT
CAACGTGGTCGTGAAGAATAAAGAGTACCGTCTGGCGACCAACGCTTCGCAGGC
GGGTGTTGAGAAAATTCTGAGCGCGTTGGAGATCCCTGATGTCGGTAATCTGAGC
CAAGTCGTGGTTATGAAGAGCAAGAACGACAAGGGTATCACTAACAAGTGCAAG
ATGAACCTGCAAGACAACAATGGTAACGACATCGGCTTTATTGGTTTCCACCAGT
TCAACAATATTGCTAAACTGGTAGCGAGCAATTGGTACAATCGTCAGATTGAGC
GCAGCAGCcGTACTTTGGGCTGTAGCTGGGAGTTTATCCCGGTCGATGATGGTTG
GGGCGAACGTCCGCTG
SEQ ID NO: 31 - Polypeptide Sequence of Modified (Cationic) BoNT/A “Cat-A”
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NIINTSILNLRYESKHLIDLSRYASKINIGSKVNFDPIDKNQIQLFNLESSKIEVILKKAIV
YNSMYENFSTSFWIRIPKYFNKISLNNEYTIINCMENNSGWKVSLNYGEIIWTLQDTK
EIKQRVVFKYSQMINISDYINRWIFVTITNNRLNKSKIYINGRLIDQKPISNLGNIHASN
KIMFKLDGCRDTHRYIWIKYFNLFDKELNEKEIKDLYDNQSNSGILKDFWGDYLQYD
KPYYMLNLYDPNKYVDVNNVGIRGYMYLKGPRGSVMTTNIYLNSSLYRGTKFIIKK
YASGNKDNIVRNNDRVYINVVVKNKEYRLATNASQAGVEKILSALEIPDVGNLSQV
VVMKSKNDKGITNKCKMNLQDNNGNDIGFIGFHQFNNIAKLVASNWYNRQIERSSR
TLGCSWEFIPVDDGWGERPL
SEQ ID NO: 32 - Nucleotide Sequence of Modified (Cationic) BoNT/A “Cat-B”
ATGCCATTCGTCAACAAGCAATTCAACTACAAAGACCCAGTCAACGGCGTCGAC
ATCGCATACATCAAGATTCCGAACGCCGGTCAAATGCAGCCGGTTAAGGCTTTTA
AGATCCACAACAAGATTTGGGTTATCCCGGAGCGTGACACCTTCACGAACCCGG
AAGAAGGCGATCTGAACCCGCCACCGGAAGCGAAGCAAGTCCCTGTCAGCTACT
ACGATTCGACGTACCTGAGCACGGATAACGAAAAAGATAACTACCTGAAAGGTG
TGACCAAGCTGTTCGAACGTATCTACAGCACGGATCTGGGTCGCATGCTGCTGAC
TAGCATTGTTCGCGGTATCCCGTTCTGGGGTGGTAGCACGATTGACACCGAACTG
AAGGTTATCGACACTAACTGCATTAACGTTATTCAACCGGATGGTAGCTATCGTA
GCGAAGAGCTGAATCTGGTCATCATTGGCCCGAGCGCAGACATTATCCAATTCG
AGTGCAAGAGCTTTGGTCACGAGGTTCTGAATCTGACCCGCAATGGCTATGGTAG
CACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTG
TCACGCTGGCCCATGAACTGATCCACGCAGGCCACCGCCTGTACGGCATTGCCAT
CAACCCAAACCGTGTGTTCAAGGTTAATACGAATGCATACTACGAGATGAGCGG
CCTGGAAGTCAGCTTCGAAGAACTGCGCACCTTCGGTGGCCATGACGCTAAATTC
ATTGACAGCTTGCAAGAGAATGAGTTCCGTCTGTACTACTATAACAAATTCAAAG
ACATTGCAAGCACGTTGAACAAGGCCAAAAGCATCGTTGGTACTACCGCGTCGT
TGCAGTATATGAAGAATGTGTTTAAAGAGAAGTACCTGCTGTCCGAGGATACCTC
CGGCAAGTTTAGCGTTGATAAGCTGAAGTTTGACAAACTGTACaAGATGCTGACC
GAGATTTACACCGAGGACAACTTTGTGAAATTCTTCAAAGTGTTGAATCGTAAAA
CCTATCTGAATTTTGACAAAGCGGTTTTCAAGATTAACATCGTGCCGAAGGTGAA
CTACACCATCTATGACGGTTTTAACCTGCGTAACACCAACCTGGCGGCGAACTTT
AACGGTCAGAATACGGAAATCAACAACATGAATTTCACGAAGTTGAAGAACTTC
ACGGGTCTGTTCGAGTTCTATAAGCTGCTGTGCGTGCGCGGTATCATCACCAGCA
AAACCAAAAGCCTGGACAAAGGCTACAACAAGGCGCTGAATGACCTGTGCATTA
AGGTAAACAATTGGGATCTGTTCTTTTCGCCATCCGAAGATAATTTTACCAACGA
CCTGAACAAGGGTGAAGAAATCACCAGCGATACGAATATTGAAGCAGCGGAAG
AGAATATCAGCCTGGATCTGATCCAGCAGTACTATCTGACCTTTAACTTCGACAA
TGAACCGGAGAACATTAGCATTGAGAATCTGAGCAGCGACATTATCGGTCAGCT
GGAACTGATGCCGAATATCGAACGTTTCCCGAACGGCAAAAAGTACGAGCTGGA
CAAGTACACTATGTTCCATTACCTGCGTGCACAGGAGTTTGAACACGGTAAAAGC
CGTATCGCGCTGACCAACAGCGTTAACGAGGCCCTGCTGAACCCGAGCCGTGTCT
ATACCTTCTTCAGCAGCGACTATGTTAAGAAAGTGAACAAAGCCACTGAGGCCG
CGATGTTCCTGGGCTGGGTGGAACAGCTGGTATATGACTTCACGGACGAGACGA
GCGAAGTGAGCACTACCGACAAAATTGCTGATATTACCATCATTATCCCGTATAT
TGGTCCGGCACTGAACATTGGCAACATGCTGTACAAAGACGATTTTGTGGGTGCC
CTGATCTTCTCCGGTGCCGTGATTCTGCTGGAGTTCATTCCGGAGATTGCGATCCC
GGTGTTGGGTACCTTCGCGCTGGTGTCCTACATCGCGAATAAGGTTCTGACGGTT
CAGACCATCGATAACGCGCTGTCGAAACGTAATGAAAAATGGGACGAGGTTTAC
AAATACATTGTTACGAATTGGCTGGCGAAAGTCAATACCCAGATCGACCTGATCC
GTAAGAAAATGAAAGAGGCGCTGGAGAATCAGGCGGAGGCCACCAAAGCAATT
ATCAACTACCAATACAACCAGTACACGGAAGAAGAGAAGAATAACATTAACTTC
AATATCGATGATTTGAGCAGCAAGCTGAATGAATCTATCAACAAAGCGATGATC
AATATCAACAAGTTTTTGAATCAGTGTAGCGTTTCGTACCTGATGAATAGCATGA
TTCCGTATGGCGTCAAACGTCTGGAGGACTTCGACGCCAGCCTGAAAGATGCGTT
GCTGAAATACATTTACGACAaTCGTGGTACGCTGATTGGCCAAGTTGACCGCTTG
AAAGACAAAGTTAACAATACCCTGAGCACCGACATCCCATTTCAACTGAGCAAG
TATGTTGATAATCAACGTCTGTTGAGCACTTTCACCGAGTATATCAAAAACATCA
TCAATACTAGCATTCTGAACCTGCGTTACGAGAGCAATCATCTGATTGATCTGAG
CCGTTATGCTAGCAAGATCAACATCGGTAGCAAGGTCAATTTTGACCCGATCGAT
AAGAACCAGATCCAGCTGTTTAATCTGGAATCGAGCAAAATTGAGGTTATCCTG
AAAAAGGCCATTGTCTACAACTCCATGTACGAGAATTTCTCCACCAGCTTCTGGA
TTCGCATCCCGAAATACTTCAAGAAGATTAGCCTGAACAACGAGTATACTATCAT
CAACTGTATGGAGAACAACAGCGGTTGGAAGGTGTCTCTGAACTATGGTGAGAT
CATTTGGACCTTGCAGGACACCAAAGAGATCAAGCAGCGCGTCGTGTTCAAGTA
CTCTCAAATGATCAACATTTCCGATTACATTAATCGTTGGATCTTCGTGACCATTA
CGAATAACCGTCTGAATAAGAGCAAGATTTACATCAATGGTCGCTTGATCGATCA
GAAACCGATTAGCAACCTGGGTAATATCCACGCAAGCAACAAGATTATGTTCAA
ATTGGACGGTTGCCGCGATACCCATCGTTATATCTGGATCAAGTATTTCAACCTG
TTTGATAAAGAACTGAATGAGAAGGAGATCAAAGATTTGTATGACAACCAATCT
AACAGCGGCATTTTGAAGGACTTCTGGGGCGATTATCTGCAATACGATAAGCCGT
ACTATATGCTGAACCTGTATGATCCGAACAAATATGTGGATGTCAATAATGTGGG
TATTCGTGGTTACATGTATTTGAAGGGTCCGCGTGGCAGCGTTATGACGACCAAC
ATTTACCTGAACTCTAGCCTGTACCGTGGTACGAAATTCATCATTAAGAAATATG
CCAGCGGCAACAAAGATAACATTGTGCGTAATAACGATCGTGTCTACATCAACG
TGGTCGTGAAGAATAAAGAGTACCGTCTGGCGACCAACGCTTCGCAGGCGGGTG
TTGAGAAAATTCTGAGCGCGTTGGAGATCCCTGATGTCGGTAATCTGAGCCAAGT
CGTGGTTATGAAGAGCAAGAACGACAAGGGTATCACTAACAAGTGCAAGATGAA
CCTGCAAGACAACAATGGTAACGACATCGGCTTTATTGGTTTCCACCAGTTCAAC
AATATTGCTAAACTGGTAGCGAGCAATTGGTACAATCGTCAGATTGAGCGCAGC
AGCCGTACTTTGGGCTGTAGCTGGGAGTTTATCCCGGTCGATGATGGTTGGGGCG
AACGTCCGCTG
SEQ ID NO: 33 - Polypeptide Sequence of Modified (Cationic) BoNT/A “Cat-B”
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NIINTSILNLRYESNHLIDLSRYASKINIGSKVNFDPIDKNQIQLFNLESSKIEVILKKAIV
YNSMYENFSTSFWIRIPKYFKKISLNNEYTIINCMENNSGWKVSLNYGEIIWTLQDTK
EIKQRVVFKYSQMINISDYINRWIFVTITNNRLNKSKIYINGRLIDQKPISNLGNIHASN
KIMFKLDGCRDTHRYIWIKYFNLFDKELNEKEIKDLYDNQSNSGILKDFWGDYLQYD
KPYYMLNLYDPNKYVDVNNVGIRGYMYLKGPRGSVMTTNIYLNSSLYRGTKFIIKK
YASGNKDNIVRNNDRVYINVVVKNKEYRLATNASQAGVEKILSALEIPDVGNLSQV
VVMKSKNDKGITNKCKMNLQDNNGNDIGFIGFHQFNNIAKLVASNWYNRQIERSSR
TLGCSWEFIPVDDGWGERPL
SEQ ID NO: 34 - Nucleotide Sequence of Modified (Cationic) BoNT/A “Cat-C”
ATGCCATTCGTCAACAAGCAATTCAACTACAAAGACCCAGTCAACGGCGTCGAC
ATCGCATACATCAAGATTCCGAACGCCGGTCAAATGCAGCCGGTTAAGGCTTTTA
AGATCCACAACAAGATTTGGGTTATCCCGGAGCGTGACACCTTCACGAACCCGG
AAGAAGGCGATCTGAACCCGCCACCGGAAGCGAAGCAAGTCCCTGTCAGCTACT
ACGATTCGACGTACCTGAGCACGGATAACGAAAAAGATAACTACCTGAAAGGTG
TGACCAAGCTGTTCGAACGTATCTACAGCACGGATCTGGGTCGCATGCTGCTGAC
TAGCATTGTTCGCGGTATCCCGTTCTGGGGGGTAGCACGATTGACACCGAACTG
AAGGTTATCGACACTAACTGCATTAACGTTATTCAACCGGATGGTAGCTATCGTA
GCGAAGAGCTGAATCTGGTCATCATTGGCCCGAGCGCAGACATTATCCAATTCG
AGTGCAAGAGCTTTGGTCACGAGGTTCTGAATCTGACCCGCAATGGCTATGGTAG
CACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTG
TCACGCTGGCCCATGAACTGATCCACGCAGGCCACCGCCTGTACGGCATTGCCAT
CAACCCAAACCGTGTGTTCAAGGTTAATACGAATGCATACTACGAGATGAGCGG
CCTGGAAGTCAGCTTCGAAGAACTGCGCACCTTCGGTGGCCATGACGCTAAATTC
ATTGACAGCTTGCAAGAGAATGAGTTCCGTCTGTACTACTATAACAAATTCAAAG
ACATTGCAAGCACGTTGAACAAGGCCAAAAGCATCGTTGGTACTACCGCGTCGT
TGCAGTATATGAAGAATGTGTTTAAAGAGAAGTACCTGCTGTCCGAGGATACCTC
CGGCAAGTTTAGCGTTGATAAGCTGAAGTTTGACAAACTGTACAAGATGCTGAC
CGAGATTTACACCGAGGACAACTTTGTGAAATTCTTCAAAGTGTTGAATCGTAAA
ACCTATCTGAATTTTGACAAAGCGGTTTTCAAGATTAACATCGTGCCGAAGGTGA
ACTACACCATCTATGACGGTTTTAACCTGCGTAACACCAACCTGGCGGCGAACTT
TAACGGTCAGAATACGGAAATCAACAACATGAATTTCACGAAGTTGAAGAACTT
CACGGGTCTGTTCGAGTTCTATAAGCTGCTGTGCGTGCGCGGTATCATCACCAGC
AAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACG
ACCTGAACAAGGGTGAAGAAATCACCAGCGATACGAATATTGAAGCAGCGGAA
GAGAATATCAGCCTGGATCTGATCCAGCAGTACTATCTGACCTTTAACTTCGACA
ATGAACCGGAGAACATTAGCATTGAGAATCTGAGCAGCGACATTATCGGTCAGC
TGGAACTGATGCCGAATATCGAACGTTTCCCGAACGGCAAAAAGTACGAGCTGG
ACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTG
TCTATACCTTCTTCAGCAGCGACTATGTTAAGAAAGTGAACAAAGCCACTGAGGC
CGCGATGTTCCTGGGCTGGGTGGAACAGCTGGTATATGACTTCACGGACGAGAC
GAGCGAAGTGAGCACTACCGACAAAATTGCTGATATTACCATCATTATCCCGTAT
ATTGGTCCGGCACTGAACATTGGCAACATGCTGTACAAAGACGATTTTGTGGGTG
CCCTGATCTTCTCCGGTGCCGTGATTCTGCTGGAGTTCATTCCGGAGATTGCGATC
CCGGTGTTGGGTACCTTCGCGCTGGTGTCCTACATCGCGAATAAGGTTCTGACGG
TTCAGACCATCGATAACGCGCTGTCGAAACGTAATGAAAAATGGGACGAGGTTT
ACAAATACATTGTTACGAATTGGCTGGCGAAAGTCAATACCCAGATCGACCTGA
TCCGTAAGAAAATGAAAGAGGCGCTGGAGAATCAGGCGGAGGCCACCAAAGCA
ATTATCAACTACCAATACAACCAGTACACGGAAGAAGAGAAGAATAACATTAAC
TTCAATATCGATGATTTGAGCAGCAAGCTGAATGAATCTATCAACAAAGCGATG
ATCAATATCAACAAGTTTTTGAATCAGTGTAGCGTTTCGTACCTGATGAATAGCA
TGATTCCGTATGGCGTCAAACGTCTGGAGGACTTCGACGCCAGCCTGAAAGATG
CGTTGCTGAAATACATTTACGACAATCGTGGTACGCTGATTGGCCAAGTTGACCG
CTTGAAAGACAAAGTTAACAATACCCTGAGCACCGACATCCCATTTCAACTGAG
CAAGTATGTTGATAATCAACGTCTGTTGAGCACTTTCACCGAGTATATCAAAAAC
ATCATCAATACTAGCATTCTGAACCTGCGTTACGAGAGCAATCATCTGATTGATC
TGAGCCGTTATGCTAGCAAGATCAACATCGGTAGCAAGGTCAATTTTGACCCGAT
CGATAAGAACCAGATCCAGCTGTTTAATCTGGAATCGAGCAAAATTGAGGTTAT
CCTGAAAAAGGCCATTGTCTACAACTCCATGTACGAGAATTTCTCCACCAGCTTC
TGGATTCGCATCCCGAAATACTTCAACAAGATTAGCCTGAACAACGAGTATACTA
TCATCAACTGTATGGAGAACAACAGCGGTTGGAAGGTGTCTCTGAACTATGGTG
AGATCATTTGGACCTTGCAGGACACCAAAGAGATCAAGCAGCGCGTCGTGTTCA
AGTACTCTCAAATGATCAACATTTCCGATTACATTAATCGTTGGATCTTCGTGAC
CATTACGAATAACCGTCTGAAGAAGAGCAAGATTTACATCAATGGTCGCTTGATC
GATCAGAAACCGATTAGCAACCTGGGTAATATCCACGCAAGCAACAAGATTATG
TTCAAATTGGACGGTTGCCGCGATACCCATCGTTATATCTGGATCAAGTATTTCA
ACCTGTTTGATAAAGAACTGAATGAGAAGGAGATCAAAGATTTGTATGACAACC
AATCTAACAGCGGCATTTTGAAGGACTTCTGGGGCGATTATCTGCAATACGATAA
GCCGTACTATATGCTGAACCTGTATGATCCGAACAAATATGTGGATGTCAATAAT
GTGGGTATTCGTGGTTACATGTATTTGAAGGGTCCGCGTGGCAGCGTTATGACGA
CCAACATTTACCTGAACTCTAGCCTGTACCGTGGTACGAAATTCATCATTAAGAA
ATATGCCAGCGGCAACAAAGATAACATTGTGCGTAATAACGATCGTGTCTACAT
CAACGTGGTCGTGAAGAATAAAGAGTACCGTCTGGCGACCAACGCTTCGCAGGC
GGGTGTTGAGAAAATTCTGAGCGCGTTGGAGATCCCTGATGTCGGTAATCTGAGC
CAAGTCGTGGTTATGAAGAGCAAGAACGACAAGGGTATCACTAACAAGTGCAAG
ATGAACCTGCAAGACAACAATGGTAACGACATCGGCTTTATTGGTTTCCACCAGT
TCAACAATATTGCTAAACTGGTAGCGAGCAATTGGTACAATCGTCAGATTGAGC
GCAGCAGCCGTACTTTGGGCTGTAGCTGGGAGTTTATCCCGGTCGATGATGGTTG
GGGCGAACGTCCGCTG
SEQ ID NO: 35 - Polypeptide Sequence of Modified (Cationic) BoNT/A “Cat-C”
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NIINTSILNLRYESNHLIDLSRYASKINIGSKVNFDPIDKNQIQLFNLESSKIEVILKKAIV
YNSMYENFSTSFWIRIPKYFNKISLNNEYTIINCMENNSGWKVSLNYGEIIWTLQDTK
EIKQRVVFKYSQMINISDYINRWIFVTITNNRLKKSKIYINGRLIDQKPISNLGNIHASN
KIMFKLDGCRDTHRYIWIKYFNLFDKELNEKEIKDLYDNQSNSGILKDFWGDYLQYD
KPYYMLNLYDPNKYVDVNNVGIRGYMYLKGPRGSVMTTNIYLNSSLYRGTKFIIKK
YASGNKDNIVRNNDRVYINVVVKNKEYRLATNASQAGVEKILSALEIPDVGNLSQV
VVMKSKNDKGITNKCKMNLQDNNGNDIGFIGFHQFNNIAKLVASNWYNRQIERSSR
TLGCSWEFIPVDDGWGERPL
SEQ ID NO: 36 - Nucleotide Sequence of Modified (Cationic) BoNT/A “Cat-D”
ATGCCATTCGTCAACAAGCAATTCAACTACAAAGACCCAGTCAACGGCGTCGAC
ATCGCATACATCAAGATTCCGAACGCCGGTCAAATGCAGCCGGTTAAGGCTTTTA
AGATCCACAACAAGATTTGGGTTATCCCGGAGCGTGACACCTTCACGAACCCGG
AAGAAGGCGATCTGAACCCGCCACCGGAAGCGAAGCAAGTCCCTGTCAGCTACT
ACGATTCGACGTACCTGAGCACGGATAACGAAAAAGATAACTACCTGAAAGGTG
TGACCAAGCTGTTCGAACGTATCTACAGCACGGATCTGGGTCGCATGCTGCTGAC
TAGCATTGTTCGCGGTATCCCGTTCTGGGGTGGTAGCACGATTGACACCGAACTG
AAGGTTATCGACACTAACTGCATTAACGTTATTCAACCGGATGGTAGCTATCGTA
GCGAAGAGCTGAATCTGGTCATCATTGGCCCGAGCGCAGACATTATCCAATTCG
AGTGCAAGAGCTTTGGTCACGAGGTTCTGAATCTGACCCGCAATGGCTATGGTAG
CACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTG
TCACGCTGGCCCATGAACTGATCCACGCAGGCCACCGCCTGTACGGCATTGCCAT
CAACCCAAACCGTGTGTTCAAGGTTAATACGAATGCATACTACGAGATGAGCGG
CCTgGAAGTCAGCTTCGAAGAACTGCGCACCTTCGGTGGCCATGACGCTAAATTC
ATTGACAGCTTGCAAGAGAATGAGTTCCGTCTGTACTACTATAACAAATTCAAAG
ACATTGCAAGCACGTTGAACAAGGCCAAAAGCATCGTTGGTACTACCGCGTCGT
TGCAGTATATGAAGAATGTGTTTAAAGAGAAGTACCTGCTGTCCGAGGATACCTC
CGGCAAGTTTAGCGTTGATAAGCTGAAGTTTGACAAACTGTACAAGATGCTGAC
CGAGATTTACACCGAGGACAACTTTGTGAAATTCTTCAAaGTGTTGAATCGTAAA
ACCTATCTGAATTTTGACAAAGCGGTTTTCaAGATTAACATCGTGCCGAAGGTGA
ACTACACCATCTATGACGGTTTTAACCTGCGTAACACCAACCTGGCGGCGAACTT
TAACGGTCAGAATACGGAAATCAACAACATGAATTTCACGAAGTTGAAGAACTT
CACGGGTCTGTTCGAGTTCTATAAGCTGCTGTGCGTGCGCGGTATCATCACCAGC
AAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACG
ACCTGAACAAGGGTGAAGAAATCACCAGCGATACGAATATTGAAGCAGCGGAA
GAGAATATCAGCCTGGATCTGATCCAGCAGTACTATCTGACCTTTAACTTCGACA
ATGAACCGGAGAACATTAGCATTGAGAATCTGAGCAGCGACATTATCGGTCAGC
TGGAACTGATGCCGAATATCGAACGTTTCCCGAACGGCAAAAAGTACGAGCTGG
ACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTG
TCTATACCTTCTTCAGCAGCGACTATGTTAAGAAAGTGAACAAAGCCACTGAGGC
CGCGATGTTCCTGGGCTGGGTGGAACAGCTGGTATATGACTTCACGGACGAGAC
GAGCGAAGTGAGCACTACCGACAAAaTTGCTGATaTTACCATCATTATCCCGTAT
ATTGGTCCGGCACTGAACATTGGCAACATGCTGTACAAAGACGATTTTGTGGGTG
CCCTGATCTTCTCCGGTGCCGTGATTCTGCTGGAGTTCATTCCGGAGATTGCGATC
CCGGTGTTGGGTACCTTCGCGCTGGTGTCCTACATCGCGAATAAGGTTCTGACGG
TTCAGACCATCGATAACGCGCTGTCGAAACGTAATGAAAAATGGGACGAGGTTT
ACAAATACATTGTTACGAATTGGCTGGCGAAAGTCaATACCCAGATCGACCTGAT
CCGTAAGAAAATGAAAGAGGCGCTGGAGAATCAGGCGGAGGCCACCAAAGCAA
TTATCAACTACCAATACAACCAGTACACGGAAGAAGAGAAGAATAACATTAACT
TCAATATCGATGATTTGAGCAGCAAGCTGAATGAATCTATCAACAAAGCGATGA
TCAATATCAACAAGTTTTTGAATCAGTGTAGCGTTTCGTACCTGATGAATAGCAT
GATTCCGTATGGCGTCAAACGTCTGGAGGACTTCGACGCCAGCCTGAAAGATGC
GTTGCTGAAATACATTTACGACAATCGTGGTACGCTGATTGGCCAAGTTGACCGC
TTGAAAGACAAAGTTAACAATACCCTGAGCACCGACATCCCATTTCAACTGAGC
AAGTATGTTGATAATCAACGTCTGTTGAGCACTTTCACCGAGTATATCAAAAACA
TCATCAATACTAGCATTCTGAACCTGCGTTACGAGAGCAATCATCTGATtGATCTG
AGCCGTTATGCAAGCAAGATCAACATCGGTAGCAAGGTCAATTTTGACCCGATC
GATAAGAACCAGATCCAGCTGTTTAATCTGGAATCGAGCAAAATTGAGGTTATC
CTGAAAAACGCCATTGTCTACAACTCCATGTACGAGAATTTCTCCACCAGCTTCT
GGATTCGCATCCCGAAATACTTCAACAGCATTAGCCTGAACAACGAGTATACTAT
CATCAACTGTATGGAGAACAACAGCGGTTGGAAGGTGTCTCTGAACTATGGTGA
GATCATTTGGACCTTGCAGGACACCCAAGAGATCAAGCAGCGCGTCGTGTTCAA
GTACTCTCAAATGATCAACATTTCCGATTACATTAATCGTTGGATCTTCGTGACC
ATTACGAATAACCGTCTGAATAACAGCAAGATTTACATCAATGGTCGCTTGATCG
ATCAGAAACCGATTAGCAACCTGGGTAATATCCACGCAAGCAACAACATTATGT
TCAAATTGGACGGTTGCCGCGATACCCATCGTTATATCTGGATCAAGTATTTCAA
CCTGTTTGATAAAGAACTGAATGAGAAGGAGATCAAAGATTTGTATGACAACCA
ATCTAACAGCGGCATTTTGAAGGACTTCTGGGGCGATTATCTGCAATACGATAAG
CCGTACTATATGCTGAACCTGTATGATCCGAACAAATATGTGGATGTCAATAATG
TGGGTATTCGTGGTTACATGTATTTGAAGGGTCCGCGTGGCAGCGTTATGACGAC
CAACATTTACCTGAACTCTAGCCTGTACCGTGGTACGAAATTCATCATTAAGAAA
TATGCCAGCGGCAACAAAGATAACATTGTGCGTAATAACGATCGTGTCTACATC
AACGTGGTCGTGAAGCGTAAAGAGTACCGTCTGGCGACCAACGCTTCGCAGGCG
GGTGTTGAGAAAATTCTGAGCGCGTTGGAGATCCCTCGTGTCCGTCGTCTGAGCC
AAGTCGTGGTTATGAAGAGCAAGAACGACCAGGGTATCACTAACAAGTGCAAGA
TGAACCTGCAAGACCGTCGTGGTAACGACATCGGCTTTATTGGTTTCCACCAGTT
CAACAATATTGCTAAACTGGTAGCGAGCAATTGGTACAATCGTCAGATTGAGCG
CCGTAGCCGTCGTTTGGGCTGTAGCTGGGAGTTTATCCCGGTCGATGATGGTTGG
GGCGAACGTCCGCTG
SEQ ID NO: 37 - Polypeptide Sequence of Modified (Cationic) BoNT/A “Cat-D”
MPFVNKQFNYKDPVNGVDIAYIKIPNAGQMQPVKAFKIHNKIWVIPERDTFTNPEEG
DLNPPPEAKQVPVSYYDSTYLSTDNEKDNYLKGVTKLFERIYSTDLGRMLLTSIVRGI
PFWGGSTIDTELKVIDTNCINVIQPDGSYRSEELNLVIIGPSADIIQFECKSFGHEVLNL
TRNGYGSTQYIRFSPDFTFGFEESLEVDTNPLLGAGKFATDPAVTLAHELIHAGHRLY
GIAINPNRVFKVNTNAYYEMSGLEVSFEELRTFGGHDAKFIDSLQENEFRLYYYNKF
KDIASTLNKAKSIVGTTASLQYMKNVFKEKYLLSEDTSGKFSVDKLKFDKLYKMLTE
IYTEDNFVKFFKVLNRKTYLNFDKAVFKINIVPKVNYTIYDGFNLRNTNLAANFNGQ
NTEINNMNFTKLKNFTGLFEFYKLLCVRGIITSKTKSLDKGYNKALNDLCIKVNNWD
LFFSPSEDNFTNDLNKGEEITSDTNIEAAEENISLDLIQQYYLTFNFDNEPENISIENLSS
DIIGQLELMPNIERFPNGKKYELDKYTMFHYLRAQEFEHGKSRIALTNSVNEALLNPS
RVYTFFSSDYVKKVNKATEAAMFLGWVEQLVYDFTDETSEVSTTDKIADITIIIPYIGP
ALNIGNMLYKDDFVGALIFSGAVILLEFIPEIAIPVLGTFALVSYIANKVLTVQTIDNAL
SKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIK
NIINTSILNLRYESNHLIDLSRYASKINIGSKVNFDPIDKNQIQLFNLESSKIEVILKNAIV
YNSMYENFSTSFWIRIPKYFNSISLNNEYTIINCMENNSGWKVSLNYGEIIWTLQDTQ
EIKQRVVFKYSQMINISDYINRWIFVTITNNRLNNSKIYINGRLIDQKPISNLGNIHASN
NIMFKLDGCRDTHRYIWIKYFNLFDKELNEKEIKDLYDNQSNSGILKDFWGDYLQYD
KPYYMLNLYDPNKYVDVNNVGIRGYMYLKGPRGSVMTTNIYLNSSLYRGTKFIIKK
YASGNKDNIVRNNDRVYINVVVKRKEYRLATNASQAGVEKILSALEIPRVRRLSQVV
VMKSKNDQGITNKCKMNLQDRRGNDIGFIGFHQFNNIAKLVASNWYNRQIERRSRR
LGCSWEFIPVDDGWGERPL
SEQ ID NO: 38 - Carrier component
GGGGGGGGGGGGGGGGGGGGRGRDDRRQRRRGGGGGGGGGGGGGGGGGGGG
SEQ ID NO: 39 - Carrier component
GGGGGGGGGGGGGGGGGGGGYGRKKRRQRRRGGGGGGGGGGGGGGGGGGGG
SEQ ID NO: 40 - Carrier component
GGGGGGGGGGGGGGGGGGGGRKKRRQRRRGGGGGGGGGGGGGGGGGGGG
SEQ ID NO: 41 - Carrier component
RKKRRQRRRGKKKKK
KKKKKGRKKRRQRRR

### EXAMPLES

The invention will be further clarified by the following examples, which are intended to be purely exemplary of the invention and are in no way limiting.

### Example 1—pH and Salt Screening to Identify Stabilising Conditions

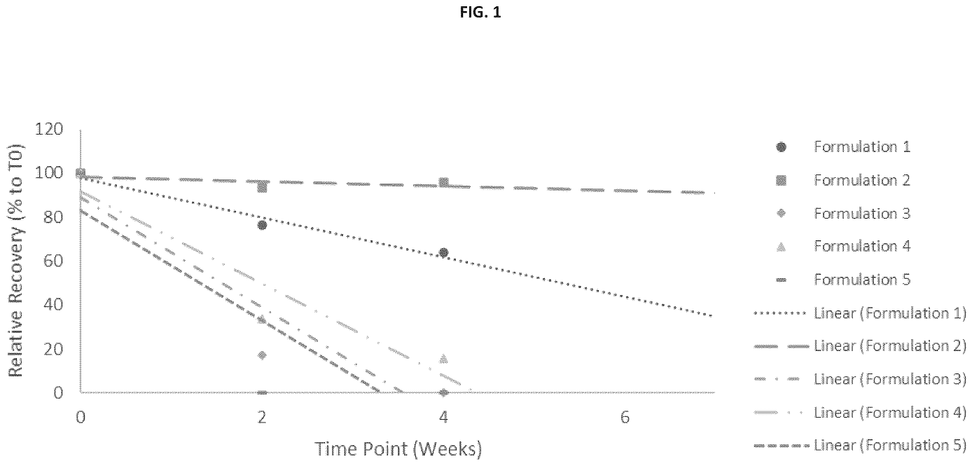

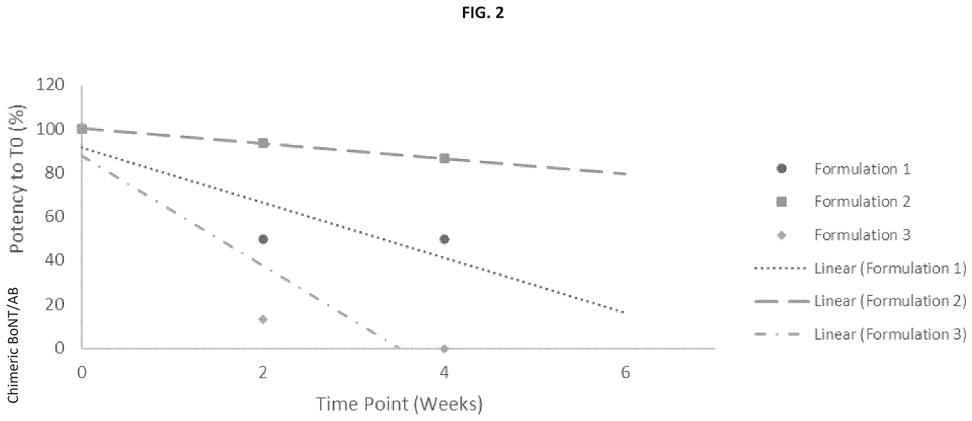

BoNT/AB chimera was formulated as a liquid using a citric acid/sodium phosphate buffer (20 mM), 10 mg/mL sucrose, and 0.01% w/v polysorbate 80 (PS80), across a range of pH values (5.5, 6.5, and 7.5), at high and low NaCl concentrations (10 mM and 100 mM), with or without ascorbic acid (0.1% w/v), as shown in Table 3 below. Formulations were incubated under accelerated stability conditions (25 °C) for 4 weeks and stability assessed by chimeric BoNT/AB protein quantification ELISA and a cell-based potency assay. Data generated from quantification and relative potency are presented in FIGS. 1 and 2, respectively.

TABLE 3
Varying pH, NaCl concentrations and additional excipients

| | | | Stabiliser | Salt | Surfactant | Additional |
|---|---|---|---|---|---|---|
| Form | Buffer (mM) | pH | (mg/mL) | (mM) | (% w/v) | Excipient |
| 1 | Cit-Phos (20) | 5.5 | Sucrose (10) | NaCl | PS80 | N/A |
| | | | | (100) | (0.01) | |
| 2 | Cit-Phos (20) | 6.5 | Sucrose (10) | NaCl | PS80 | N/A |
| | | | | (100) | (0.01) | |
| 3 | Cit-Phos (20) | 7.5 | Sucrose (10) | NaCl | PS80 | N/A |
| | | | | (100) | (0.01) | |
| 4 | Cit-Phos (20) | 6.5 | Sucrose (10) | NaCl | PS80 | Ascorbic Acid |
| | | | | (100) | (0.01) | (0.1% w/v) |
| 5 | Cit-Phos (20) | 6.5 | Sucrose (10) | NaCl | PS80 | N/A |
| | | | | (10) | (0.01) | |

As shown in FIG. 1, formulations with a pH of 6.5 showed superior recovery of chimeric BoNT/AB compared with formulations at a pH of 5.5 or 7.5. In addition adding ascorbic acid (as in Formula 4) was detrimental to product recovery.

In addition, as illustrated in FIG. 2, Formulation 2 was shown to demonstrate superior potency over time compared with Formulations 1 (pH 5.5) and 3 (pH 7.5). Formulations 4 & 5 were not investigated for potency due to poor recovery observed from quantification analysis.

As such, pH 6.5 with an NaCl concentration of 100 mM was taken forward for further investigation.

### Example 2—Screening to Identify Stabilising Excipients

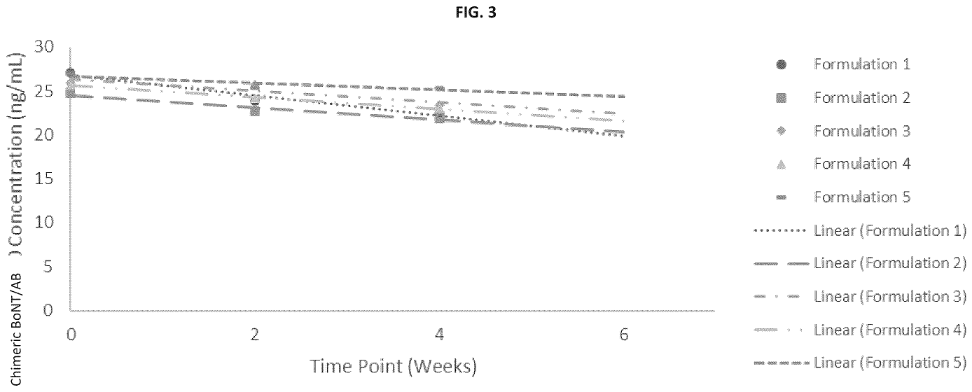

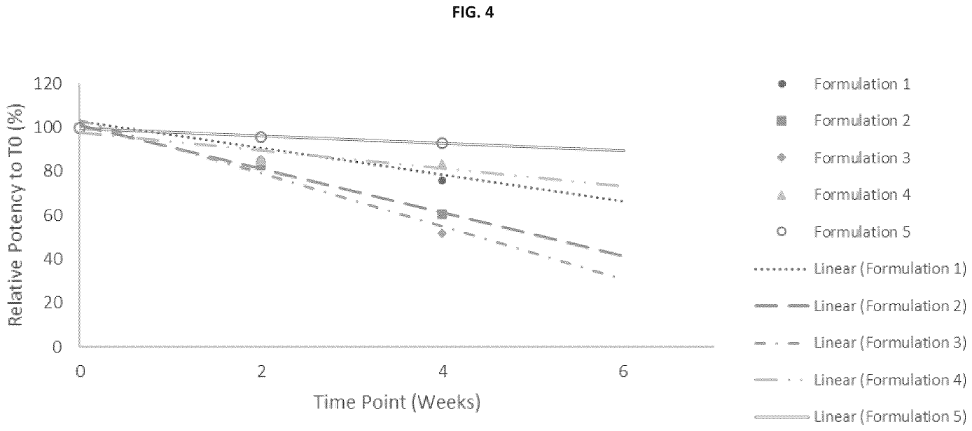

BoNT/AB chimera was formulated as a liquid at a target concentration of 30 ng/ml using a citric acid/sodium phosphate buffer (20 mM) or a dual L-Histidine & Histidine Monohydrochloride Monohydrate buffer (20 mM) 100 mM NaCl at a pH of 6.5 with different stabilisers (sucrose 10 mg/mL, propylene glycol 1% w/v, or glycine (20 mg/mL)+arginine (20 mg/mL), and different surfactants (PS80 0.02% w/v, poloxamer 188 (P188) 0.04% w/v, or PS80 0.01% w/v), as shown in Table 4 below. Formulations were incubated under accelerated stability conditions (25 °C) for 4 weeks and stability assessed by chimeric BoNT/AB protein quantification ELISA and chimeric BoNT/AB cell-based potency assay. Data generated from quantification and relative potency are presented in FIGS. 3 and 4, respectively.

TABLE 4
Varying stabilisers and surfactants

| | | | Stabiliser | | Surfactant |
|---|---|---|---|---|---|
| Form | Buffer (mM) | pH | (mg/mL) | Salt (mM) | (% w/v) |
| 1 | Cit-Phos (20) | 6.5 | Sucrose (10) | NaCl (100) | PS80 (0.02) |
| 2 | Cit-Phos (20) | 6.5 | Sucrose (10) | NaCl (100) | P188 (0.04) |
| 3 | Cit-Phos (20) | 6.5 | Propylene | NaCl (100) | PS80 (0.01) |
| | | | Glycol | | |
| | | | (1% w/v) | | |
| 4 | Cit-Phos (20) | 6.5 | Glycine (20) & | NaCl (100) | PS80 (0.01) |
| | | | Arginine (20) | | |
| 5 | Dual L-Hist./ | 6.5 | Sucrose (10) | NaCl (100) | PS80 (0.01) |
| | Hist.-HCl (20) | | | | |

Based on the above stability study, Formulation 5 with a dual L-Histidine and Histidine monohydrochloride monohydrate buffer at a pH of 6.5 demonstrated excellent stability, as determined by quantification (FIG. 3) and potency (FIG. 4) data.

In contrast, Formulation 1 (with increased PS80) and Formulation 4 (with a combination of glycine and arginine as stabiliser) were both inferior in performance to Formulation 5, both in terms of recovery and potency.

Formulation 3 (with propylene glycol as stabiliser) and Formulation 2 (with P188 as surfactant) were still further inferior to Formulations 1 and 4.

As such, formulations with a dual L-Histidine and Histidine monohydrochloride monohydrate buffer and PS80 at 0.01% w/v were taken forward for further investigation.

### Example 3—Surfactant Screening to Identify Stabilising Conditions

BoNT/AB chimera was formulated as a liquid using a dual L-Histidine and Histidine monohydrochloride monohydrate buffer (20 mM), 100 mM NaCl, 10 mg/mL sucrose, at a pH of 6.5. Alternative surfactants at high and low concentrations were included in the formulations to assess impact on product stability. Specifically, these were PS80 (0.01 and 0.02% w/v), P188 (0.04 and 0.08% w/v), Tertiary-Dodecyl Mercaptan (TDM) (0.01 and 0.02% w/v), and n-dodecyl-β-D-maltoside (DDM) (0.01 and 0.02% w/v), as shown in Table 5 below.

TABLE 5
Varying stabilisers and surfactants

| | | | Stabiliser | | Surfactant |
|---|---|---|---|---|---|
| Form | Buffer (mM) | pH | (mg/mL) | Salt (mM) | (% w/v) |
| 1 | Dual L-Hist./ | 6.5 | Sucrose (10) | NaCl (100) | PS80 (0.01) |
| | Hist.-HCl (20) | | | | |
| 2 | Dual L-Hist./ | 6.5 | Sucrose (10) | NaCl (100) | PS80 (0.02) |
| | Hist.-HCl (20) | | | | |
| 3 | Dual L-Hist./ | 6.5 | Sucrose (10) | NaCl (100) | P188 (0.04) |
| | Hist.-HCl (20) | | | | |
| 4 | Dual L-Hist./ | 6.5 | Sucrose (10) | NaCl (100) | P188 (0.08) |
| | Hist.-HCl (20) | | | | |
| 5 | Dual L-Hist./ | 6.5 | Sucrose (10) | NaCl (100) | TDM (0.01) |
| | Hist.-HCl (20) | | | | |
| 6 | Dual L-Hist./ | 6.5 | Sucrose (10) | NaCl (100) | TDM (0.02) |
| | Hist.-HCl (20) | | | | |
| 7 | Dual L-Hist./ | 6.5 | Sucrose (10) | NaCl (100) | DDM (0.01) |
| | Hist.-HCl (20) | | | | |
| 8 | Dual L-Hist./ | 6.5 | Sucrose (10) | NaCl (100) | DDM (0.02) |
| | Hist.-HCl (20) | | | | |

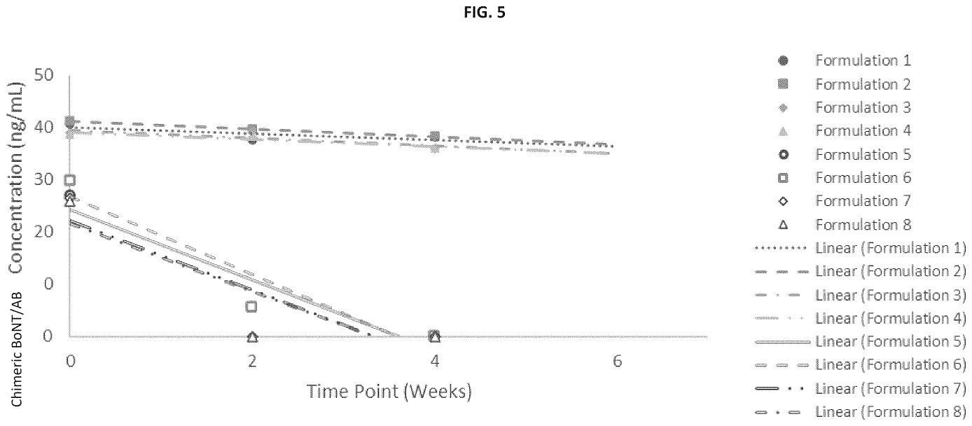

As shown in FIG. 5, use of either TDM or DDM resulted in significant loss of BoNT/AB after two weeks. Use of PS80 or P188 demonstrated satisfactory recovery over a six-week period at accelerated stability conditions.

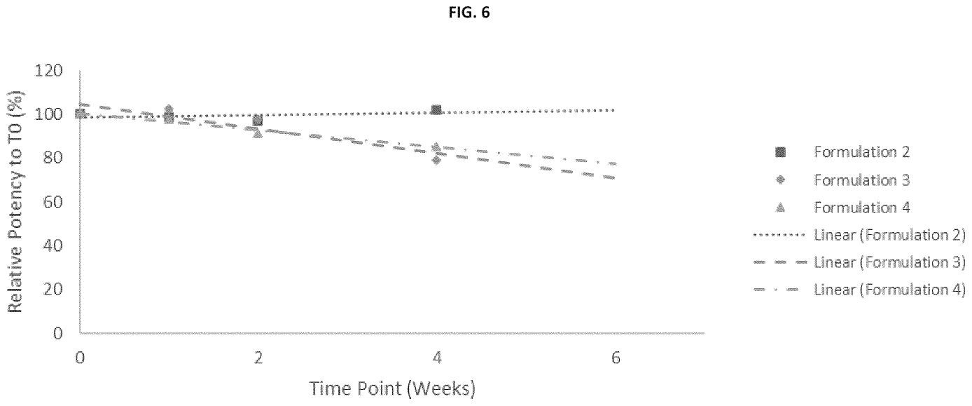

In terms of potency (FIG. 6) a 20% loss of potency was observed in Formulations 3 and 4 (comprising P188) after 4 weeks at 25 °C.

As such, dual L-Histidine formulations with PS80 as surfactant were taken forward for further investigation.

### Example 4—Long-Term Stability of Lyophilised Compositions

Using the preferred stabilising components identified in Examples 1-3, BoNT/AB chimera was formulated using a dual L-Histidine and Histidine monohydrochloride monohydrate buffer (20 mM), 10 mM NaCl, 10 mg/mL sucrose, and 0.01% w/v PS80, targeted at a pH of 5.5, 6.5, and 7.6 (see, Table 6, below).

TABLE 6
Varying pH

| | Formulation 1 | Formulation 2 | Formulation 3 |
|---|---|---|---|
| Buffer (mM) | Dual L-Hist./ | Dual L-Hist./ | Histidine only |
| | Hist.-HCl (20) | Hist.-HCl (20) | (20) |
| pH | 6.5 | 5.5 | 7.6 |
| Stabiliser (mg/mL) | Sucrose (10) | Sucrose (10) | Sucrose (10) |
| Salt (mM) | NaCl (10) | NaCl (10) | NaCl (10) |
| Surfactant (% w/v) | PS80 (0.01) | PS80 (0.01) | PS80 (0.01) |

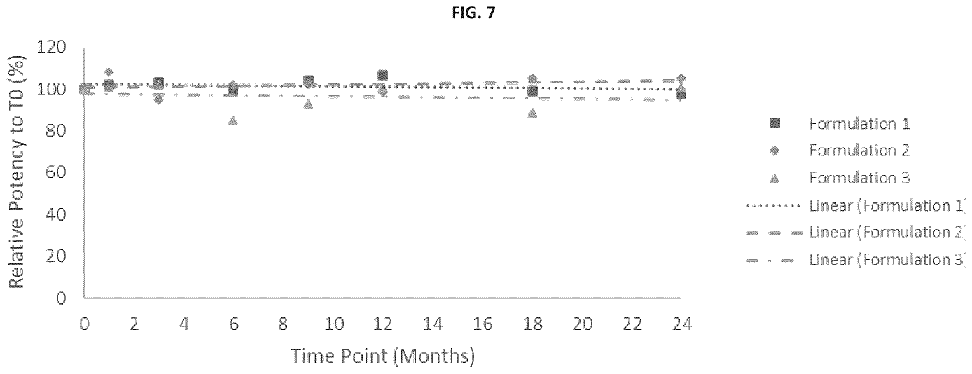

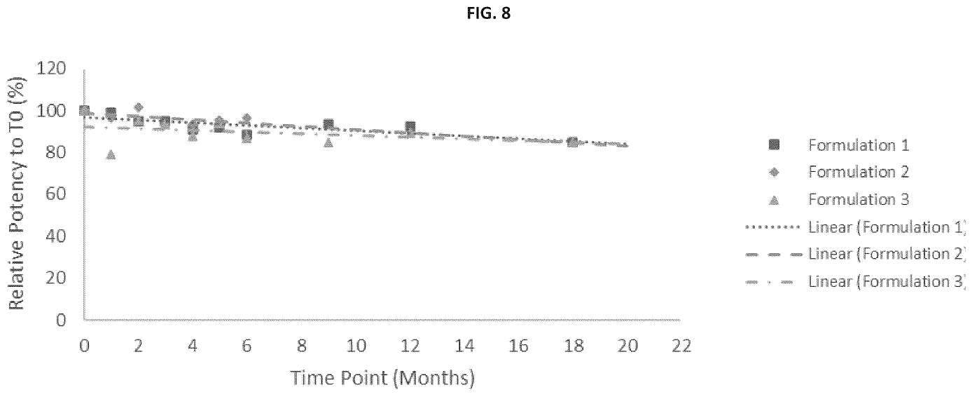

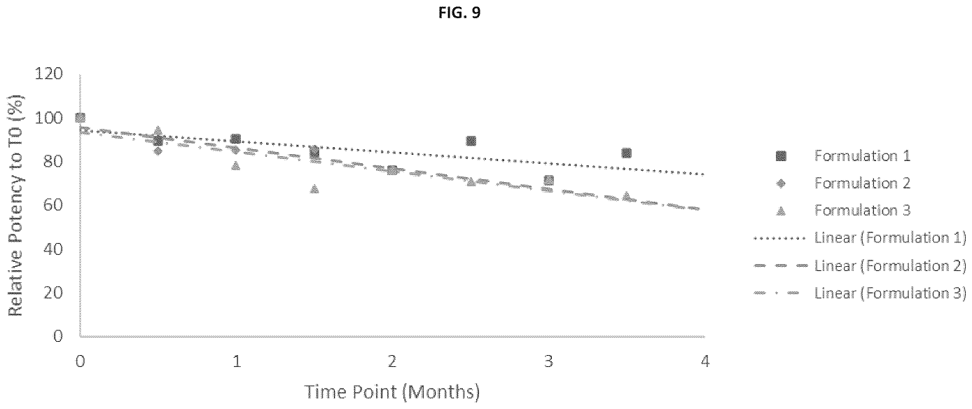

These formulations were then lyophilised and placed on long-term stability at 5 °C (real-time stability), 25 °C (accelerated stability), and 40 °C (forced stability) over a period of 24, 18, and 4 months, respectively. At each time point, the lyophilised drug product samples were reconstituted in 0.5 mL 0.9% w/v saline and their stability profile assessed by quantification ELISA and cell-based potency assay. Selection of a histidine-based buffer is favourable as a citric acid/sodium phosphate buffer exhibits an acidic pH shift on freezing. Data from stability studies at 5, 25, and 40 °C are presented in FIG. 7, FIG. 8, and FIG. 9, respectively. Whilst the tested formulations exhibited similar stability profiles at 5 °C and 25 °C, Formulation 1 (with a pH of 6.5) was more stable at 40 °C. Recovery data for the same formulations and time course are quantified below (see, Table 7).

Therefore, a dual-histidine formulation, particularly at a pH of 6.5 is particularly effective at stabilising chimeric BoNT/AB over a prolonged period of time.

TABLE 7
recovery data for lyophilised formulations
at different timepoints/temperatures

| | Quantification by ELISA (ng/mL) per | | | | |
|---|---|---|---|---|---|
| | timepoint/temperature | | | | |
| | | T2 m at | T3 m at | T6 m at | T6 m at |
| Formulation | T0 | 40 °C | 25 °C | 25 °C | 5 °C |
| Formulation | 25.1 | 26.6 | 27.5 | 27.2 | 26.3 |
| 1 (pH 6.5) | | | | | |
| Formulation | 22.7 | 21.5 | 24.3 | 24.4 | 23.3 |
| 2 (pH 5.5) | | | | | |
| Formulation | 24.8 | 25.6 | 27.2 | 26.1 | 25.6 |
| 3 (pH 7.6) | | | | | |

### Example 5—Comparison of Long-Term Stability of Dual-Histidine Buffer-Based Lyophilised Compositions of the Invention with Commercially Available Liquid and Solid Formulations

The stability of a dual-histidine buffer-based lyophilised chimeric BoNT/AB composition of the invention was compared with that of Alluzience (a liquid formulation of BoNT/A1), Dysport (a lyophilised formulation of BoNT/A1) and a cationic BoNT/A formulation (a long-acting liquid formulation of BoNT/A). The components of these compositions are set out in Table 8 below.

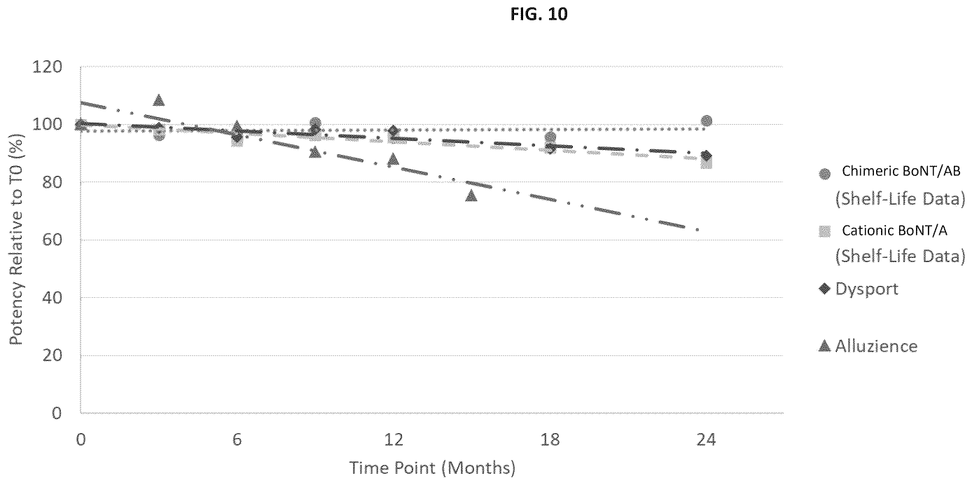

As shown in FIG. 10, the lyophilised chimeric BoNT/AB formulation of the invention exhibited superior potency compared with commercially available BONT formulations, including liquid BoNT/A1 (Alluzience), and liquid long-acting BoNT/A (cationic BoNT/A), as well as lyophilised BoNT/A1 (Dysport). Surprisingly, therefore, in addition to the data in Examples 1-4 demonstrating improved stability of the formulations of the invention over other chimeric BONT formulations, the particular combination of components (buffer, stabilising agent, surfactant and tonicity agent) at the tested pH (pH 6.5) elicited an observable improvement in the stability of chimeric BONT compared with commercially available BONT formulations.

TABLE 8
Compositions of chimeric BoNT/A, cationic BoNT/A, Dysport and Alluzience

| | | | | | | Dysport | | |
|---|---|---|---|---|---|---|---|---|
| | | Alluzience (liquid | | IPN10200 | | (Lyophilised | IPN59011 | |
| | | BoNT/A1) | | (Lyophilised AB) | | BoNT/A1) | (Lyophilised A) | |
| | MW | Conc. | Conc. | Conc. | Conc. | Conc. | Conc. | Conc. |
| Component | (g/mol) | (mg/mL) | (mM) | (mg/mL) | (mM) | (mg/mL) | (mg/mL) | (mM) |
| L-Histidine | 155.1546 | 1.55 | 9.99 | 2.28 | 14.7 | N/A | 3.10 | 20 |
| Sucrose | 342.3 | 4 | 11.7 | 10 | 29.2 | N/A | 10.0 | 29.2 |
| NaCl | 58.44 | 8.76 | 149.9 | 0.58 | 10 | N/A | 0.58 | 10 |
| PS80 | 1,310 | 0.1 (0.01 | 0.076 | 0.1 | 0.08 | N/A | 0.1 | 0.08 |
| | | % v/v) | | | | | | |
| Hydrochloric | | q.s. to pH | q.s. to pH | N/A | N/A | N/A | N/A | N/A |
| acid (pH) | | 6.5 | 6.5 | | | | | |
| Water for | | q.s. to 1 | q.s. to 1 | q.s. to 1 | q.s. to 1 | q.s. to 1 mL | q.s. to 1 | q.s. to 1 |
| Injection | | mL | mL | mL | mL | | mL | mL |
| Histidine- | 209.63 | N/A | N/A | 1.11 | 5.3 | N/A | N/A | N/A |
| HCl | | | | | | | | |
| HSA | | N/A | N/A | N/A | N/A | 0.5 | N/A | N/A |
| Lactose | | N/A | N/A | N/A | N/A | 10 | N/A | N/A |
| BoNT | | 200 U/vial | 200 U/vial | (TBD) | (TBD) | 300 or | (TBD) | (TBD) |
| | | | | | | 500 U/vial | | |
| | | | | | | (0.25 mL fill | | |
| | | | | | | per vial) | | |
| pH | | pH 6.5 | pH 6.5 | pH 6.5 | pH 6.5 | pH 5.85 | pH 7.6 | pH 7.6 |

### Example 6—Exemplary Chimeric BONT Dual-Histidine Buffer-Based Compositions of the Invention

Exemplary compositions (pre-lyophilisation, lyophilised and post-reconstitution) are set out in Table 9 below.

The volume of reconstitution solvent used to reconstitute lyophilised compositions of the invention may depend on the intended use. The reconstitution volume will determine the concentration of the various components in the reconstituted solution. Exemplary reconstitution schedules and the resulting final component concentrations are set out in Table 10 below.

As indicated in Tables 9 and 10, the amount of chimeric clostridial neurotoxin comprised in a composition of the invention will typically depend on the intended use of said composition. Exemplary chimeric neurotoxin amounts/concentrations for the compositions of the invention are set out in Table 11 below.

Exemplary compositions (pre-lyophilisation and post-reconstitution) and their reconstitution values are set out in Table 12 below.

Exemplary compositions, including vial content and fill volume, are set out in Table 13 below.

TABLE 9
exemplary compositions of the invention

| | | | | | Lyophilised Cake | | | Reconstituted DP | | |
|---|---|---|---|---|---|---|---|---|---|---|
| | | Formulation Solution | | | (Solid) | | | (in 0.5 mL 0.9% w/v | | |
| Compo- | MW | (Pre-Lyo) | | | (Equiv. | | | Saline) | | |
| nent | (g/mol) | mM | mg/mL | % w/w | mM) | mg/vial | % w/w | mM | mg/ml | % w/v |
| L-Histidine | 155.1546 | 14.7 | 2.28 | 0.227 | 14.7 | 1.14 | 16.2 | 14.7 | 2.28 | 0.228 |
| Histidine- | 209.63 | 5.3 | 1.11 | 0.110 | 5.3 | 0.555 | 7.9 | 5.3 | 1.11 | 0.111 |
| HCl | | | | | | | | | | |
| Sucrose | 342.3 | 29.2 | 10 | 0.99 | 29.2 | 5 | 71.1 | 29.2 | 10 | 1.0 |
| NaCl | 58.44 | 10 | 0.58 | 0.058 | 10 | 0.29 | 4.1 | 164 | 9.58 | 0.958 |
| PS80 | 1,310 | 0.08 | 0.1 | 0.01 | 0.08 | 0.05 | 0.7 | 0.08 | 0.1 | 0.01 |
| BoNT | Use dependent | | | | | | | | | |
| pH | | pH 6.5 | | | N/A | | | pH 6.5 | | |

TABLE 10
exemplary reconstitution schedules and the resulting final component
concentrations

| | | Reconstituted | | | | | | | | | | | |
|---|---|---|---|---|---|---|---|---|---|---|---|---|---|
| | | DP | | | Reconstituted DP | | | Reconstituted DP | | | Reconstituted DP | | |
| | | (in 0.5 mL 0.9 % | | | (in 1.0 mL 0.9% | | | (in 2.0 mL 0.9% w/v | | | (in 3.0 mL 0.9% | | |
| Compo- | MW | w/v Saline) | | | w/v Saline) | | | Saline) | | | w/v Saline) | | |
| nent | (g/mol) | mM | mg/mL | % w/v | mM | mg/mL | % w/v | mM | mg/mL | % w/v | mM | mg/ml | % w/v |
| L- | 155.1546 | 14.7 | 2.28 | 0.228 | 7.35 | 1.14 | 0.114 | 3.675 | 0.57 | 0.057 | 2.45 | 0.38 | 0.038 |
| Histidine | | | | | | | | | | | | | |
| Histidine- | 209.63 | 5.3 | 1.11 | 0.111 | 2.65 | 0.555 | 0.0555 | 1.325 | 0.2775 | 0.02775 | 0.883 | 0.185 | 0.0185 |
| HCl | | | | | | | | | | | | | |
| Sucrose | 342.3 | 29.2 | 10 | 1.0 | 14.6 | 5 | 0.5 | 7.3 | 2.5 | 0.25 | 4.87 | 1.7 | 0.17 |
| NaCl | 58.44 | 164 | 9.58 | 0.958 | 159 | 9.29 | 0.479 | 156 | 9.145 | 0.2395 | 155.7 | 9.097 | 0.160 |
| PS80 | 1,310 | 0.08 | 0.1 | 0.01 | 0.04 | 0.05 | 0.005 | 0.02 | 0.025 | 0.0025 | 0.013 | 0.0167 | 0.0017 |

| | |
|---|---|
| BoNT | Use dependent |

| | | | | |
|---|---|---|---|---|
| pH | pH 6.5 | pH 6.5 | pH 6.5 | pH 6.5 |

TABLE 12
Exemplary concentrations of the invention based on reconstitution value

| | | | 6R Post Recon | | 2R Post Recon | |
|---|---|---|---|---|---|---|
| | 6R Pre-Lyo Conc. | | Conc. (mM) | | Conc. (mM) | |
| Com- | (mM) | | low | high | low | high |
| ponent | low | high | (0.5 mL) | (10 mL) | (0.25 mL) | (4 mL) |
| L- | 14.70 | 29.40 | 29.40 | 0.735 | 29.40 | 0.735 |
| Histidine | | | | | | |
| Histidine- | 5.30 | 10.60 | 10.60 | 0.265 | 10.60 | 0.265 |
| HCl | | | | | | |
| Sucrose | 29.20 | 58.40 | 58.40 | 1.46 | 58.40 | 1.46 |
| NaCl | 10.00 | 20.00 | 163.92 | 154.5 | 163.92 | 154.5 |
| PS80 | 0.08 | 0.1600 | 0.1600 | 0.004 | 0.1600 | 0.004 |

TABLE 13
Exemplary concentrations per fill volume

Vial Content (mg/vial) per Fill Volume (mL)

| Component | 0.5 (e.g., 2R) | 1 (e.g., 6R) | 1.5 |
|---|---|---|---|
| L-Histidine | 1.140 | 2.280 | 3.420 |
| Histidine-HCl | 0.555 | 1.110 | 1.665 |
| Sucrose | 5.000 | 10.000 | 15.000 |
| NaCl | 0.290 | 0.580 | 0.870 |
| PS80 | 0.050 | 0.100 | 0.150 |

1. A solid composition comprising:
(a) a chimeric botulinum neurotoxin (BoNT);
(b) L-histidine; and
(c) histidine-HCl.

2. The composition of claim 1, which comprises:
(a) a relative weight amount of L-histidine of between about 10% to about 20% (w/w); and
(b) a relative weight amount of histidine-HCl of between about 5% to about 10% (w/w).

3. The solid composition of claim 1 or 2, which further comprises a stabiliser, which is preferably a disaccharide, more preferably sucrose.

4. The solid composition of claim 3, wherein the disaccharide is present at a relative weight amount of between about 70% to about 80% (w/w).

5. The solid composition of any one of the preceding claims, which further comprises a surfactant, preferably a non-ionic surfactant.

6. The solid composition of claim 5, wherein the non-ionic surfactant is a polysorbate or a poloxamer, preferably polysorbate 80 or poloxamer-188.

7. The solid composition of claim 5 or 6, wherein the surfactant is present at a relative weight amount of between about 0.5% to about 1.0% (w/w).

8. The solid composition of any one of the preceding claims, which further comprises a tonicity agent, preferably sodium chloride; wherein the tonicity agent is present at a relative weight amount of between about 2% to about 7% (w/w).

9. The solid composition of any one of the preceding claims, which does not comprise an additional excipient.

10. The solid composition of any one of the preceding claims, wherein the chimeric BONT is present at an absolute weight amount of between about 0.2 ng to about 500 ng, preferably between about 50 ng to about 300 ng, more preferably between about 50 ng to about 200 ng.

11. The solid composition of any one of the preceding claims, wherein the chimeric BONT is a BoNT/AB chimera.

12. The solid composition of any one of the preceding claims, which comprises or consists of:
(a) a chimeric BoNT, preferably a BoNT/AB chimera, optionally at an absolute weight amount of between about 0.2 ng to about 500 ng, preferably between about 50 ng to about 300 ng, more preferably between about 50 ng to about 200 ng;
(b) L-histidine, optionally at a relative weight a relative weight amount of between about 10% to about 20% (w/w), preferably between about 16% to about 17% (w/w);
(c) histidine-HCl, optionally at a relative weight a relative weight amount of between about 5% to about 10% (w/w), preferably between about 7.5% to about 8% (w/w);
(d) sucrose, optionally at a relative weight amount of between about 70% to about 80% (w/w), preferably between about 70% to about 72% (w/w);
(e) polysorbate 80 or poloxamer-188, optionally at a relative weight amount of between about 0.5% to about 1.0% (w/w), preferably between about 0.70% to about 0.75% (w/w); and
(f) sodium chloride, optionally at a relative weight amount of between about 2% to about 7% (w/w), preferably between about 4% to about 5%.

13. The solid composition of any one of the preceding claims, which is a lyophilised or vacuum-dried powder.

14. The solid composition of any one of the preceding claims, which has a pH between about 6.0 and about 7.0, preferably about 6.5, when reconstituted in saline and measured at 20-25 °C.

15. The solid composition of any one of the preceding claims, which is stable:
(a) for at least 18 months at 25 °C; and/or
(b) for at least 24 months at 5 °C.

16. A liquid composition comprising
(a) a chimeric botulinum neurotoxin;
(b) L-histidine; and
(c) histidine-HCl.

17. The liquid composition of claim 16, which comprises:
(a) a chimeric BoNT, preferably a BoNT/AB chimera, optionally at an amount of between about 0.2 ng to about 500 ng, preferably between about 50 ng to about 300 ng, more preferably between about 50 ng to about 200 ng;
(b) L-histidine, optionally at a concentration of between about 1.41 mg/mL to about 2.82 mg/mL preferably between about 2.0 mg/mL to about 2.5 mg/mL;
(c) histidine-HCl, optionally at a concentration of between about 0.70 mg/mL to about 1.41 mg/mL, preferably between about 1.00 mg/mL to about 1.20 mg/mL;
(d) sucrose, optionally at a concentration of between about 9.80 mg/mL to about 11.30 mg/mL, preferably between about 9.80 mg/mL to about 10.20 mg/mL;
(e) polysorbate 80 or poloxamer-188, optionally at a concentration of between about 0.070 mg/mL to about 0.140 mg/mL, preferably between about 0.095 mg/mL to about 0.120 mg/mL; and
(f) sodium chloride, optionally at a concentration of between about 0.28 mg/mL to about 11.7 mg/mL, optionally between about 5.8 mg/mL to about 11.7 mg/mL, between about 0.28 mg/mL to about 1.00 mg/mL or between about 0.56 mg/mL to about 0.70 mg/mL;
wherein the pH of the composition is between about 6.0 to about 7.0, preferably about 6.5.

18. The liquid composition of claim 16 or 17, which is stable for at least 6 months at 5 °C.

19. The liquid composition of any one of claims 16 to 18, which is reconstituted from a solid composition as defined in any one of claims 1 to 15 using water or saline.

20. A solid composition of any one of claims 1 to 15 or a liquid composition of any one of claims 16 to 19 for use in therapy, wherein the solid composition is reconstituted in water or saline before use.

21. A method of treatment comprising administration of a solid composition of any one of claims 1 to 15 or a liquid composition of any one of claims 16 to 19 to a subject in need thereof, wherein the solid composition is reconstituted in water or saline before administration.

22. A solid or liquid composition according to claim 21, for use in treating a condition associated with unwanted neuronal activity, for example a condition selected from the group consisting of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhidrosis, excessive salivation, excessive gastrointestinal secretions, secretory disorders, pain from muscle spasms, headache pain, migraine and dermatological conditions.

23. Non-therapeutic use of a solid composition of any one of claims 1 to 15 or a liquid composition of any one of claims 16 to 19, for treating an aesthetic or cosmetic condition, wherein the solid composition is reconstituted in water or saline before use.

24. A kit comprising a solid composition of any one of claims 1 to 15 or a liquid composition of any one of claims 16 to 19 and instructions for therapeutic or cosmetic administration of said composition to a subject in need thereof.

25. A method of producing a liquid composition according to any one of claims 16 to 19, comprising reconstitution of a solid composition of any one of claims 1 to 15 in water or saline.
